(19) United States
(12) Patent Application Publication
(10) Pub. No.: US 2024/0239847 A1
(43) Pub. Date: Jul. 18, 2024

(54) PROTEINACEOUS PARTICLE

(71) Applicant: Oxford University Innovation Limited, Oxford ( Oxfordshire) (GB)

(72) Inventors: Michael DUSTIN, Oxford (Oxfordshire) (GB); Stefen BALINT, Oxford (Oxfordshire) (GB)

(21) Appl. No.: 17/919,756

(22) PCT Filed: Apr. 22, 2021
(86) PCT No.: PCT/GB2021/050974

§ 371 Date: Oct. 19, 2022

(30) Foreign Application Priority Data
Apr. 22, 2020 (GB) 2005892.1

(51) Int. Cl. C07K 14/435; A61K 38/00; C12N 9/64

(52) U.S. Cl. CPC C07K 14/435; C12N 9/6467; A61K 38/00; C07K 2319/30

(57) ABSTRACT

The invention relates to an isolated proteinaceous particle comprising a core of perform and/or granzyme, the core being surrounded by a glycoprotein shell comprising thrombospondin-1 (TSP-1) or a fragment thereof, a variant thereof or an orthologue thereof. The invention further relates to nn engineered proteinaceous particle comprising a core of perform and/or granzyme, the core being surrounded by a glycoprotein shell comprising a thrombospondin protein, or a fragment thereof, a variant thereof or an orthologue thereof, wherein the granzyme and/or the thrombospondin is genetically modified. Further related materials, medical uses and manufacture are also contemplated.

### CROSS-REFERENCE TO RELATED APPLICATIONS

This application is the National Stage of International Application No. PCT/GB2021/050974, filed Apr. 22, 2021, which claims priority to GB 2005892.1, filed Apr. 22, 2020, which are entirely incorporated herein by reference.

### FUNDING STATEMENT

The project leading to this application has received funding from the European Research Council (ERC) under the European Union's Horizon 2020 research and innovation programme (grant agreement No 670930).

### SEQUENCE LISTING

This application contains a sequence listing filed in electronic form as an ASCII.txt file entitled “Seq listing_ST25.TXT”, created on Jun. 7, 2023, and having a size of 377 kb. The content of the sequence listing is incorporated herein in its entirety.

### FIELD OF THE INVENTION

The present invention relates to a proteinaceous particle, a cell and a composition comprising the proteinaceous particle, a method of producing a cell capable of producing an engineered proteinaceous particle, a method of isolating the proteinaceous particle, the proteinaceous particle for use as a medicament and a method of treatment using the proteinaceous particle.

### BACKGROUND

Cancer immunotherapy, using checkpoint blockade, tumour-infiltrating lymphocytes, or CAR-T cells, has had major impacts on specific subtypes of cancer, but immunotherapy has been unsuccessful for brain cancer (particularly glioblastoma), oesophageal cancer, ovarian cancer, and pancreatic cancer among others. Challenges associated with treating these and other types of cancer include entry of effector cells into tumours and the immunosuppressive tumour microenvironment (TME). Glioblastoma is a particularly challenging disease to treat and has a limited number of treatment options due to tumours sitting in an immune-privileged site that is not well accessed by conventional immunotherapies or cells. There is therefore a need for alternative immunotherapies that are capable of overcoming these challenges.

### STATEMENTS OF INVENTION

Thus, according to a first aspect of the invention, there is provided an isolated proteinaceous particle comprising a core of perform and/or granzyme, the core being surrounded by a glycoprotein shell comprising thrombospondin-1 (TSP-1) or a fragment thereof, a variant thereof or an orthologue thereof.

According to another aspect of the invention, there is provided an engineered proteinaceous particle comprising a core of perform and/or granzyme, the core being surrounded by a glycoprotein shell comprising a thrombospondin protein, or a fragment thereof, a variant thereof or an orthologue thereof, wherein the granzyme and/or the thrombospondin is genetically modified.

It has been found that activation of a variety of cells types, particularly T-lymphocytes or Natural Killer (NK) cells, results in the release of proteinaceous particles (also referred to herein as supramolecular attack particles (SMAPs) that are distinct from lipid-formed exosomes that have been previously observed from the extracellular release of such cells. The proteinaceous particles are capable of binding to local target cells. Once bound, the SMAPs usually release, from their core, at least one granzyme (i.e. granzyme A, B, H, M or K) and one pore forming protein (perforin1). The enzyme and the pore forming protein are cytotoxic to their target cell (i.e. the cell to which they bind to). This ultimately results in the death of the SMAP-bound cell. Thus, the SMAP of the invention may be used to treat or cure a variety of diseases or conditions by killing appropriate cells associated with the condition. For example, the SMAP may be used to treat a cancer by killing malignant tumour cells (e.g. glioblastoma), or it may be used to treat a bacterial or viral infection by killing infected cells, or it may be used to treat a bacterial infection by directly killing bacteria. The SMAP of the invention is also advantageous because, unlike conventional biologics and cell therapies, it is not susceptible to the effects of hostile extracellular environments (e.g. the immunosuppressive microenvironment of a tumour), and thus very stable.

The particles may remain stable (i.e. not degrade/disintegrate) extracellularly, for example for at least 1, 2, 5, 12, 24, or 48 hours, or for more than 1 day. The particles may remain stable extracellularly for between 1-5 hours or more. The particles may remain stable extracellularly for at least 72 hours. The particles may remain stable extracellularly for between 1-5 days or more.

The proteinaceous particle of the second aspect of the invention could be engineered to form a fusion polypeptide with any globular polypeptide. The proteinaceous particle of the second aspect of the invention could be engineered to form a fusion polypeptide, for example with a ligand (e.g. a targeting peptide) that specifically recognises a protein (e.g. a receptor) expressed on a target cell of interest. Thus, the ligand renders the proteinaceous particle specific for cells of a disease or condition, and could reduce/prevent potential off-target effects that may be associated with the use of the natural (i.e. non-engineered) proteinaceous particles. In one embodiment, the fusion polypeptide comprises a thrombospondin fused to a heterologous polypeptide.

The proteinaceous particle may have a diameter of less than 500 nm e.g. about 1 nm to 500 nm. Thus, the particle may be spherical in shape. For example, the proteinaceous particle may have a diameter of less than about 500 nm, less than about 400 nm, less than about 300 nm, less than about 200 nm or less than about 150 nm, or less than about 100 nm. The proteinaceous particle may have a diameter of about 80 to about 500 nm, or about 90 nm to about 400 nm, or about 100 nm to about 300 nm, or about 50 nm to about 200 nm, or about 50 nm to about 180 nm, or about 70 nm to about 180 nm, or about 70 nm to about 170 nm, or about 70 nm to about 150 nm, or about 70 nm to about 140 nm, or about 90 nm to about 150 nm, or about 90 nm to about 140 nm, or about 100 nm to about 130 nm, or about 110 nm to about 130 nm. In one embodiment, the diameter of the proteinaceous particle may be about 120 nm. The proteinaceous particle may not have a diameter greater than about 200 nm. Preferably, the proteinaceous particle does not have a diameter greater than about 150 nm. In a further preferred embodiment, the proteinaceous particle is between about 50 nm and about 150 nm. In an embodiment, wherein a plurality of proteinaceous particles are present, for example in composition or population, the size of the proteinaceous particle discussed herein refers to the average size in the population/composition of the proteinaceous particles.

The proteinaceous particle of the invention may be an isolated proteinaceous particle. The term “isolated” can refer to a proteinaceous particle that has been separated from cells (such as NK cells and T cells) and cellular structures, including exosomes and the phospholipid plasma membrane. The proteinaceous particle may be an extracellular particle. In one embodiment the proteinaceous particle is harvested from extracellular plasma. The proteinaceous particle may not be an intracellular particle and/or may not be harvested from intracellular plasma.

The proteinaceous particle of the invention may be an engineered proteinaceous particle. The proteinaceous particle of the invention may be an engineered and isolated proteinaceous particle. The proteinaceous particle may be functional at a purity ranging from about 10% to about 100%. Thus, the proteinaceous particle or composition according to the invention may be about 10% to about 100% pure, about 20% to about 100% pure, about 30% to about 100% pure, about 40% to about 100% pure, about 50% to about 100% pure, about 60% to about 100% pure, about 70% to about 100% pure, about 80% to about 100% pure or about 90% to about 100% pure. In one embodiment, the proteinaceous particle is isolated to at least about 90% purity. Preferably the proteinaceous particle or a composition of proteinaceous particles is substantially pure. However, in some embodiments, minor fractions of impurities such as exosomes may be present in a composition of proteinaceous particles. There may be less than 30% exosomes present. Preferably there are less than 20% or more preferably less than 10% exosomes present. The isolated proteinaceous particle(s) may be free from cells.

The core of the proteinaceous particle may comprise a granzyme enzyme. Granzyme refers to a family of cytotoxic serine proteases that are capable of cleaving extracellular and intracellular proteins. Granzymes are found in the secretory lysosomes of lymphocytes, particularly cytotoxic T cells, and natural killer (NK) cells. They are released by exocytosis but generally must gain entry into the cytoplasm of target cells to cleave intracellular proteins and induce cell death.

In humans, there are five members of the granzyme family, which are referred to as granzyme A, B, H, M and K. Human granzymes A, B, H, M and K are capable of inducing cell-death.

Granzyme A induces death of target cells in a mitochondrial-dependent fashion. The polypeptide sequence of the precursor of granzyme A is 262 amino acids long and is provided herein as SEQ ID NO. 1, as follows:

[SEQ ID NO. 1]
MRNSYRFLASSLSVVVSLLLIPEDVCEKIIGGNEVTPHSRPYMVLLSLD
RKTICAGALIAKDWVLTAAHCNLNKRSQVILGAHSITREEPTKQIMLVK
KEFPYPCYDPATREGDLKLLQLMEKAKINKYVTILHLPKKGDDVKPGTM
CQVAGWGRTHNSASWSDTLREVNITIIDRKVCNDRNHYNFNPVIGMNMV
CAGSLRGGRDSCNGDSGSPLLCEGVFRGVTSFGLENKCGDPRGPGVYIL
LSKKHLNWIIMTIKGAV

The bold amino acids of SEQ ID NO. 1 correspond to the signal peptide. The underlined amino acids of SEQ ID NO. 1 correspond to the amino acids of the propeptide of granzyme A. Amino acids 29 to 262 correspond to the polypeptide chain of granzyme A.

Of all of the granzymes, granzyme B is the most characterised. It induces programmed cell death (apoptosis) of target cells. Apoptosis is achieved by activating mitochondrial/caspase-dependent and caspase-independent pathways. Granzyme B also induces anoikis (death due to lack of extracellular contact) of target cells. The polypeptide sequence of the precursor of granzyme B is 247 amino acid long and is provided herein as SEQ ID NO. 2, as follows:

[SEQ ID NO. 2]
MQPILLLLAFLLLPRADAGEIIGGHEAKPHSRPYMAYLMIWDQKSLKRC
GGFLIRDDFVLTAAHCWGSSINVTLGAHNIKEQEPTQQFIPVKRPIPHP
AYNPKNFSNDIMLLQLERKAKRTRAVQPLRLPSNKAQVKPGQTCSVAGW
GQTAPLGKHSHTLQEVKMTVQEDRKCESDLRHYYDSTIELCVGDPEIKK
TSFKGDSGGPLVCNKVAQGIVSYGRNNGMPPRACTKVSSFVHWIKKTMK
RY

The bold amino acids of SEQ ID NO. 2 correspond to the signal peptide. The underlined amino acids of SEQ ID NO. 2 correspond to the amino acids of the propeptide of granzyme B. Amino acids 21 to 247 correspond to the polypeptide chain of granzyme B.

Granzyme H mediates caspase-independent killing of target cells. The polypeptide sequence of the precursor of granzyme H is 246 amino acids long and is provided herein as SEQ ID NO. 3, as follows:

[SEQ ID NO. 3]
MQPFLLLLAFLLTPGAGTEEIIGGHEAKPHSRPYMAFVQFLQEKSRKRC
GGILVRKDFVLTAAHCQGSSINVTLGAHNIKEQERTQQFIPVKRPIPHP
AYNPKNFSNDIMLLQLERKAKWTTAVRPLRLPSSKAQVKPGQLCSVAGW
GYVSMSTLATTLQEVLLTVQKDCQCERLFHGNYSRATEICVGDPKKTQT
GFKGDSGGPLVCKDVAQGILSYGNKKGTPPGVYIKVSHFLPWIKRTMKR
L

The bold amino acids of SEQ ID NO. 3 correspond to the signal peptide. The underlined amino acids of SEQ ID NO. 3 correspond to the amino acids of the propeptide of granzyme H. Amino acids 21-246 correspond to the polypeptide chain of granzyme H.

Granzyme M induced cell death in a caspase- and mitochondrial-independent fashion. The polypeptide sequence of the precursor of granzyme M is 257 amino acids long and is provided herein as SEQ ID NO. 4, as follows:

[SEQ ID NO. 4]
MEACVSSLLVLALGALSVGSSFGTQIIGGREVIPHSRPYMASLQRNGSH
LCGGVLVHPKWVLTAAHCLAQRMAQLRLVLGLHTLDSPGLTFHIKAAIQ
HPRYKPVPALENDLALLQLDGKVKPSRTIRPLALPSKRQVVAAGTRCSM
AGWGLTHQGGRLSRVLRELDLQVLDTRMCNNSRFWNGSLSPSMVCLAAD
SKDQAPCKGDSGGPLVCGKGRVLARVLSFSSRVCTDIFKPPVATAVAPY
VSWIRKVTGRSA

The bold amino acids of SEQ ID NO. 4 correspond to the signal peptide. The underlined amino acids of SEQ ID NO. 4 correspond to the amino acids of the propeptide of granzyme M. Amino acids 26 to 257 correspond to the polypeptide chain of granzyme M.

Granzyme K has been shown to be required for killing of T-lymphocytes by NK cells. The polypeptide sequence of the precursor of granzyme K is 264 amino acids long and is provided herein as SEQ ID NO. 5, as follows:

[SEQ ID NO. 5]
MTKFSSFSLFFLIVGAYMTHVCFNMEIIGGKEVSPHSRPFMASIQYGGH
HVCGGVLIDPQWVLTAAHCQYRFTKGQSPTVVLGAHSLSKNEASKQTLE
IKKFIPFSRVTSDPQSNDIMLVKLQTAAKLNKHVKMLHIRSKTSLRSGT
KCKVTGWGATDPDSLRPSDTLREVTVTVLSRKLCNSQSYYNGDPFITKD
MVCAGDAKGQKDSCKGDSGGPLICKGVFHAIVSGGHECGVATKPGIYTL
LTKKYQTWIKSNLVPPHTN

The bold amino acids of SEQ ID NO. 5 correspond to the signal peptide. The underlined amino acids of SEQ ID NO. 5 correspond to the amino acids of the propeptide of granzyme K. Amino acids 27 to 264 correspond to the polypeptide chain of granzyme K.

Accordingly, the granzyme of the proteinaceous particle may comprise granzyme A, B, H, M and/or K, or a variant or fragment or orthologue thereof. Furthermore, the granzyme of the proteinaceous particle may comprise a polypeptide sequence substantially as set out in the polypeptide chain of SEQ ID NO. 1, 2, 3, 4 and/or 5, or a variant or fragment or orthologue thereof. The granzyme of the proteinaceous particle may comprise the mature (i.e. non-precursor) polypeptide sequence substantially as set out in the polypeptide chain of SEQ ID NO. 1, 2, 3, 4 and/or 5, or a variant or fragment or orthologue thereof. Preferably the granzyme of the proteinaceous particle comprises the polypeptide chain of granzyme B. Preferably the granzyme of the proteinaceous particle comprises a polypeptide sequence substantially as set out in the polypeptide chain of SEQ ID NO. 2.

Although granzymes are capable of inducing cell death by cleaving intracellular proteins, they may require the assistance of other enzymes to gain intracellular access. Perform is one such enzyme. Perform facilitates entry of granzymes into the cytoplasm of target cells. Perform oligomerises to form a pore/channel in the plasma membrane of a target cell. The channel enables free, non-selective, passive transport of ions, water, small-molecule substances and protein (such as granzymes) into the target cell, which results in the disruption of the plasma membrane and protective effects provided by it. Perforin may also trigger a response in the target cells that causes the target cell to endocytosis granzymes and then the endosome containing the granzymes to burst once inside the cell, releasing granzymes into the cytoplasm where they can induce target cell death.

In one embodiment, the amino acid sequence of the human perform monomer is provided herein as SEQ ID NO. 6, as follows:

[SEQ ID NO. 6]
MAARLLLLGILLLLLPLPVPAPCHTAARSECKRSHKFVPGAWLAGEGVDVTSLRRSGSFPVDTQRFLRPD
GTCTLCENALQEGTLQRLPLALTNWRAQGSGCQRHVTRAKVSSTEAVARDAARSIRNDWKVGLDVTPKPT
SNVHVSVAGSHSQAANFAAQKTHQDQYSFSTDTVECRFYSFHVVHTPPLHPDFKRALGDLPHHFNASTQP
AYLRLISNYGTHFIRAVELGGRISALTALRTCELALEGLTDNEVEDCLTVEAQVNIGIHGSISAEAKACE
EKKKKHKMTASFHQTYRERHSEVVGGHHTSINDLLFGIQAGPEQYSAWVNSLPGSPGLVDYTLEPLHVLL
DSQDPRREALRRALSQYLTDRARWRDCSRPCPPGRQKSPRDPCQCVCHGSAVTTQDCCPRQRGLAQLEVT
FIQAWGLWGDWFTATDAYVKLFFGGQELRTSTVWDNNNPIWSVRLDFGDVLLATGGPLRLQVWDQDSGRD
DDLLGTCDQAPKSGSHEVRCNLNHGHLKFRYHARCLPHLGGGTCLDYVPQMLLGEPPGNRSGAVW

The perform of the proteinaceous particle may be a variant thereof or fragment thereof or orthologue thereof, which is able to form a pore/channel in the plasma membrane of a target cell. Furthermore, the perform may comprise a polypeptide sequence substantially as set out in SEQ ID NO. 6, or a variant thereof or fragment thereof or orthologue thereof.

The core refers to the interior of the proteinaceous particle, which is surrounded by the glycoprotein shell. In one embodiment, the core comprises or consists of perform and granzyme. The core comprises perform and/or a granzyme (e.g. granzyme B) but may further comprise other proteins (e.g. IFN gamma, CCL5, XCL2, serglycin (SRGN)).

Proteoglycans, such as serglycin (a short polypeptide with attached, long negatively charged glycosaminoglacan chains), improve the stability and retention of granzyme and perform within cytotoxic T cells and NK cells. Serglycin may or may not be required by a proteinaceous particle to kill a target cell. Thus, the core may further comprise serglycin complexed with granzyme and/or perform. Granzyme and/or perform may form a complex with other negatively charged proteins (other than serglycin). In addition, serglycin may stabilise a complex formed by granzyme and/or perform with other enzymes within the core of the proteinaceous particle.

The shell of the proteinaceous particle has several functions. For example, the glycoprotein shell selectively protects the contents of the core from the extracellular environment. Thus, the shell may improve and keep the core stable when the proteinaceous particle has been released extracellularly. The shell may act as a vector for the core. The shell may keep the core concentrated and prevent release of the core contents until the proteinaceous particle reaches a target cell. The shell provides a surface for several proteins to reside (e.g. TSP-1). The glycoprotein shell may have a higher density of organic material than the core. The shell may be a non-uniform carbon-dense shell (unlike exosomes which have a uniform lipid and transmembrane glycoprotein based limiting membrane).

The proteinaceous particle may not comprise an outer plasma membrane or phospholipid/cholesterol membrane. The glycoprotein shell of the proteinaceous particle may not be a plasma membrane or phospholipid/cholesterol membrane. Thus, the glycoprotein shell may not comprise transmembrane glycoproteins (such as CD45, CD81, T cell antigen receptors, and major histocompatibility complex proteins), or secretory lysosome transmembrane glycoproteins or “degranulation markers” (e.g. CD57 or CD107a). In one embodiment, the glycoprotein shell may not comprise CD47, ICAM-1 and/or extracellular fragments thereof. The shell may further comprise other proteins, such as one or more of galectin-1, galectin-7, or thrombospondin-4 (TSP-4) The shell may be porous. The pores may be at most about 13 nm in diameter (based on hydrodynamic diameter of IgG). The pores may be dynamic and selective. The pores in the shell enable IgG type antibodies to bind perform and granzymes within the core without using a detergent, a pore-forming agent, like saponin, or proteases.

TSP-1 is an adhesion protein, which mediates cell-to-cell interactions and cell-to-ECM (extracellular matrix) interactions, possibly by binding to ICAM-1, CD47 and/or intergrins. Thus, TSP-1 mediates binding of the proteinaceous particle to target cells or extracellular matrix proteins. TSP-1 belongs to a family of glycoproteins referred to as Thrombospondins. Thrombospondin family members include TSP-1, thrombospondin-2 (TSP-2), thrombospondin-3 (TSP-3), TSP-4 and thrombospondin-5 (TSP-5). The signature domain of thrombospondins is at the C-terminus and contains the Ca²⁺ binding “wire” domain (also called Type-3 repeats) and lectin-like “globe” domain.

Thrombospondins have several functions within the proteinaceous particle of the invention. For example, TSP-1 contributes to induction of target cell death, it is needed to release of granzyme and/or perform in the proteinaceous particles, and it stabilises proteinaceous particles once they have been released extracellularly.

In humans, TSP-1 is encoded by the gene THBS1. The genomic DNA sequence (introns and exons) encoding one embodiment of thrombospondin-1 is referred to herein as SEQ ID NO. 7 and can be found under the gene ID: 7057 (https://www.ncbi.nlm.nih.gov/gene?db=gene&Cmd=DetailsSearch&Term=7057).

The cDNA sequence (exons only) encoding one embodiment of THBS1 is provided herein as SEQ ID NO. 8, as follows:

[SEQ ID NO. 8]
GGACGCACAGGCATTCCCCGCGCCCCTCCAGCCCTCGCCGCCCTCGCCACCGCTCCCGGCCGCCGCGCTC
CGGTACACACAGGATCCCTGCTGGGCACCAACAGCTCCACCATGGGGCTGGCCTGGGGACTAGGCGTCCT
GTTCCTGATGCATGTGTGTGGCACCAACCGCATTCCAGAGTCTGGCGGAGACAACAGCGTGTTTGACATC
TTTGAACTCACCGGGGCCGCCCGCAAGGGGTCTGGGCGCCGACTGGTGAAGGGCCCCGACCCTTCCAGCC
CAGCTTTCCGCATCGAGGATGCCAACCTGATCCCCCCTGTGCCTGATGACAAGTTCCAAGACCTGGTGGA
TGCTGTGCGGGCAGAAAAGGGTTTCCTCCTTCTGGCATCCCTGAGGCAGATGAAGAAGACCCGGGGCACG
CTGCTGGCCCTGGAGCGGAAAGACCACTCTGGCCAGGTCTTCAGCGTGGTGTCCAATGGCAAGGCGGGCA
CCCTGGACCTCAGCCTGACCGTCCAAGGAAAGCAGCACGTGGTGTCTGTGGAAGAAGCTCTCCTGGCAAC
CGGCCAGTGGAAGAGCATCACCCTGTTTGTGCAGGAAGACAGGGCCCAGCTGTACATCGACTGTGAAAAG
ATGGAGAATGCTGAGTTGGACGTCCCCATCCAAAGCGTCTTCACCAGAGACCTGGCCAGCATCGCCAGAC
TCCGCATCGCAAAGGGGGGCGTCAATGACAATTTCCAGGGGGTGCTGCAGAATGTGAGGTTTGTCTTTGG
AACCACACCAGAAGACATCCTCAGGAACAAAGGCTGCTCCAGCTCTACCAGTGTCCTCCTCACCCTTGAC
AACAACGTGGTGAATGGTTCCAGCCCTGCCATCCGCACTAACTACATTGGCCACAAGACAAAGGACTTGC
AAGCCATCTGCGGCATCTCCTGTGATGAGCTGTCCAGCATGGTCCTGGAACTCAGGGGCCTGCGCACCAT
TGTGACCACGCTGCAGGACAGCATCCGCAAAGTGACTGAAGAGAACAAAGAGTTGGCCAATGAGCTGAGG
CGGCCTCCCCTATGCTATCACAACGGAGTTCAGTACAGAAATAACGAGGAATGGACTGTTGATAGCTGCA
CTGAGTGTCACTGTCAGAACTCAGTTACCATCTGCAAAAAGGTGTCCTGCCCCATCATGCCCTGCTCCAA
TGCCACAGTTCCTGATGGAGAATGCTGTCCTCGCTGTTGGCCCAGCGACTCTGCGGACGATGGCTGGTCT
CCATGGTCCGAGTGGACCTCCTGTTCTACGAGCTGTGGCAATGGAATTCAGCAGCGCGGCCGCTCCTGCG
ATAGCCTCAACAACCGATGTGAGGGCTCCTCGGTCCAGACACGGACCTGCCACATTCAGGAGTGTGACAA
AAGATTTAAACAGGATGGTGGCTGGAGCCACTGGTCCCCGTGGTCATCTTGTTCTGTGACATGTGGTGAT
GGTGTGATCACAAGGATCCGGCTCTGCAACTCTCCCAGCCCCCAGATGAATGGGAAACCCTGTGAAGGCG
AAGCGCGGGAGACCAAAGCCTGCAAGAAAGACGCCTGCCCCATCAATGGAGGCTGGGGTCCTTGGTCACC
ATGGGACATCTGTTCTGTCACCTGTGGAGGAGGGGTACAGAAACGTAGTCGTCTCTGCAACAACCCCGCA
CCCCAGTTTGGAGGCAAGGACTGCGTTGGTGATGTAACAGAAAACCAGATCTGCAACAAGCAGGACTGTC
CAATTGATGGATGCCTGTCCAATCCCTGCTTTGCCGGCGTGAAGTGTACTAGCTACCCTGATGGCAGCTG
GAAATGTGGTGCTTGTCCCCCTGGTTACAGTGGAAATGGCATCCAGTGCACAGATGTTGATGAGTGCAAA
GAAGTGCCTGATGCCTGCTTCAACCACAATGGAGAGCACCGGTGTGAGAACACGGACCCCGGCTACAACT
GCCTGCCCTGCCCCCCACGCTTCACCGGCTCACAGCCCTTCGGCCAGGGTGTCGAACATGCCACGGCCAA
CAAACAGGTGTGCAAGCCCCGTAACCCCTGCACGGATGGGACCCACGACTGCAACAAGAACGCCAAGTGC
AACTACCTGGGCCACTATAGCGACCCCATGTACCGCTGCGAGTGCAAGCCTGGCTACGCTGGCAATGGCA
TCATCTGCGGGGAGGACACAGACCTGGATGGCTGGCCCAATGAGAACCTGGTGTGCGTGGCCAATGCGAC
TTACCACTGCAAAAAGGATAATTGCCCCAACCTTCCCAACTCAGGGCAGGAAGACTATGACAAGGATGGA
ATTGGTGATGCCTGTGATGATGACGATGACAATGATAAAATTCCAGATGACAGGGACAACTGTCCATTCC
ATTACAACCCAGCTCAGTATGACTATGACAGAGATGATGTGGGAGACCGCTGTGACAACTGTCCCTACAA
CCACAACCCAGATCAGGCAGACACAGACAACAATGGGGAAGGAGACGCCTGTGCTGCAGACATTGATGGA
GACGGTATCCTCAATGAACGGGACAACTGCCAGTACGTCTACAATGTGGACCAGAGAGACACTGATATGG
ATGGGGTTGGAGATCAGTGTGACAATTGCCCCTTGGAACACAATCCGGATCAGCTGGACTCTGACTCAGA
CCGCATTGGAGATACCTGTGACAACAATCAGGATATTGATGAAGATGGCCACCAGAACAATCTGGACAAC
TGTCCCTATGTGCCCAATGCCAACCAGGCTGACCATGACAAAGATGGCAAGGGAGATGCCTGTGACCACG
ATGATGACAACGATGGCATTCCTGATGACAAGGACAACTGCAGACTCGTGCCCAATCCCGACCAGAAGGA
CTCTGACGGCGATGGTCGAGGTGATGCCTGCAAAGATGATTTTGACCATGACAGTGTGCCAGACATCGAT
GACATCTGTCCTGAGAATGTTGACATCAGTGAGACCGATTTCCGCCGATTCCAGATGATTCCTCTGGACC
CCAAAGGGACATCCCAAAATGACCCTAACTGGGTTGTACGCCATCAGGGTAAAGAACTCGTCCAGACTGT
CAACTGTGATCCTGGACTCGCTGTAGGTTATGATGAGTTTAATGCTGTGGACTTCAGTGGCACCTTCTTC
ATCAACACCGAAAGGGACGATGACTATGCTGGATTTGTCTTTGGCTACCAGTCCAGCAGCCGCTTTTATG
TTGTGATGTGGAAGCAAGTCACCCAGTCCTACTGGGACACCAACCCCACGAGGGCTCAGGGATACTCGGG
CCTTTCTGTGAAAGTTGTAAACTCCACCACAGGGCCTGGCGAGCACCTGCGGAACGCCCTGTGGCACACA
GGAAACACCCCTGGCCAGGTGCGCACCCTGTGGCATGACCCTCGTCACATAGGCTGGAAAGATTTCACCG
CCTACAGATGGCGTCTCAGCCACAGGCCAAAGACGGGTTTCATTAGAGTGGTGATGTATGAAGGGAAGAA
AATCATGGCTGACTCAGGACCCATCTATGATAAAACCTATGCTGGTGGTAGACTAGGGTTGTTTGTCTTC
TCTCAAGAAATGGTGTTCTTCTCTGACCTGAAATACGAATGTAGAGATCCCTAATCATCAAATTGTTGAT
TGAAAGACTGATCATAAACCAATGCTGGTATTGCACCTTCTGGAACTATGGGCTTGAGAAAACCCCCAGG
ATCACTTCTCCTTGGCTTCCTTCTTTTCTGTGCTTGCATCAGTGTGGACTCCTAGAACGTGCGACCTGCC
TCAAGAAAATGCAGTTTTCAAAAACAGACTCATCAGCATTCAGCCTCCAATGAATAAGACATCTTCCAAG
CATATAAACAATTGCTTTGGTTTCCTTTTGAAAAAGCATCTACTTGCTTCAGTTGGGAAGGTGCCCATTC
CACTCTGCCTTTGTCACAGAGCAGGGTGCTATTGTGAGGCCATCTCTGAGCAGTGGACTCAAAAGCATTT
TCAGGCATGTCAGAGAAGGGAGGACTCACTAGAATTAGCAAACAAAACCACCCTGACATCCTCCTTCAGG
AACACGGGGAGCAGAGGCCAAAGCACTAAGGGGAGGGCGCATACCCGAGACGATTGTATGAAGAAAATAT
GGAGGAACTGTTACATGTTCGGTACTAAGTCATTTTCAGGGGATTGAAAGACTATTGCTGGATTTCATGA
TGCTGACTGGCGTTAGCTGATTAACCCATGTAAATAGGCACTTAAATAGAAGCAGGAAAGGGAGACAAAG
ACTGGCTTCTGGACTTCCTCCCTGATCCCCACCCTTACTCATCACCTTGCAGTGGCCAGAATTAGGGAAT
CAGAATCAAACCAGTGTAAGGCAGTGCTGGCTGCCATTGCCTGGTCACATTGAAATTGGTGGCTTCATTC
TAGATGTAGCTTGTGCAGATGTAGCAGGAAAATAGGAAAACCTACCATCTCAGTGAGCACCAGCTGCCTC
CCAAAGGAGGGGCAGCCGTGCTTATATTTTTATGGTTACAATGGCACAAAATTATTATCAACCTAACTAA
AACATTCCTTTTCTCTTTTTTCCGTAATTACTAGGTAGTTTTCTAATTCTCTCTTTTGGAAGTATGATTT
TTTTAAAGTCTTTACGATGTAAAATATTTATTTTTTACTTATTCTGGAAGATCTGGCTGAAGGATTATTC
ATGGAACAGGAAGAAGCGTAAAGACTATCCATGTCATCTTTGTTGAGAGTCTTCGTGACTGTAAGATTGT
AAATACAGATTATTTATTAACTCTGTTCTGCCTGGAAATTTAGGCTTCATACGGAAAGTGTTTGAGAGCA
AGTAGTTGACATTTATCAGCAAATCTCTTGCAAGAACAGCACAAGGAAAATCAGTCTAATAAGCTGCTCT
GCCCCTTGTGCTCAGAGTGGATGTTATGGGATTCCTTTTTTCTCTGTTTTATCTTTTCAAGTGGAATTAG
TTGGTTATCCATTTGCAAATGTTTTAAATTGCAAAGAAAGCCATGAGGTCTTCAATACTGTTTTACCCCA
TCCCTTGTGCATATTTCCAGGGAGAAGGAAAGCATATACACTTTTTTCTTTCATTTTTCCAAAAGAGAAA
AAAATGACAAAAGGTGAAACTTACATACAAATATTACCTCATTTGTTGTGTGACTGAGTAAAGAATTTTT
GGATCAAGCGGAAAGAGTTTAAGTGTCTAACAAACTTAAAGCTACTGTAGTACCTAAAAAGTCAGTGTTG
TACATAGCATAAAAACTCTGCAGAGAAGTATTCCCAATAAGGAAATAGCATTGAAATGTTAAATACAATT
TCTGAAAGTTATGTTTTTTTTCTATCATCTGGTATACCATTGCTTTATTTTTATAAATTATTTTCTCATT
GCCATTGGAATAGAATATTCAGATTGTGTAGATATGCTATTTAAATAATTTATCAGGAAATACTGCCTGT
AGAGTTAGTATTTCTATTTTTATATAATGTTTGCACACTGAATTGAAGAATTGTTGGTTTTTTCTTTTTT
TTGTTTTTTTTTTTTTTTTTTTTTTTTTTGCTTTTGACCTCCCATTTTTACTATTTGCCAATACCTTTTT
CTAGGAATGTGCTTTTTTTTGTACACATTTTTATCCATTTTACATTCTAAAGCAGTGTAAGTTGTATATT
ACTGTTTCTTATGTACAAGGAACAACAATAAATCATATGGAAATTTATATTT

[SEQ ID NO. 9]
MGLAWGLGVLFLMHVCGTNRIPESGGDNSVFDIFELTGAARKGSGRRLVKGPDPSSPAFRIEDANLIPPV
PDDKFQDLVDAVRAEKGFLLLASLRQMKKTRGTLLALERKDHSGQVFSVVSNGKAGTLDLSLTVQGKQHV
VSVEEALLATGQWKSITLFVQEDRAQLYIDCEKMENAELDVPIQSVFTRDLASIARLRIAKGGVNDNFQG
VLQNVRFVFGTTPEDILRNKGCSSSTSVLLTLDNNVVNGSSPAIRTNYIGHKTKDLQAICGISCDELSSM
VLELRGLRTIVTTLQDSIRKVTEENKELANELRRPPLCYHNGVQYRNNEEWTVDSCTECHCQNSVTICKK
VSCPIMPCSNATVPDGECCPRCWPSDSADDGWSPWSEWTSCSTSCGNGIQQRGRSCDSLNNRCEGSSVQT
RTCHIQECDKRFKQDGGWSHWSPWSSCSVTCGDGVITRIRLCNSPSPQMNGKPCEGEARETKACKKDACP
INGGWGPWSPWDICSVTCGGGVQKRSRLCNNPTPQFGGKDCVGDVTENQICNKQDCPIDGCLSNPCFAGV
KCTSYPDGSWKCGACPPGYSGNGIQCTDVDECKEVPDACFNHNGEHRCENTDPGYNCLPCPPRFTGSQPF
GQGVEHATANKQVCKPRNPCTDGTHDCNKNAKCNYLGHYSDPMYRCECKPGYAGNGIICGEDTDLDGWPN
ENLVCVANATYHCKKDNCPNLPNSGQEDYDKDGIGDACDDDDDNDKIPDDRDNCPFHYNPAQYDYDRDDV
GDRCDNCPYNHNPDQADTDNNGEGDACAADIDGDGILNERDNCQYVYNVDQRDTDMDGVGDQCDNCPLEH
NPDQLDSDSDRIGDTCDNNQDIDEDGHQNNLDNCPYVPNANQADHDKDGKGDACDHDDDNDGIPDDKDNC
RLVPNPDQKDSDGDGRGDACKDDFDHDSVPDIDDICPENVDISETDFRRFQMIPLDPKGTSQNDPNWVVR
HQGKELVQTVNCDPGLAVGYDEFNAVDFSGTFFINTERDDDYAGFVFGYQSSSRFYVVMWKQVTQSYWDT
NPTRAQGYSGLSVKVVNSTTGPGEHLRNALWHTGNTPGQVRTLWHDPRHIGWKDFTAYRWRLSHRPKTGF
IRVVMYEGKKIMADSGPIYDKTYAGGRLGLFVFSQEMVFFSDLKYECRDP

Accordingly, the coding sequence, which encodes the TSP-1 polypeptide, may comprise a nucleic acid sequence substantially as set out in either SEQ ID NO. 7 or SEQ ID NO. 8, or a variant thereof or fragment thereof or orthologue thereof. The TSP-1 of the proteinaceous particle may comprise a polypeptide sequence substantially as set out in SEQ ID NO. 9 or a variant thereof or fragment thereof or orthologue thereof. A variant or fragment of a thrombospondin (e.g. TSP-1 and/or, TSP-4) may be an amino acid sequence that is not capable of binding to CD47.

A variant of TSP-1 that is not capable of binding to CD47 may be mutated in a selection of the eight amino acids responsible for TSP-1's ability to binds to CD47. The eight amino acids responsible for TSP-1's ability to binds to CD47 are shown in bold in SEQ ID NO. 9 (i.e. RFYVVMWK (SEQ ID NO: 35), which is the sequence that corresponds to a 4N-1 peptide). A mutation in the amino acids RFYVVMWK (SEQ ID NO: 35) would still allow TSP-1 to fold correctly and incorporate into the proteinaceous particles of the invention. Thus, a variant of TSP-1 may be or comprise or consist of a mutant of 4N-1.

TSP-1 comprises Ca²⁺-binding repeats, which include amino acids 691 to 954 of SEQ ID NO. 9 (see the underlined amino acids of SEQ ID NO. 9 correspond to Ca²⁺-binding repeats of TSP-1). Thus, a fragment of TSP-1 may comprise amino acids 691 to 1170 of SEQ ID NO. 9. A fragment of TSP-1 may comprise the N-terminal or C-terminal region of TSP-1. Preferably the N-terminal or the C-terminal region of TSP-1 comprises the Ca²⁺-binding repeats of TSP-1. An N-terminal region of TSP-1 may comprise amino acids 19 to 270, 19 to 373, 19 to 547 or 19 to 690 of SEQ ID NO. 9. A C-terminal region of TSP-1 may comprise amino acids 547 to 1170, 646 to 1170, 691 to 1170 or 727 to 1170 of SEQ ID NO. 9. The TSP-1 of the proteinaceous particle may comprise or consist of the TSP-1 amino acid sequence of any one of SEQ ID NO. 25 and 27 to 30.

The shell of the proteinaceous particle according to the invention may further comprise other members of the thrombospondin family, such as TSP-2, TSP-3, TSP-4 and/or TSP-5. Preferably, the shell of the proteinaceous particle according to the invention further comprises TSP-4.

In humans, thrombospondin-4 is encoded by the gene THBS4. Thus, the genomic DNA sequence (introns and exons) encoding one embodiment of thrombospondin-4 is referred to herein as SEQ ID NO. 10 and can be found under the gene ID: 7060 (https://www.ncbi.nlm.nih.gov/gene?db=gene&Cmd=DetailsSearch&Term=7060).

The cDNA sequence (exons only) encoding one embodiment of THBS4 is provided herein as SEQ ID NO. 11, as follows:

[SEQ ID NO. 11]
CTGGTCCGTCCAGGCTCCTTCCCATCCTCACACCCGCGCCTTTCTCCCTGCGGCCCCGGCTCGCTGCTCC
AGCTGCCCAGCTCTTCCCCCGCCCGGCCGCACCATAAAGCGCCCGGCCGCTGCCGCGGAGCCCAGCAGCC
AGCTCCCCAGCACCGCGCGGCGGGGACGCGAGCGCGCCCCCGACGGCAGCCCGGACGCCGAGCACGGGTC
ACCTGCGGCGCCGGCCCGGGCGCCGACCGAGGTTCAACGCACGGCCCGGGGACCCCCAGGCGGGGCCAAC
GCCGCCGTCGCCCCCGGCCTCGCGGGGAGCAGGAAGAGCCAACATGCTGGCCCCGCGCGGAGCCGCCGTC
CTCCTGCTGCACCTGGTCCTGCAGCGGTGGCTAGCGGCAGGCGCCCAGGCCACCCCCCAGGTCTTTGACC
TTCTCCCATCTTCCAGTCAGAGGCTAAACCCAGGCGCTCTGCTGCCAGTCCTGACAGACCCCGCCCTGAA
TGATCTCTATGTGATTTCCACCTTCAAGCTGCAGACTAAAAGTTCAGCCACCATCTTCGGTCTTTACTCT
TCAACTGACAACAGTAAATATTTTGAATTTACTGTGATGGGACGCTTAAACAAAGCCATCCTCCGTTACC
TGAAGAACGATGGGAAGGTGCATTTGGTGGTTTTCAACAACCTGCAGCTGGCAGACGGAAGGCGGCACAG
GATCCTCCTGAGGCTGAGCAATTTGCAGCGAGGGGCCGGCTCCCTAGAGCTCTACCTGGACTGCATCCAG
GTGGATTCCGTTCACAATCTCCCCAGGGCCTTTGCTGGCCCCTCCCAGAAACCTGAGACCATTGAATTGA
GGACTTTCCAGAGGAAGCCACAGGACTTCTTGGAAGAGCTGAAGCTGGTGGTGAGAGGCTCACTGTTCCA
GGTGGCCAGCCTGCAAGACTGCTTCCTGCAGCAGAGTGAGCCACTGGCTGCCACAGGCACAGGGGACTTT
AACCGGCAGTTCTTGGGTCAAATGACACAATTAAACCAACTCCTGGGAGAGGTGAAGGACCTTCTGAGAC
AGCAGGTTAAGGAAACATCATTTTTGCGAAACACCATAGCTGAATGCCAGGCTTGCGGTCCTCTCAAGTT
TCAGTCTCCGACCCCAAGCACGGTGGTGCCCCCGGCTCCCCCTGCACCGCCAACACGCCCACCTCGTCGG
TGTGACTCCAACCCATGTTTCCGAGGTGTCCAATGTACCGACAGTAGAGATGGCTTCCAGTGTGGGCCCT
GCCCCGAGGGCTACACAGGAAACGGGATCACCTGTATTGATGTTGATGAGTGCAAATACCATCCCTGCTA
CCCGGGCGTGCACTGCATAAATTTGTCTCCTGGCTTCAGATGTGACGCCTGCCCAGTGGGCTTCACAGGG
CCCATGGTGCAGGGTGTTGGGATCAGTTTTGCCAAGTCAAACAAGCAGGTCTGCACTGACATTGATGAGT
GTCGAAATGGAGCGTGCGTTCCCAACTCGATCTGCGTTAATACTTTGGGATCTTACCGCTGTGGGCCTTG
TAAGCCGGGGTATACTGGTGATCAGATAAGGGGATGCAAAGCGGAAAGAAACTGCAGAAACCCAGAGCTG
AACCCTTGCAGTGTGAATGCCCAGTGCATTGAAGAGAGGCAGGGGGATGTGACATGTGTGTGTGGAGTCG
GTTGGGCTGGAGATGGCTATATCTGTGGAAAGGATGTGGACATCGACAGTTACCCCGACGAAGAACTGCC
ATGCTCTGCCAGGAACTGTAAAAAGGACAACTGCAAATATGTGCCAAATTCTGGCCAAGAAGATGCAGAC
AGAGATGGCATTGGCGACGCTTGTGACGAGGATGCTGACGGAGATGGGATCCTGAATGAGCAGGATAACT
GTGTCCTGATTCATAATGTGGACCAAAGGAACAGCGATAAAGATATCTTTGGGGATGCCTGTGATAACTG
CCTGAGTGTCTTAAATAACGACCAGAAAGACACCGATGGGGATGGAAGAGGAGATGCCTGTGATGATGAC
ATGGATGGAGATGGAATAAAAAACATTCTGGACAACTGCCCAAAATTTCCCAATCGTGACCAACGGGACA
AGGATGGTGATGGTGTGGGGGATGCCTGTGACAGTTGTCCTGATGTCAGCAACCCTAACCAGTCTGATGT
GGATAATGATCTGGTTGGGGACTCCTGTGACACCAATCAGGACAGTGATGGAGATGGGCACCAGGACAGC
ACAGACAACTGCCCCACCGTCATTAACAGTGCCCAGCTGGACACCGATAAGGATGGAATTGGTGACGAGT
GTGATGATGATGATGACAATGATGGTATCCCAGACCTGGTGCCCCCTGGACCAGACAACTGCCGGCTGGT
CCCCAACCCAGCCCAGGAGGATAGCAACAGCGACGGAGTGGGAGACATCTGTGAGTCTGACTTTGACCAG
GACCAGGTCATCGATCGGATCGACGTCTGCCCAGAGAACGCAGAGGTCACCCTGACCGACTTCAGGGCTT
ACCAGACCGTGGTCCTGGATCCTGAAGGGGATGCCCAGATCGATCCCAACTGGGTGGTCCTGAACCAGGG
CATGGAGATTGTACAGACCATGAACAGTGATCCTGGCCTGGCAGTGGGGTACACAGCTTTTAATGGAGTT
GACTTCGAAGGGACCTTCCATGTGAATACCCAGACAGATGATGACTATGCAGGCTTTATCTTTGGCTACC
AAGATAGCTCCAGCTTCTACGTGGTCATGTGGAAGCAGACGGAGCAGACATATTGGCAAGCCACCCCATT
CCGAGCAGTTGCAGAACCTGGCATTCAGCTCAAGGCTGTGAAGTCTAAGACAGGTCCAGGGGAGCATCTC
CGGAACTCCCTGTGGCACACGGGGGACACCAGTGACCAGGTCAGGCTGCTGTGGAAGGACTCCAGGAATG
TGGGCTGGAAGGACAAGGTGTCCTACCGCTGGTTCCTACAGCACAGGCCCCAGGTGGGCTACATCAGGGT
ACGATTTTATGAAGGCTCTGAGTTGGTGGCTGACTCTGGCGTCACCATAGACACCACAATGCGTGGAGGC
CGACTTGGCGTTTTCTGCTTCTCTCAAGAAAACATCATCTGGTCCAACCTCAAGTATCGCTGCAATGACA
CCATCCCTGAGGACTTCCAAGAGTTTCAAACCCAGAATTTCGACCGCTTCGATAATTAAACCAAGGAAGC
AATCTGTAACTGCTTTTCGGAACACTAAAACCATATATATTTTAACTTCAATTTTCTTTAGCTTTTACCA
ACCCAAATATATCAAAACGTTTTATGTGAATGTGGCAATAAAGGAGAAGAGATCATTTTTAAAAAAAAAA
AAAAA

The polypeptide sequence of thrombospondin-4 is provided herein as SEQ ID NO. 12, as follows:

[SEQ ID NO. 12]
MLAPRGAAVLLLHLVLQRWLAAGAQATPQVFDLLPSSSQRLNPGALLPVLTDPALNDLYVISTFK
LQTKSSATIFGLYSSTDNSKYFEFTVMGRLNKAILRYLKNDGKVHLVVFNNLQLADGRRHRILLR
LSNLQRGAGSLELYLDCIQVDSVHNLPRAFAGPSQKPETIELRTFQRKPQDFLEELKLVVRGSLF
QVASLQDCFLQQSEPLAATGTGDFNRQFLGQMTQLNQLLGEVKDLLRQQVKETSFLRNTIAECQA
CGPLKFQSPTPSTVVPPAPPAPPTRPPRRCDSNPCFRGVQCTDSRDGFQCGPCPEGYTGNGITCI
DVDECKYHPCYPGVHCINLSPGFRCDACPVGFTGPMVQGVGISFAKSNKQVCTDIDECRNGACVP
NSICVNTLGSYRCGPCKPGYTGDQIRGCKAERNCRNPELNPCSVNAQCIEERQGDVTCVCGVGWA
GDGYICGKDVDIDSYPDEELPCSARNCKKDNCKYVPNSGQEDADRDGIGDACDEDADGDGILNEQ
DNCVLIHNVDQRNSDKDIFGDACDNCLSVLNNDQKDTDGDGRGDACDDDMDGDGIKNILDNCPKF
PNRDQRDKDGDGVGDACDSCPDVSNPNQSDVDNDLVGDSCDTNQDSDGDGHQDSTDNCPTVINSA
QLDTDKDGIGDECDDDDDNDGIPDLVPPGPDNCRLVPNPAQEDSNSDGVGDICESDFDQDQVIDR
IDVCPENAEVTLTDFRAYQTVVLDPEGDAQIDPNWVVLNQGMEIVQTMNSDPGLAVGYTAFNGVD
FEGTFHVNTQTDDDYAGFIFGYQDSSSFYVVMWKQTEQTYWQATPFRAVAEPGIQLKAVKSKTGP
GEHLRNSLWHTGDTSDQVRLLWKDSRNVGWKDKVSYRWFLQHRPQVGYIRVRFYEGSELVADSGV
TIDTTMRGGRLGVFCFSQENIIWSNLKYRCNDTIPEDFQEFQTQNFDRFDN

Accordingly, the coding sequence, which encodes the TSP-4 polypeptide, may comprise a nucleic acid sequence substantially as set out in either SEQ ID NO. 10 or SEQ ID NO. 11, or a variant thereof or fragment thereof or orthologue thereof. TSP-4 may therefore comprise a polypeptide sequence substantially as set out in SEQ ID NO. 12 or a variant thereof or fragment thereof or orthologue thereof.

TSP-4 comprises Ca²⁺-binding repeats, which include amino acids 463 to 727 of SEQ ID NO. 12 (see the underlined amino acids of SEQ ID NO. 12 correspond to Ca²⁺-binding repeats of TSP-4). Thus, a fragment of TSP-4 may comprise amino acids 463 to 727 of SEQ ID NO. 12. A fragment of TSP-4 may comprise the N-terminal or C-terminal region of TSP-4. Preferably a fragment of TSP-4 comprises the N-terminal or C-terminal region of TSP-4. Preferably an N-region fragment or a C-terminal region of TSP-4 comprises the Ca²⁺-binding repeats of TSP-4. An N-terminal region of TSP-4 may comprise amino acids 27 to 192, 27 to 325, 27 to 363, 27 to 419 or 27 to 462 of SEQ ID NO. 12. A C-terminal region of TSP-4 comprises amino acids 420 to 945, 463 to 945 or 496 to 945 of SEQ ID NO. 12.

Thus, the shell of the proteinaceous particle of the invention may further comprise TSP-2, TSP-3, TSP-4 and/or TSP-5. Preferably, the shell of the proteinaceous particle according to the invention further comprises an amino acid sequence substantially as set out in SEQ ID NO. 12, or a variant thereof or fragment thereof or orthologue thereof.

The shell of the proteinaceous particle of the invention may further comprise a galectin. A galectin is a family of beta-galactosidase-binding proteins that mediate cell-to-cell interactions and cell-to-ECM (extracellular matrix) interactions. There are several members in the family, two of which are galectin-1 and galectin-7.

Human galectin-1 is encoded by the gene LGALS1. Thus, in one embodiment, the genomic DNA sequence (introns and exons) encoding one embodiment of galectin-1 is referred to herein as SEQ ID NO. 13, as follows:

[SEQ ID NO. 13]
ATCTCTCTCGGGTGGAGTCTTCTGACAGCTGGTGCGCCTGCCCGGGAACATCCTCCTGGACTCAATCATG
GCTTGTGTGAGTGTGGGGACCCCCCCCCAAGGTCCAGGGGATAGGGCAGGAACTGATGGCCAGAGGAGAG
CTGGGCAGATCGGGAGCAGATTCTAGCCCCAGCTGTGTGGCCTGGAACCAGTGCCTTCTCTTTTCTGGAC
CTCAGTGGCCACATCTGTAAAATGGGGGTGGGCGCCATGGTCCCTCAAGGCCTTCTCTGCATTGATAATT
GTCTGGATTCCTCCAGGGTCTGAAAGCACAGTTATTTCTGCCCAGGGTTGACATTCTGCAGCTCTCTGAG
AAGTGAGCGTGGGAAGGGTGTGGCCAACTGGGGGACACCCAGGCCACTATCCCTTTCCCCCTCCTCCACC
CCAAAGAGCCTCCTGTCCCCTCCCCCCTGCAGCTGTCCCGGTCACCAGGCCAGGGCAGAGTTACCCTCTG
CTCCAGAGAACGCTGAAAAGTTGCCAGGACCCGAGACAAGCTGCCCAGGATGGGCCCTTCTAGGTCGGGG
GTGGAGGGTGGTTGTGTCCAGGCTGGTGGCGGGGGGGGCGGGGGAAATTCCCTTCCACCACCCCCAAGCT
GGGAGGTTGGGGTGGCAGGGAGGTGAGAATCTTCCTCGGGCCCCAGGGAAAGGGTTCAAGTTTCTGGCAG
AAGAAACCACTCAAACCAGTTAACTCTTGCCCACCCACTCGGGGTGCCAGGGAACAGCAGTGCCCAGCAG
TTTGCTCAATCTTGTTAACCCTGAGCCAGCCCAGCCACAGCCCGACTGCAGGGCTATTCTCCCTAATGCA
GAGAGGCCGTGTTTTTCAGGGTCTCCTCTCTAGCCCCTGGGCCTCTTTGCAGAGAGGGGCTTGAAGGAGA
ATAGTGGGGTCGCCCGCCCCTAGCCACTTCCTCTCCAGCAGAGGGGCCGGCCCCTCCATTCAGTGTGACG
GTGGGCCAAGTGTCGGCCCCTCCCCAGCCTGATCCTCTCCATCTGCGATGGGACAGAGCACCCCATCTCC
CAAGTCACTCTTGAGTCCAAAATTCCCAAGCCAATCTGCAAAATCTTCTAGAGCCTGTCTTCTAGAACCT
TCACGTTACAGACTGAAGCCAACCCCGGTGGGAATAGGGACTTTCCCAGGACCACATAGACAATCGGAGG
CTGGAAATTACAGCTCAATCCTTTCCCCAGGCTTCCCCTTGGCTTGGTCAGAGGATGCCGGGCGGGAACA
ACCCCACTCCCACCCCCAGCCACCCCCGGACACTTCGAGCAGTGGAGGCCTTGTCCTCTAACCCGGCTGG
GCCGGGGCTTGTCTGTGCAGGGTCTGGTCGCCAGCAACCTGAATCTCAAACCTGGAGAGTGCCTTCGAGT
GCGAGGCGAGGTGGCTCCTGACGCTAAGAGGTGAGAAGTGAAGTCGGGGTGGTGGGCGGCAGGGACGGGC
TTGGTCCAGCAGGGAGGGCGTGGCCGGCCAAGCCCACATCTCCTCCCTGGCCGGGAGCGGGTTAACGGCC
AGCCGCCGATGCTGCGTTTTCTGGGTGACTCACTTCCCCCGCAGGGTCTGGGCGCCCCCACCGTTGCCGC
CCCCTCCCCCGCTCCCTCCCTGCTGTAGCCTCTTAAACTAAACCAGCTGCAGCCTCGTCATCTGTAATAC
CTTGACTCCCCCGCCCCCACCCCTTTCCGGTCTGGGCGGGACCTGTCGCTGGGGGGGGGGGGAGAGGAG
TGGGGCGGGCGTCACCGCCGCCTTCCCCCTGAGTCCCTCCTTCCTGCGTCTGGTCATTCATTCATTCACT
GGCTCAGTCCGGTCCTTCTTCCTTCACTTCTCATTCACTCAGACGGCTGCCTTATTTTCTCGGCAGTTAG
GTGACCTTGGACCAGTCAACCAACCTCACCTAGCCTCAGTTTGTTAGAAAAACAAGGGGGGAGGTGGTTG
TGTGGAGGAAGTGAGATGCGGCTGGCGCAGTGACAGTGAGGAGGATGAGGATCAGCTGATATTGTTAAGA
GCCGCACACTTCTCATGCACTAATTTCATTTCAACCAAACCCTCAGAGGCAGGTATTGCTATTATCACCA
CTCAACAAATGTATTTATTATTTTATTTATTTATTTATTATTTTATTATTTTATTTATTTATTTTTTGAG
ACACAGTCTCACCCTGTCGCCCAGGCTGGAGTGCAATGGCGTGATCTCGGCTCACCACAACCTCCGCCTC
CCGGGTTCAAACGATTCTCCGGCTTCAGCCTCCTGAGTAGCCGGGATTACAGGGGCCCACCACAACGCCC
AGCTAATTTATGTACTTTTAGTAGAGACGGGGTTTCACCATGTTGGCCAGGCTGGTCTCGAACTCCTGAC
CTCATGATCTGCCCGCTCGCCTCGGCCTCCCAAAGTGCTGGGGTTACAGGCATGAGCCACCGTGCCCGGG
AGTATTATTATTATTATTTGTTAATTCGCCCCATAATTACTAAGCATCTTTTTCTGGTGTGCCCCACTTG
TGCTGGGCACCGGGAATACAGAGATGTACAAGACAGGACGGGAGGTCACCATCTGGAGGGAGTCACTGAC
CTTGACCAAACGGGCTAGGATGCTAATGACTTCAAGAATCAAGCGAGCCCTCCTGTGCAGCCCCCATTGT
ACAGATGAGCAAACAGGGGAAGAGGGGCAGGAGCAGGTGGCATGGCCAGAGCTAGAATCCAGGTTTCTTG
TCTCTGTTAGTGAGTTCTTCCAGCAAGGTGCGTTCATGGGATACTGAGTGACAGATTAGTCGGTCAGTGG
GGCTGGAGCTGGCCGAGGTGGCCTCATGCCCACCCGTTACCCCCCAGCTTCGTGCTGAACCTGGGCAAAG
ACAGCAACAACCTGTGCCTGCACTTCAACCCTCGCTTCAACGCCCACGGCGACGCCAACACCATCGTGTG
CAACAGCAAGGACGGCGGGGCCTGGGGGACCGAGCAGCGGGAGGCTGTCTTTCCCTTCCAGCCTGGAAGT
GTTGCAGAGGTGGGCTGCAGACCGGAACCGGGGACCAGGGACAGGGGCTGGGTGGGCTGGGGCGGGGCTG
GGTTAGTGACTAGAGACCTTGGCCCTGCCTGCTCTTTCCCCTCCCCTTCCCTCCCTTCCTGTGTGATGGC
CAGTGTCTGCCCCTCTTTGAACCTCAGTGGTTGATTACAATAAAACGAAGGGGAAAAAAAAAGGCTGGGC
TTGGTGGCTCATGCCTGTAATCCCAGCACTTTGGGAGGCCGAGGCGGGCGGATCACCTGAGGTCGGGAGT
TCGAGACCAGCCTGACCAACATGGAGAAACCCTGTCTCGACTAAAAATACAAAAAATTAGCTAGGCGTGA
TGGCGCATGCCTGTAATCCCAGCTACTCAGGAGGCTGAGGCAGGAGAATTGGTTGAACCCGGGAGGCGGA
GGTTGCAGTGAGCCGAGATTGCACCACTGCACTCCAGCCTGGCCAACAAGAGCAAAACTCTGTCTCAAAA
AAAAAAAAAAAAATTAGCCAGCGTGCTGGCTCATGCCTGTAATCCTGGCACTTTGGGAGACCAAAGTGGG
TGGATCACCTGAGGTCAGGAGTTCGAGACCAGCCTGACCAACATGGTGAAACCCCGTCTCTATATAAATA
CAAAAATTAGCTGGGCGTGGTGGCACACAACTGTAGTCCTAGCTACTCAGGAGGCTGAGACAGGAGAATC
ACTTGAACCTGGGAGGCGGAGGTTGCAGTGAGCCGAGATTATGCCACTGTACTCCAGCCTGGGCGACAGA
GGAGACTCCATCTCAAAAAAAAAAAAAAATCTATCATAGGATTAGAGTAAAAAGAAAGAAAAAATATTAT
AAATGTACCTCCGCAGGCTCAGCCACAAACTGGGGCTGTGTCAGGGCCACATGAGGAACGGGTTCTGGAA
GGGCCCATGGCATGTGGGCCCGGCTCACTGCTCTCCTCTACCCCCAGGTGTGCATCACCTTCGACCAGGC
CAACCTGACCGTCAAGCTGCCAGATGGATACGAATTCAAGTTCCCCAACCGCCTCAACCTGGAGGCCATC
AACTACATGGCAGCTGACGGTGACTTCAAGATCAAATGTGTGGCCTTTGACTGAAATCAGCCAGCCCATG
GCCCCCAATAAAGGCAGCTGCCTCTGCTCCCTCTGAA

The cDNA sequence (exons only) encoding one embodiment of galectin-1 is provided herein as SEQ ID NO. 14, as follows:

[SEQ ID NO. 14]
ATCTCTCTCGGGTGGAGTCTTCTGACAGCTGGTGCGCCTGCCCGGGAACATCCTCCTGGACTCAATCATG
GCTTGTGGTCTGGTCGCCAGCAACCTGAATCTCAAACCTGGAGAGTGCCTTCGAGTGCGAGGCGAGGTGG
CTCCTGACGCTAAGAGCTTCGTGCTGAACCTGGGCAAAGACAGCAACAACCTGTGCCTGCACTTCAACCC
TCGCTTCAACGCCCACGGCGACGCCAACACCATCGTGTGCAACAGCAAGGACGGCGGGGCCTGGGGGACC
GAGCAGCGGGAGGCTGTCTTTCCCTTCCAGCCTGGAAGTGTTGCAGAGGTGTGCATCACCTTCGACCAGG
CCAACCTGACCGTCAAGCTGCCAGATGGATACGAATTCAAGTTCCCCAACCGCCTCAACCTGGAGGCCAT
CAACTACATGGCAGCTGACGGTGACTTCAAGATCAAATGTGTGGCCTTTGACTGAAATCAGCCAGCCCAT
GGCCCCCAATAAAGGCAGCTGCCTCTGCTCCCTCTGAAAAAAAAAAAAAAAAAAAAAAAAAAAAAA

The polypeptide sequence of an immature galectin-1 is provided herein as SEQ ID NO. 15, as follows:

[SEQ ID NO. 15]
MACGLVASNLNLKPGECLRVRGEVAPDAKSFVLNLGKDSNNLCLHFNPR
FNAHGDANTIVCNSKDGGAWGTEQREAVFPFQPGSVAEVCITFDQANLT
VKLPDGYEFKFPNRLNLEAINYMAADGDFKIKCVAFD

Amino acids 2 to 135 of SEQ ID NO. 15 correspond to the mature polypeptide chain of galectin-1. Galectin-1 comprises two discontinuous sequences that make up the active β-galactoside binding motif, which include amino acids 45-49 and 69-72 of SEQ ID NO. 15 (the underlined amino acids of SEQ ID NO. 15 correspond to the active β-galactoside binding motifs). Thus, a fragment of galectin-1 may comprise amino acids 45-49 and/or 69-72 of SEQ ID NO. 15. A fragment of galectin-1 may comprise an N-terminal region or a C-terminal region of galectin-1. Preferably an N-terminal region or a C-terminal region comprises the active β-galactoside binding motif.

Accordingly, the coding sequence, which encodes the galectin-1 polypeptide, may comprise a nucleic acid sequence substantially as set out in either SEQ ID NO. 13 or SEQ ID NO. 14, or a variant thereof or fragment thereof or orthologue thereof. The galectin-1 of the proteinaceous particle may comprise a polypeptide sequence substantially as set out in the mature polypeptide chain of SEQ ID NO.15 or a variant thereof or fragment thereof or orthologue thereof.

Human galectin-7 is encoded by the gene LGALS7. The cDNA sequence (exons only) encoding one embodiment of galectin-7 is provided herein as SEQ ID NO. 16, as follows:

[SEQ ID NO. 16]
ACGGCTGCCCAACCCGGTCCCAGCCATGTCCAACGTCCCCCACAAGTCCTCACTGCCCGAGGGCATCCGC
CCTGGCACGGTGCTGAGAATTCGCGGCTTGGTTCCTCCCAATGCCAGCAGGTTCCATGTAAACCTGCTGT
GCGGGGAGGAGCAGGGCTCCGATGCCGCGCTGCATTTCAACCCCCGGCTGGACACGTCGGAGGTGGTCTT
CAACAGCAAGGAGCAAGGCTCCTGGGGCCGCGAGGAGCGCGGGCCGGGCGTTCCTTTCCAGCGCGGGCAG
CCCTTCGAGGTGCTCATCATCGCGTCAGACGACGGCTTCAAGGCCGTGGTTGGGGACGCCCAGTACCACC
ACTTCCGCCACCGCCTGCCGCTGGCGCGCGTGCGCCTGGTGGAGGTGGGCGGGGACGTGCAGCTGGACTC
CGTGAGGATCTTCTGAGCAGAAGCCCAGGCGGGCCCGGGGCCTTGGCTGGCAAATAAAGCGTTAGCCCGC
AGCGAAAAAAAAAAAAAAAAAAAAAGGCCACATGTGC

The polypeptide sequence of an immature galectin-7 is 136 amino acids long and is provided herein as SEQ ID NO. 17, as follows:

[SEQ ID NO. 17]
MSNVPHKSSLPEGIRPGTVLRIRGLVPPNASRFHVNLLCGEEQGSDAAL
HFNPRLDTSEVVFNSKEQGSWGREERGPGVPFQRGQPFEVLIIASDDGF
KAVVGDAQYHHFRHRLPLARVRLVEVGGDVQLDSVRIF

Amino acids 6 to 136 of SEQ ID NO. 17 correspond to the mature polypeptide chain of galectin-7. Galectin-7 comprises an active β-galactoside binding motif, which includes amino acids 70-76 of SEQ ID NO. 17 (the underlined amino acids of SEQ ID NO. 17 corresponds to the active β-galactoside binding motif). Thus, a fragment of galectin-7 may comprise amino acids 70-76 of SEQ ID NO. 17. A fragment of galectin-7 may comprise an N-terminal region or a C-terminal region of galectin-1. Preferably an N-terminal region or a C-terminal region comprises the active β-galactoside binding motif.

Accordingly, the coding sequence, which encodes the galectin-7 polypeptide, may comprise a nucleic acid sequence substantially as set out in either SEQ ID NO. 16, or a variant thereof or fragment thereof or orthologue thereof. The galectin-7 of the proteinaceous particle may comprise a polypeptide sequence substantially as set out in the mature polypeptide chain of SEQ ID NO.17 or a variant thereof or fragment thereof or orthologue thereof.

The core of the proteinaceous particle may further comprise a protein selected from the group comprising: IFN gamma, CCL5 and XCL2 or a fragment, a variant or an orthologue thereof.

The inventors have found that the proteinaceous particle of CD8+ T cells contact membrane vesicles/phospholipid particles containing FasL. Thus, the glycoprotein shell of the proteinaceous particle may contact a vesicle/phospholipid particle containing FasL to form a hybrid particle. The proteinaceous particle of the invention may attach to the membrane vesicles/phospholipid particles containing FasL (via TSP-1 on the proteinaceous particle and CD47 or ICAM-1 on the membrane vesicles/phospholipid particles). The hybrid particle may kill a target cell using mechanisms based on granzymes and/or perform, and FasL.

FasL is a transmembrane protein that is part of the TNF superfamily. It is a ligand of the receptor Fas, which may be found on target cells. Activation of Fas leads to apoptosis of the target cell. Thus, binding of a hybrid particle to a target cell via FasL may induce cell death (i.e. apoptosis) by an additional mechanism.

In one embodiment, the polypeptide sequence of FasL is provided herein as SEQ ID NO. 18, as follows:

[SEQ ID NO. 18]
MQQPFNYPYPQIYWVDSSASSPWAPPGTVLPCPTSVPRRPGQRRPPPPP
PPPPLPPPPPPPPLPPLPLPPLKKRGNHSTGLCLLVMFFMVLVALVGLG
LGMFQLFHLQKELAELRESTSQMHTASSLEKQIGHPSPPPEKKELRKVA
HLTGKSNSRSMPLEWEDTYGIVLLSGVKYKKGGLVINETGLYFVYSKVY
FRGQSCNNLPLSHKVYMRNSKYPQDLVMMEGKMMSYCTTGQMWARSSYL
GAVFNLTSADHLYVNVSELSLVNFEESQTFFGLYKL

Accordingly, the FasL of the hybrid may comprise a polypeptide sequence substantially as set out in SEQ ID NO. 18 or a variant thereof or a fragment thereof or an orthologue thereof.

The shell of the proteinaceous particle of the invention may further comprise other proteins, such as one or more of IFN gamma, CCL5, XCL2 and a toxin.

IFN gamma (type II Interferon) is an immunomodulatory cytokine. It stimulates cells to produce an antiviral or anti-tumour response by binding to a heterodimeric receptor consisting of interferon gamma receptor 1 (IFNGR1) and interferon gamma receptor 2 (IFNGR2). The polypeptide sequence of IFN gamma is provided herein as SEQ ID NO.19, as follows:

[SEQ ID NO. 19]
QDPYVKEAENLKKYFNAGHSDVADNGTLFLGILKNWKEESDRKIMQSQI
VSFYFKLFKNFKDDQSIQKSVETIKEDMNVKFFNSNKKKRDDFEKLTNY
SVTDLNVQRKAIHELIQVMAELSPAAKTGKRKRSQMLFRG

Accordingly, the IFN gamma of the proteinaceous particle may comprise a polypeptide sequence substantially as set out in SEQ ID NO. 19 or a variant thereof or a fragment thereof or an orthologue thereof.

CCL5 (RANTES) is a chemokine. It regulates inflammation by attracting leukocytes (e.g. one or more of T cells, eosinophils and basophils). The polypeptide sequence of CCL5 is provided herein as SEQ ID NO. 20, as follows:

[SEQ ID NO. 20]
SPYSSDTTPCCFAYIARPLPRAHIKEYFYTSGKCSNPAVVFVTRKNRQV
CANPEKKWVREYINSLEMS

Accordingly, the CCL5 of the proteinaceous particle may comprise a polypeptide sequence substantially as set out in SEQ ID NO. 20 or a variant thereof or fragment thereof or orthologue thereof.

XCL2 is a chemokine. It is expressed by T cells and may attract cells expressing the XCL2 receptor (i.e. chemokine receptor XCR1). The polypeptide sequence of XCL2 is provided herein as SEQ ID NO. 21, as follows:

[SEQ ID NO. 21]
VGSEVSHRRTCVSLTTQRLPVSRIKTYTITEGSLRAVIFITKRGLKVCA
DPQATWVRDVVRSMDRKSNTRNNMIQTKPTGTQQSTNTAVTLTG

Accordingly, the XCL2 of the proteinaceous particle may comprise a polypeptide sequence substantially as set out in SEQ ID NO. 21 or a variant thereof or fragment thereof or orthologue thereof.

The shell and/or core of the proteinaceous particle may further comprise a toxin, such as chlorotoxin. This toxin may assist with killing a target cell of the proteinaceous particle. The polypeptide sequence of one embodiment of chlorotoxin is provided herein as SEQ ID NO. 22, as follows:

[SEQ ID NO. 22]
MCMPCFTTDHQMARKCDDCCGGKGRGKCYGPQCLCR

The chlorotoxin of the proteinaceous particle may comprise a polypeptide sequence substantially as set out in SEQ ID NO. 22 or a variant thereof or a fragment thereof or an orthologue thereof. Preferably chlorotoxin is joined to a protein of the shell (e.g. TSP-1 or a fragment thereof, TSP-4 or a fragment thereof, galectin-1 or a fragment thereof, or galectin-7 or a fragment thereof). Thus, chlorotoxin may be joined to a shell protein via a linker (e.g. GGGS (SEQ ID NO: 36)). Thus, in further embodiments, the polypeptide sequence of chlorotoxin is provided herein as SEQ ID NO. 23 or SEQ ID NO 24, as follows:

[SEQ ID NO. 23]
MCMPCFTTDHQMARKCDDCCGGKGRGKCYGPQCLCRGGGS
[SEQ ID NO. 24]
GGGSMCMPCFTTDHQMARKCDDCCGGKGRGKCYGPQCLCR

Thus, the shell of the proteinaceous particle may further comprise a protein selected from the group comprising: IFN-gamma, CCL5, von Willebrand's Factor, XCL2, FasL (via a vesicle/phospholipid particle), a toxin (e.g. chlorotoxin) or a fragment thereof or an orthologue thereof.

A proteinaceous particle may be engineered by incorporating a genetically modified protein into the particle. A genetically modified protein may be a genetically modified shell protein (e.g. a fusion protein based on a protein of the glycoprotein shell, or a fragment, a variant or an orthologue of a shell protein, such as a thrombospondin or a galectin), a genetically modified core protein (e.g. a granzyme fusion protein, or a fragment, a variant or an orthologue of a granzyme), a heterologous protein, such as a transgenic protein (e.g. a transgenic ligand) and/or an antibody or a fragment thereof. Thus, a shell protein, such as a thrombospondin (e.g. TSP-1 and/or TSP-4) a galectin (e.g., galectin-1 and/or galectin-7) and/or a protein within the core of the proteinaceous particle (e.g. granzyme) may be a fusion protein. A fusion protein may be a granzyme B fusion protein. The proteinaceous particle may comprise one or more, two or more, three or more or four or more fusion proteins. Preferably a shell protein is a fusion protein. Most preferably the thrombospondin (e.g. TSP-1 and/or TSP-4) is a fusion protein.

The fusion protein may be formed from a full-length protein/polypeptide of a proteinaceous particle or a fragment thereof and another polypeptide, such as a ligand of a target cell. For example, the proteinaceous particle may be modified so that galectin-1, galectin-7, granzyme B, TSP-1 and/or TSP-4 form(s) a fusion protein with another polypeptide, such as a ligand of a target cell. A fusion protein comprising TSP-1 may comprise the full length TSP-1 protein (such as SEQ ID NO. 9) or a fragment thereof (such as amino acids 691 to 1170 of SEQ ID NO. 9, or amino acids 19 to 690 of SEQ ID NO. 9) and a another polypeptide, such as a ligand. In another embodiment, a fusion protein comprising TSP-4 may comprise the full-length TSP-4 protein (such as SEQ ID NO. 12) or a fragment thereof (such as amino acids 463 to 945 of SEQ ID NO. 12, or amino acids 27 to 462 of SEQ ID NO. 12) and another polypeptide, such as a ligand. In another embodiment, a fusion protein comprising galectin-1 may comprise the full-length galectin-1 protein (such as SEQ ID NO. 15) or a fragment thereof (such as amino acids 4 to 135 of SEQ ID NO. 15, or amino acids 2 to 135 of SEQ ID NO. 15) and another polypeptide, such as a ligand. In another embodiment, a fusion protein comprising galectin-7 may comprise the full-length galectin-7 protein (such as SEQ ID NO. 17) or a fragment thereof (such as amino acids 6 to 136 of SEQ ID NO. 17, or amino acids 1 to 136 of SEQ ID NO. 17) and another polypeptide, such as a ligand. The polypeptide/protein of the proteinaceous particle may be N-terminal to the other polypeptide fusion partner, such as a ligand. A linker sequence, for example between 1 and 10 residues may also be provided between the fused polypeptides. The linker may be about 5 residues in length. Preferably the linker comprises or consists of a GGGGS (SEQ ID NO: 37) linker, which doesn't undergo processing.

In one embodiment the thrombospondin, such as TSP-1, is engineered to form a fusion protein with another polypeptide. In one embodiment, the genetically modified TSP-1 may comprise the sequence of the TSP-1/GFP fusion described herein (SEQ ID NO. 25), wherein the GFP fusion is substituted for an alternative polypeptide molecule, such as a ligand or receptor of a target cell.

Thus, the proteinaceous particle may be an engineered proteinaceous particle comprising a core of a perform and/or a granzyme, the core being surrounded by a glycoprotein shell comprising a thrombospondin-1 (TSP-1) fusion protein, and optionally galectin-1 or galectin-7 or a fragment thereof, a variant thereof or an orthologue thereof.

A TSP-1 fusion protein may be a TSP-1/GFP fusion protein. A polypeptide sequence of a TSP-1/GFP fusion protein is provided herein as SEQ ID NO. 25, as follows:

[SEQ ID NO. 25]
MGLAWGLGVLFLMHVCGTNRIPESGGDNSVFDIFELTGAARKGSGRRLVKGPDPSSPAFRIEDANLIPPV
PDDKFQDLVDAVRAEKGFLLLASLRQMKKTRGTLLALERKDHSGQVFSVVSNGKAGTLDLSLTVQGKQHV
VSVEEALLATGQWKSITLFVQEDRAQLYIDCEKMENAELDVPIQSVFTRDLASIARLRIAKGGVNDNFQG
VLQNVRFVFGTTPEDILRNKGCSSSTSVLLTLDNNVVNGSSPAIRTNYIGHKTKDLQAICGISCDELSSM
VLELRGLRTIVTTLQDSIRKVTEENKELANELRRPPLCYHNGVQYRNNEEWTVDSCTECHCQNSVTICKK
VSCPIMPCSNATVPDGECCPRCWPSDSADDGWSPWSEWTSCSTSCGNGIQQRGRSCDSLNNRCEGSSVQT
RTCHIQECDKRFKQDGGWSHWSPWSSCSVTCGDGVITRIRLCNSPSPQMNGKPCEGEARETKACKKDACP
INGGWGPWSPWDICSVTCGGGVQKRSRLCNNPTPQFGGKDCVGDVTENQICNKQDCPIDGCLSNPCFAGV
KCTSYPDGSWKCGACPPGYSGNGIQCTDVDECKEVPDACFNHNGEHRCENTDPGYNCLPCPPRFTGSQPF
GQGVEHATANKQVCKPRNPCTDGTHDCNKNAKCNYLGHYSDPMYRCECKPGYAGNGIICGEDTDLDGWPN
ENLVCVANATYHCKKDNCPNLPNSGQEDYDKDGIGDACDDDDDNDKIPDDRDNCPFHYNPAQYDYDRDDV
GDRCDNCPYNHNPDQADTDNNGEGDACAADIDGDGILNERDNCQYVYNVDQRDTDMDGVGDQCDNCPLEH
NPDQLDSDSDRIGDTCDNNQDIDEDGHQNNLDNCPYVPNANQADHDKDGKGDACDHDDDNDGIPDDKDNC
RLVPNPDQKDSDGDGRGDACKDDFDHDSVPDIDDICPENVDISETDFRRFQMIPLDPKGTSQNDPNWVVR
HQGKELVQTVNCDPGLAVGYDEFNAVDFSGTFFINTERDDDYAGFVFGYQSSSRFYVVMWKQVTQSYWDT
NPTRAQGYSGLSVKVVNSTTGPGEHLRNALWHTGNTPGQVRTLWHDPRHIGWKDFTAYRWRLSHRPKTGF
IRVVMYEGKKIMADSGPIYDKTYAGGRLGLFVFSQEMVFFSDLKYECRDPGGGGSVSKGEELFTGVVPIL
VELDGDVNGHKFSVSGEGEGDATYGKLTLKFICTTGKLPVPWPTLVTTLTYGVQCFSRYPDHMKKHDFFK
SAMPEGYVQERTIFFKDDGNYKTRAEVKFEGDTLVNRIELKGIDFKEDGNILGHKLEYNYNSHNVYIMAD
KQKNGIKANFKVRHNIEDGSVQLADHYQQNTPIGDGPVLLPDNHYLSTQSALSKDPNEKRDHMVLLEFVT
AAGITLGMDELYK

The amino acid sequence MGLAWGLGVLFLMHVCGT (SEQ ID NO: 38) of SEQ ID NO. 25 corresponds to the signal peptide. The underlined amino acids of SEQ ID NO. 25 correspond to the TSP-1 amino acids. The italicized amino acids of SEQ ID NO. 25 correspond to a linker. The bold amino acids of SEQ ID NO. 25 correspond to the GFP amino acids.

Accordingly, a TSP-1 fusion protein may comprise a polypeptide sequence substantially as set out in SEQ ID NO. 25 or a variant thereof or a fragment thereof or an orthologue thereof.

Furthermore, the skilled person will appreciate that the GFP sequence of SEQ ID NO. 25 can be replaced by an amino acid sequence of a globular protein or a peptide tag. Also, one or more of the shell proteins, galectin-1, galectin-7 and TSP-4 may form a fusion protein with GFP. The amino acid sequence of GFP is shown in bold in SEQ ID NO. 25.

The proteinaceous particle may be an engineered proteinaceous particle comprising a core of a perform and/or a granzyme, the core being surrounded by a glycoprotein shell comprising thrombospondin-1 or a fragment thereof, a variant thereof or an orthologue thereof, and a galectin fusion protein (e.g. a galectin-1 or galectin-7 fusion protein). Galectin-1 and galectin-7 are made in the cell cytoplasm and the N-terminal methionine and N-terminal 5 amino acids, respectively, are removed after synthesis and before export. Thus, addition of sequences will preferentially be to the fixed C-terminus, with a linker. Preferably the linker comprises or consists of a GGGGS (SEQ ID NO: 37) linker, which doesn't undergo processing.

In one embodiment, the proteinaceous particle may be an engineered proteinaceous particle comprising a core of a perform and/or a granzyme, the core being surrounded by a glycoprotein shell comprising:
a TSP-1, or a fragment thereof, a variant thereof or an orthologue thereof, wherein the TSP-1 is a fusion polypeptide with a ligand; and optionally
a galectin or a fragment thereof, a variant thereof or an orthologue thereof.

In another embodiment, the proteinaceous particle may be an engineered proteinaceous particle comprising a core of a perform and/or a granzyme, the core being surrounded by a glycoprotein shell comprising:
a TSP-1 or a fragment thereof, a variant thereof or an orthologue thereof;
a TSP-4 fusion protein; and optionally
a galectin or a fragment thereof, a variant thereof or an orthologue thereof.

The TSP-4 fusion protein may be a fusion protein with a ligand.

In one embodiment, the proteinaceous particle may be an engineered proteinaceous particle comprising a core of granzyme wherein the granzyme is a fusion protein with a ligand, the core being surrounded by a glycoprotein shell comprising:
a TSP-1, or a fragment thereof, a variant thereof or an orthologue thereof; and optionally
a galectin or a fragment thereof, a variant thereof or an orthologue thereof.

The engineered proteinaceous particle according to the invention may further comprise a genetically modified galectin. Thus, the engineered proteinaceous particle according to the invention may further comprise a galectin fusion protein, such as a galectin-1 fusion protein or a galectin-7 fusion protein. The galectin fusion protein (e.g. the galectin-1 fusion protein or the galectin-7 fusion protein) may be a galectin fusion protein with a ligand.

The engineered proteinaceous particle according to the invention may further or alternatively comprise a granzyme fusion protein, such as a granzyme A, B, H, M and/or K fusion protein. In one embodiment, the polypeptide sequence of a granzyme B fusion protein with mCherry and SEpHluorin is provided herein as SEQ ID NO. 26, as follows:

[SEQ ID NO. 26]
MQPILLLLAFLLLPRADAGEIIGGHEAKPHSRPYMAYLMIWDQKSLKRCGGFLIRDDFVLTAAHC
WGSSINVTLGAHNIKEQEPTQQFIPVKRPIPHPAYNPKNFSNDIMLLQLERKAKRTRAVQPLRLP
SNKAQVKPGQTCSVAGWGQTAPLGKHSHTLQEVKMTVQEDRKCESDLRHYYDSTIELCVGDPEIK
KTSFKGDSGGPLVCNKVAQGIVSYGRNNGMPPRACTKVSSFVHWIKKTMKRYGGGGSVSKGEEDN
MAIIKEFMRFKVHMEGSVNGHEFEIEGEGEGRPYEGTQTAKLKVTKGGPLPFAWDILSPQFMYGS
KAYVKHPADIPDYLKLSFPEGFKWERVMNFEDGGVVTVTQDSSLQDGEFIYKVKLRGTNFPSDGP
VMQKKTMGWEASSERMYPEDGALKGEIKQRLKLKDGGHYDAEVKTTYKAKKPVQLPGAYNVNIKL
DITSHNEDYTIVEQYERAEGRHSTGGMDELYKGGGGSSKGEELFTGVVPILVELDGDVNGHKFSV
SGEGEGDATYGKLTLKFICTTGKLPVPWPTLVTTFSYGVQCFSRYPDHMKQHDFFKSAMPEGYVQ
ERTIFFKDDGNYKTRAEVKFEGDTLVNRIELKGIDFKEDGNILGHKLEYNYNDHQVYIMADKQKN
GIKVNFKIRHNIEDGSVQLADHYQQNTPIGDGPVLLPDNHYLFTTSTLSKDPNEKRDHMVLLEFV
TAAGITHGMDELYK

The italicized amino acids of SEQ ID NO. 26 correspond to a linker (i.e. GGGGS (SEQ ID NO: 37)). The bold amino acids of SEQ ID NO. 26 correspond to the amino acids of mCherry. The underlined amino acids of SEQ ID NO. 26 correspond to the amino acids of SEpHluorin.

The granzyme fusion protein may comprise a fusion with a marker protein, such as a fluorescent marker protein. An example of a granzyme fusion protein with a marker protein is provided in SEQ ID NO. 26 and may be used in the present invention. In this example, the fusion is with mCherry and SEpHluorin (GFP like proteins). In one embodiment, the mCherry and/or SEpHluorin sequence may be replaced with an alternative polypeptide sequence.

The skilled person will appreciate that as granzymes are part of the core of the particle, any polypeptides, such as ligands (e.g. target ligands) that are attached to a granzyme to form a fusion protein will only be accessible to receptors on a target cell via pores in the shell of the proteinaceous particle.

In an alternative embodiment, the shell of a proteinaceous particle according to the invention comprises a ligand (i.e. a non-fusion protein polypeptide). Thus, the shell of the proteinaceous particle of the invention may further comprise a ligand of a target cell.

A ligand refers to an agent or moiety that (specifically) binds to a protein (e.g. receptor or ion channel) or marker on a target cell. Preferably, the ligand binds specifically to the protein or marker. The ligand may be a polypeptide. Preferably the ligand is heterologous, such as transgenic (e.g. a heterologous/transgenic polypeptide). The ligand may be an antibody or a fragment thereof (e.g. a scFv, a VL, a VH a Fd; an Fv, an Fab, a Fab′, a F(ab′)2, an Fc fragment, or a bispecific antibody) that binds specifically to a protein expressed on a target cell. Preferably the antibody is a scFv. In another embodiment, the ligand may comprise an antibody mimetic.

Thus, another embodiment of a TSP-1 fusion protein may be a TSP-1/T1-scFv fusion protein. T1-scFv is a single chain antibody that binds to neoantigen HLA-A2 NYESO-1 peptide 157-165. NYESO-1 protein can be expressed in glioblastoma cells and thus the addition of the T1-scFv, or its variants with modified affinity, will improve targeting of the proteinaceous particle to glioblastoma and other tumours that express NYESO-1 protein.

Another embodiment of a TSP-1 fusion protein may comprise a polypeptide sequence of a TSP-1/T1-scFv fusion protein. The polypeptide sequence is provided herein as SEQ ID NO. 27, as follows:

[SEQ ID NO. 27]
MGLAWGLGVLFLMHVCGTNRIPESGGDNSVFDIFELTGAARKGSGRRLVKGPDPSSPAFRIEDANLIPPV
PDDKFQDLVDAVRAEKGFLLLASLRQMKKTRGTLLALERKDHSGQVFSVVSNGKAGTLDLSLTVQGKQHV
VSVEEALLATGQWKSITLFVQEDRAQLYIDCEKMENAELDVPIQSVFTRDLASIARLRIAKGGVNDNFQG
VLQNVRFVFGTTPEDILRNKGCSSSTSVLLTLDNNVVNGSSPAIRTNYIGHKTKDLQAICGISCDELSSM
VLELRGLRTIVTTLQDSIRKVTEENKELANELRRPPLCYHNGVQYRNNEEWTVDSCTECHCQNSVTICKK
VSCPIMPCSNATVPDGECCPRCWPSDSADDGWSPWSEWTSCSTSCGNGIQQRGRSCDSLNNRCEGSSVQT
RTCHIQECDKRFKQDGGWSHWSPWSSCSVTCGDGVITRIRLCNSPSPQMNGKPCEGEARETKACKKDACP
INGGWGPWSPWDICSVTCGGGVQKRSRLCNNPTPQFGGKDCVGDVTENQICNKQDCPIDGCLSNPCFAGV
KCTSYPDGSWKCGACPPGYSGNGIQCTDVDECKEVPDACFNHNGEHRCENTDPGYNCLPCPPRFTGSQPF
GQGVEHATANKQVCKPRNPCTDGTHDCNKNAKCNYLGHYSDPMYRCECKPGYAGNGIICGEDTDLDGWPN
ENLVCVANATYHCKKDNCPNLPNSGQEDYDKDGIGDACDDDDDNDKIPDDRDNCPFHYNPAQYDYDRDDV
GDRCDNCPYNHNPDQADTDNNGEGDACAADIDGDGILNERDNCQYVYNVDQRDTDMDGVGDQCDNCPLEH
NPDQLDSDSDRIGDTCDNNQDIDEDGHQNNLDNCPYVPNANQADHDKDGKGDACDHDDDNDGIPDDKDNC
RLVPNPDQKDSDGDGRGDACKDDFDHDSVPDIDDICPENVDISETDFRRFQMIPLDPKGTSQNDPNWVVR
HQGKELVQTVNCDPGLAVGYDEFNAVDFSGTFFINTERDDDYAGFVFGYQSSSRFYVVMWKQVTQSYWDT
NPTRAQGYSGLSVKVVNSTTGPGEHLRNALWHTGNTPGQVRTLWHDPRHIGWKDFTAYRWRLSHRPKTGF
IRVVMYEGKKIMADSGPIYDKTYAGGRLGLFVFSQEMVFFSDLKYECRDPGGGGSMAEVQLLESGGGLVQ
PGGSLRLSCAASGFTFSTYQMSWVRQAPGKGLEWVSGIVSSGGSTAYADSVKGRFTISRDNSKNTLYLQM
NSLRAEDTAVYYCAGELLPYYGMDVWGQGTTVTVSSAKTTPKLEEGEFSEARVQSELTQPRSVSGSPGQS
VTISCTGTERDVGGYNYVSWYQQHPGKAPKLIIHDVIERSSGVPDRFSGSKSGNTASLTISGLQAEDEAD
YYCWSFAGGYYVFGTGTDVTVLGQPKANPTVD

The underlined amino acids of SEQ ID NO. 27 correspond to the TSP-1 amino acids. The italicized amino acids of SEQ ID NO. 27 correspond to a linker. The bold amino acids of SEQ ID NO. 27 correspond to the T1-scFV amino acids.

Accordingly, the proteinaceous particle may comprise a polypeptide sequence substantially as set out in SEQ ID NO. 27 or a variant thereof or a fragment thereof.

Another embodiment of a TSP-1 fusion protein may be a T1-scFv/TSP-1 fusion protein. The fusion protein may comprise a polypeptide sequence provided herein as SEQ ID NO. 28, as follows:

[SEQ ID NO. 28]
MDFQVQIFSFLLISASVIMSRMAEVQLLESGGGLVQPGGSLRLSCAASGFTFSTYQMSWVRQAPG
KGLEWVSGIVSSGGSTAYADSVKGRFTISRDNSKNTLYLQMNSLRAEDTAVYYCAGELLPYYGMD
VWGQGTTVTVSSAKTTPKLEEGEFSEARVQSELTQPRSVSGSPGQSVTISCTGTERDVGGYNYVS
WYQQHPGKAPKLIIHDVIERSSGVPDRFSGSKSGNTASLTISGLQAEDEADYYCWSFAGGYYVFG
TGTDVTVLGQPKANPTVDGGGGSNRIPESGGDNSVFDIFELTGAARKGSGRRLVKGPDPSSPAFRIED
ANLIPPVPDDKFQDLVDAVRAEKGFLLLASLRQMKKTRGTLLALERKDHSGQVFSVVSNGKAGTLDLSLT
VQGKQHVVSVEEALLATGQWKSITLFVQEDRAQLYIDCEKMENAELDVPIQSVFTRDLASIARLRIAKGG
VNDNFQGVLQNVRFVFGTTPEDILRNKGCSSSTSVLLTLDNNVVNGSSPAIRTNYIGHKTKDLQAICGIS
CDELSSMVLELRGLRTIVTTLQDSIRKVTEENKELANELRRPPLCYHNGVQYRNNEEWTVDSCTECHCQN
SVTICKKVSCPIMPCSNATVPDGECCPRCWPSDSADDGWSPWSEWTSCSTSCGNGIQQRGRSCDSLNNRC
EGSSVQTRTCHIQECDKRFKQDGGWSHWSPWSSCSVTCGDGVITRIRLCNSPSPQMNGKPCEGEARETKA
CKKDACPINGGWGPWSPWDICSVTCGGGVQKRSRLCNNPTPQFGGKDCVGDVTENQICNKQDCPIDGCLS
NPCFAGVKCTSYPDGSWKCGACPPGYSGNGIQCTDVDECKEVPDACFNHNGEHRCENTDPGYNCLPCPPR
FTGSQPFGQGVEHATANKQVCKPRNPCTDGTHDCNKNAKCNYLGHYSDPMYRCECKPGYAGNGIICGEDT
DLDGWPNENLVCVANATYHCKKDNCPNLPNSGQEDYDKDGIGDACDDDDDNDKIPDDRDNCPFHYNPAQY
DYDRDDVGDRCDNCPYNHNPDQADTDNNGEGDACAADIDGDGILNERDNCQYVYNVDQRDTDMDGVGDQC
DNCPLEHNPDQLDSDSDRIGDTCDNNQDIDEDGHQNNLDNCPYVPNANQADHDKDGKGDACDHDDDNDGI
PDDKDNCRLVPNPDQKDSDGDGRGDACKDDFDHDSVPDIDDICPENVDISETDFRRFQMIPLDPKGTSQN
DPNWVVRHQGKELVQTVNCDPGLAVGYDEFNAVDFSGTFFINTERDDDYAGFVFGYQSSSRFYVVMWKQV
TQSYWDTNPTRAQGYSGLSVKVVNSTTGPGEHLRNALWHTGNTPGQVRTLWHDPRHIGWKDFTAYRWRLS
HRPKTGFIRVVMYEGKKIMADSGPIYDKTYAGGRLGLFVFSQEMVFFSDLKYECRDP

The amino acid sequence MGLAWGLGVLFLMHVCGT (SEQ ID NO: 38) of SEQ ID NO. 28 corresponds to the signal peptide. The underlined amino acids of SEQ ID NO. 28 correspond to the TSP-1 amino acids. The italicized amino acids of SEQ ID NO. 28 correspond to a linker. The bold amino acids of SEQ ID NO. 28 correspond to the T1-scFV amino acids.

Accordingly, the TSP-1 of the proteinaceous particle may comprise a polypeptide sequence substantially as set out in SEQ ID NO.28 or a variant thereof or fragment thereof or orthologue thereof.

Another embodiment of a TSP-1 fusion protein may be a TSP-1/chlorotoxin fusion protein. The chlorotoxin peptide interacts with the chloride channels expressed selectively on glioblastoma cells. Thus a TSP-1/chlorotoxin fusion protein would improve targeting of a proteinaceous particle to glioblastoma and other tumours that have a chlorotoxin binding phenotype. A polypeptide sequence of on embodiment of a TSP-1/chlorotoxin fusion protein is provided herein as SEQ ID NO. 29, as follows:

[SEQ ID NO. 29]
MGLAWGLGVLFLMHVCGTNRIPESGGDNSVFDIFELTGAARKGSGRRLVKGPDPSSPAFRIEDANLIPPV
PDDKFQDLVDAVRAEKGFLLLASLRQMKKTRGTLLALERKDHSGQVFSVVSNGKAGTLDLSLTVQGKQHV
VSVEEALLATGQWKSITLFVQEDRAQLYIDCEKMENAELDVPIQSVFTRDLASIARLRIAKGGVNDNFQG
VLQNVRFVFGTTPEDILRNKGCSSSTSVLLTLDNNVVNGSSPAIRTNYIGHKTKDLQAICGISCDELSSM
VLELRGLRTIVTTLQDSIRKVTEENKELANELRRPPLCYHNGVQYRNNEEWTVDSCTECHCQNSVTICKK
VSCPIMPCSNATVPDGECCPRCWPSDSADDGWSPWSEWTSCSTSCGNGIQQRGRSCDSLNNRCEGSSVQT
RTCHIQECDKRFKQDGGWSHWSPWSSCSVTCGDGVITRIRLCNSPSPQMNGKPCEGEARETKACKKDACP
INGGWGPWSPWDICSVTCGGGVQKRSRLCNNPTPQFGGKDCVGDVTENQICNKQDCPIDGCLSNPCFAGV
KCTSYPDGSWKCGACPPGYSGNGIQCTDVDECKEVPDACFNHNGEHRCENTDPGYNCLPCPPRFTGSQPF
GQGVEHATANKQVCKPRNPCTDGTHDCNKNAKCNYLGHYSDPMYRCECKPGYAGNGIICGEDTDLDGWPN
ENLVCVANATYHCKKDNCPNLPNSGQEDYDKDGIGDACDDDDDNDKIPDDRDNCPFHYNPAQYDYDRDDV
GDRCDNCPYNHNPDQADTDNNGEGDACAADIDGDGILNERDNCQYVYNVDQRDTDMDGVGDQCDNCPLEH
NPDQLDSDSDRIGDTCDNNQDIDEDGHQNNLDNCPYVPNANQADHDKDGKGDACDHDDDNDGIPDDKDNC
RLVPNPDQKDSDGDGRGDACKDDFDHDSVPDIDDICPENVDISETDFRRFQMIPLDPKGTSQNDPNWVVR
HQGKELVQTVNCDPGLAVGYDEFNAVDFSGTFFINTERDDDYAGFVFGYQSSSRFYVVMWKQVTQSYWDT
NPTRAQGYSGLSVKVVNSTTGPGEHLRNALWHTGNTPGQVRTLWHDPRHIGWKDFTAYRWRLSHRPKTGF
IRVVMYEGKKIMADSGPIYDKTYAGGRLGLFVFSQEMVFFSDLKYECRDPGGGGSMCMPCFTTDHQMAR
KCDDCCGGKGRGKCYGPQCLCR

The amino acid sequence MGLAWGLGVLFLMHVCGT (SEQ ID NO: 38) of SEQ ID NO. 29 corresponds to the signal peptide. The underlined amino acids of SEQ ID NO. 29 correspond to the TSP-1 amino acids. The italicized amino acids of SEQ ID NO. 29 correspond to a linker. The bold amino acids of SEQ ID NO. 30 correspond to the chlorotoxin amino acids.

Accordingly, the TSP-1 of the proteinaceous particle may comprise a polypeptide sequence substantially as set out in SEQ ID NO. 29 or a variant thereof or fragment thereof or orthologue thereof.

Another embodiment of a TSP-1 fusion protein may be a chlorotoxin/TSP-1 fusion protein. A polypeptide sequence of a chlorotoxin/TSP-1 fusion protein is provided herein as SEQ ID NO. 30, as follows:

[SEQ ID NO. 30]
MGLAWGLGVLFLMHVCGTMCMPCFTTDHQMARKCDDCCGGKGRGKCYGPQCLCRGGGGSNRIPESG
GDNSVFDIFELTGAARKGSGRRLVKGPDPSSPAFRIEDANLIPPVPDDKFQDLVDAVRAEKGFLLLASLR
QMKKTRGTLLALERKDHSGQVFSVVSNGKAGTLDLSLTVQGKQHVVSVEEALLATGQWKSITLFVQEDRA
QLYIDCEKMENAELDVPIQSVFTRDLASIARLRIAKGGVNDNFQGVLQNVRFVFGTTPEDILRNKGCSSS
TSVLLTLDNNVVNGSSPAIRTNYIGHKTKDLQAICGISCDELSSMVLELRGLRTIVTTLQDSIRKVTEEN
KELANELRRPPLCYHNGVQYRNNEEWTVDSCTECHCQNSVTICKKVSCPIMPCSNATVPDGECCPRCWPS
DSADDGWSPWSEWTSCSTSCGNGIQQRGRSCDSLNNRCEGSSVQTRTCHIQECDKRFKQDGGWSHWSPWS
SCSVTCGDGVITRIRLCNSPSPQMNGKPCEGEARETKACKKDACPINGGWGPWSPWDICSVTCGGGVQKR
SRLCNNPTPQFGGKDCVGDVTENQICNKQDCPIDGCLSNPCFAGVKCTSYPDGSWKCGACPPGYSGNGIQ
CTDVDECKEVPDACFNHNGEHRCENTDPGYNCLPCPPRFTGSQPFGQGVEHATANKQVCKPRNPCTDGTH
DCNKNAKCNYLGHYSDPMYRCECKPGYAGNGIICGEDTDLDGWPNENLVCVANATYHCKKDNCPNLPNSG
QEDYDKDGIGDACDDDDDNDKIPDDRDNCPFHYNPAQYDYDRDDVGDRCDNCPYNHNPDQADTDNNGEGD
ACAADIDGDGILNERDNCQYVYNVDQRDTDMDGVGDQCDNCPLEHNPDQLDSDSDRIGDTCDNNQDIDED
GHQNNLDNCPYVPNANQADHDKDGKGDACDHDDDNDGIPDDKDNCRLVPNPDQKDSDGDGRGDACKDDFD
HDSVPDIDDICPENVDISETDFRRFQMIPLDPKGTSQNDPNWVVRHQGKELVQTVNCDPGLAVGYDEFNA
VDFSGTFFINTERDDDYAGFVFGYQSSSRFYVVMWKQVTQSYWDTNPTRAQGYSGLSVKVVNSTTGPGEH
LRNALWHTGNTPGQVRTLWHDPRHIGWKDFTAYRWRLSHRPKTGFIRVVMYEGKKIMADSGPIYDKTYAG
GRLGLFVFSQEMVFFSDLKYECRDP

The amino acid sequence MGLAWGLGVLFLMHVCGT (SEQ ID NO: 38) of SEQ ID NO. 30 corresponds to the signal peptide. The underlined amino acids of SEQ ID NO. 30 correspond to the TSP-1 amino acids. The italicized amino acids of SEQ ID NO. 30 correspond to a linker. The bold amino acids of SEQ ID NO. 30 correspond to the chlorotoxin amino acids.

Accordingly, the TSP-1 of the proteinaceous particle may comprise a polypeptide sequence substantially as set out in SEQ ID NO. 30 or a variant thereof or a fragment thereof or an orthologue thereof.

A TSP-1 fusion protein may comprise a linker to connect TSP-1 or a fragment thereof to another protein. The linker may be the linker of any one of SEQ ID NOS. 25 to 30.

In another embodiment, the proteinaceous particle comprises a fusion protein formed from a shell protein (e.g. a TSP-1 fusion protein, a TSP-4 fusion protein, a galectin-1 fusion protein or a galectin-7 fusion protein) and/or a ligand of a target cell (e.g. chloride channels targeted by chlorotoxin) and/or an antibody (such as a scFv) that binds specifically to a protein expressed on a target cell (e.g. CD19).

The proteinaceous particle of the invention can be used to treat a variety of diseases. This may be achieved with a proteinaceous particle according to the first or second aspect of the invention. However, an advantage of the particle according to the second aspect is that it may be engineered to improve its specificity for a protein (e.g. a biomarker or receptor) expressed on target cells of a disease of interest. For example, the proteinaceous particle of the invention may comprise a ligand, fusion protein and/or antibody that targets specific cancer/tumour cells. Alternatively, the proteinaceous particle may comprise a specific ligand, fusion protein and/or antibody that targets (bacterial and/or virally) infected target cells. The skilled person would appreciate which ligand, fusion protein and/or antibody would provide the proteinaceous particle with targeting specificity for a target cell. Similarly, the skilled person would appreciate which cells must be targeted to treat a disease or condition of a subject. Target proteins that are specific to the tumour or infected cells, and only shared with non-essential normal cells, include (i) CD19 or CD20, which may be targeted on B cell leukemias, (ii) shared tumour-testes antigens and neoantigen peptides bound to MHC molecules that are characteristic of specific types of tumours, (iii) pathogen associated peptides that are not found in the host, (iv) metabolic sensors, like Mr1 proteins, with tumour or microbe associated metabolites bound to generate unique molecular patterns at the surface of cancer or infected cells, and (v) any peptide or polypeptide that is found empirically to bind to tumour cells and not normal cells, for example, chlorotoxin. Thus, the proteinaceous particle of the invention may be engineered to target the protein of any one of (i) to (v).

The shell of the proteinaceous particle of the invention may or may not bind to a target cell comprising CD47 (also known as Integrin Associated Protein (IAP)). Thus, the particle of the invention may bind to CD47 via TSP-1 or other thrombospondins, such as TSP-2, TSP-3, TSP-4 or TSP-5. CD47 also acts as a signal that prevents phagocytic cells of the immune system from phagocytosing cells that express CD47. Thus, target cells that lack CD47 may not be targeted by proteinaceous particles according to the invention but are more likely to be phagocytosed. This property of CD47 makes evasion of the proteinaceous particles by loss of CD47 expression on tumour cells or infected cells less likely to be successful for survival of the tumour or infected cells.

CD47 is encoded by the gene CD47. Thus, the genomic DNA sequence (introns and exons) encoding one embodiment of CD47 is referred to herein as SEQ ID NO. 31 and can be found under the gene ID: 961 (https://www.ncbi.nlm.nih.gov/gene?db=gene&Cmd=DetailsSearch&Term=961).

The polypeptide sequence of CD47 is provided herein as SEQ ID NO. 32, as follows:

[SEQ ID NO. 32]
MWPLVAALLLGSACCGSAQLLFNKTKSVFFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSPNENILIVI
FPIFAILLFWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVAS
NQKTIQPPRKAVEEPLNAFKESKGMMNDE

Accordingly, a proteinaceous particle may or may not target a cell comprising a polypeptide sequence substantially as set out in SEQ ID NO. 32 or a variant thereof or fragment thereof or orthologue thereof. Furthermore, the coding sequence, which encodes the CD47 polypeptide, may comprise a nucleic acid sequence substantially as set out in either SEQ ID NO. 31, or a variant thereof or fragment thereof or an orthologue thereof.

The shell of the proteinaceous particle of the invention may or may not bind to target cells that comprise the protein ICAM-1 (also known as intercellular adhesion molecule-1). ICAM-1 is a polypeptide that may act as receptor for a proteinaceous particle according to the invention. ICAM-1 expression is increased on many cells by cellular activation or inflammatory cytokines, which may render target cells more susceptible to killing by proteinaceous particles.

ICAM-1 protein encoded by the gene ICAM1. Thus, the genomic DNA sequence (introns and exons) encoding one embodiment of ICAM-1 is referred to herein as SEQ ID NO. 33 and can be found under the gene ID: 3383 (https://www.ncbi.nlm.nih.gov/gene?db=gene&Cmd=DetailsSearch&Term=3383).

The polypeptide sequence of ICAM-1 is provided herein as SEQ ID NO. 34, as follows:

[SEQ ID NO. 34]
MAPSSPRPALPALLVLLGALFPGPGNAQTSVSPSKVILPRGGSVLVTCS
TSCDQPKLLGIETPLPKKELLLPGNNRKVYELSNVQEDSQPMCYSNCPD
GQSTAKTFLTVYWTPERVELAPLPSWQPVGKNLTLRCQVEGGAPRANLT
VVLLRGEKELKREPAVGEPAEVTTTVLVRRDHHGANFSCRTELDLRPQG
LELFENTSAPYQLQTFVLPATPPQLVSPRVLEVDTQGTVVCSLDGLFPV
SEAQVHLALGDQRLNPTVTYGNDSFSAKASVSVTAEDEGTQRLTCAVIL
GNQSQETLQTVTIYSFPAPNVILTKPEVSEGTEVTVKCEAHPRAKVTLN
GVPAQPLGPRAQLLLKATPEDNGRSFSCSATLEVAGQLIHKNQTRELRV
LYGPRLDERDCPGNWTWPENSQQTPMCQAWGNPLPELKCLKDGTFPLPI
GESVTVTRDLEGTYLCRARSTQGEVTRKVTVNVLSPRYEIVIITVVAAA
VIMGTAGLSTYLYNRQRKIKKYRLQQAQKGTPMKPNTQATPP

Accordingly, a proteinaceous particle may or may not target a cell comprising a polypeptide sequence substantially as set out in SEQ ID NO. 34 or a variant thereof or fragment thereof or orthologue thereof.

The antibody may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR). The constant region consists of one of five heavy chain sequences (μ, γ, ξ, a, or ε) and one of two light chain sequences (κ or λ). The heavy chain constant region sequences determine the isotype of the antibody and the effector functions of the molecule.

In one embodiment, the antibody or antigen-binding fragment thereof comprises a polyclonal antibody, or an antigen-binding fragment thereof. The antibody or antigen-binding fragment thereof maybe generated in a rabbit, mouse or rat.

In another embodiment, the antibody or antigen-binding fragment thereof may comprise a monoclonal antibody or an antigen-binding fragment thereof. Preferably, the antibody is a human antibody. As used herein, the term “human antibody” can mean an antibody, such as a monoclonal antibody, which comprises substantially the same heavy and light chain CDR amino acid sequences as found in a particular human antibody exhibiting immunospecificity for an antigen, or a variant or fragment thereof. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody.

Substantially the same heavy and light chain CDR amino acid sequence can have, for example, minor modifications or conservative substitutions of amino acids. Such a human antibody maintains its function of selectively binding to an antigen or a variant or fragment thereof.

The term “human monoclonal antibody” can include a monoclonal antibody with substantially or entirely human CDR amino acid sequences produced, for example by recombinant methods such as production by a phage library, by lymphocytes or by hybridoma cells. The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen. Preferably, the fragment is an epitope. The binding region may be a hypervariable CDR or a functional portion thereof. The term “functional portion” of a CDR can mean a sequence within the CDR which shows specific affinity for the target antigen. The functional portion of a CDR may comprise a ligand which specifically binds to an antigen or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “(functional) fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)2; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-i. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about no amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain. The antibody fragment may alternatively comprise a Fab′2 fragment comprising the hinge portion of an antibody.

The term “F(ab) fragment” can mean a bivalent antigen-binding fragment of a human monoclonal antibody. An F(ab) fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity, e.g. target binding activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof may be produced using methods known to those skilled in the art. The polynucleotide encoding the antibody or antigen-binding fragment thereof may be directly synthesized by methods of oligonucleotide synthesis known in the art. Alternatively, smaller fragments maybe synthesized and joined to form a larger functional fragment using recombinant methods known in the art.

As used herein, the term “immunospecificity” can mean the binding region is capable of immunoreacting with an antigen, or a variant or fragment thereof, by specifically binding therewith.

The term “immunoreact” can mean the binding region is capable of eliciting an immune response upon binding with an antigen, or an epitope thereof.

The inventors have found that proteinaceous particles may be engineered so that they comprise proteins of interest. This was achieved by creating modified cells that transcribe specific RNA (e.g. mRNA or tRNA or miRNA) and/or express certain proteins, which in turn are incorporated into proteinaceous particles within the cells. Thus, a cell (e.g. a CD8 T cell/cytotoxic T cell or NK cell) may be genetically modified to comprise a nucleic acid sequence, which encodes a heterologous protein, such as a ligand, that is capable of being expressed on the shell of a proteinaceous particle, and which is also specific for a protein (e.g. a receptor) expressed on a target cell/tissue, so as to enable targeted delivery of the proteinaceous particle thereto.

Thus, according to another aspect of the invention, there is provided a modified cell capable of producing an engineered proteinaceous particle according to the invention, the modified cell comprising, or comprising nucleic acid encoding:
perform and/or granzyme;
thrombospondin-1 (TSP-1) or a fragment thereof, a variant thereof or an orthologue thereof; and
a heterologous polypeptide, such as a transgenic ligand in the form of a fusion protein with a thrombospondin, a galectin or a granzyme.

In an embodiment wherein the fusion protein comprises a thrombospondin and a heterologous polypeptide, the thrombospondin may comprise the TSP-1. In particular, the TSP-1 may be a fusion protein with a heterologous polypeptide, such as a ligand.

The cell may further comprise a shell protein selected from the group comprising galectin-1, galectin-7, TSP-4, a fragment thereof, a variant thereof or an orthologue thereof.

Cells that do not naturally produce the proteinaceous particle according to the invention may also be modified to produce the naturally occurring (i.e. non-engineered) proteinaceous particle.

Therefore, according to another aspect of the invention, there is provided a modified cell capable of producing a proteinaceous particle according to the invention, the modified cell comprising, or comprising nucleic acid encoding:
perform and/or granzyme;
thrombospondin-1 (TSP-1) or a fragment thereof, a variant thereof or an orthologue thereof.

The perform, granzyme and/or TSP-1 may be recombinant. The perform, granzyme and/or TSP-1 may be heterologous to the cell.

According to another aspect of the invention, there is provided a method of producing a modified cell capable of producing an engineered proteinaceous particle according to the invention, the method comprising introducing a nucleotide sequence encoding a fusion protein into a cell comprising or capable of expressing:
perform and/or granzyme; and
thrombospondin-1 (TSP-1) or a fragment thereof, a variant thereof or an orthologue thereof,
   in order to produce a modified cell that expresses the fusion protein encoded by the nucleotide sequence,
wherein the fusion protein comprises a thrombospondin, a galectin or a granzyme and a heterologous polypeptide, such as a transgenic ligand.

According to another aspect of the invention, there is provided a method of producing a modified cell capable of producing an engineered proteinaceous particle according to the invention, the method comprising providing a cell capable of producing a proteinaceous particle according to the invention, and introducing a nucleotide sequence encoding a fusion protein, wherein the fusion protein comprises a heterologous polypeptide, such as a transgenic ligand, and a thrombospondin, a galectin or a granzyme.

According to another aspect of the invention, there is provided a method of producing a modified cell capable of producing an engineered proteinaceous particle according to the invention, the method comprising introducing nucleotide sequences encoding:
a heterologous polypeptide, such as a transgenic ligand; and/or
perform and/or granzyme; and/or
thrombospondin-1 (TSP-1) or a fragment thereof, a variant thereof or an orthologue thereof,
   into the cell for expression therein, optionally wherein the heterologous polypeptide is encoded as a fusion protein comprising a thrombospondin, a galectin and/or granzyme.

According to another aspect of the invention, there is provided a method of producing a modified cell capable of producing a proteinaceous particle according to the invention, the method comprising introducing nucleotide sequences encoding:
perform and/or granzyme; and
thrombospondin-1 (TSP-1) or a fragment thereof, a variant thereof or an orthologue thereof,
   into the cell for expression therein.

The heterologous polypeptide, such as a transgenic ligand, may be encoded as a fusion protein with a thrombospondin and/or granzyme. In one embodiment, the heterologous polypeptide, such as a transgenic ligand, is encoded as a fusion protein with a thrombospondin. In another embodiment, the heterologous polypeptide, such as a transgenic ligand, is encoded as a fusion protein with a granzyme. The fusion protein with a thrombospondin may be a fusion protein of the heterologous polypeptide, such as a transgenic ligand, with TSP-1.

In embodiments of the invention where a fusion polypeptide/protein is provided, the heterologous polypeptide (such as a transgenic peptide) may be C-terminal to its fusion partner. For example the thrombospondin may be N-terminal to the heterologous polypeptide (such as a transgenic peptide). The galectin may be N-terminal to the heterologous polypeptide (such as a transgenic peptide). The granzyme may be N-terminal to the heterologous polypeptide (such as a transgenic peptide).

According to another aspect of the invention, there is provided a modified cell, wherein the modified cell comprises nucleic acid encoding the components of the engineered proteinaceous particle according to the invention.

According to another aspect of the invention, there is provided a modified cell, wherein the modified cell comprises nucleic acid encoding the components of the proteinaceous particle according to the invention.

The nucleotide sequence(s) introduced into the cell may comprise DNA. In one embodiment the nucleotide sequence(s) introduced into the cell are provided in the form of a vector for transfection into the cell. nucleotide sequence(s) introduced into the cell may be stably transformed (e.g. chromosomally integrated) into the cell. In an embodiment wherein the nucleotide sequence introduced into the cell is a fusion protein with a thrombospondin, galectin or granzyme, the nucleotide sequence may replace or knockout (e.g. by insertion into) any existing sequence of the thrombospondin, galectin or granzyme respectively. In particular, existing nucleotide sequences (genes) encoding wild-type thrombospondin, galectin or granzyme may be replaced or knocked out (e.g. by insertion) with a fusion protein equivalent, wherein the fusion protein is a heterologous polypeptide. The insertion of nucleotide sequence(s) may comprise the use of homologous recombination, for example by providing sequences that are homologous to the insert site flanking the nucleotide sequence(s) to be inserted. The skilled person will be familiar with a number of techniques and methods to transform cells with nucleotide sequences, for their expression in a cell.

A ligand refers to an agent or moiety that (specifically) binds to a protein (e.g. receptor or ion channel) or marker on a target cell. Preferably, the ligand binds specifically to the protein or marker. A ligand may be a protein or a peptide. The ligand may be a transgenic ligand (e.g. a transgenic polypeptide). The transgenic ligand may be an antibody, or antibody fragment (e.g. scFv) or a fusion protein. The ligand may be chlorotoxin or T1-scFv.

The cell may be a T cell (T lymphocyte), a CD3+ cell, a CD8+ cell or a Natural Killer (NK) cell. Preferably the cell is a CD8+ T cell (cytotoxic T cell or a CD3+CD8+ cell). The cell may be a CD57+ cell. Most preferably, the cell is a CD3+CD8+CD57+ T cell. The cell may be an activated CD3+ cell, an activated CD8+ cell or an activated Natural Killer (NK) cell. Most preferably the cell is an activated CD3+CD8+ T cell, or an activated CD3+CD8+CD57+ T cell. The cell may be a cell that comprises proteinaceous particles. The cell may be a human embryonic kidney (HEK) cell, a Chinese hamster ovary (CHO) cell, Natural killer-like cell lines including NK92 and YT. The cell may be a cell capable of producing or that comprises a proteinaceous particle according to the invention.

The nucleotide sequence may encode a heterologous ligand, such as a transgenic ligand. Thus, the nucleotide sequence may encode the amino acid sequence of one or more of SEQ ID NOS. 28 to 31.

Preferably the method is used to create a modified cell according to the invention.

Proteinaceous particles according to the invention are of a similar size to exosomes. Consequently, they typically co-purify with exosomes from the supernatants of NK cells and T cells. The inventors have therefore developed a method to isolate and purify proteinaceous particle according to the invention.

According to another aspect of the invention, there is provided a method of isolating a proteinaceous particle according to the invention from cells, the method comprising:
(i) providing the cell in a liquid;
(ii) centrifuging the cell and liquid in order to pellet the cell, or filtering out the cell, thereby forming a cell-fee liquid;
(iii) collecting released proteinaceous particles by centrifuging or filtering the cell-free liquid to collect the proteinaceous particles,
wherein any exosomes released from the cell are depleted before or after centrifuging or filtering the cell-free liquid to collect the proteinaceous particles.

Cells that produce proteinaceous particles of the invention may also produce exosomes. However, the exosomes can co-purify at the same centrifugal forces, or in the same filter as the proteinaceous particles of the invention. Therefore, depletion of the exosomes may be necessary for a substantially pure or purer collection of the proteinaceous particles. The depletion of the exosomes after centrifugation or filtering of the proteinaceous particles for their collection can advantageously increase the concentration of any exosomes, which can make the depletion, such as immunodepletion, more efficient and convenient.

The proteinaceous particle may be natural/wild type proteinaceous particle according to the invention or engineered proteinaceous particle according to the invention.

The cell may be a cell that is capable of producing the proteinaceous particle according to the invention or the engineered proteinaceous particle according to the invention. The cell may be an engineered cell according to the invention, which has been modified to produce a natural or engineered proteinaceous particle. The cell may be a T cell (T lymphocyte), a CD3+ cell, a CD8+ cell or a Natural Killer (NK) cell. The cell may be a CD57+ cell. Most preferably, the cell is a CD3+CD8+CD57+ T cell. The cell may be an activated CD3+ cell, an activated CD8+ cell or an activated Natural Killer (NK) cell. Most preferably the cell is an activated CD3+CD8+ T cell, or an activated CD3+CD8+CD57+ T cell. The cell may be a human embryonic kidney (HEK) cell, a Chinese hamster ovary (CHO) cell, Natural killer-like cell lines including NK92 and YT. The cell may be cells that comprise or express a proteinaceous particle according to the invention.

The cell may spontaneously release proteinaceous particles. However, the method according to the invention may comprise the step of activating the cells to increase the release of the proteinaceous particle. The cells may be activated using any techniques known in the art. However, the skilled person will appreciated that the way in which the cells are activated will depend on the type of cells. For example, a CD3+ cell may be activated by an anti-CD3 antibody, optionally with an anti-CD28 antibody and/or Fas. An NK cell may be activated by an anti-CD16 antibody.

The liquid may be media, such as cell culture media. Preferably step (i) comprises providing the cells in a culture media. The composition of the media will be controlled so that it is free of exosomes and other particles of similar size to proteinaceous particle. The medium may be a fully defined formulation with low protein to facilitate proteinaceous particle purification.

Step (ii) comprises centrifuging the cells in the liquid (e.g. the culture media) to create a centrifuged cell-free liquid. Centrifuging the cells (e.g. culture media) may comprise spinning at a speed sufficient to pellet the cells within the liquid, such that they can be separated from proteinaceous particle and exosomes within the supernatant. The centrifugation to pellet the cells may be at 100-1000 g. After cells are gently removed, the supernatant may be subjected to an additional 10,000 g centrifugation to remove subcellular particles, which have been pelleted because they are >500 nm. Alternatively, step (ii) may comprise the filtering out of cells from the liquid. For example the cells may be filtered by passing the liquid through a filter having a pore size that prevents the passage of cells, but not the proteinaceous particles, or impedes the passage of cells greater than the proteinaceous particles, such they can be fractionated. More specifically, the cells may be filtered out from the liquid by culturing them in a hollow fibre cell culture system with pores large enough that proteinaceous particles can pass through, but small enough that cells cannot pass though, such that the proteinaceous particles are collected in the filtrate of the hollow fibre cell culture system. The pore size would be about 0.45 μm, preferably greater than about 0.2 μm in diameter but less than about 1 μm in diameter.

Centrifuging the cell-free liquid to collect released proteinaceous particles may comprise centrifugation to pellet the proteinaceous particles. Such a pellet may be subsequently resuspended, for example in a buffer or other media, after the cell-free liquid has been discarded. Centrifuging the cell-free liquid to collect/pellet released proteinaceous particles may comprise ultracentrifugation. The ultracentrifugation may be at sufficient speed and time to pellet the proteinaceous particles according to the invention. For example, the ultracentrifugation may be sufficient to pellet proteinaceous particle of between 50 and 100 nm in size. In one embodiment, the ultracentrifugation may be at about 25,000 g to about 400,000 g, or about 50,000 g to about 200,000 g. Most preferably the ultracentrifugation is at 100,000 g. In one embodiment the ultracentrifugation is at least 25,000 g.

The ultracentrifugation may be for at least about 15 minutes, at least about 30 minutes, at least about 1 hour, at least about 2 hours. The ultracentrifugation may be for about 15 minutes to about 4 hours, about 30 minutes to about 2 hours, or at least about 1 hour.

In one embodiment, the ultracentrifugation is for about 30 minutes to 2 hours at about 50,000 g to about 200,000 g. Most preferably the ultracentrifugation is for at least about 1 hour at 100,000 g.

In one embodiment, step (ii) (i.e. filtering the cells to form a cell free liquid) comprises ultrafiltration. In one embodiment, step (iii) (i.e. filtering the cell-free liquid to collect released proteinaceous particles) comprises gel filtration, such that the proteinaceous particles are separated into a fraction that is free of smaller components (i.e. components less than about 80 nm in diameter). In another embodiment, step (ii) comprises ultrafiltration and step (iii) comprises gel filtration.

Ultrafiltration involves filtering the cell-free liquid to collect released proteinaceous particles may comprise filtering the proteinaceous particles to entrap them on the filter. For example, the pores of the filter may be sized to allow the passage of liquid and molecules smaller than the proteinaceous particles, but prevent passage of the proteinaceous particles. For example, the pores may be less than 50 nm in diameter. Filtering the cell-free liquid to collect released proteinaceous particles may comprise the use of size exclusion chromatography. In another embodiment, a combined bind-elute and size exclusion chromatography may be used. The skilled person will be familiar with filtration techniques for isolating proteinaceous particles, for example based on their size, charge, and/or binding properties. Such methods are described for isolating exosomes of similar size by Corso et al. (2017 Scientific Reports 17: 11561| DOI:10.1038/s41598-017-10646-x 3, which is herein incorporated by reference) and Vader et al. (2017. Andrew F. Hill (ed.), Exosomes and Microvesicles: Methods and Protocols. Methods in Molecular Biology, vol. 1545, DOI 10.1007/978-1-4939-6728-5_14), which may be applied to the proteinaceous particles of the present invention.

For example such techniques may use liquid chromatography, such as core bead chromatography.

The exosomes may be depleted by using any technique known in the art. The skilled person will appreciate that there are a variety of techniques that can be used to deplete exosomes, for example in centrifuged media. In one embodiment, the exosomes are immunodepleted. Preferably the exosomes are depleted using antibodies raised against exosome markers, such as CD81, CD63 and/or CD9. The exosomes may be depleted using magnetic beads coated in antibodies immunospecific for exosome markers, such as CD81, CD63 and/or CD9. As exosomes are membrane based, they can also be destroyed by mild, non-ionic detergents that are non-destructive to proteinaceous particles and easy to remove (e.g. octyl-β-D glucopyranoside). Therefore, in one embodiment, exosomes are depleted by disrupting (i.e. breaking) the membrane of the exosomes with a detergent. In one embodiment the detergent comprises or consist of octyl-β-D glucopyranoside. The skilled person will readily identify alternative detergents that can disrupt exosome membranes, but will not affect (e.g. denature) proteinaceous particles.

The method according to the invention may further comprise centrifuging the exosome depleted liquid to pellet the proteinaceous particle, for example for collection. The exosome depleted liquid may be spun at sufficient speed and time to pellet the proteinaceous particle according to the invention. For example, the centrifugation may be sufficient to pellet proteinaceous particle of between 50 and 100 nm in size. In one embodiment, the exosome depleted liquid may be spun at about 25,000 g to about 400,000 g, or about 50,000 g to about 200,000 g. Most preferably the liquid is spun at 100,000 g. In one embodiment the exosome depleted liquid may be centrifuged at least at 25,000 g.

The exosome depleted liquid may be centrifuged (spun) for at least about 15 minutes, at least about 30 minutes, at least about 1 hour, at least about 2 hours. The exosome depleted liquid may be spun for about 15 minutes to about 4 hours, about 30 minutes to about 2 hours, or at least about 1 hour.

In one embodiment, the exosome depleted liquid is spun for about 30 minutes to 2 hours at about 50,000 g to about 200,000 g. Most preferably the exosome depleted liquid is spun for at least about 1 hour at 100,000 g.

Prior to step (i) the cell (e.g. an activated CD3+CD8+ T cell) may be cultured in culture media for at least about 6 hours, at least about 12 hours, at least about 24 hours, at least about 48 hours. The cell may be cultured in culture media for about 6 hours to 192 hours, or about 12 hours to 96 hours, or about 24 hours to 48 hours.

The inventors have developed an alternative method of isolating a proteinaceous particle according to the invention.

Thus, according to another aspect of the invention, there is provided a method of isolating proteinaceous particle according to the invention from cells, the method comprising:
(a) adhering the cells to a substrate, whereby the proteinaceous particle released from the cells also adheres to the substrate;
(b) unadhering the cells from the substrate, to leave adhered proteinaceous particle; and
(c) collecting the proteinaceous particle by eluting the proteinaceous particle from the substrate.

Advantageously, it has been found that cells adhered to substrate such as a lipid bilayer can be activated and release the proteinaceous particle according to the invention which can adhere to the substrate, such a lipid bilayer. The adhered proteinaceous particle can then be collected. This process has a benefit of being capable of quickly producing and isolating the desired proteinaceous particle, for example in hours (less than a day).

### Step (a) of Adhering the Cell to a Substrate

This step may comprise contacting the cell with a substrate. The cell may be a cell as referred to in the previous aspect (i.e. the previous method of isolating a proteinaceous particle from a cell).

The substrate may be a surface to which cells (e.g. T cell or NK cells) can be adhered and unadhered. The substrate may be a model lipid bilayer, such as a supported lipid bilayer (SLB), or a glass surface, preferably a planar glass surface, or a glass bead so that an SLB can be formed on the glass bead.

The substrate may be coated with one or more, two or more, or three or more proteins for cell adhesion and/or activation, such as ICAM-1 and MICA. Preferably the substrate (e.g. an SLB or separation beads) is coated with ICAM-1 and MICA when the cell is an NK cell. Preferably the substrate (e.g. SLB) is coated with ICAM-1 when the cell is a T cell (T lymphocyte), a CD3+ cell or a CD8+ cell. The substrate (e.g. an SLB or separation beads) may be coated with CD47. The substrate (e.g. an SLB or separation beads) may be coated with CD47, ICAM-1 and MICA, or may be coated with CD47 and ICAM-1.

The substrate (e.g. SLB) may further be coated with one, two, three or more cell activating agents, such as anti-CD16 (for NK cells) and/or anti-CD3 (for T cells), so that the substrate is activatory. The cell activating agents promote the exocytosis of proteinaceous particle. Preferably the activatory substrate (e.g. SLB) is coated in ICAM-1 and anti-CD3 (for T cells). The activatory substrate may further comprise anti-CD28. The activatory substrate may further comprise Fas receptor, such that the core and/or a hybrid particle comprise(s) FasL. Thus, the activatory substrate for a T cell may comprise ICAM-1 and anti-CD3, and/or Fas receptor. Preferably the activatory substrate (e.g. SLB) comprises ICAM-1, MICA and anti-CD16 (for NK cells). More preferably the activatory substrate is a lipid bilayer surface comprising ICAM-1, MICA and anti-CD16 (for activating NK cells) or CD3 (for activating T cells). The activatory substrate may be further coated with CD58 to improve activation of T cells and/or NK cells. CD58 binds to the adhesion molecule and may increase activation of T cells and/or NK cells and promote the release of proteinaceous particles.

The step of adhering the cell to a substrate may be for at least about at 20 minutes, at least about 30 minutes, at least about 45 minutes, at least about at 60 minutes or at least about 90 minutes. The step of adhering the cell to a substrate may be for about 20 minutes to about 4 hours, for about 30 minutes to about 3 hours, for about 45 minutes to about 3 hours, for about 60 minutes to about 2 hours or for about 90 minutes. Preferably the step of adhering the cell to a substrate is performed for about 90 minutes.

The step of adhering the cell to a substrate may be at at least about 20° C., at least about 30° C., at least about 35° C. or at least about 37° C. Preferably the step of adhering the cell to a substrate is performed at about 37° C.

In one embodiment, the step of adhering the cell to a substrate is performed for at least about 60 minutes (e.g. for about 60 minutes to about 2 hours or for about 90 minutes) at about 37° C. or at least about 90 minutes at about 37° C.

Preferably step of adhering the cell (and subsequently the proteinaceous particle) to a substrate is performed at a pH of about 6.5-7.5.

Step (b) of Unadhering the Cell from the Substrate

The cell may be unadhered from the substrate by washing. The proteinaceous particles may remain adhered to the surface, for example bound to ICAM-1 and/CD47. The washing step may comprise a shock and mechanical flush mechanism to release the cells, which the skilled person will be familiar with. Washing may be performed with a buffer, such as phosphate-buffered saline (PBS), preferably cold PBS. Cold PBS may be PBS at a temperature of less than about 15° C., less than about 14° C., less than about 13° C., less than about 12° C., less than about 11° C., less than about 10° C., less than about 9° C., less than about 8° C., less than about 7° C., less than about 6° C., less than about 5° C., less than about 4° C., less than about 3° C., less than about 2° C. or less than about 1° C. Preferably the PBS is less than about 4° C.

Step (c) of Eluting the Proteinaceous Particles from the Substrate

The step of eluting the proteinaceous particles from the substrate may comprise washing the substrate with a solvent to obtain an eluate of the proteinaceous particle. The solvent may comprise an agent capable of freeing the proteinaceous particles from the substrate surface. In one embodiment, the substrate surface is treated with imidazole. Chelating agents may also be used to release the proteinaceous particle from the substrate surface. The chelating agent may be an agent that chelates Ca²⁺. Thus, the chelating agent may be EDTA. Additionally or alternatively, the step of eluting the proteinaceous particles from the substrate (e.g. separation beads) may comprise a change in pH. For example the pH may be increased to less than about pH 5.5, less than about pH 5, less than about pH 4.5, less than about pH 4, less than about pH 3.5 or less than about pH 3 to elute the proteinaceous particle from the substrate. Preferably, pH is increased to between about pH 5.5 and about pH 3.

Advantageously, imidazole is capable of releasing ICAM-1 from the substrate surface, which is retaining the proteinaceous particles to be eluted. Co-eluted ICAM-1 can then be separated from the proteinaceous particles by ultracentrifugation or gel filtration. Even though ICAM-1 binds to TSP-1 on the proteinaceous particles, the affinity is low (Kd>1 μM) and the vast majority of ICAM-1 will not be bound to the TSP-1 at concentrations of ICAM-1 present in the system (<10 nM).

The step of eluting the proteinaceous particles from the substrate may comprise washing the substrate, for example with an agent capable of freeing the proteinaceous particle (e.g. imidazole), for at least about 5 minutes, at least about 10 minutes, at least about 10 minutes, at least about 15 minutes, at least about 20 minutes, at least about 25 minutes, at least about 30 minutes, at least about 35 minutes, at least about 40 minutes or at least about 45 minutes.

The step of eluting the proteinaceous particles from the substrate may comprise washing the substrate, for example with an agent capable of freeing the proteinaceous particle (e.g. imidazole), for no more than about 5 minutes, no more than about 10 minutes, no more than about 10 minutes, no more than about 15 minutes, no more than about 20 minutes, no more than about 25 minutes, no more than about 30 minutes, no more than about 35 minutes, no more than 40 minutes, or no more than 45 minutes. Preferably the step of eluting the proteinaceous particles from the substrate comprises washing the substrate, for example with an agent capable of freeing the proteinaceous particle (e.g. imidazole), for about 10, 20 or 30 minutes.

The step of eluting the proteinaceous particles from the substrate may be followed by a step of centrifuging the eluate and/or depleting the eluate. Centrifuging may comprise ultracentrifugation. The eluate may be spun (centrifuged) for at least about 15 minutes, at least about 30 minutes, at least about 1 hour, at least about 2 hours. The eluate may be spun for about 15 minutes to about 4 hours, about 30 minutes to about 2 hours, or about 1 hour.

Prior to step (a) the cells (e.g. an activated CD3+CD8+ T cell) may be cultured in culture media for at least about 6 hours, at least about 12 hours, at least about 24 hours, at least about 48 hours. The cell may be cultured in culture media for about 6 hours to 192 hours, or about 12 hours to 96 hours, or about 24 hours to 48 hours.

The isolation of the proteinaceous particles of the invention by one of these methods may increase their ability to kill cancer cells, infected cells or bacteria (without further engineering). The method according to the invention may be used to produce a proteinaceous particle with a purity ranging from about 10% to about 100%. Thus, the method according to the invention may be used to produce a proteinaceous particle that is about 10% to about 100% pure, about 20% to about 100% pure, about 30% to about 100% pure, about 40% to about 100% pure, about 50% to about 100% pure, about 60% to about 100% pure, about 70% to about 100% pure, about 80% to about 100% pure or about 90% to about 100% pure. In one embodiment, the method may be used to produce a proteinaceous particle that is at least about 90% pure or at least about 95% pure. Preferably the method according to the invention is used to produce a proteinaceous particle that is substantially pure. However, in some embodiments, minor fractions of impurities such as exosomes may be present in a composition of proteinaceous particles. There may be less than 30% exosomes present. Preferably there are less than 20% or more preferably less than 10% exosomes present. The isolated proteinaceous particle(s) may be free from cells. Thus, a proteinaceous particle that has been isolated/purified using a method according to the invention may be used in therapy.

References to isolation and production of the proteinaceous particle according to the invention may also refer to isolation and production of the hybrid particle, for example from CD8+ cells. Purification of hybrid particle, which comprises vesicle/phospholipid particle containing FasL, would not comprise immunodepletion using anti-CD81, anti-CD63 or anti-CD9.

According to another aspect, there is provided a composition comprising a proteinaceous particle of the invention, optionally wherein the composition is a pharmaceutical composition.

According to another aspect, there is provided a kit comprising a cell according to the invention and a substrate.

According to another aspect, there is provided a proteinaceous particle according to the invention or a composition according to the invention for use as a medicament.

According to another aspect, there is provided a proteinaceous particle according to the invention or a composition according to the invention for use in treatment of a disease or a condition of a subject.

According to another aspect, there is provided a proteinaceous particle according to the invention or a composition according to the invention for use in treating cancer.

The cancer may be a cancer selected from the group comprising renal cancer, bladder cancer, ovarian cancer, breast cancer, endometrial cancer, pancreatic cancer, lymphoma, thyroid cancer, bone cancer, CNS cancer, leukaemia, liver cancer, prostate cancer, lung cancer, oesophageal cancer, colon cancer, rectal cancer, brain cancer (e.g. glioblastoma) or melanoma.

According to another aspect, there is provided an engineered proteinaceous particle according to the invention or a composition according to the invention for use in targeted cell killing in a subject.

The proteinaceous particle of the composition, or the proteinaceous particle for use according to the invention may be isolated by a method according to the invention.

According to another aspect, there is provided a method of treating cancer, the method comprising administering the proteinaceous particle according to the invention or a composition according to the invention to a subject.

According to another aspect, there is provided a method of targeted cell killing, the method comprising administering the engineered proteinaceous particle according to the invention or a composition according to the invention to a subject.

It will be appreciated that the term “treatment” and “treating” as used herein means the management and care of a subject for the purpose of combating a condition, such as a disease or a disorder. The term is intended to include the full spectrum of treatments for a given condition from which the subject is suffering, including alleviating symptoms or complications, delaying the progression of the disease, disorder or condition, alleviating or relieving the symptoms and complications, and/or to cure or eliminating the disease, disorder or condition as well as to prevent the condition, wherein prevention is to be understood as the management and care of a subject for the purpose of combating the disease, condition, or disorder and includes the administration of the ligand to prevent the onset of the symptoms or complications.

The subject to be treated is preferably a mammal, in particular a human, but it may also include animals, such as dogs, cats, horses, cows, sheep and pigs.

Pharmaceutical compositions according to the invention may further comprise a pharmaceutically acceptable salt or other form thereof. Pharmaceutical compositions according to the invention may comprise one or more pharmaceutically acceptable excipients, such as carriers, diluents, fillers, disintegrants, lubricating agents, binders, colorants, pigments, stabilizers, preservatives, antioxidants, and/or solubility enhancers. Pharmaceutical compositions according to the invention may comprise pharmaceutically acceptable salt and one or more pharmaceutically acceptable excipients.

The pharmaceutical compositions can be formulated by techniques known in the art. The pharmaceutical compositions can be formulated as dosage forms for oral, parenteral, such as intramuscular, intravenous, subcutaneous, intradermal, intraarterial, intracardial, nasal or aerosol administration. The pharmaceutical composition may be formulated as a dosage form for oral administration.

Exposure to the cytotoxic proteinaceous particles according to the invention may cause release of IGFBP-3 from the cells. In one embodiment, IGFBP-3 may be used as a marker of cells exposed to the cytotoxic proteinaceous particles according to the invention. Therefore, following contact or administration with proteinaceous particles according to the invention, the presence and/or level of IGFBP-3 produced by the cells may be determined.

The term “isolated” can refer to biological material that has been isolated from its natural environment, preferably be means of a technical process. The term isolated may comprise isolated from the extracellular excretions of a cell, i.e. a producing cell.

The term “genetically modified” can refer to a biological molecule or cell that has an altered nucleotide (e.g. protein) and/or amino acid sequence so that the molecule or cell is not found naturally in nature.

The adjective “transgenic” can refer to an organism, tissue or cell comprising genetic information from another organism. Thus, a transgenic nucleotide sequence refers to a nucleotide sequence that has been transferred from one organism to a cell, tissue or organism of the invention. Similarly, a transgenic ligand refers to a ligand whose nucleotide sequence has been transferred from one organism to a cell, tissue or organism of the invention.

The term “orthologue” may refer to a gene that has diverged from another due to speciation (i.e. when a population becomes distinct species).

A nucleotide sequence within the genetic construct of the invention may be DNA (such as cDNA) or RNA (such as mRNA). Preferably, the first and second nucleotide sequences referred to herein are the same type of nucleotide sequence, for example, both DNA or both RNA.

The term “comprising” is an open term, which refers to all of the features following the term but is not limited to those features only. However, the term “comprising” also encompasses the term “consisting of”, which is a closed term, and “consisting essentially of”. “Consisting of” refers to all of the features following the term and is limited to those features only. “Consisting essentially of” refers to all of the features following the term but also may include features not explicitly recited, which do not materially affect the essential characteristics of the invention. Thus, the term “comprising” may refer to “consisting of” or “consisting essentially of”.

The term a proteinaceous particle may refer to a hybrid particle.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the nucleic acids or polypeptides described herein. Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein. The amino acid/polynucleotide/polypeptide sequence may have 100% identity with any of the sequences referred to herein.

Where reference is made to a variant polypeptide or nucleotide sequence, the skilled person will understand that one or more amino acid residue or nucleotide substitutions, deletions or additions, may be tolerated, optionally two substitutions may be tolerated in the sequence, such that it maintains its function. The skilled person will appreciate that 1, 2, 3, 4, 5 or more amino acid residues or nucleotides may be substituted, added or removed without affecting function References to sequence identity may be determined by BLAST sequence alignment (www.ncbi.nlm.nih.gov/BLAST/) using standard/default parameters. For example, the sequence may have at least 99% identity and still function according to the invention. In other embodiments, the sequence may have at least 98% identity and still function according to the invention. In another embodiment, the sequence may have at least 95% identity and still function according to the invention. In another embodiment, the sequence may have at least 90%, 85%, or 80% identity and still function according to the invention. In one embodiment, the variation and sequence identity may be according the full length sequence. In other embodiments, the variation may be limited to non-conserved sequences and/or sequences outside of active sites, such as binding domains. Therefore, an active site or binding site of a protein may be 100% identical, whereas the flanking sequences may comprise the stated variations in identity. Such variants may be termed “conserved active site variants”.

Amino acid substitutions may be conservative substitutions. For example, a modified residue may comprise substantially similar properties as the wild-type substituted residue. For example, a substituted residue may comprise substantially similar or equal charge or hydrophobicity as the wild-type substituted residue. For example, a substituted residue may comprise substantially similar molecular weight or steric bulk as the wild-type substituted residue. With reference to “variant” nucleic acid sequences, the skilled person will appreciate that 1, 2, 3, 4, 5 or more codons may be substituted, added or removed without affecting function. For example, conservative substitutions may be considered.

Preferably the term “fragment” refers to a “functional fragment”. A functional fragment may refer to a fragment that has amino acids/nucleotides essential for performing a function of the full length fragment/polypeptide.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local versus global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: —Sequence Identity=(N/T)*100. Alternative methods for identifying similar sequences will be known to those skilled in the art. For example, a substantially similar nucleotide sequence will be encoded by a sequence which hybridizes to any sequences referred to herein or their complements under stringent conditions. By stringent conditions, we mean the nucleotide hybridises to filter-bound DNA or RNA in 3× sodium chloride/sodium citrate (SSC) at approximately 45° C. followed by at least one wash in o.2×SSC/0.1% SDS at approximately 20-65° C. Alternatively, a substantially similar polypeptide may differ by at least 1, but less than 5, 10, 20, 50 or 100 amino acids from the polypeptide sequences described herein.

Due to the degeneracy of the genetic code, it is clear that any nucleic acid sequence described herein could be varied or changed without substantially affecting the sequence of the protein encoded thereby, to provide a variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

Where a reference to a polypeptide sequence refers to a sequence comprising a precursor or propeptide sequence, the skilled person will recognise that, in some embodiments, reference to the sequence may refer only to the mature polypeptide. For example, the precursor residues and signal peptide may not be part of the mature polypeptide that is in the proteinaceous particle according to the invention. Accordingly, reference to variants of such sequences, may only refer to the mature polypeptide part of the given sequence.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the invention, and to show embodiments of the invention may be put into effect, reference will now be made, by way of example, to the accompanying drawings, in which:

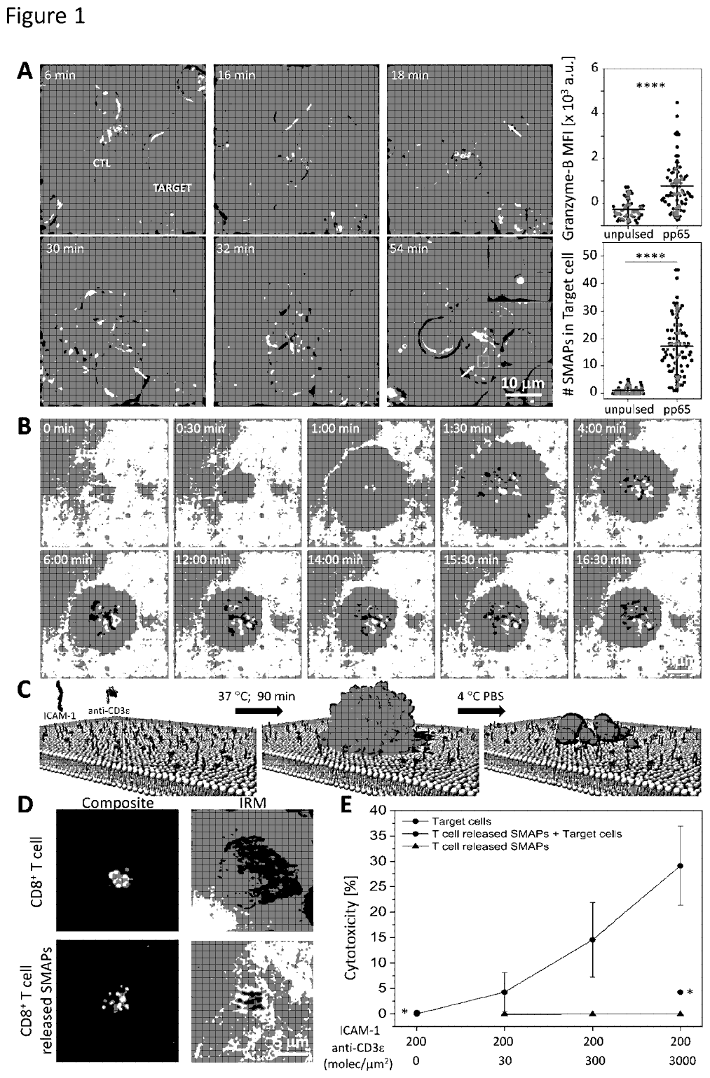

FIG. 1 shows SMAPs were released at the IS and displayed autonomous cytotoxicity. (A) Time-lapse confocal images depicting the transfer of Gzmb-mCherry® (green) and WGA (magenta) labeled SMAPs from an antigen-specific CTL clone into pp65-pulsed JY target cells (Target). Arrows and inset indicate the presence of SMAPs inside the target. Scale bar, 10 μm. Quantification of Gzmb mean fluorescence intensity (MFI) and number of double-positive particles inside the target cell in CTL conjugates with unpulsed or pulsed target cells. Each dot represents one target cell (<50 cells). Horizontal lines and error bars represent mean±SD from 2 independent experiments. ****, p<0.0001 (B) Live cell imaging of SMAPs release by CD8⁺ T-cells transfected with Gzmb-mCherry-SEpHluorin (magenta/green) on activating SLB. IRM, interference reflection microscopy. Scale bar, 5 μm. (C) Schematic of the working model for capturing SMAPs released by activated CD8⁺ T-cells. CD8⁺ T-cells (grey) were incubated on SLB presenting activating ligands for the indicated time. Cells were removed with cold PBS leaving the released SMAPs (purple) on the SLB. Elements are not drawn to scale. (D) TIRFM images of CD8⁺ T-cells incubated on activating SLB in the presence of anti-Prf1 (green) and anti-Gzmb (magenta) antibodies (top panels). After cell removal, Prf1⁺ and Gzmb⁺ SMAPs remained on the SLB (bottom panels). The formation of a mature IS is indicated by an ICAM-1 ring (blue). IRM, interference reflection microscopy. Scale bar, 5 μm. (E) Target cell cytotoxicity induced by density-dependent release of SMAPs captured on SLB measured by LDH release assay. Data points and error bars represent mean SEM from 3 independent experiments.

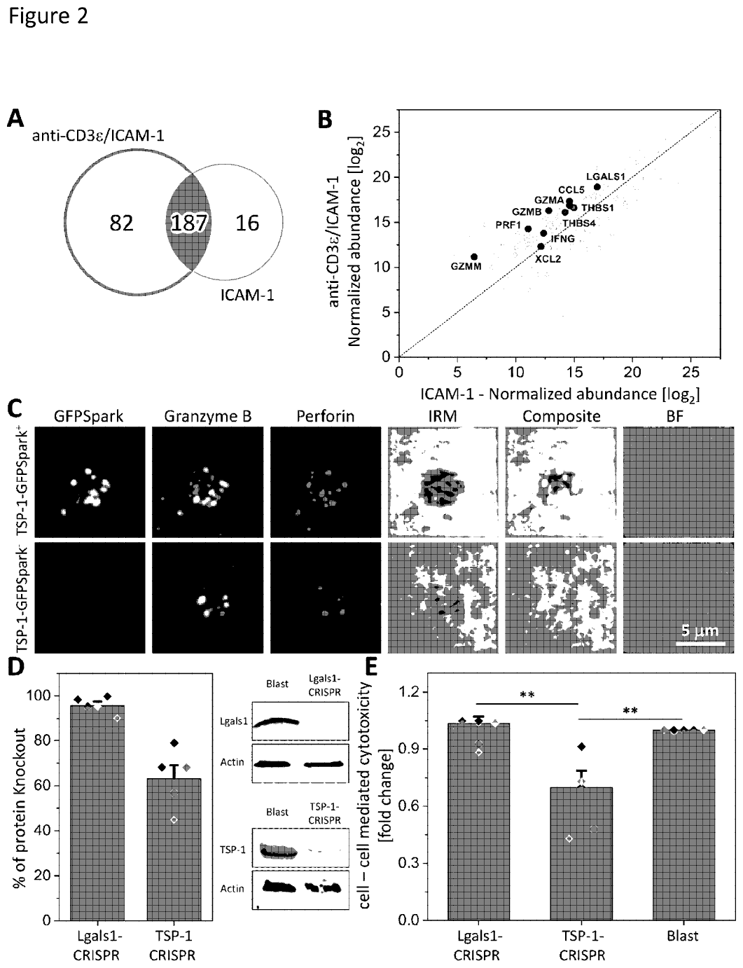

FIG. 2 shows TSP-1 is a major constituent of SMAPs and contributed to CTL killing of targets. (A) Two-set Venn diagram showing the number of individual and common proteins identified by MS analysis of material released by CD8⁺ T-cells incubated on non-activating (ICAM-1) or activating (ICAM-1+anti-CD3ε) SLB. Representative of 3 independent experiments with 8 donors. (B) Normalized abundance of the 285 proteins identified by MS in each condition. Cytotoxic proteins are highlighted in red (GZMM, PRF1, GZMB, GZMA), chemokine/cytokines in blue (CCL5, IFNG, XCL2) and adhesion proteins in green LGALS1, THBS1, THBS4). (C) TIRFM images of SMAPs released from CD8⁺ T-cells transfected with TSP-1-GFPSpark (green; top row) or non-transfected cells (bottom row). Released SMAPs were further stained with anti-Gzmb (yellow) and anti-Prf1 (magenta) antibodies. IRM, interference reflection microscopy. BF, bright field microscopy. Scale bar, 5 μm. (D) Percentage of galectin-1 and TSP-1 knockout in CD8⁺ T-cells by CRISPR/Cas9 genome editing measured from immuno-blotting analysis (left). Each colored dot represents one donor. Bars represent mean±SEM. Representative immuno-blot for galectin-1 (Lgals1) and TSP-1 in Lgals1 and TSP-1, respectively edited CD8⁺ T-cells (right). CD8⁺ T-cells (Blast) were analyzed in parallel as a control. (E) Target cell cytotoxicity mediated by galectin-1 (Lgals1-CRISPR) or TSP-1 (TSP-I-CRISPR) gene edited CD8⁺ T-cells measured by LDH release assay. T cell blasts were used as a control. Bars represent mean±SEM. **, p<0.01. Donors are the same as in (D).

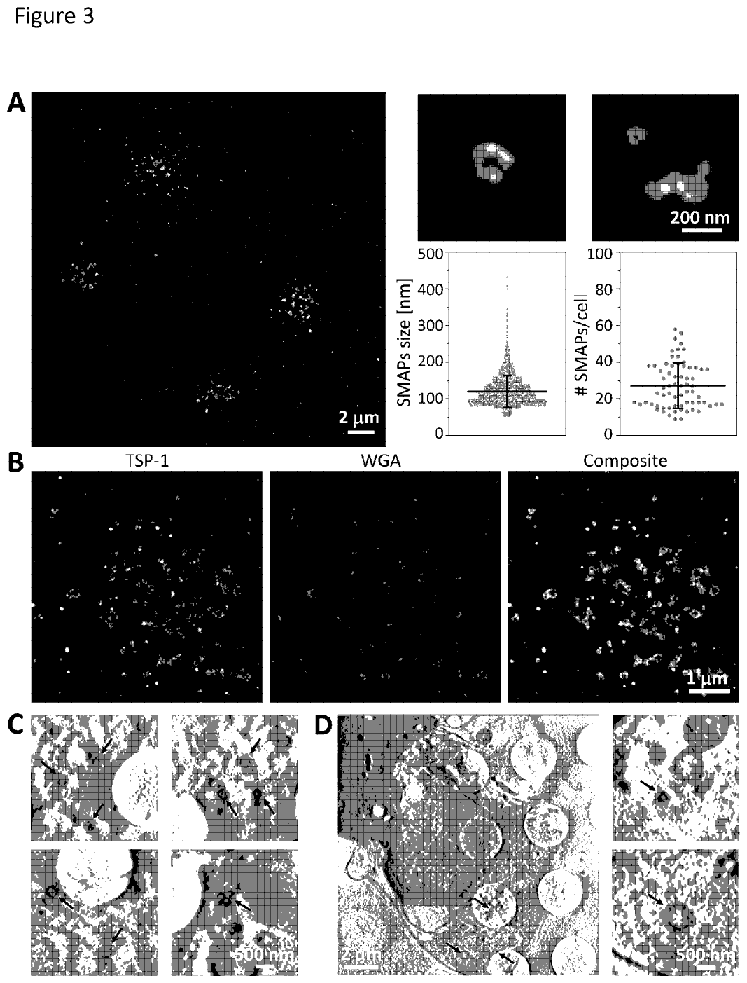

FIG. 3 shows that SMAPs shell was rich in glycoproteins, TSP-1 and organic material. (A) dSTORM image of SMAPs released on activating SLB by multiple cells (left; scale bar, 2 μm) and two examples of individual SMAPs (top right; scale bar, 200 nm), showing their heterogeneity in size. SMAPs were labeled with WGA. Quantification of SMAPs size and number released per cell (bottom right; n>1800 and n=67, respectively). Horizontal lines and error bars represent mean±SD from five donors. (B) dSTORM images of SMAPs (labeled with WGA, magenta) positive for TSP-1 (green) released on activating SLB. Scale bar, 1 μm. (C) Multiple CSXT examples of released SMAPs after cell removal. Scale bar, 500 nm. (D) CSXT of CD8⁺ T-cells interacting with carbon coated EM grids (note grid holes in C and D) containing ICAM-1 and anti-CD3ε. Scale bar, 2 μm or 500 nm for zoomed in regions (right). Arrows indicate SMAPs.

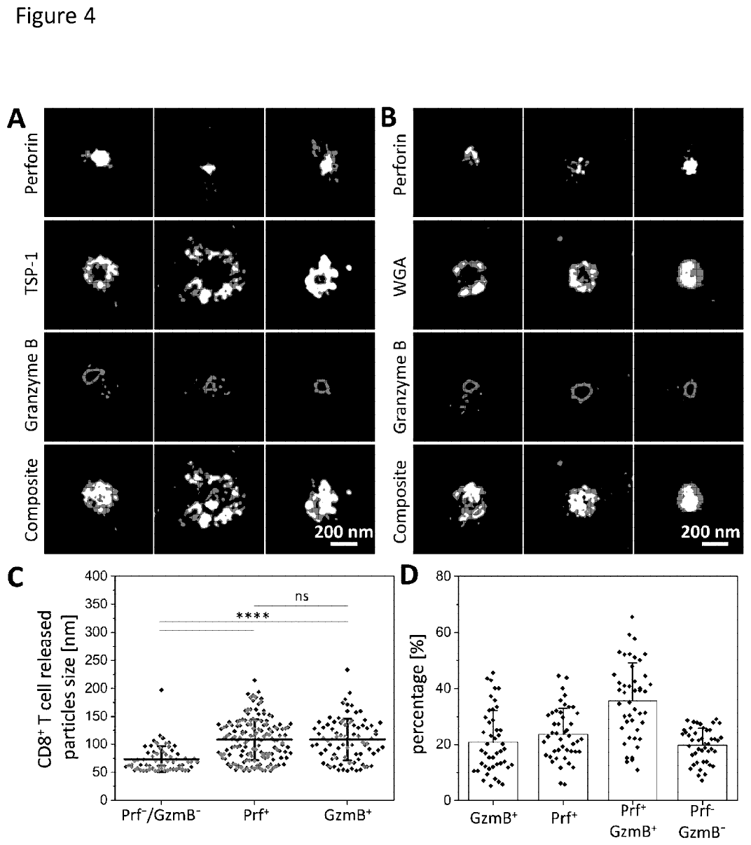

FIG. 4 shows that SMAPs have a TSP-1 shell and a core of cytotoxic proteins. (A and B) dSTORM images of individual SMAPs positive for Prf1 (green), Gzmb (magenta) and TSP-1 (A, orange) or stained with WGA (B, orange). Scale bar, 200 nm. (C) Quantification of the size of cytotoxic particles based on their protein composition (n=64 for Prf1⁻ and Gzmb⁻ cytotoxic particles, n=149 and n=83 for Prf1⁺ and Gzmb⁺ cytotoxic particles, respectively). ****, p<0.0001. n.s, not significant. (D) Quantification of the percentage of particles positive and negative for Prf1 or Gzmb. (C-D) Horizontal lines/bars and error bars represent mean±SD from five donors.

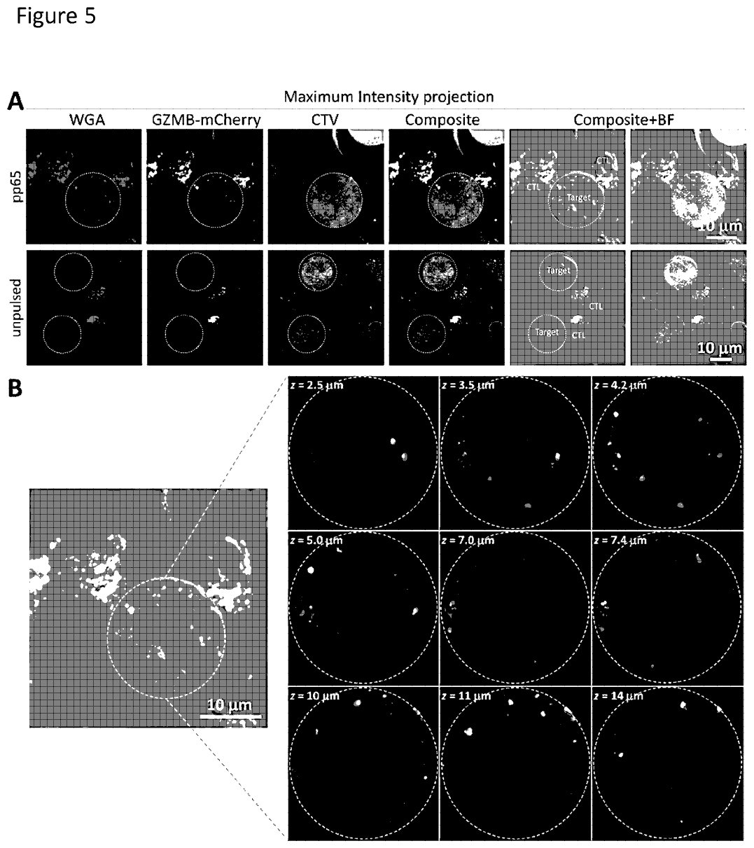

FIG. 5 shows the transfer of Gzmb-mCherry⁺ SMAPs from antigen-specific CTLs to target cells. Maximum intensity projection of confocal z-stack images depicting the transfer of Gzmb-mCherry⁺ (green) and WGA (magenta) labeled SMAPs from an antigen-specific CTL clone into pp65-pulsed JY target cells (A, top row). CTLs were also incubated with unpulsed JY target cells (A, bottom row). Target cells were labeled with CTV and are highlighted by dashed circles (Target). BF, bright field microscopy. Scale bar, 10 m. (B) 3D z stack mosaic demonstrating the presence of SMAPs at different z planes from the pp65-pulsed target cell in panel A. SMAPs were labelled with Gzmb-mCherry⁺ (green) and WGA (magenta). A dashed circle demarcates the target cell. Scale bar, 10 μm.

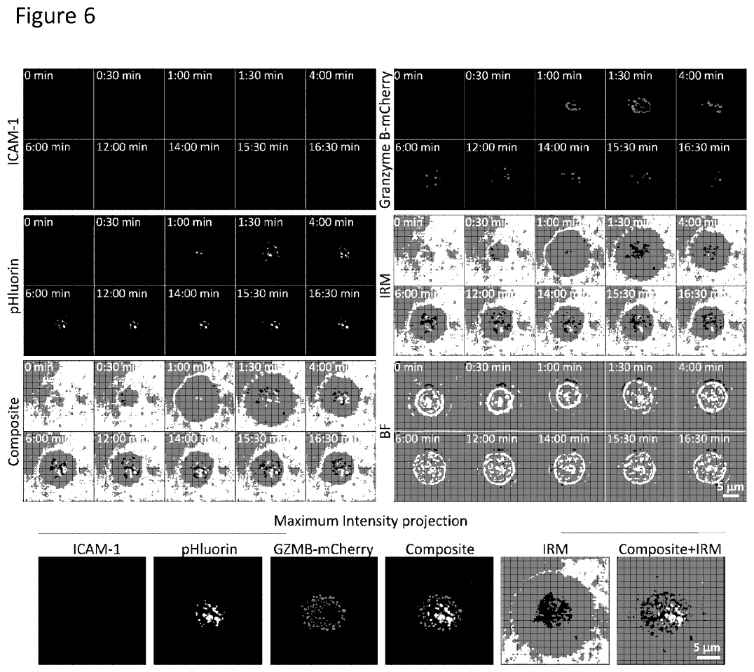

FIG. 6 shows live imaging of the release of SMAPs by Gzmb-mCherry-SEpHluorin transfected CD8⁺ T-cells. CD8⁺ T-cells transfected with Gzmb-mCherry-SEpHluorin (magenta/green) were incubated on activating (ICAM-1⁺ anti-CD3ε) SLB and imaged live by TIRFM. Snapshots of different time points are shown. The formation of a mature IS is indicated by an ICAM-1 ring (blue). Maximum intensity projection of the time lapse (bottom row). Interference reflection microscopy (IRM) and composite images are shown. BF, bright field microscopy. Scale bar, 5 μm.

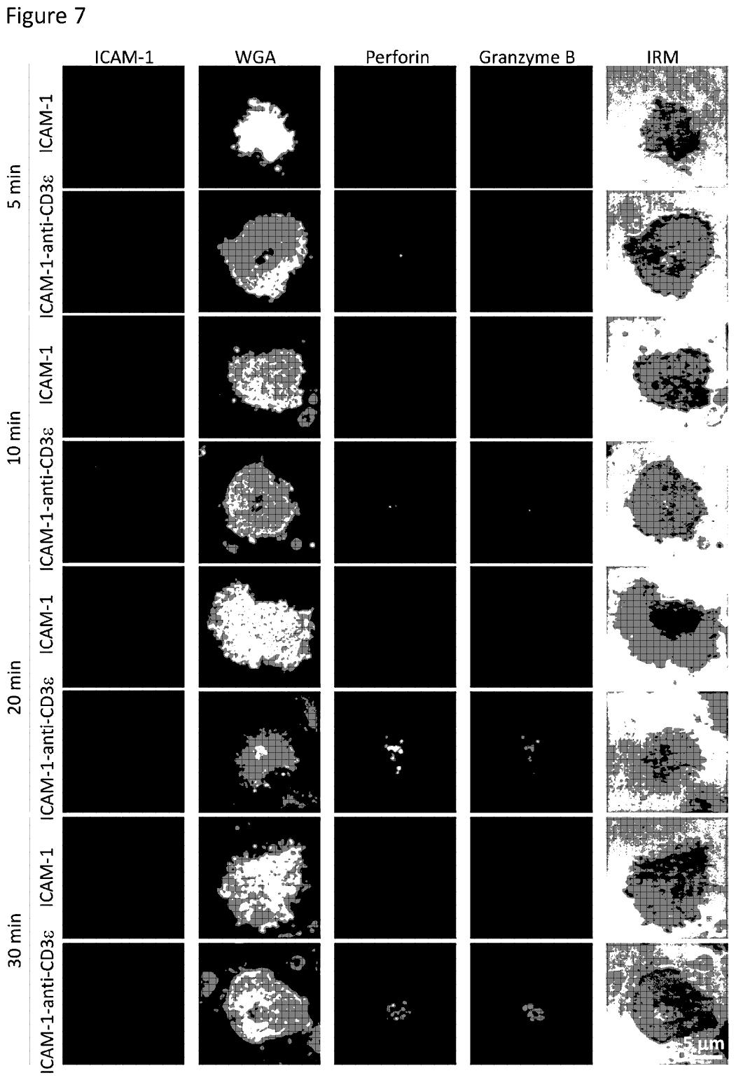

FIG. 7 shows time-dependent release of Prf1⁺ and Gzmb⁺ SMAPs at the IS. TIRFM images of CD8⁺ T-cells incubated for the indicated times on non-activating (ICAM-1) or activating (ICAM-1⁺ anti-CD3ε) SLB in the presence of anti-Prf1 (green) and anti-Gzmb (magenta) antibodies. After fixation, cells were stained with WGA (yellow) to visualize the cell membrane. The formation of a mature IS is indicated by an ICAM-1 ring (blue). IRM, interference reflection microscopy. Scale bar, 5 μm.

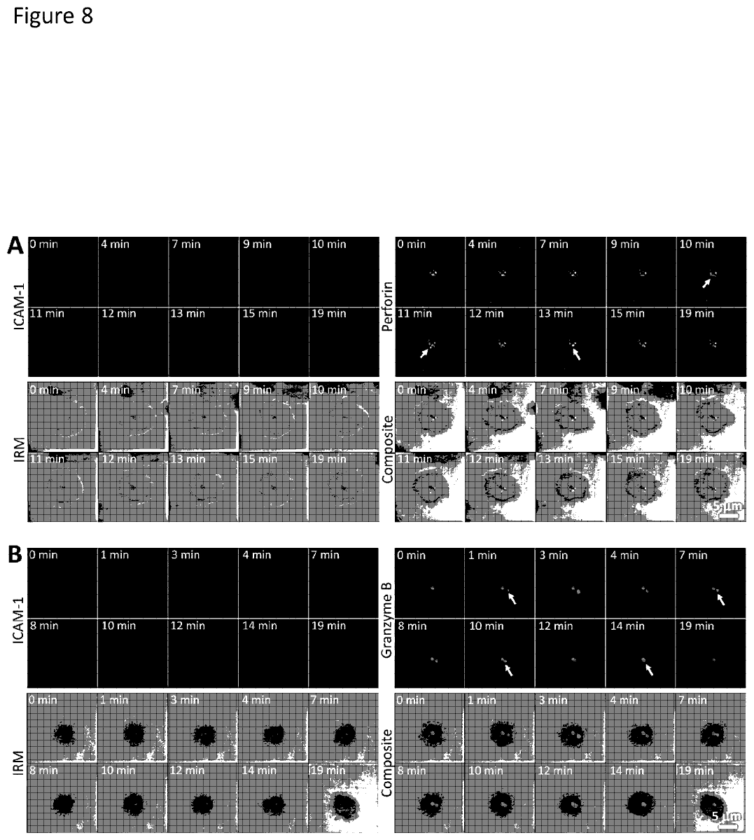

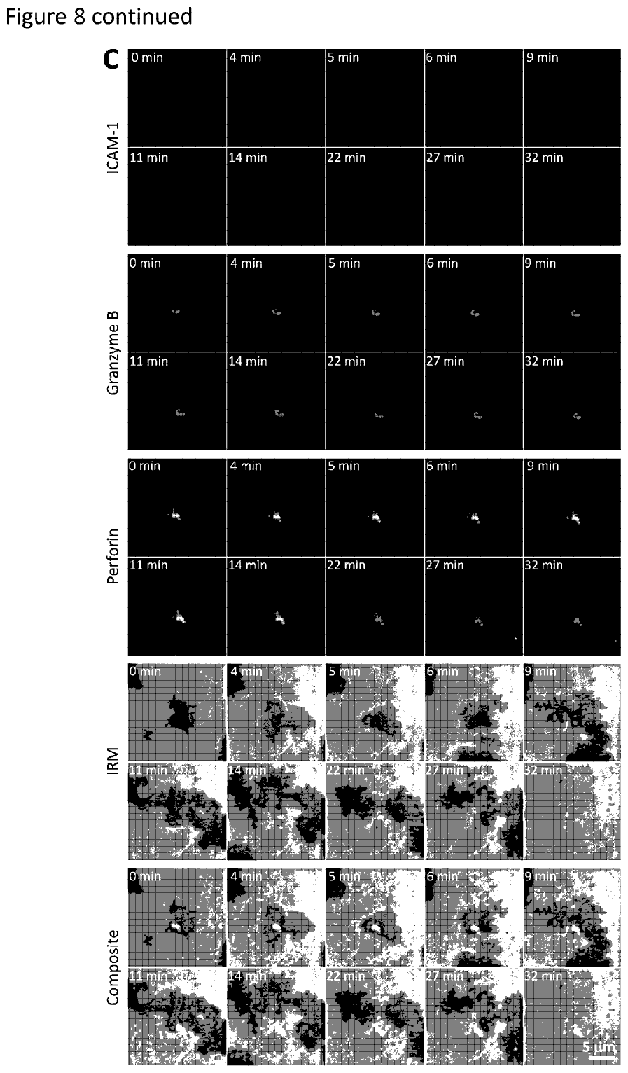

FIG. 8 shows live imaging of the release of Prf1⁺ and Gzmb⁺ SMAPs by CD8⁺ T-cells. CD8⁺ T-cells were incubated on activating (ICAM-1+anti-CD3ε) SLB in the presence of anti-Prf1 (A, green), anti-Gzmb (B, red) or both (C) antibodies and imaged live by TIRFM for 50 minutes. Snapshots of different time points are shown. Time zero refers to the start of imaging after CTLs have had 20 min to interact with SLB. The formation of a mature IS is indicated by an ICAM-1 ring (blue). Arrows indicate the presence of SMAPs. Interference reflection microscopy (IRM) and composite images are shown. Scale bar, 5 μm.

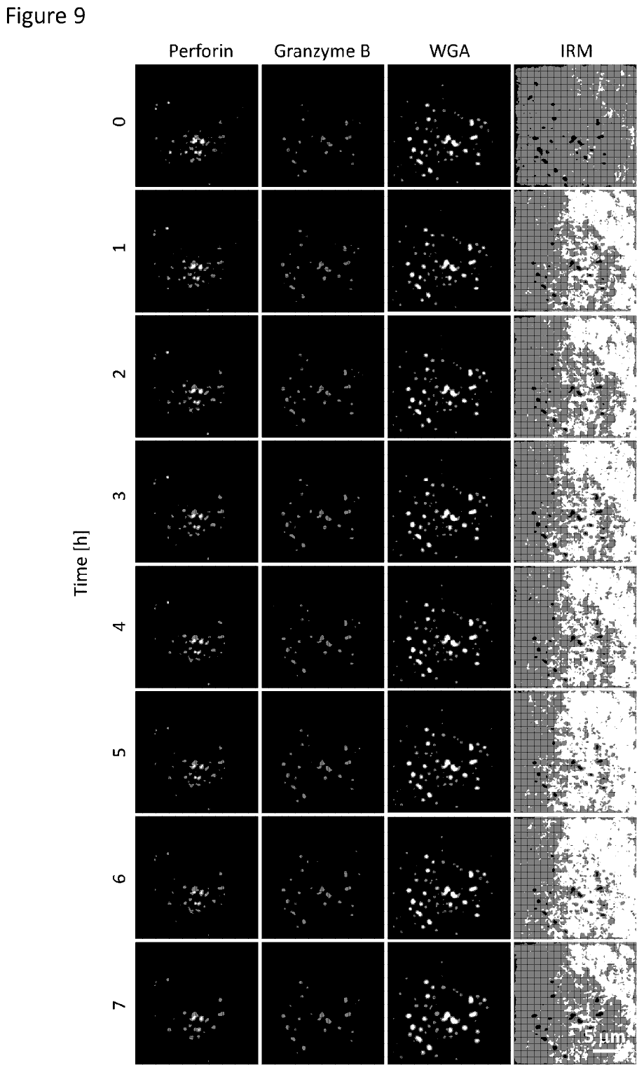

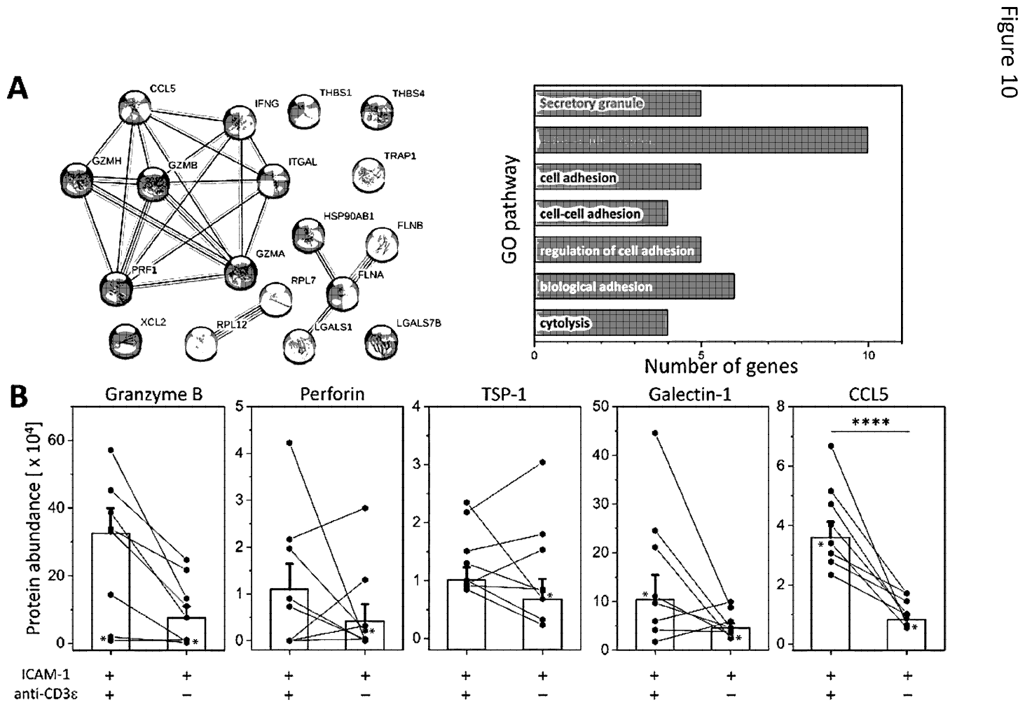

FIG. 9 shows Prf1 and Gzmb are components of SMAPs released by CD8⁺ T-cells. TIRFM images of CD8⁺ T-cell released SMAPs captured on activating (ICAM-1⁺ anti-CD3ε) SLB over a time course of seven hours. Images of the same area were taken every hour. Time zero refers to the start of imaging after SMAPs release and CD8⁺ T-cell removal. SMAPs were labeled with anti-Prf1 (green) and anti-Gzmb (magenta) antibodies, and with WGA (yellow). IRM, interference reflection microscopy. Scale bar, 5 μm.

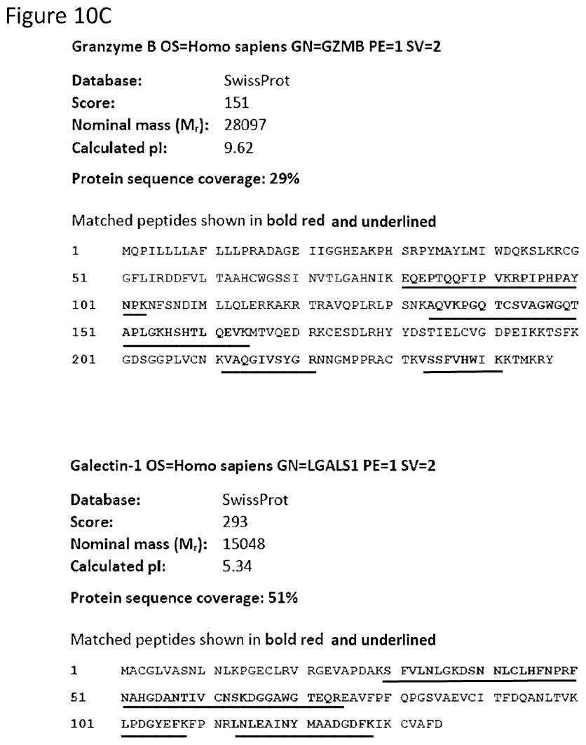

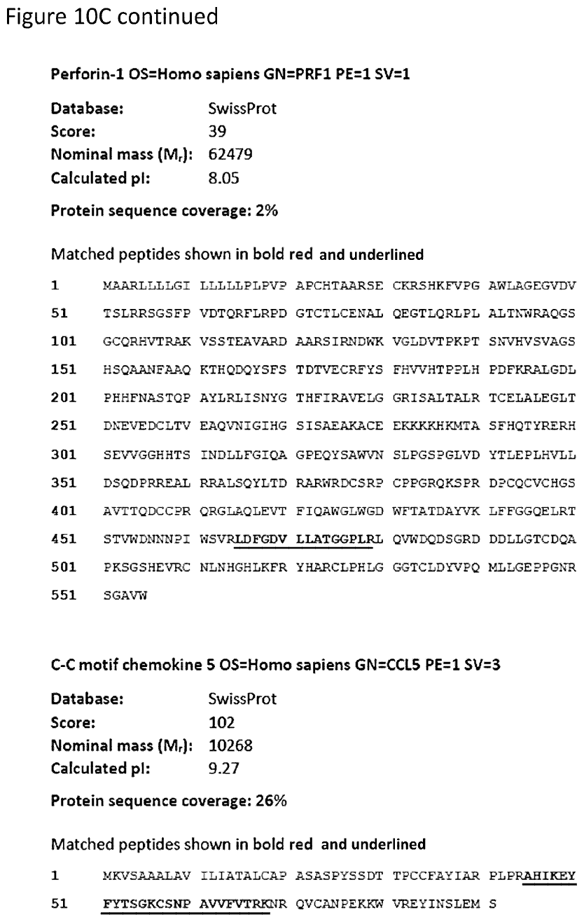

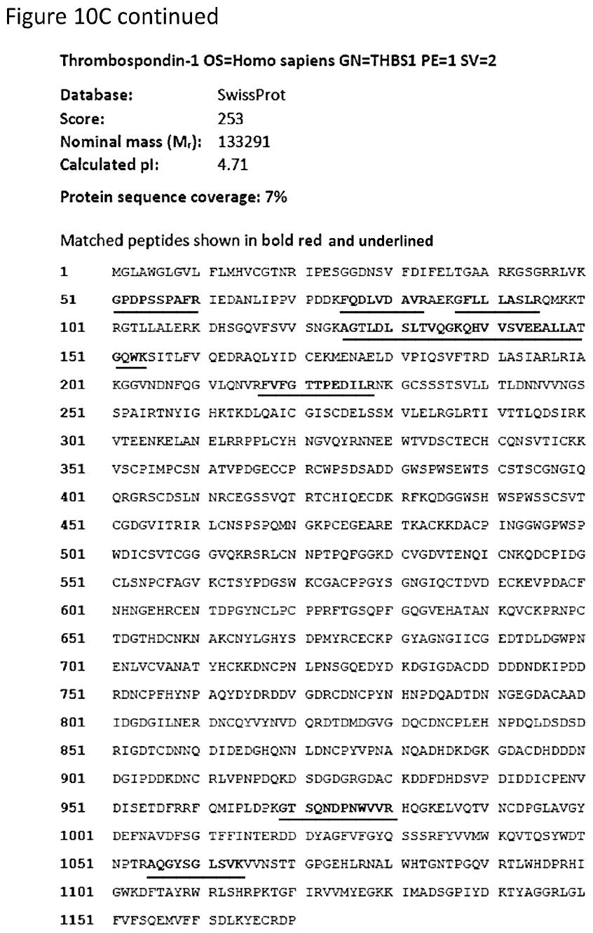

FIG. 10 shows protein abundance of major proteins identified by mass spectrometry in CD8⁺ T-cell released SMAPs. (A) Network plot and GO pathway of the proteins identified specifically in SMAPs released on activating SLB. (B) Protein abundance of five major proteins detected in SMAPs released from CD8⁺ T-cells on non-activating (ICAM-1) or activating (ICAM-1+anti-CD3) SLB. Each dot represents one donor. The red color dot (*) marks the donor that was used as an example in FIG. 2B. Horizontal lines and error bars represent mean±SEM. (C) Peptides detected in proteomics analysis with 1% FDR and score cut-off of 20 for proteins in (B) (SEQ ID NOs: 39-43). The peptides sequence is highlighted in red and bold. ****, p<0.0001. Not significant differences are not shown.

FIG. 11 shows detection of Prf1, Gzmb and β2-integrin on CD8⁺ T-cell released SMAPs by immuno-blotting. (A) SMAPs released on non-activating (ICAM-1) or activating (ICAM-1+anti-CD3ε) SLB were lysed and analyzed by immuno-blotting with the indicated antibodies (right of panels). Whole cell lysates (WCL) were analyzed in parallel and control for the absence of contamination with cellular membranes. MW, molecular weight (left of panels). (B) Quantification of the expression of components of SMAPs from immuno-blot data. Each colored dot represents one donor. Horizontal lines and error bars represent mean±SEM.

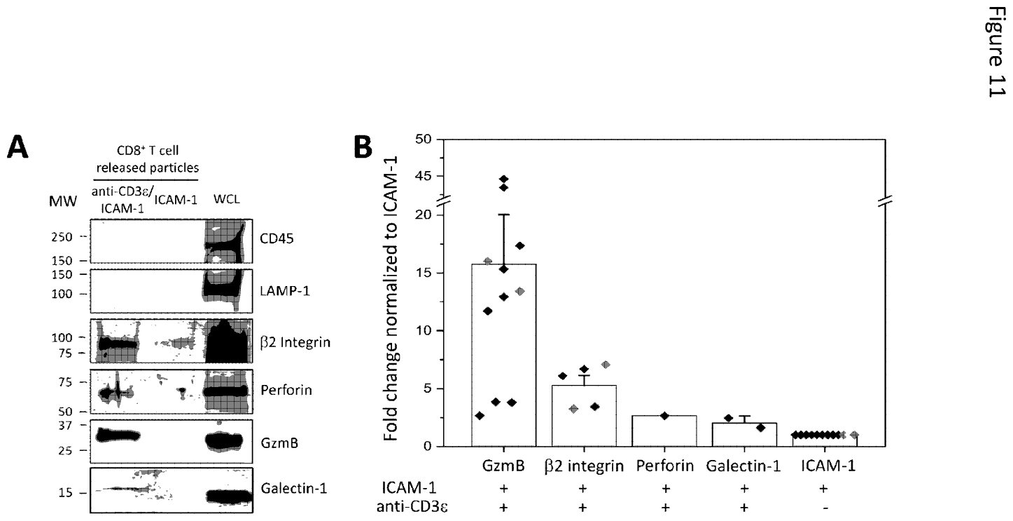

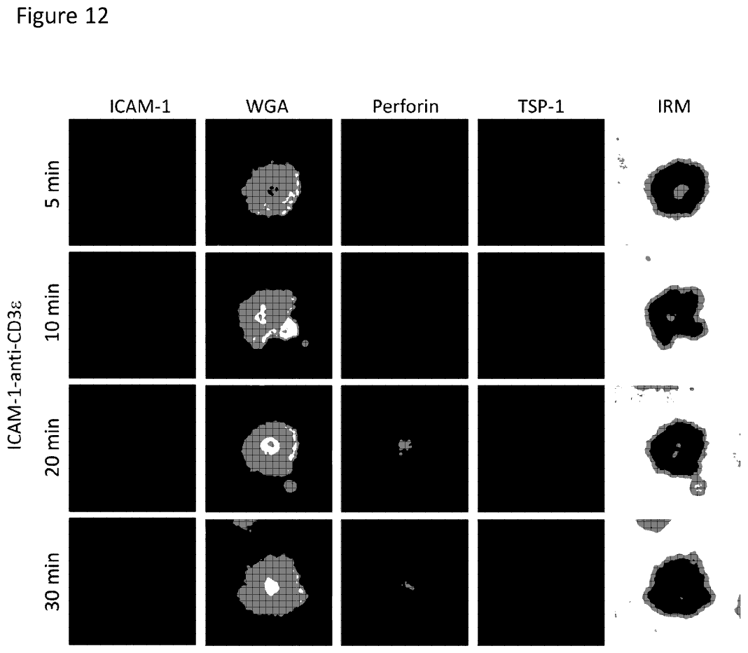

FIG. 12 shows TSP-1 containing SMAPs were released at the IS and co-localized with Prf1. TIRFM images of CD8⁺ T-cells incubated for the indicated times on activating (ICAM-1+anti-CD3ε) SLB in the presence of anti-Prf1 (green) and anti-TSP-1 (magenta) antibodies. After fixation, cells were stained with WGA (yellow) to visualize the cell membrane. The formation of a mature IS is indicated by an ICAM-1 ring (blue). IRM, interference reflection microscopy. Scale bar, 5 μm.

FIG. 13 shows TSP-1-GFPSpark transfected CD8⁺ T-cells released GFP⁺ SMAPs. (A) TIRFM images of TSP-1-GFP⁺ SMAPs (green) released from CD8⁺ T-cells transfected with TSP-1-GFPSpark. Released SMAPs were further stained with anti-Gzmb (yellow) and anti-Prf1 (magenta) antibodies. (B) SMAPs released from non-transfected CD8⁺ T-cells lacked GFP signal but were still positive for Gzmb (yellow) and Prf1 (magenta). IRM, interference reflection microscopy. BF, bright field microscopy. Scale bar, 5 μm.

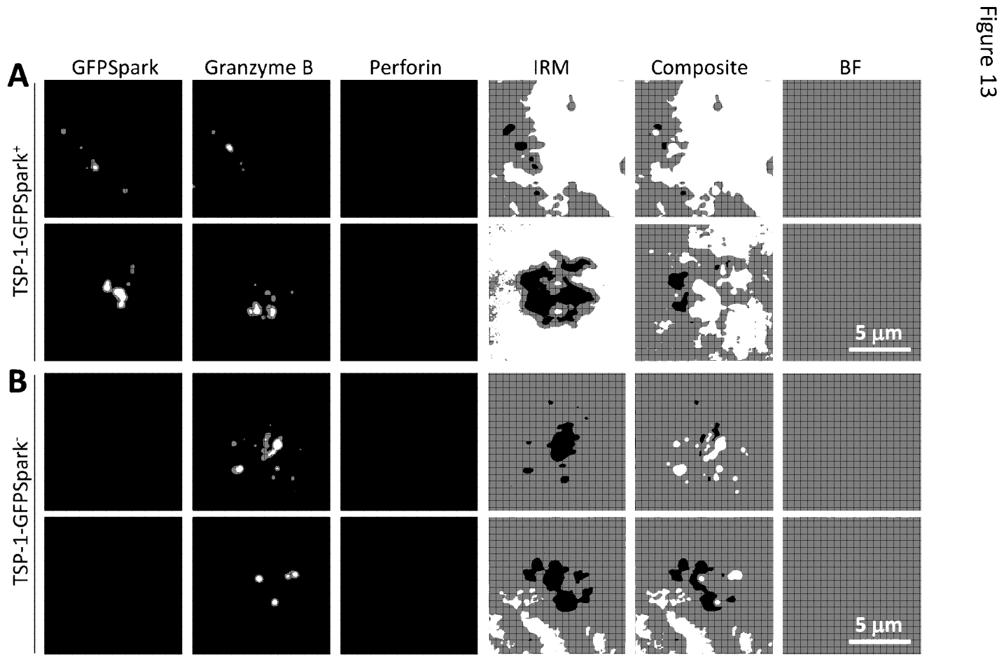

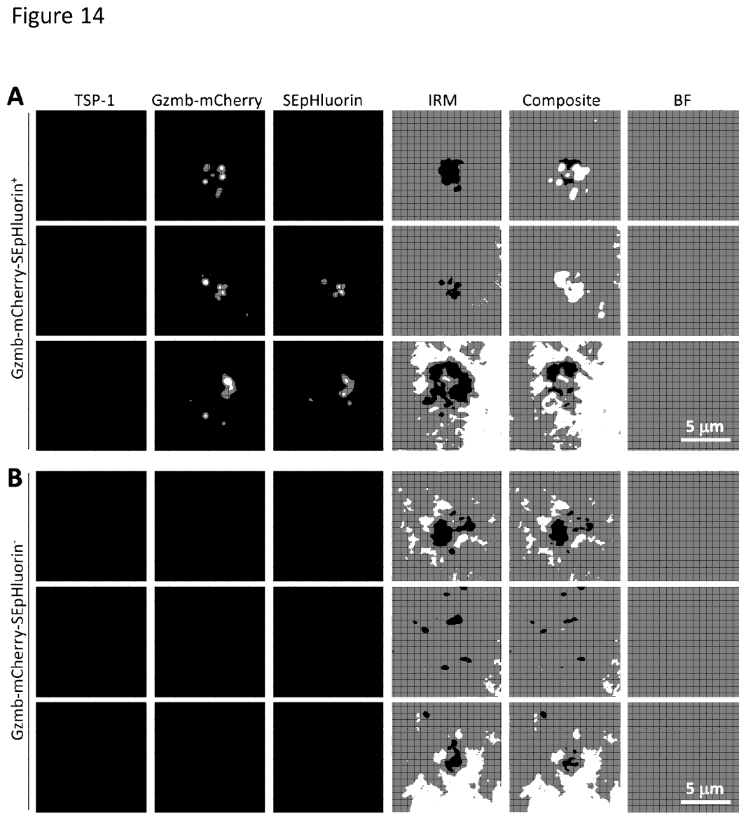

FIG. 14 shows Gzmb-mCherry-SEpHluorin transfected CD8⁺ T-cells released TSP-1⁺ SMAPs. (A) TIRFM images of Gzmb⁺ SMAPs (yellow/green) released from CD8⁺ T-cells transfected with Gzmb-mCherry-SEpHluorin. Released SMAPs were further stained with anti-TSP-1 (magenta) antibody. (B) SMAPs released from non-transfected CD8⁺ T-cells lacked mCherry and pHluorin signals but were still positive for TSP-1 (magenta). IRM, interference reflection microscopy. BF, bright field microscopy. Scale bar, 5 μm.

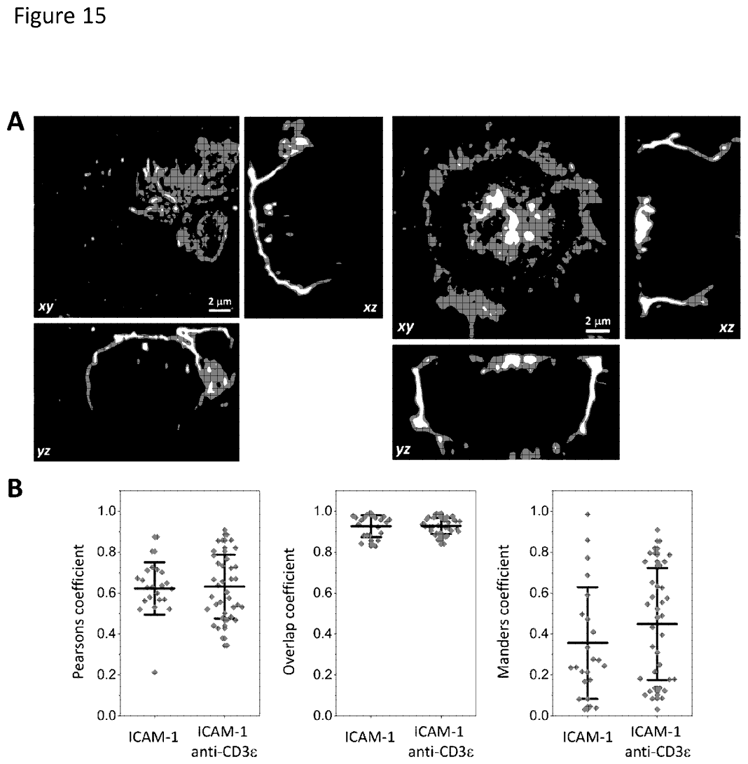

FIG. 15 shows Gzmb and TSP-1 were already associated in SMAPs in non-activating conditions. (A) 3D confocal z-stack projection and orthogonal views of CD8⁺ T cells co-transfected with Gzmb-mCherry-SEpHluorin (magenta) and TSP-1-GFPSpark (green) on non-activating (ICAM-1; left) or activating (ICAM-1+anti-CD3ε; right) SLB. pHluorin is non-fluorescent in the secretory lysosomes. Thus, co-localization between GFPSpark and mCherry signals represents TSP-1 and Gzmb. Cells were stained with WGA (yellow) to visualize the cell membrane. The formation of a mature IS is indicated by an ICAM-1 ring (blue). Scale bar, 2 μm. (B) Quantification of the colocalization between Gzmb and TSP-1 staining in non-activating (ICAM-1) and activating (ICAM-1+anti-CD3ε) conditions assessed by Pearsons coefficient (left), Overlap coefficient (middle) and Manders coefficient (right). Each dot represents one cell. Horizontal line and error bar represent mean±SD; n=1 donor. Not significant differences are not shown.

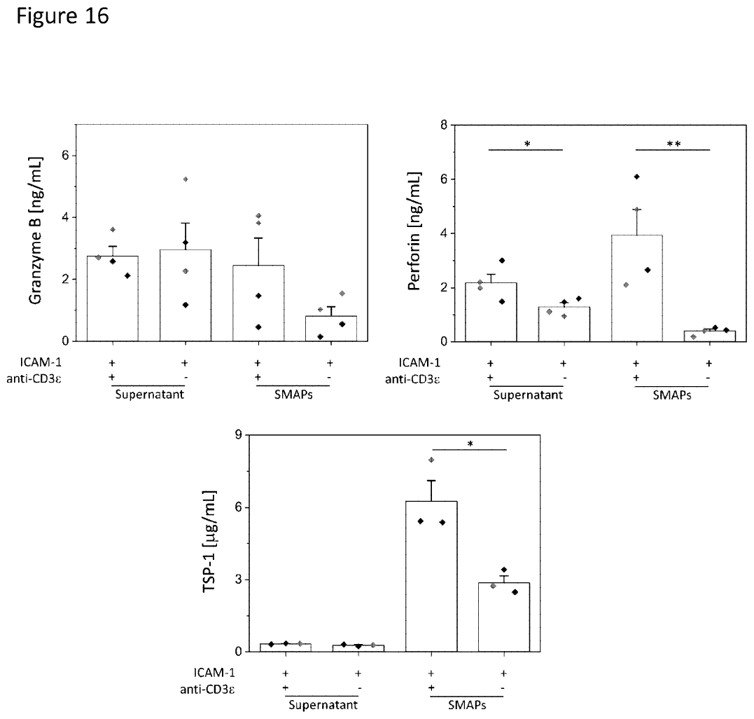

FIG. 16 shows detection of Gzmb, Prf1 and TSP-1 on CD8⁺ T-cell released SMAPs by ELISA. SMAPs released on non-activating (ICAM-1) or activating (ICAM-1⁺ anti-CD3ε) SLB were lysed and analyzed by ELISA. Supernatants from non-activating and activating conditions were analyzed in parallel. Each coloured dot represents one donor. Bars represent mean±SEM. *, p<0.05, **, p<0.01. Not significant differences are not shown.

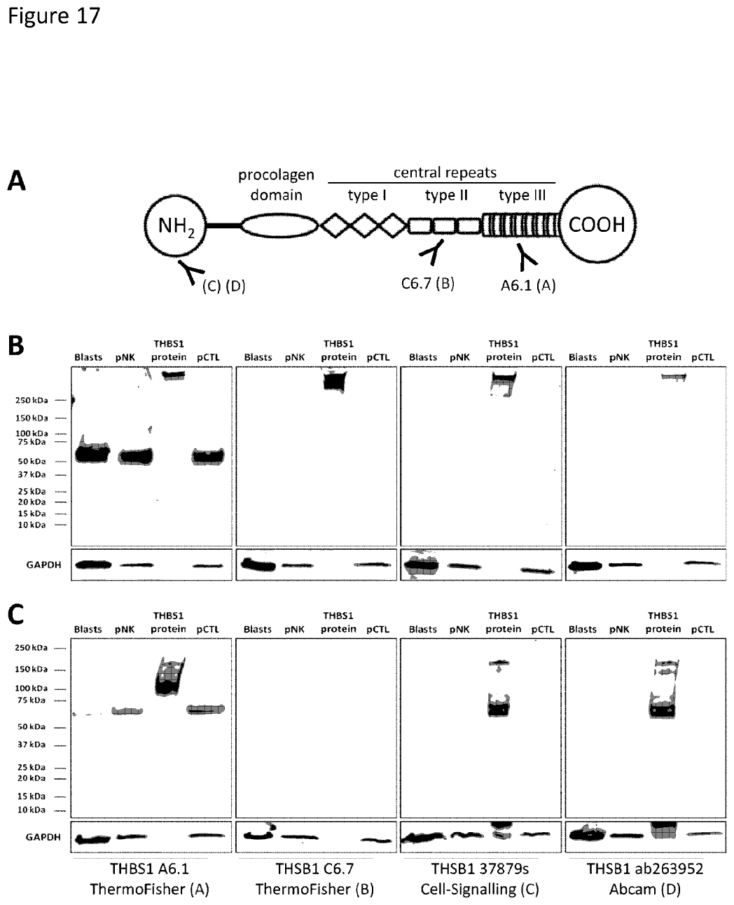

FIG. 17 shows detection of TSP-1 in CD8⁺ T-cells and primary NK cells by immuno-blotting. (A) Schematic representation of epitopes placement along human TSP-1 protein. A to D marks the binding sites for the anti-TSP-1 antibodies used in this experiment. (B, C) Immuno-blotting analysis of TSP-1 in blasted CD8⁺ T-cells (Blasts), primary NK cells (pNK) and primary CTLs (CD8⁺ CD57⁺ T-cells; pCTL) under non-reducing (B) and reducing (C) conditions with different anti-TSP-1 antibodies (as indicated below the panels). Purified full human TSP-1 protein isolated from platelets was used as a control. Note that the platelet material shows evidence of proteolysis to generate a 100 kDa C-terminal fragment and 60 kDa N-terminal fragment, but none of these match the C-terminal fragment found in CTLs and NK cells. Although we detected N-terminal peptides of TSP-1 in the mass spectrometry analysis (FIG. 10, SF6C) these were not associated with immunoreactive domains in the SMAPs on SLB.

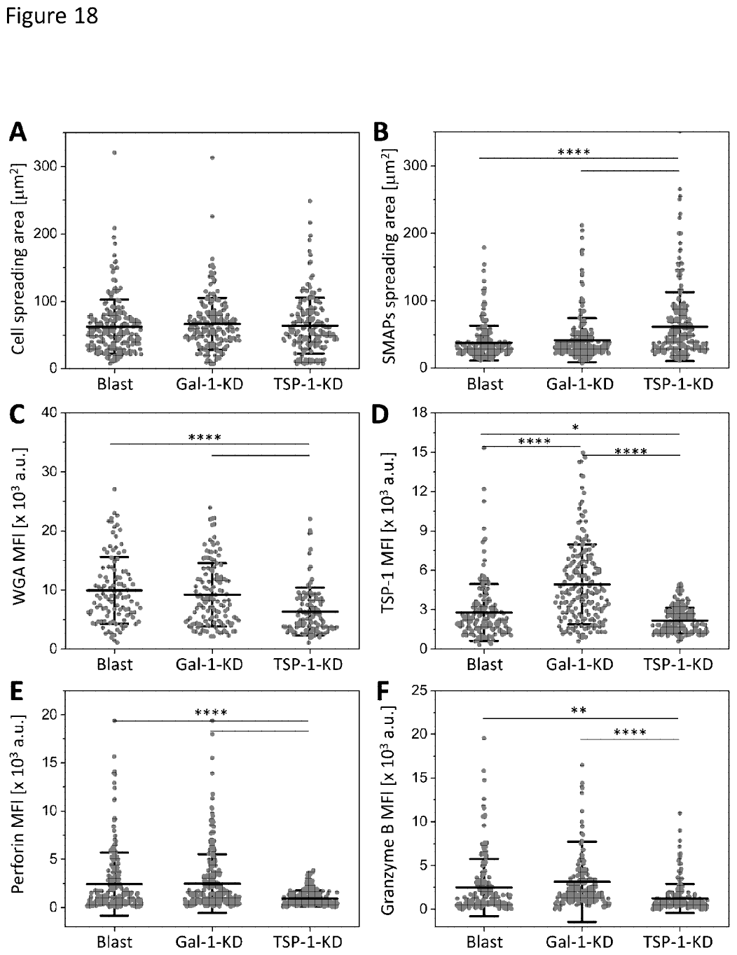

FIG. 18 shows SMAPs released from TSP-1 knockout CD8⁺ T-cells contained less perform and granzyme B. (A-B) CD8⁺ T-cell blasts (Blast), galectin-1 (Lgals1-CRISPR) and TSP-1 (TSP-1-CRISPR) genome edited CD8+ T-cell spreading area (A) and corresponding CD8⁺ T-cell released SMAPs spreading area (B) on activating SLB. (C-F) Mean fluorescent intensity (MFI) of WGA (C), TSP-1 (D), Prf1 (E), and Gzmb (F) on released SMAPs. Each dot represents one cell (A) or the area occupied by the released SMAPs from one cell (B-F). Horizontal lines and error bars represent mean±SD. *, p<0.05, **, p<0.01, ****, p<0.0001. Not significant differences are not shown.

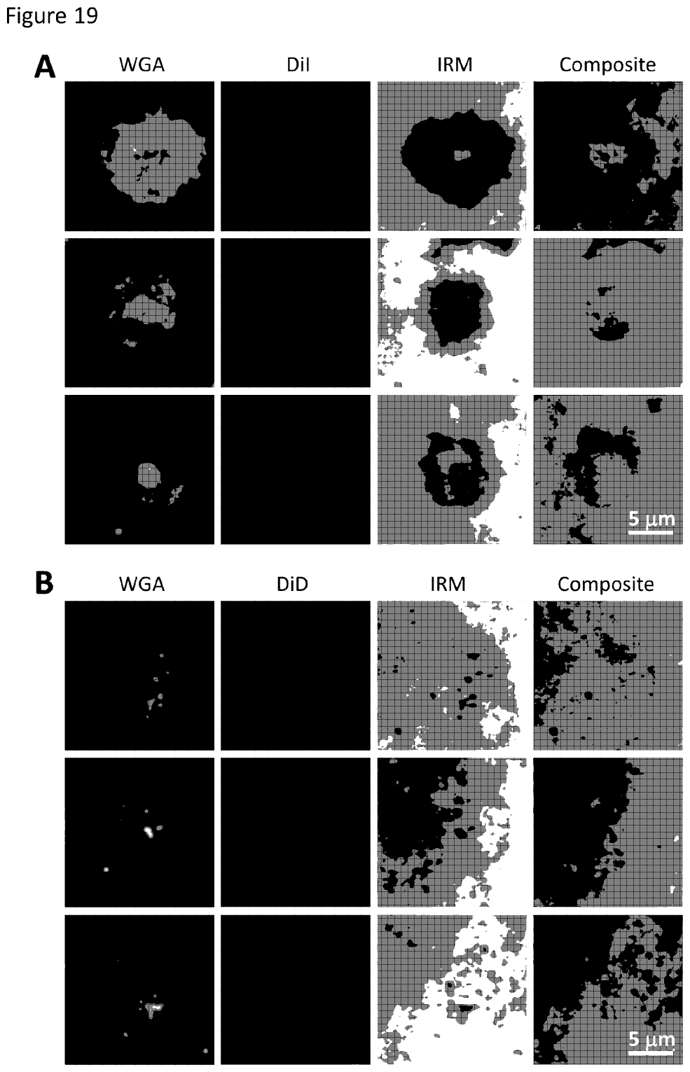

FIG. 19 shows CD8⁺ T-cells released SMAPs that contained glycoproteins but did not have a phospholipid membrane. Examples of TIRFM images of CD8⁺ T-cells (A) and released SMAPs (B) captured on activating (ICAM-1+anti-CD3ε) SLB labeled with WGA (green) or with a membrane dye (DiI or DiD; red). Interference reflection microscopy (IRM) and composite images between WGA and IRM are shown. Scale bar, 5 μm.

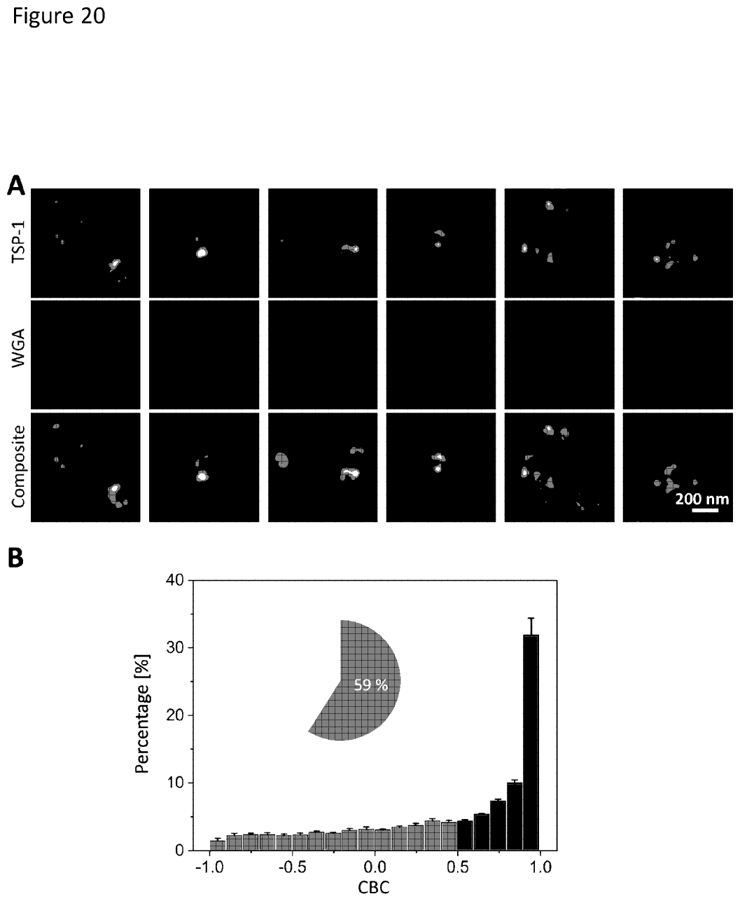

FIG. 20 shows TSP-1 is a major constituent of SMAPs. (A) Examples of dSTORM images of individual SMAPs (labeled with WGA, magenta) positive for TSP-1 (green) released on activating (ICAM-1⁺ anti-CD3ε) SLB. Scale bar, 200 nm. (B) Quantification of the percentage of colocalization between TSP-1 and WGA staining assessed by CBC analysis. Bars represent mean±SD. The percentage of colocalization is the sum of percentages (59±3%) from +0.5 to +1 and is highlighted in dark grey.

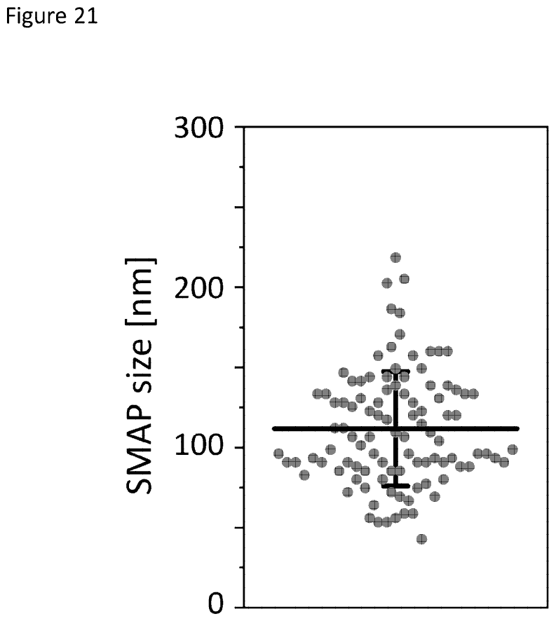

FIG. 21 shows SMAPs sizes quantified from CSXT analysis. The average SMAP diameter was 111±36 nm from n=101. Horizontal line and error bar represent mean±SD.

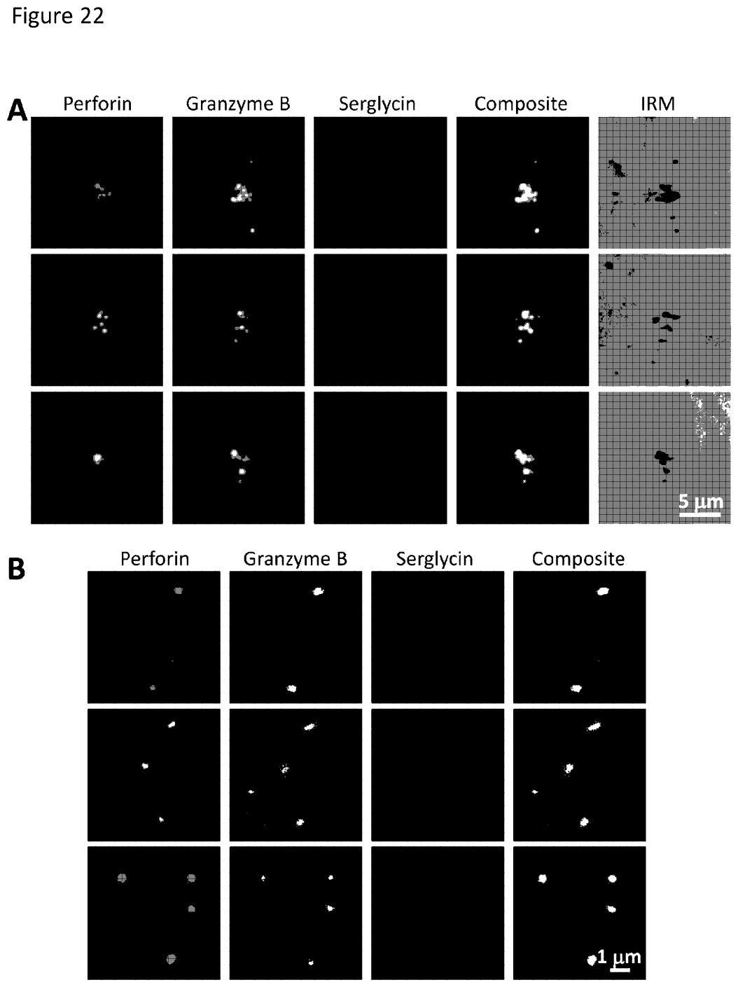

FIG. 22 shows Srgn is a component of SMAPs. TIRFM (A) and dSTORM (B) images of CTL released SMAPs captured on activating SLB. SMAPs were labeled with anti-Prf1 (green), anti-Gzmb (yellow) and anti-Srgn (magenta) antibodies. Interference reflection microscopy (IRM) and composite images are shown. Three examples from different field of views are shown for each condition. Representative data from 2 experiments. Scale bar, 1 μm.

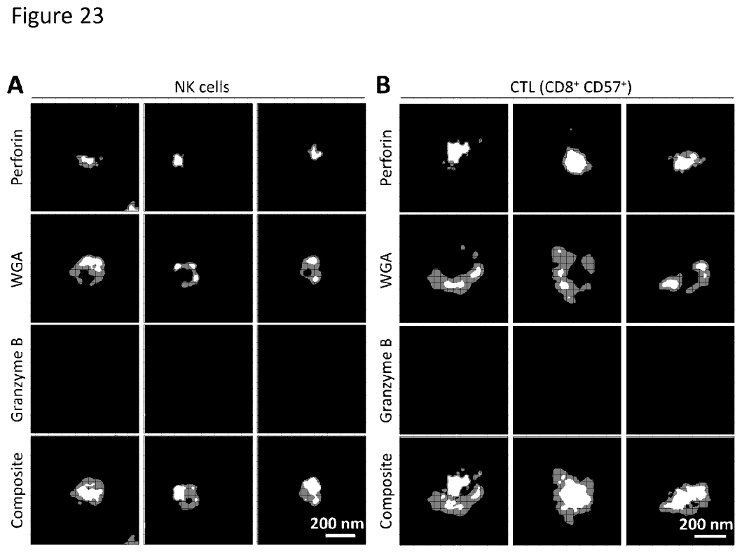

FIG. 23 shows SMAPs released by primary NK and CTLs. dSTORM images of individual SMAPs positive for Prf1 (green), WGA (orange) and Gzmb (magenta) released by pNK cells (A) or primary CTLs (B). Scale bar, 200 nm.

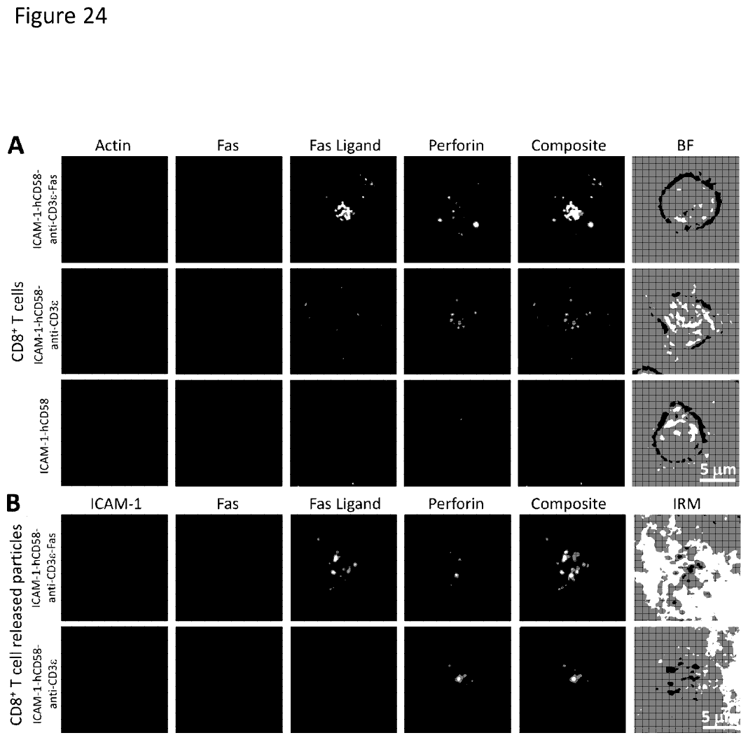

FIG. 24 shows CTLs released particles containing FasL in response to Fas signal (A) Confocal images of CTLs captured on SLB loaded with hCD58 and ICAM-1 in the presence or absence of Fas-AlexaFluor647 (magenta) and anti-CD3c (top panel). Cells were labeled with phalloidin to visualize actin (blue) and with anti-Fas Ligand (yellow) and anti-Prf1 (green) antibodies. Composite and bright field microscopy (BF) images are shown. (B) TIRFM images of CTL released particles captured on activating SLB (hCD58+ICAM-1-AF405 (blue)) in the presence or absence of Fas-AlexaFluor647 (magenta). Particles were labeled with anti-Fas Ligand (yellow) and anti-Prf1 (green) antibodies. Interference reflection microscopy (IRM) and composite images are shown. Scale bar, 5 μm.

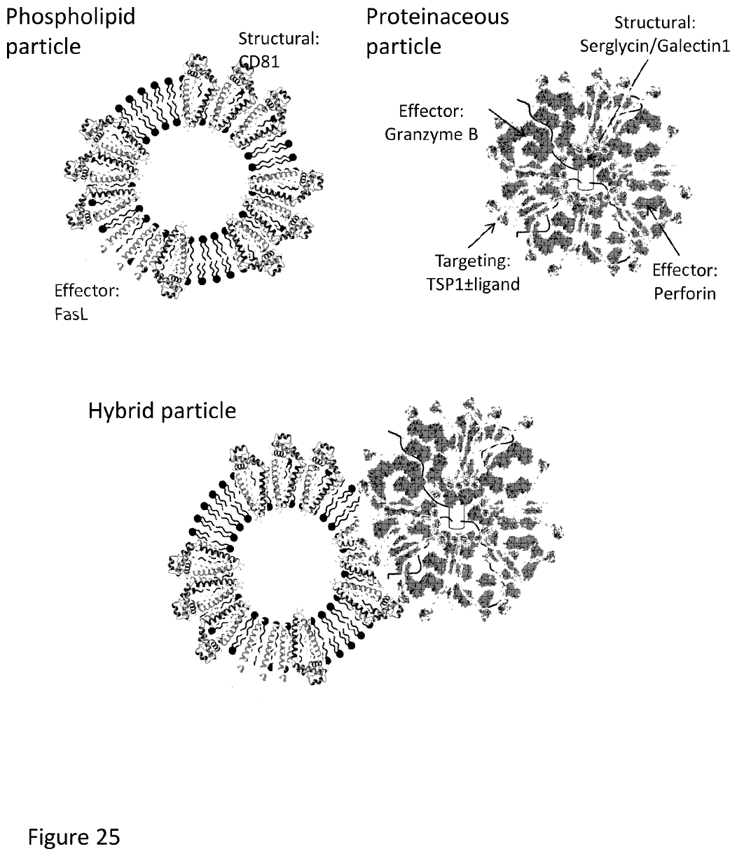

FIG. 25 shows a hybrid particle according to the invention. The hybrid particle comprise a SMAP particle contacted with a phospholipid particle expressing FasL.

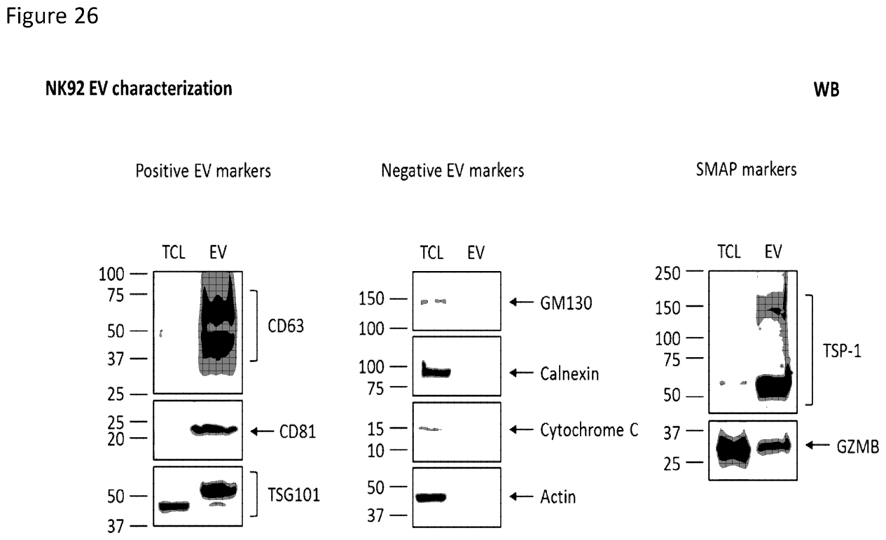

FIG. 26 NK92 EV characterization. Extracellular vesicles (EVs=Exosomes+SMAPs) were isolated from NK92 cell line and looked by Western Blot for positive and negative EV markers and for SMAPs markers such as TSP-1 and Granzyme B TCL=total cell lysate.

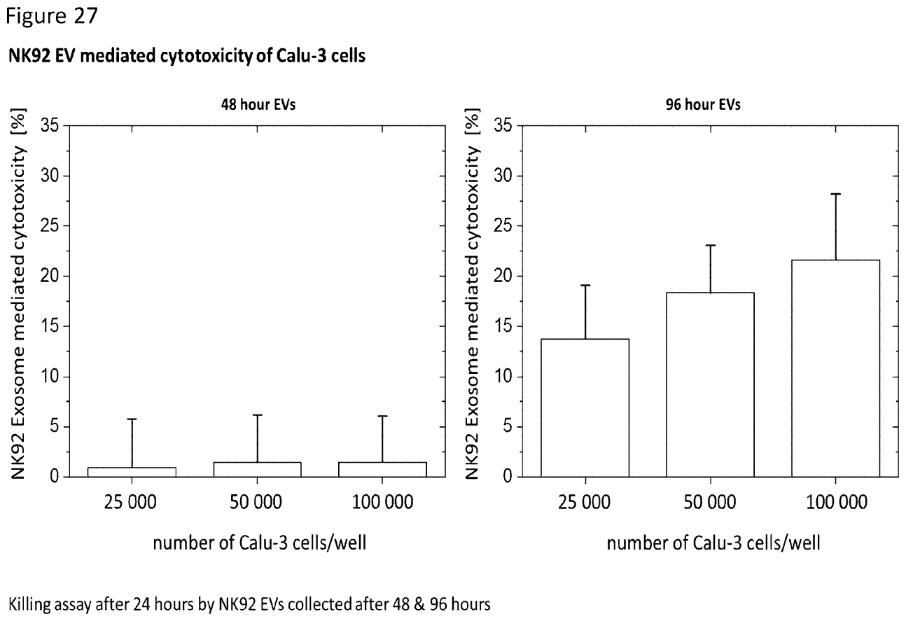

FIG. 27 NK92 EV mediated cytotoxicity of Calu-3 cells. Data shows that EVs containing SMAPs from NK92 cell line are able to kill Calu-3 cells. Calu-3 is a lung adenocarcinoma cell line. EVs from NK92 cells at 48 hours do not produce SMAPs (based on WB) and therefor the level of killing is lower compare to the EV mediated killing from 96 h EVs.

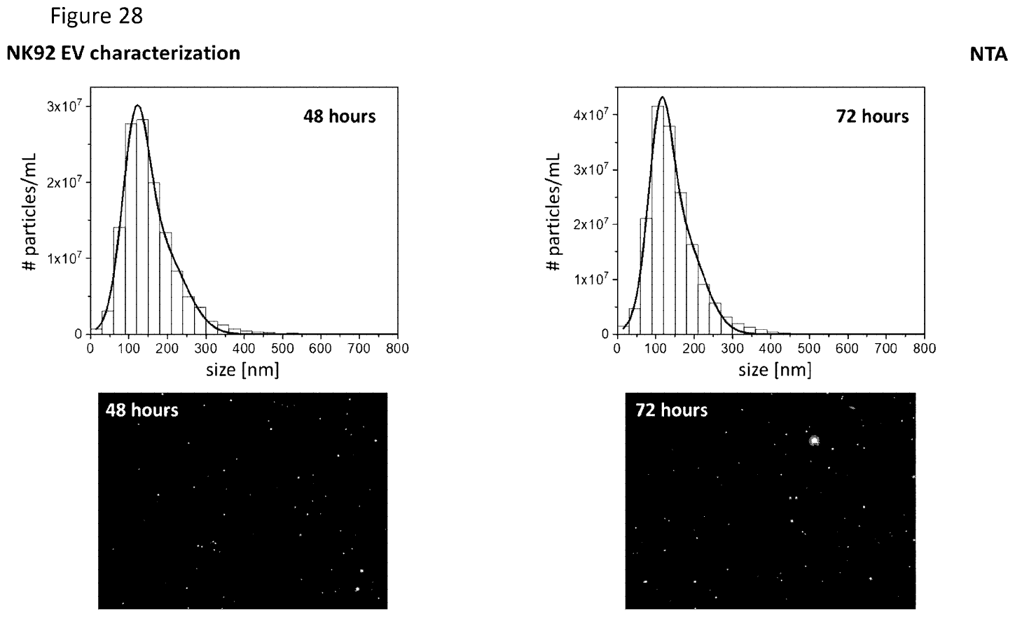

FIG. 28 NK92 EV characterization by Nanoparticle Tracking Analysis (NTA). Data shows that the EVs from NK92 cells have similar size distribution properties as exosomes and SMAPs.

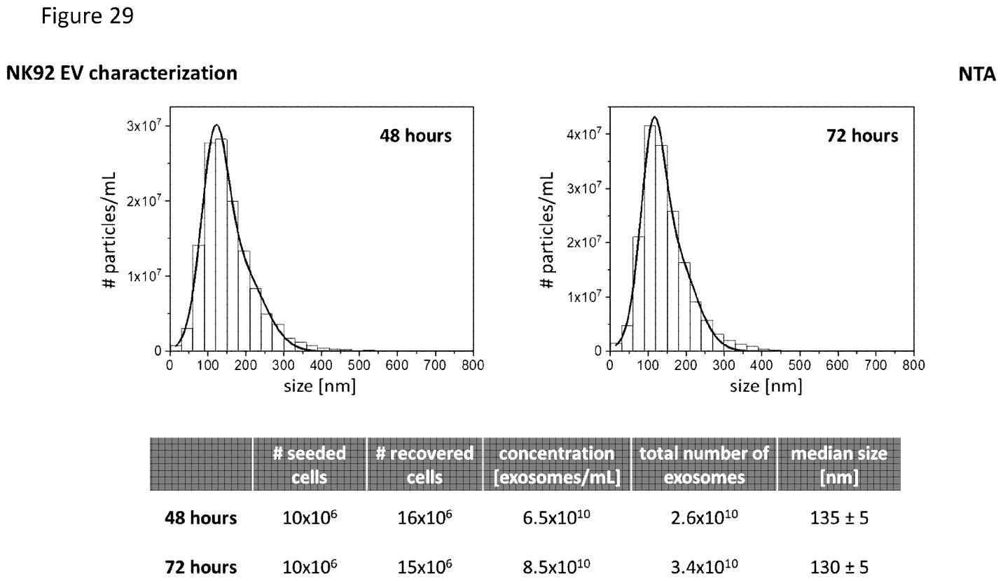

FIG. 29 NK92 EV characterization by Nanoparticle Tracking Analysis (NTA). Data shows that the EVs from NK92 cells have similar size distribution as exosomes and SMAPs and are counted as “exosomes” with a mean diameter of 130±5 nm at 96 hours when SMAPs are present.

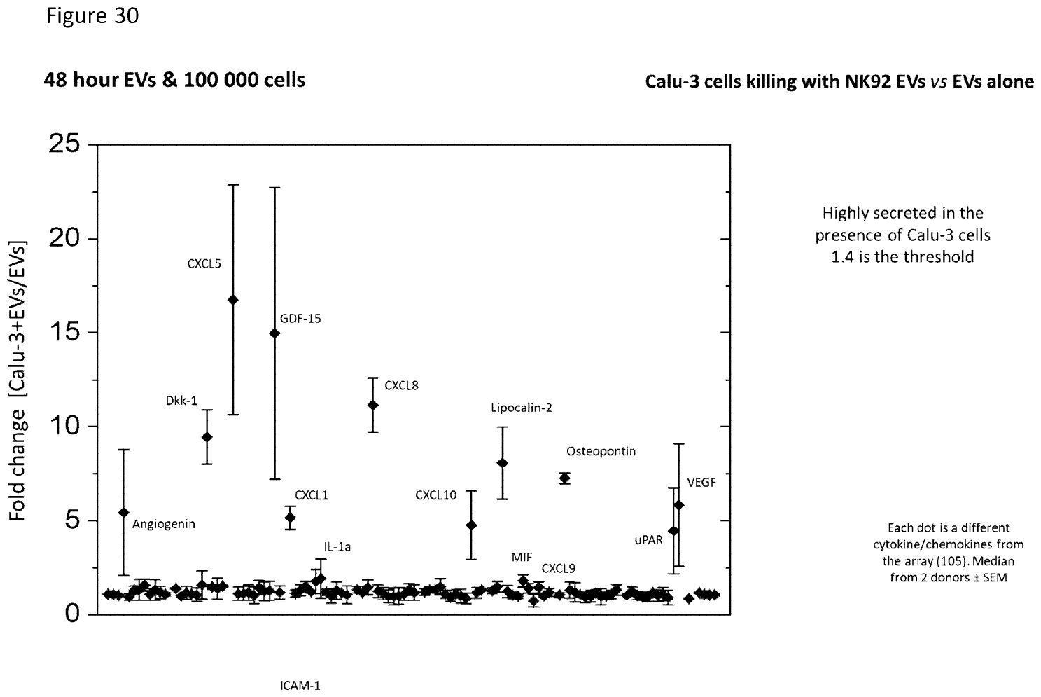

FIG. 30 Calu-3 cell response to 48 hr EVs from NK92. At 48 hours, cytotoxic protein content and killing of the NK92 EV are low. Calu-3 cells are induced by 48 hr EV to release a number of secreted proteins including chemokines including CXCL5 and CXCL10.

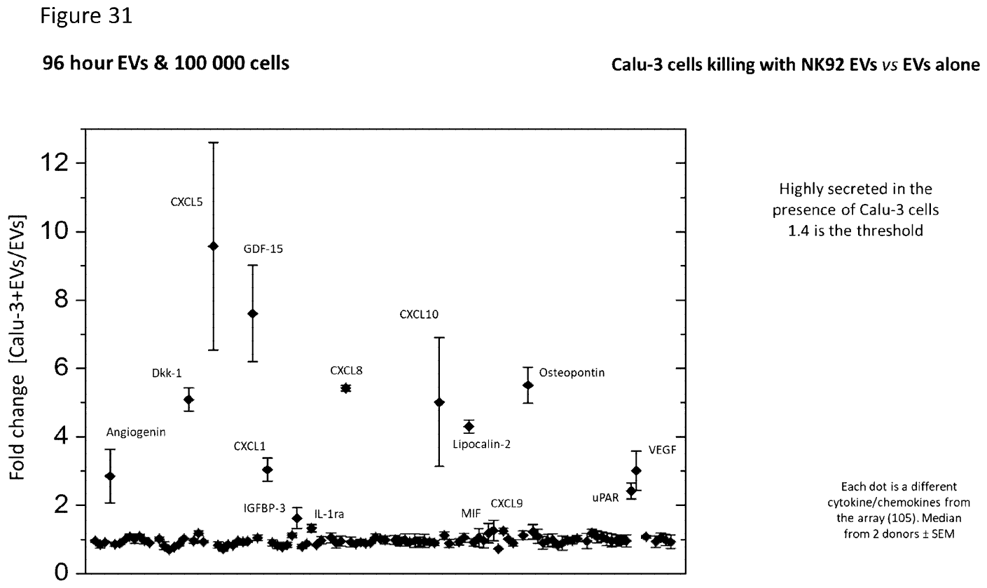

FIG. 31 Calu-3 cell response to 96 hr EVs from NK92. At 96 hours, cytotoxic protein content and killing are high. The spectrum of proteins released by surviving Calu-3 cells in response to 96 hr EVs is similar to those released in response to 48 hours EVs, except for the selective increase in IGFBP-3.

### EXAMPLES

### Materials and Methods

### Generation of Cytotoxic T-Cells (CTLs)

Peripheral blood from healthy donors was acquired from the National Health Service blood service under ethics license REC 11/H0711/7 (University of Oxford). CD8⁺ T-cells were isolated by negative selection (RosetteSep™ Human CD8⁺ T-cell Enrichment Cocktail, STEMCELL technologies; #15023) following the manufacturer's protocol. Cytotoxic CD8⁺ T-cells were activated by using anti-CD3/anti-CD28 T-cell activation and expansion beads (Dynabeads ThermoFisher Scientific; #11132D) in complete R10 medium (RPMI 1640 (#31870074), 10% FBS (ThermoFisher Scientific; #A3160801), 1% Penicillin-Streptomycin (#15140122), 1% L-Glutamine (#25030024), 25 mM HEPES (#15630080), 1% Non-essential amino acids (#11140035) all from ThermoFisher Scientific) supplemented with 50 Units/mL of recombinant human IL-2 (PreproTech; #200-02). After three days of incubation the beads were removed, and the cells were seeded with 35 Units/mL of IL-2 in complete R10 medium at 10⁶ cells/mL for further two days. The activated and rested cytotoxic CD8⁺ T-cells were used within the following two days.

### Isolation of Primary NK Cells and Primary CTLs

Primary NK cells were isolated by negative selection (RosetteSep™ Human NK cell Enrichment Cocktail, STEMCELL technologies; #15065) following the manufacturer's protocol. Primary CTLs, defined as CD8⁺ CD57⁺ T-cells, were isolated from total CD8⁺ T-cells, as described above, by positive selection with CD57⁺ magnetic beads (Miltenyi Biotec; #130-092-073) following the manufacturer's protocol. Cells were kept in complete R10 medium without IL-2 and used immediately.

### NK92 Cell Line

NK92 cells were cultured in complete NK92 medium (RPMI 1640 (#31870074), 5% FBS (ThermoFisher Scientific; #A3160801), 5% Human Serum (Sigma Aldrich; #H4522), 50 M 2-Mercaptoethanol (Sigma Aldrich; #M3148), 1% Penicillin-Streptomycin (#15140122), 2 mM L-Glutamine (#25030024), 10 mM HEPES (#15630080), 1 mM Sodium pyruvate (#11360070) all from ThermoFisher Scientific) supplemented with 100 Units/mL of recombinant human IL-2 (PreproTech; #200-02). Cells were split every two days.

### Calu-3 Cell Line

Calu-3 cells were cultured in complete Calu medium (DMEM (#31966047), Hams F12 (#21765029), 1 mM Sodium pyruvate (#11360070), 1% Non-essential amino acids (#11140035), 1% Penicillin-Streptomycin (#15140122) all from ThermoFisher Scientific). Cells were split every five days when 90% confluency was achieved.

### Generation of CTL Clones

Human CD8⁺ T-cells were purified from healthy donor blood samples using the RosetteSep Human CD8⁺ T Cell Enrichment Cocktail. For cloning, HLA-A2-restricted CD8⁺ T-cells specific for the NLVPMVATV (SEQ ID NO: 44) peptide of the cytomegalovirus protein pp65 were tetramer stained and single cell sorted into 96-U-bottom plates using a BD FACSAria II cell sorter. Cells were cultured in RPMI 1640 medium supplemented with 5% human AB serum (Inst. Biotechnologies J.BOY), minimum essential amino acids, HEPES and sodium pyruvate, 150 Units/mL human recombinant IL-2 and 50 ng/mL human recombinant IL-15. CD8⁺ T-cell clones were stimulated in complete RPMI/HS medium containing 1 mg/mL PHA with 1×10⁶/mL 35 Gy irradiated allogeneic peripheral blood mononuclear cells (isolated on Ficoll Paque Gradient from fresh heparinized blood samples of healthy donors, obtained from EFS) and 1×10⁵/mL 70 Gy irradiated EBV-transformed B cells. Re-stimulation of clones was performed every 2 weeks. Blood samples were collected and processed following standard ethical procedures (Helsinki protocol), after obtaining written informed consent from each donor and approval by the French Ministry of the Research (transfer agreement AC-2014-2384). Approbation by the ethical department of the French Ministry of the Research for the preparation and conservation of cell lines and clones starting from healthy donor human blood samples has been obtained (authorization No DC-2018-3223).

EBV-transformed B cells (JY) HLA-A2⁺ were used as target cells and cultured in RPMI 1640 GlutaMAX supplemented with 10% FCS and 50 μM 2-mercaptoethanol, 10 mM HEPES, 1×MEM NEAA, 1× Sodium pyruvate, 10 μg/mL ciprofloxacine.

All cell lines are routinely screened for mycoplasma contamination using MycoAlert mycoplasma detection kit (Lonza).

### Supported Lipid Bilayer (SLB)

Preparation of liposomes and mobile SLB formation is described in detail elsewhere. In brief, SLB were formed by incubation with mixtures of small unilamellar vesicles to generate a final lipid composition of 12.5 mol % DOGS-NTA and a mol % of DOPE-CAP-Biotin to yield 30 molecules/μm² anti-CD3ε(UCHT1)-Fab in DOPC at a total lipid concentration of 0.4 mM. Lipid droplets were deposited onto clean glass coverslip (SCHOTT; #1472315) of the flow chamber (sticky-Slide VI 0.4, Ibidi; #80608). After 20 min incubation the flow chamber was flooded with Hepes Buffered Saline (HBS) supplemented with 0.1% Human Serum Albumin (HSA) (Merck-Millipore; #12667-50 mL) and flushed to remove excess liposomes. After blocking with 5% casein in PBS containing 100 μM NiSO₄, to saturate NTA sites, 10 μg/mL unlabeled streptavidin (Europa Bioproducts Ltd; #PZSA10-100) was coupled to biotin head groups for 15 min. SLB were flushed with HSA/HBS and incubated for 20 min with 200 molecules/μm² of ICAM-1-AlexaFluor405-His tagged protein (unstimulated condition) or with an addition of 5 μg/mL of anti-CD3ε-Fab (stimulated condition). Unbound proteins were flushed out by HSA/HBS and the SLB were ready to use. SLB were uniformly fluid as determined by fluorescence recovery after photobleaching. Protein concentrations required to achieve desired densities on bilayers were calculated from calibration curves constructed from flow cytometric measurements of bilayer-associated fluorescence of attached proteins on bilayers formed on glass beads, compared with reference beads containing known numbers of the appropriate fluorophore (Bangs Laboratories; #647-A). All lipids were purchased from Avanti Polar Lipids, Inc.

### Release of Supramolecular Attack Particles (SMAPs)

CD8⁺ T-cells, primary NK cells and primary CTLs were plated onto stimulated or unstimulated SLB for 90 min at 37° C. After incubation, cells were flushed out for a minimum of three times with ice-cold PBS. The released SMAPs captured on SLB were further analysed by ELISA, immunostaining or immuno-blotting.

Isolation of Extracellular Vesicles (EVs) from NK92 Cell Line

NK92 cells were seeded (10×10⁶ cells) for 48 and 96 hours in modified NK92 cell media (5% of Human serum and 5% of FBS was replaced by 10% Exosome depleted FBS (ThermoFisher Scientific; Ser. No. 15/624,559)). EVs were isolated by using an EXO-Prep one step isolation reagent from cell media (HansaBioMed, #HBM-EXP-C25) following the manufacturer instructions. EVs were resuspended in PBS and used for immuno-blotting, NTA analysis and cytotoxicity assay.

### Transfection of CD8⁺ T-Cells

CD8⁺ T-cells were activated with anti-CD3/anti-CD28 T-cell activation and expansion beads in complete R10 medium supplemented with 50 Units/mL of IL-2. After three days of incubation the beads were removed and the cells were transfected with mRNA or cDNA, and cultured with 35 Units/mL of IL-2 in complete R10 medium at 10⁶ cells/mL. 0.2×10⁶ CD8⁺ T-cells were transfected with 2 μg Gzmb-mCherry-SEpHluorin mRNA or 2 μg TSP-1-GFPSpark cDNA (Sino Biological; #HG10508-ACG) by using the Neon Transfection system (ThermoFisher Scientific), electrical pulse 1600V, 10 ms and 3 pulses in 10 μL buffer R. The transfection levels were assessed after 24 hours.

### Transfection of CTL Clones

For efficient transfection of human CTLs with tagged molecules, we synthetized capped and tailed poly(A) mCherry-tagged Gzmb mRNA by in vitro transcription from the plasmid pGzmb-mCherry-SEpHluorin. 1 μg of pGzmb-mCherry-SEpHluorin was first linearized by NotI digestion to be used as template for in vitro transcription by the T7 RNA polymerase using mMESSAGE mMACHINE T7 Ultra kit as per manufacturer's protocol.

Human CTLs were transfected using a GenePulser Xcell electroporation system (BioRad). 1×10⁶ CTLs (5 days after restimulation and therefore in expansion phase) were washed and resuspended in 100 μL Opti-MEM medium at room temperature with 2 μg mCherry-tagged Gzmb mRNA (square wave electrical pulse at 300V, 2 ms, 1 pulse). 16 hours after transfection the efficacy was verified by FACS analysis (typically 50-80% of cells were transfected).

### Total Internal Reflection Fluorescent Microscopy (TIRFM) Imaging

TIRFM imaging was performed with an Olympus IX83 inverted microscope (Olympus) equipped with a 150×1.45 NA oil-immersion objective. For TIRFM imaging, cells were plated onto stimulated or unstimulated SLB for 5, 10, 20 or 30 min and then fixed with 4% PFA/PBS for 30 min at room temperature. After fixation the cells were stained for one hour with 10 μg/mL directly conjugated anti-Gzmb-AlexaFluor647 (BD Biosciences; #560212), in-house labeled anti-TSP-1-AlexaFluor647 (Abcam; #1823) and anti-Prf1-AlexaFluor488 (BD Biosciences; #563764) primary antibodies after blocking with 5% BSA/PBS for one hour. Wheat Germ Agglutinin (WGA) conjugated with CF568 (Biotium; #29077-1) or AlexaFluor488 (ThermoFisher Scientific; #W11261), or DiD/DiI (ThermoFisher Scientific; #V22887/#V22888) membrane dyes were used to label the cell membrane or the CD8⁺ T-cell released SMAPs. Fluorescent emission was collected by the same objective onto an electron-multiplying charge-coupled device camera (Evolve Delta, Photometrics). Post processing of the fluorescence images was performed with ImageJ (National Institute of Health).

### Live Cell TIRFM Imaging

Live cell TIRFM imaging was performed with an Olympus IX83 inverted microscope (Olympus) equipped with a 150×1.45 NA oil-immersion objective at 37° C. CD8⁺ T-cells were pre-incubated with anti-Prf1-AlexaFluor488 and anti-Gzmb-AlexaFluor647 or with in house labeled anti-TSP-1-AlexaFluor647 for 20 min on stimulated SLB before live cell imaging. Cells were recorded every minute for approximately 50 minutes before being flushed out on the stage with ice-cold PBS. A focus lock system was used to keep the sample in focal plane.

For live cell imaging of the fluorescently tagged Gzmb-mCherry-SEpHluorin, the transfected CTLs were plated on stimulated SLB 24 hours after transfection. The fluorescent emission was recorded every 30 seconds for approximately 20 minutes. Post processing of the fluorescence images and video creation was performed with ImageJ (National Institute of Health).

### Confocal Imaging

CTLs and JY cells were prepared as for time-lapse live cell confocal microscopy. Transfected CTLs were conjugated with target cells (1 min, 1500 rpm centrifugation) and incubated for 2 h at 37° C., 5% CO₂, in 5% FCS/RPMI/10 mM HEPES. Cells were resuspended and seeded on poly-L-lysine coated slides, fixed with 3% PFA/PBS for 15 min at room temperature. Cells were mounted in 90% glycerol/PBS containing 2.5% DABCO (Sigma Aldrich) and inspected by using laser scanning confocal microscope (LSM780 or LSM880, Zeiss, Germany) with a 63×oil-immersion objective. Post processing of the fluorescence images and z-stack creation was performed with ImageJ (National Institute of Health). The number of SMAPs within a target cell was counted manually from 2 independent experiments. Mean fluorescent intensity of the Gzmb-mCherry signal was quantified from the maximum intensity projection of the confocal z-stacks highlighting the target cell area.

3D Confocal imaging of the Fas-Fas Ligand was performed by using a Nikon A1R HD25 confocal system with a 60× oil-immersion objective (Nikon, UK). Cells were plated onto stimulated or unstimulated SLB in the presence or absence of in house labeled Fas-AlexaFluor647 and/or unlabeled human CD58 at the concentration of ˜200 and/or ˜100 molecules/μm², respectively. After 20 min incubation at 37° C. and 5% CO₂ the cells were fixed with 4% PFA/PBS for 30 min at room temperature. After fixation the cells were stained for one hour with 10 μg/mL directly conjugated in house labeled anti-FasLigand-AlexaFluor568 (Abcam; #134401) and anti-Prf1-AlexaFluor488 (BD Biosciences; #563764) primary antibodies after blocking with 5% BSA/PBS for one hour. Phalloidin conjugated with AlexaFluor405 (ThermoFisher Scientific; #A30104) was used to label the CTLs actin cytoskeleton. Fluorescent emission was collected in sequential manner. Post processing of the fluorescence images was performed with ImageJ (National Institute of Health).

### Live Cell Confocal Imaging

Transfected CTLs were loaded with 1 μg/mL AlexaFluor647 conjugated Wheat Germ Agglutinin (WGA, Invitrogen) for 4 h and extensively washed with 5% FCS/RPMI/10 mM HEPES. JY cells were left unpulsed or pulsed with 10 μM peptide, loaded with CTV (Invitrogen), washed and seeded at 2×10⁴ cells per well on poly-D-lysine-coated 15-well chambered slides (Ibidi) before imaging. Chambered slides were mounted on a heated stage within a temperature-controlled chamber maintained at 37° C. and constant CO₂ concentrations (5%) and inspected by time-lapse laser scanning confocal microscopy (LSM 780 or LSM880, Zeiss, Germany).

dSTORM Imaging and Analysis

Multicolor dSTORM imaging was performed with primary antibodies directly conjugated with AlexaFluor488 and AlexaFluor647 acquired in sequential manner by using the TIRFM imaging system (Olympus). Antibodies used were anti-Prf1 (BD Biosciences; #563764), anti-Gzmb (BD Biosciences; #560212), anti-TSP-1 (Abcam; #1823) and anti-galectin-1 (ThermoFisher Scientific; #43-7400). CD8⁺ T-cell released SMAPs were additionally stained with WGA-CF568 (Biotium; #29077-1) or WGA-AlexaFluor647 (ThermoFisher Scientific; #W32466). Fab₂ conjugated secondary antibodies with CF568 (Sigma Aldrich; #SAB4600309) were used to enhance and better resolve the released SMAPs. Firstly, 640-nm laser light was used to excite the AlexaFluor647 dye and switch it to the dark state. Secondly, 488-nm laser light was used to excite the AlexaFluor488 dye and switch it to the dark state. Thirdly, 560-nm laser light was used to excite the CF568 dye and switch it to the dark state. An additional 405-nm laser light was used to reactivate the AlexaFluor647, AlexaFluor488 and CF568 fluorescence. The emitted light from all dyes was collected by the same objective and imaged with an electron-multiplying charge-coupled device camera at a frame rate of 10 ms per frame. A maximum of 5,000 frames for AlexaFluor647 and AlexaFluor488 and a minimum of 50,000 frames for CF568 were acquired.

As multicolor dSTORM imaging is performed in sequential mode by using three different optical detection paths (same dichroic but different emission filters), an image registration is required to generate the final three-color dSTORM image. Therefore, fiducial markers (TetraSpeck™ Microspheres, ThermoFisher Scientific; #T7279) of 100 nm, which were visible in 488-nm, 561-nm and 640-nm channels, were used to align the 488-nm channel to 640-nm channel. The difference between 561-nm channel and 640-nm channel was negligible and therefore transformation was not performed for 561-nm channel. The images of the beads in both channels were used to calculate a polynomial transformation function that maps the 488-nm channel onto the 640-nm channel, using the MultiStackReg plug-in of ImageJ (National Institute of Health), to account for differences in magnification and rotation, for example. The transformation was applied to each frame of the 488-nm channel. dSTORM images were analysed and rendered using custom-written software (Insight3, provided by B. Huang, University of California, San Francisco). In brief, peaks in single-molecule images were identified based on a threshold and fit to a simple Gaussian to determine the x and y positions. Only localizations with photon count ≥2000 photons were included, and localizations that appeared within one pixel in five consecutive frames were merged together and fitted as one localization. The final images were rendered by representing the x and y positions of the localizations as a Gaussian with a width that corresponds to the determined localization precision. Sample drift during acquisition was calculated and subtracted by reconstructing dSTORM images from subsets of frames (500 frames) and correlating these images to a reference frame (the initial time segment). ImageJ was used to merge rendered high-resolution images (National Institute of Health).

### Coordinate-Based Colocalization (CBC) Analysis

Coordinate-based colocalization (CBC) analysis between TSP-1 and WGA was performed using an algorithm. To assess the correlation function for each localization, the x-y coordinate list from TSP-1 and WGA dSTORM channels was used. For each localization from the TSP-1 channel, the correlation function to each localization from the WGA channel was calculated. This parameter can vary from −1 (perfectly segregated) to 0 (uncorrelated distributions) to +1 (perfectly colocalized). The correlation coefficients were plotted as a histogram of percentage of occurrences with a 0.1 binning. The percentage of TSP-1 positive signal that colocalizes with WGA signal is the sum of percentages from +0.5 to +1.

### Mass Spectrometry

CD8⁺ T-cell released SMAPs captured on stimulated or unstimulated SLB were lysed with 1× ice-cold lysis buffer (Cell Signaling Technology; #9806S) supplemented with 1× Protease/Phosphatase inhibitor cocktail (Cell Signaling Technology; #5872). Lysates were cleared by centrifugation, digested with trypsin and analysed on a LC-MS/MS platform consisting of Orbitrap Fusion Lumos coupled to a UPLC ultimate 3000 RSLCnano (ThermoFisher Scientific). Proteomic data was analysed in Maxquant (V1.5.7.4) and Progenesis QI 4.1 (Waters, ID: Mascot 2.5 (Matrix Science)) using default parameters and Label Free Quantitation. The data was searched against the human Uniprot database (15 Oct. 2014). Only proteins that were detected as distinctive for the stimulated condition compared to unstimulated condition were identified. STRING version 11.0 (http/string-db.org/) database was used to visualize the network plot of the proteins identified specifically in SMAPs released on activating SLB and that were present in at least two from three independent experiments. The list of all identified proteins is available (Data. S1).

### Cryo-Soft X-Ray Tomography (CSXT)

Carbon coated transmission electron microscopy (TEM) grids (Quantifoil, TAAB Laboratories equipment Ltd; #G255) were coated with 0.01% poly-L-lysine (PLL) (Sigma Aldrich; #P8920) for 20 min. After PLL coating the TEM grids were incubated with 2.5 μg/mL of ICAM-1-Fc (R&D Systems; #720-IC) and 5 μg/mL of anti-CD3ε (BioLegend; #317302) in PBS for two hours at 37° C., followed by extensive rinse with PBS. CD8⁺ T-cells were incubated on the TEM grids for two hours and flushed out with ice-cold PBS, and the released SMAPs were immediately plunge-frozen in liquid ethane. Tilt series were collected on the Xradia UltraXRM-S220c X-ray microscope (Zeiss) at the B24 beamline of the Diamond synchrotron with a Pixis-XO:1024B CCD camera (Princeton Instruments) and a 40 nm zone plate with X-rays of 500 eV. Tilt series were collected from −70° to +700 with an increment of 0.5°.

X-ray tomograms were reconstructed using etomo part of the IMOD package. Manual segmentation of the CD8⁺ T-cell released SMAPs was performed by using the TrakEM2 plugin in ImageJ (National Institute of Health).

### CRISPR Cas9 Genome Editing

Freshly isolated CD8⁺ T-cells were washed three times in Opti-MEM (Gibco; Ser. No. 11/058,021). For 1.5×10⁶ cells, RNP complexes were prepared by mixing trans-activating CRISPR RNA (Alt-R Cas9 tracrRNA) and target-specific CRISPR-Cas9 gRNA for TSP-1 (IDT; Hs.Cas9.THBS1.1.AC; sequence: GTCTTCAGCGTGGTGTCCAA (SEQ ID NO: 45)) or galectin-1 (IDT; Hs.Cas9.LGALS1.1.AA; sequence: CGCACTCGAAGGCACTCTCC (SEQ ID NO: 46)) in equimolar amounts (200 pmol) prior to incubation at 95° C. for 5 min. 150 pmol of Alt-R S.p. Cas9 Nuclease V3 (IDT; #1081058) and the duplexed gRNA were mixed in IDT nuclease-free duplex buffer and assembled for 15 min at 37° C. Alt-R Cas9 Electroporation Enhancer (IDT; #1075915) (200 pmol) was added to the resultant RNP complexes and mixed with the cells in 50 μL of Opti-MEM prior to electroporation in an ECM 880 Square Wave Electroporator (BTX Harvard Apparatus). The cells were expanded with anti-CD3/anti-CD28 T-cell activation and expansion beads for 3 days in complete R10 medium supplemented with 50 Units/mL of IL-2. After three days of incubation the beads were removed, and the cells were seeded with 35 Units/mL of IL-2 in complete R10 medium at 10⁶ cells/mL for further two days. The activated and rested cytotoxic CD8⁺ T-cells were used next day. The percentage of knockout cells was assessed by immuno-blotting.

### Nanoparticle Tracking Analysis (NTA)

NTA analysis of the NK92 cells derived EVs was performed with a ZetaView (Particle Metrix) instrument. Five 30s videos of each sample were recorded and from these the EVs mean diameter, total number of EVs and EVs concentration was calculated. Each sample was measured in duplicate.

### LDH Cytotoxicity Assay

CD8⁺ T-cells were plated onto stimulated or unstimulated SLB with increased amounts of anti-CD3ε-Fab (30, 300 and 3000 molecules/μm²) for 90 min at 37° C. After incubation, the cells were flushed out with ice-cold PBS and the released SMAPs captured on SLB were incubated for further four hours with target cells (CHO). After incubation, the supernatant was collected, spun down to remove cells and cell debris, and used to assess the cytotoxicity levels by measuring the amount of released lactate dehydrogenase (LDH) following the manufacturer's protocol (TaKaRa Bio; #MK401). For cell-cell mediated cytotoxicity assays, 5×10⁶ target cells (K562) were pulsed with 10 μg/mL of anti-CD3ε (BioLegend; #317326) for 1 hour at 4° C. After washing out the unbound anti-CD3ε, target cells were incubated with CD8⁺ T-cell blasts, or with TSP-1 or galectin-1 knockout CD8⁺ T-cells at 1:1 ratio for 2 hours at 37° C.

After incubation, cells were spun down and the cytotoxicity levels were quantified by measuring the amount of released LDH in the supernatant following the manufacturer's protocol. Data were normalized to the control condition (CD8⁺ T-cell blasts).

### Enzyme-Linked Immunosorbent Assay (ELISA)

CD8⁺ T-cells were plated onto stimulated or unstimulated SLB for 90 min at 37° C. After incubation, supernatants were recovered, and cells were removed with ice-cold PBS. CD8⁺ T-cell released SMAPs were rinsed twice in ice-cold PBS and disrupted with 1× ice-cold lysis buffer (Cell Signaling Technology; #9806S) supplemented with 1× Protease/Phosphatase inhibitor cocktail (Cell Signaling Technology; #5872). Cell supernatants and CD8⁺ T-cell released SMAPs lysates were cleared by centrifugation. TSP-1, Prf1 and Gzmb presence was quantified by sandwich ELISA (Abcam; ab193716; ab46068; ab235635; respectively), according to manufacturer's instructions. Absorbance was measured at 450 nm.

### Cytokine Array

Calu-3 cells were seeded on 8 well μ-slide IBIDI well (IBIDI; #80821) (25×10³, 50×10³ and 100×10³ cells/well). After three days EVs from NK92 cell line (48 and 96 hours) were incubated with Calu-3 cells for four hours. Cell supernatants were recovered and centrifuged at 350 g for 5 min at RT to remove cells and cell debris. Cytokine and chemokine production was quantified in the supernatants by Human XL Cytokine Array kit (R&D Systems; #ARY022B), according to the manufacturer's instructions. The positive signal from cytokines was determined by measuring the average signal of the pair of duplicate spots by using ImageJ (National Institute of Health). Differences between arrays were corrected by using the average intensity of positive spots within the array. Fold change of the cytokine and chemokine production between conditions was determined by normalizing the data to EVs alone at 48 and 96 hours.

### Immuno-Blotting

CD8⁺ T-cells were plated onto stimulated or unstimulated SLB for 90 min at 37° C. After incubation and cell removal with ice-cold PBS, the CD8⁺ T-cell released SMAPs were rinsed twice in ice-cold PBS and disrupted with 1× ice-cold lysis buffer (Cell Signaling Technology; #9806S) supplemented with 1× Protease/Phosphatase inhibitor cocktail (Cell Signaling Technology; #5872). Lysates were cleared by centrifugation and reduced in protein sample loading buffer (Li-Cor; #928-40004), resolved by 4-15% Mini-PROTEAN SDS-PAGE gel (Bio-Rad; #4561084), transferred to nitrocellulose membrane, and immuno-blotted with anti-Gzmb (Cell Signaling Technology; #4275S), anti-CD45 (Cell Signaling Technology; #13917S), anti-LAMP-1 (Cell Signaling Technology; #9091S), anti-β2-Integrin (Cell Signaling Technology; #73663S), anti-TSP-1 (ThermoFisher Scientific; #MA5-11330), anti-galectin-1 (Cell Signaling Technology; #12936) and anti-Prf1 (Abeam; #Ab97305) antibodies. Immuno-blotting analysis of TSP-1 in whole cell lysates of CD8⁺ T-cells, primary NK cells and primary CTLs, under reducing or non-reducing conditions, was performed with anti-TSP-1 antibodies binding to different epitopes of TSP-1 (Abeam; #263952; Cell Signaling Technology; #37879s; ThermoFisher Scientific; #MA5-11330, #MA5-13390). Purified full length human TSP-1 protein isolated from platelets (Sigma Aldrich; #605225-25UG) was used as a control.

For the characterization of the EVs from NK92 cells the following primary antibodies were used: anti-CD63 (Biolegend; #353017), anti-CD81 (Biolegend; #349514), anti-TSG101 (Sigma Aldrich; #T5701), anti-Cytochrome C (Cell Signaling Technology; #11940S), anti-Calnexin (Cell Signaling Technology; #2679S), anti-GM130 (Cell Signaling Technology; #12480S) and anti-β-actin (Cell Signaling Technology; #3700S).

Near-Infrared Western Blot Quantitative Detection was performed using the Odyssey CLx system (Li-Cor) and the images were quantified using the Image Studio Lite software.

### Statistical Analysis

Samples were tested for normality with a Kolmogorov-Smirnov test. The statistical significance for multiple comparisons was assessed with one-way analysis of variance (ANOVA) with Tukey's post hoc test. All statistical analyses were performed with OriginPro 9.1 (OriginLab) analysis software.

### Example 1—the Kinetics of SMAPs (Proteinaceous Particle) Release

First, the kinetics of SMAPs release were investigated. Gzmb-mCherry-SEpHluorin transfected human CD8⁺ T-cells were incubated on a supported lipid bilayers (SLB) coated with laterally mobile ICAM-1 and anti-CD3ε (FIG. 1B, FIG. 6, SF2). Total internal reflection fluorescence microscopy (TIRFM) demonstrated that CTLs recruited acidic SLs displaying only mCherry fluorescence to the IS with activating SLB. This was rapidly followed (within 1 min) by appearance of SEpHluorin puncta in the IS (FIG. 1B, FIG. 6, SF2, Movie S4). Consistent with release of Gzmb in a SMAP, the SEpHluorin signal persisted in the IS for 20 minutes rather than dispersing.

### Example 2—SMAPs Remained Attached to the SLB after Removal of the CTLs

It was next determined if the SMAPs remained attached to the SLB after removal of the CTLs (FIG. 1C, Movie S5). Untransfected CTLs were incubated on the activating SLB, and either directly prepared for immunofluorescence detection of Prf1 and Gzmb or the cells were removed prior to analysis (FIG. 1D). Prf1 and Gzmb immunoreactivity were detected in the IS within 20 minutes, due to the kinetics of antibody binding (FIGS. 7-8, SF3-4; Movies S6-9), and remained as discrete particles attached to the SLB after the CTLs were removed (FIG. 1D). The SMAPs were stable without loss of Prf1 and Gzmb for hours without fixation (FIG. 9, SF5).

### Example 3—Target Cell Killing Ability of SMAPs

The ability of SMAPs to kill target cells was tested using a cytotoxicity assay based on release of the cytoplasmic enzyme lactate dehydrogenase (LDH). Target cells were killed by SLB immobilized SMAPs (FIG. 1E, black circles) after correction for “spontaneous release” of LDH by target cells (FIG. 1E, red circles (*)). It was also confirmed that SMAPs lacked LDH activity (FIG. 1E, blue triangles). Thus, SMAPs are stable after release from CTLs and can kill cells autonomously.

### Example 4—SMAP Characterisation

SMAPs captured on SLB (as discussed in Example 3) were subjected to mass spectrometry (MS) analysis. Over 285 proteins that were consistently present in SMAPs (FIG. 2A, B) were identified. Of these, 82 were unique to SMAPs on SLB with ICAM-1 and anti-CD3ε versus ICAM-1 alone and 18 proteins were detected in a majority of experiments (FIG. 10, SF6). One peptide from Prf1 was detected in multiple experiments and multiple Gzmb peptides were identified in all experiments (Figure S6). A number of proteins involved in cell signaling (cytokines and chemokines) were identified (FIG. 10, SF6). The presence of Prf1 and Gzmb in SMAPs was further confirmed by SDS-PAGE and immuno-blotting (FIG. 11, SF7). Plasma membrane proteins such as the phosphatase CD45 and the degranulation marker LAMP-1 (CD107a) were not detected (FIG. 11, SF7). This suggested minimal contamination with cellular membranes. LFA-1 was confirmed by immune-blotting, but not by immunofluorescence of SMAPs and thus may represent adhesion sites left on the SLB in parallel with SMAPs. Thrombospondin-1 (TSP-1) stood out as a candidate based on its signature Ca²⁺ binding repeats, which resonated with well-established Ca²⁺ dependent steps in CTL mediated killing. Live imaging of the release of SMAPs on activating SLB showed that TSP-1 and Prf1 are released together (FIG. 12, SF8; Movie S10). In addition, TIRFM on SMAPs from CTLs transfected with full length TSP-1 with a C-terminal GFPSpark revealed co-localization of the GFP signal with Gzmb and Prf1 antibody staining in the SMAPs (FIG. 2C; FIG. 13, SF9), and anti-TSP-1 antibody staining co-localized with mCherry and pHluorin signals from CTLs transfected with Gzmb-mCherry-pHluorin (FIG. 14, SF10). TSP-1-GFPSpark and Gzmb-mCherry-SEpHluorin were co-localized within cytoplasmic compartments in co-transfected CTLs (FIG. 15, SF11). This result suggested that SMAPs were preformed and stored in SLs. Enzyme-linked immunosorbent assays on soluble and SLB fractions from stimulation of primary CD8⁺ CD57⁺ CTLs revealed similar levels of Gzmb and Prf1 in both fractions, but the dependence on anti-CD3ε stimulation was higher for the SLB fraction (FIG. 16, SF12). In contrast, TSP-1 was almost exclusively in the SLB fraction, and displayed significant dependence on anti-CD3ε stimulation (FIG. 16, SF12). When we analysed TSP-1 protein by SDS-PAGE and immuno-blotting we found that CTLs and SMAPs contained not the full-length, 145 kDa species stored in platelets, but a C-terminal 60 kDa fragment under non-reducing and reducing conditions, which included the Ca²⁺ binding repeats (FIG. 17, SF13). CRISPR/Cas9 mediated knockout of TSP-1 by 60% in CTLs reduced anti-CD3ε redirected killing of K562 cells by 30% (n=5, p<0.001), whereas knockout of another similarly enriched protein, galectin-1, by 90% had no effect on killing (FIG. 2D, E). While TSP-1 is associated with T cell adhesion to extracellular matrix, TSP-1 knockout did not alter T cell adhesion to activating SLB, but did reduce the signals for TSP-1, Prf1 and Gzmb in SMAPs (FIG. 18, SF14). These results suggested that the C-terminal domain of TSP-1 was a component of SMAPs and is important in CTL mediated killing.

### Example 5—the Organization of Molecules within SMAPs

The organization of molecules within SMAPs was investigated at 20 nm resolution by direct Stochastic Optical Reconstruction Microscopy (dSTORM). SMAPs were detected with WGA in clusters of 27±12 SMAPs per IS (FIG. 3A). On closer inspection, WGA staining appeared as a dense ring in the 2D projections, which indicated a spherical shell with an average diameter of 120±43 nm (FIG. 3A). Many supramolecular assemblies use phospholipid bilayers as a scaffold and thus we asked if SMAPs stain with the lipophilic membrane dye DiD, which brightly stains extracellular vesicles or lipoproteins. DiD did not stain SMAPs, consistent with the paucity of membrane proteins detected in the mass spectrometry (FIG. 19, SF15). Thus, the WGA staining pattern was most consistent with a shell of glycoproteins, rather than a phospholipid-based membrane surrounding SMAPs. The location of TSP-1 in SMAPs was investigated by multicolor dSTORM. Strikingly, TSP-1 co-localizes with WGA (59±3%) and similarly highlights the shape of the SMAPs (FIG. 3B; FIG. 20, SF16). Thus, SMAPs from CTLs have a glycoprotein shell that includes TSP-1.

### Example 6—Further SMAP Characterisation

The structure of SMAPs was further investigated using used Cryo-Soft X-ray Tomography (CSXT), a non-destructive 3D method based on the preferential absorption of X-rays by carbon rich cellular structures within unstained, vitrified specimens with a resolution of 40 nm. For this, CTLs were incubated on EM grids coated with ICAM-1 and anti-CD3ε. After incubation, samples were plunge-frozen with the T-cells in place or removed to leave only the SMAPs. Released SMAPs captured on the grid after cell removal (FIG. 3C; Movie S12) were readily resolved and had an average diameter of 111±36 nm (FIG. 21, SF17). The slightly larger size of SMAPs by dSTORM reflects the contribution of −9 nm based on the 2.45 nm hydrodynamic radius of WGA. The carbon dense shell observed in CSXT was consistent with the TSP-1/WGA shell observed by dSTORM. The CSXT analysis further emphasized intracellular multicore granules in the CTLs that appeared to be tightly packed with SMAPs, where the lower density cores were resolved (Movie S13). These multicore granules were associated with the basal surface of CTLs near activating grids (FIG. 3D; Movie S14), as expected.

### Example 7—the Location of Cytotoxic Proteins within SMAPs

3-color dSTORM was used to determine the location of cytotoxic proteins within SMAPs. The TSP-1/WGA shell enclosed partly overlapping Prf1 and Gzmb positive areas across the 2D projection (FIG. 4A,B). Srgn was also detected in the core of SMAPs (FIG. 22, SF18). Given the apparent density of material in the shell and stability of SMAPs, it was surprising that 150 kDa antibodies had access to components in the core. SMAPs containing Prf1 and/or Gzmb were bigger and more abundant than WGA⁺ particles devoid of cytotoxic proteins (FIG. 4C, D). Primary CD8⁺ CD57⁺ CTLs and NK cells from peripheral blood also released SMAPs with Prf1, Gzmb and TSP-1 (FIG. 23, S1F9). These results confirmed that SMAPs are autonomously cytotoxic, −120 nm in diameter with a dense shell including TSP-1, a core of Prf1, Gzmb and Srgn and surprising accessibility to antibodies.

### Example 8—Hybrid Particle

CTLs can also use the ligand for the death receptor Fas (FasL) to kill targets expressing Fas. We only detected FasL in the CTL IS when Fas glycoprotein was incorporated in the SLB with ICAM-1 and anti-CD3ε (FIG. 24. SF20). In these cases, FasL distribution in the IS was in puncta distinct from Prf1 and Gzmb. The related protein CD40L is released in a CD40 dependent manner in helper T-cell IS. Synaptic ectosomes are a type of extracellular vesicle similar to exosomes, but generated by budding from the plasma membrane of the T-cell in the IS. These results suggested that there were two types of cytotoxic particles released by CTLs in contact with Fas expressing targets—vesicles with FasL and SMAPs.

### CONCLUSION

The working model for SMAPs function is that they act as autonomous killing entities with innate targeting through TSP-1 and potentially other shell components. While SMAPs transferred through the IS may only impact one target, CTLs can kill without an IS using a process involving rapid motility. The ability of SMAPs to autonomously select targets may become important in situations where delivery is less precise. SMAPs may have other modes of action potentially including chemoattraction through CCL5 and immune modulation through IFNγ. The TSP-1 C-terminus contains the binding site for the ubiquitous “don't eat me” signal CD47. SMAPs may thus partner with myeloid cells to ensure that any cell that cannot be killed by SMAPs is culled by phagocytosis.

1. An isolated proteinaceous particle comprising a core of perform and/or granzyme, the core being surrounded by a glycoprotein shell comprising thrombospondin-1 (TSP-1) or a fragment thereof, or a variant TSP-1 having at least 85% identity to SEQ ID NO: 9, 25, or 27-30.

2. An proteinaceous particle according to claim 1, wherein the granzyme and/or the thrombospondin is genetically modified.

3. A proteinaceous particle according to claim 1, comprising granzyme A, B, H, M and/or K, or a variant or fragment or orthologue thereof.

4. A proteinaceous particle according to claim 1, wherein the shell comprises a mature polypeptide sequence substantially as set out in the polypeptide chain of SEQ ID NO. 1, 2, 3, 4 and/or 5, or a variant or fragment or orthologue thereof.

5. A proteinaceous particle according to claim 1, wherein the perforin comprises a polypeptide sequence substantially as set out in SEQ ID NO. 6, or a variant thereof or fragment thereof or orthologue thereof.

6. A proteinaceous particle according to claim 1, wherein the glycoprotein shell is not a plasma membrane or phospholipid/cholesterol membrane.

7. A proteinaceous particle according to claim 1, wherein the TSP-1 comprises a polypeptide sequence as set out in SEQ ID NO. 9 or a variant thereof or fragment thereof or orthologue thereof.

8. A proteinaceous particle according to claim 1, wherein the glycoprotein shell further comprises other members of the thrombospondin family, optionally TSP-2, TSP-3, TSP-4 and/or TSP-5.

9. (canceled)

10. A proteinaceous particle according to claim 1, wherein the glycoprotein shell further comprises galectin-1 and/or galectin-7.

11-12. (canceled)

13. A proteinaceous particle according to claim 1, wherein the proteinaceous particle is attached to a membrane vesicle/phospholipid particle comprising FasL.

14. A proteinaceous particle according to claim 1, wherein the glycoprotein shell and/or core of the proteinaceous particle further comprise a toxin, such as chlorotoxin joined to the TSP-1, or joined to one of TSP-4, galectin-1 or galectin-7 also provided in the proteinaceous particle.

15. (canceled)

16. A proteinaceous particle according to claim 1, comprising a genetically modified shell protein, a genetically modified core protein a transgenic protein and/or an antibody or a fragment thereof.

17. A proteinaceous particle according to claim 16, wherein the genetically modified shell protein is a thrombospondin fusion protein, a galectin fusion protein and/or a granzyme fusion protein.

18. A proteinaceous particle according to claim 17, wherein the fusion protein comprises an antibody or antibody fragment, optionally a scFv, a VL and/or VH, a Fd, an Fv, an Fab, a Fab′, a F(ab′)2, an Fc fragment, an antibody mimetic, or a bispecific antibody.

19. A proteinaceous particle according to claim 17, wherein the thrombospondin fusion protein is a TSP-1 fusion protein or a TSP-4 fusion protein.

20. A proteinaceous particle according to claim 19, wherein the TSP-1 fusion protein is a TSP-1/T1-scFv fusion protein, a T1-scFv/TSP-1 fusion protein, a TSP-1/chlorotoxin fusion protein or a chlorotoxin/TSP-1 fusion.

21. (canceled)

22. A modified cell capable of producing a proteinaceous particle according to claim 1, the modified cell comprising:
A) nucleic acid encoding:
perform and/or granzyme;
thrombospondin-1 (TSP-1) or a fragment thereof, or a variant thereof having at least 85% identity to SEQ ID NO: 9, 25, or 27-30; and
a heterologous polypeptide, such as a transgenic ligand in the form of a fusion protein with a thrombospondin, a galectin or a granzyme, or
B).

23. The modified cell according to claim 22,
wherein the perform, granzyme and/or TSP-1 are recombinant.

24. The modified cell according to claim 22, wherein the modified cell further comprises a shell protein selected from the group comprising galectin-1 or a variant thereof having at least 85% identity to SEQ ID NO: 15, galectin-7 or a variant thereof having at least 85% identity to SEQ ID NO: 17, or TSP-4 or a variant thereof having at least 85% identity to SEQ ID NO: 12.

25-43. (canceled)

44. A method of treating cancer, the method comprising administering the proteinaceous particle according to claim 1 to a subject.

45. (canceled)


SEQUENCE LISTING (46 sequences)

SEQ ID NO: 1 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 2 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 3 - protein, 246 aa, Homo sapiens
MQPFLLLLAFLLTPGAGTEEIIGGHEAKPHSRPYMAFVQFLQEKSRKRCGGILVRKDFVLTAAHCQGSSINVTLGAHNIKEQERTQQFIPVKRPIPHPAYNPKNFSNDIMLLQLERKAKWTTAVRPLRLPSSKAQVKPGQLCSVAGWGYVSMSTLATTLQEVLLTVQKDCQCERLFHGNYSRATEICVGDPKKTQTGFKGDSGGPLVCKDVAQGILSYGNKKGTPPGVYIKVSHFLPWIKRTMKRL

SEQ ID NO: 4 - protein, 257 aa, Homo sapiens
MEACVSSLLVLALGALSVGSSFGTQIIGGREVIPHSRPYMASLQRNGSHLCGGVLVHPKWVLTAAHCLAQRMAQLRLVLGLHTLDSPGLTFHIKAAIQHPRYKPVPALENDLALLQLDGKVKPSRTIRPLALPSKRQVVAAGTRCSMAGWGLTHQGGRLSRVLRELDLQVLDTRMCNNSRFWNGSLSPSMVCLAADSKDQAPCKGDSGGPLVCGKGRVLARVLSFSSRVCTDIFKPPVATAVAPYVSWIRKVTGRSA

SEQ ID NO: 5 - protein, 264 aa, Homo sapiens
MTKFSSFSLFFLIVGAYMTHVCFNMEIIGGKEVSPHSRPFMASIQYGGHHVCGGVLIDPQWVLTAAHCQYRFTKGQSPTVVLGAHSLSKNEASKQTLEIKKFIPFSRVTSDPQSNDIMLVKLQTAAKLNKHVKMLHIRSKTSLRSGTKCKVTGWGATDPDSLRPSDTLREVTVTVLSRKLCNSQSYYNGDPFITKDMVCAGDAKGQKDSCKGDSGGPLICKGVFHAIVSGGHECGVATKPGIYTLLTKKYQTWIKSNLVPPHTN

SEQ ID NO: 6 - protein, 555 aa, Homo sapiens
MAARLLLLGILLLLLPLPVPAPCHTAARSECKRSHKFVPGAWLAGEGVDVTSLRRSGSFPVDTQRFLRPDGTCTLCENALQEGTLQRLPLALTNWRAQGSGCQRHVTRAKVSSTEAVARDAARSIRNDWKVGLDVTPKPTSNVHVSVAGSHSQAANFAAQKTHQDQYSFSTDTVECRFYSFHVVHTPPLHPDFKRALGDLPHHFNASTQPAYLRLISNYGTHFIRAVELGGRISALTALRTCELALEGLTDNEVEDCLTVEAQVNIGIHGSISAEAKACEEKKKKHKMTASFHQTYRERHSEVVGGHHTSINDLLFGIQAGPEQYSAWVNSLPGSPGLVDYTLEPLHVLLDSQDPRREALRRALSQYLTDRARWRDCSRPCPPGRQKSPRDPCQCVCHGSAVTTQDCCPRQRGLAQLEVTFIQAWGLWGDWFTATDAYVKLFFGGQELRTSTVWDNNNPIWSVRLDFGDVLLATGGPLRLQVWDQDSGRDDDLLGTCDQAPKSGSHEVRCNLNHGHLKFRYHARCLPHLGGGTCLDYVPQMLLGEPPGNRSGAVW

SEQ ID NO: 7 - DNA, 18388 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggtaagtcgcccccggcggccgccgaggaccaaagctgcccgggacatccacctggagcgctgaggcttcagtccctctggtggaccccggaacctacactctccccgctcgcctaccccagcccgctcctctcagccgctggaggactcttcagggcaaggctccagagccatcctctccagccttgaggttcacaaaccaactcatcaggacaccccaagatttccttactctctgaagtcctccttaagcctttgtatcagcactccagggaagagtctgtacttcccctgccctccctgcaaccccaaactacagttcctgatcttgctcaccttcgacttcccaaaagcccccaaattgttggtcttgcgccccccacactttaaaaccagcatctctttcctccacctctctctctctctctctctctctctctcatgctctctggaaagtaatcctgttgtccttcatttcagcaaacgcggggtcaaagagggatggtcccggccctgtccccatgcccagccccgtttctgcgcaccgtccctccctgcctagctgatctctgaccctcggctcttgtgcttcctgctacaggatccctgctgggcaccaacagctccaccatggggctggcctggggactaggcgtcctgttcctgatgcatgtgtgtggcaccaaccgcattccaggtgagtttgtgtggcacctttaggggaaggagggacaaggaagaggtgctggctatcccaggtgtgccccctcacgtgctgtcctctccctcttgagcctgactggacatcaggacgcagcttcactctgatcctggtatttattcacctctttcagtggttgccaggagttttcaccccaaccctttgtctccacccctaaggactcagccccctactgctggtcccagcctagaaagctcactttgtgttctctcctgtctaacagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccaggtgaggcttcttctctgagccctgctccgtgggatcatctgctagacaggtgacctgccaggagggctacaggaaatcctgtctgtaaactaacgcagcagttctcagattttaggcagcatgagcatcacctggagggcttgtgaaagctcaccctgctgcaccccagccccagagtctgggattctgtaaaactgcaactctaacaagtaccaggtgatgctgatgccggtggtctgggaagcacactttgaaaactactcaaggaaacaagaaatggttttcctttagcagtcagcctgttcaagccttagatactccttaggctcttaagaaaagagcttatttcaaatgcattcccccaattttaatcttgtttgaaatggatactttctatgggcagaatcagtaccactttgctattggattcctcatggggatgcagctggtctcacataaaaggttaaaatagcagttagttgttgctgttacagcttcatctgtgccttaacttctagatctttgcaacggtggctttgaactcccacatttaactctttgacactggaaagtgtcattatatcctctaggcttaggatcagattccatttcctgtgtacccaagtaggctgataactgaattctttgtaaaatactcaagacttggcagaagtcacaattaagattttgaagttgtttggcgattaatgttgctgtgttattttgacatagtgatcttaagacagctctttgaagcttttagaacatctattgtatgcttttcctagcatacagacagtctcccccaaccccatccccaccccgctctgcattctacaagtaatgtgtgtcctctgcccacagggggtgctgcagaatgtgaggtttgtctttggaaccacaccagaagacatcctcaggaacaaaggctgctccagctgtgagtacccctctatttttagggcacatagggaatcagggggaattccaccaaaaacaaactgaggaatttagcaatagtggttatacataaattacccccagtgagatcatcaagaggcatacagaagtgactccaaggggtattatacacacacacgtatacacacgtgcgcatacacgcaacccctctacctgcatccttcacaccaaatgaaacgtcttctaccactaagaactcaagaggctggttggtaagagtatctatttgaagttttcattttgtttcttgccagctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtggtcagtggcctctgcacccagcccgttagcatgaaccctggaaggtttatcgcagatggtcccaaatgactgaaaatgcggggggacactaatgatattctctcccatttagactgaagagaacaaagagttggccaatgagctgaggcggcctcccctatgctatcacaacggagttcagtacagaaataacgaggaatggactgttgatagctgcactgagtgtcactgtcaggtaagggaccttcaccagccagaataagaatcgacggcttttgtttgaacctacatctttggggaaataccctaatcgccacagagattctttttatgttccatggcctgataacaaagtgttttttttttctttaagatgcaattatggcatctataagttcttctttaaactctcagatggtttatcaaagtattaattataccccaggctaagtatctaagagaatgaatctactaagacatctgaataatttacctaccttagttcaaagactaagtgttgaatgtcaggtaaataaaacctggttggaacattcatttttttttcccaaccctgatttatattcagctttggcataggttcttttagtacagtagcaattgtttttcaaatcacttagtctgacactgttaggatgttactgtctgctttgctcagatggcactgccaggtgtgtgattcactctagcatccccgcatgccagtggccagtgagtttggaggttcacctggcttatctattaatagcaaagtatttgaatttgcttaaatacaaatcacagatgactggctgtgggaaccaacttcattaaattccctttcttttccaatataaagacagatcacagatctatagatgaggaaaaagtgcttatagatcttccttcctccccccaaacacataatataaaaatgtaaaagtcctggctgaattttctgagtttgatttcagacatagatttgacccatgatgtttgttggttcctctgctataaattctttgtactgtaacttccacccttgactaagtagcaattaaaaagaactttccaaggaatggagtgacacgtaaactagcactagaactgtctctacgatagtggtggctgcaaatatgacagagacagatctatgaaaatcattccactagagcattgacagccctgaccatgttttggcttgtaaaggaaatgttacaggatgtagacttctgtatgcaacatgctcagcagcctgttcccctctcactctcttgccctgcagaactcagttaccatctgcaaaaaggtgtcctgccccatcatgccctgctccaatgccacagttcctgatggagaatgctgtcctcgctgttggcgtaagtttctcaaatggcatagctattcttcagtgactaggtcagttctacatacatcctagacagccgagcacaacactggaaccccacccccatctccatcatgcttccattcagtttcttgctgcagcagctggttaactgacagcttatctgctgtacagagccaaaactccattatgagtacatcgtaattacccatctggattgcagtaatggcagtgaagatcttggtatagctcattaattctgattttttgctttgaggaaaagagatgaagacgtggcagcaacagctctaagttgactgcttttagaatttgaccttagtggcacagagttaagaaaaatctgctcaccagagatattgacaggcttactgtgtaagcattggcctgatcgggcctggaagttttgcccctttcatctgtagggataagagggcctaagagaatagcagcaatctgagctctagagcaccaaaagaaataaggcacgatatcgttccatggcacgttatccctggcttttgttaccagtaaaatgaatgactttgctgagacaatacttagtgtagcaggtacaagttataaaagtgattcattacaactaagataaggaagaaaacaggaaaacagcacttgcccttcacacacacagaaacacacatgcacacacacacaaacacacccttgtcacagctcagaataaccaattccatccagggatcatattagcgatattggcactgtaatggtcgtttttacatttattttctgttccgcattcacttaacatgtgcgttaaaaggaacaaagctagctggaggtagcaaagttttgaacttgaatcagggattgctaagaaaacttacaactaatgaccacagaaagtaagttctttgtcttctgtctgatacagctagatgtgattgtagccgtggtgcctgggcagatggtaaacaaacatgctgtcctcttatgacaatatgtgcagagaaggccccaaacgctggggtgagaggaacaacaaactgactaactcacggccgagttggagccagaaccaacaacagctcggctgccttcacactcatctgtcaaacacatggtgtatggctcccctgacagaaatgactcctataagcttcctcatactcgcgcattcttggaagtctgagactggaaatgcctacccagatatgttaagtcagtaaaggaaaactcatttgcaatgctgagaggagaaagaaaatcaatgaataggagagcatacactgtgggaaggctcagctgtactgagccaggcaaagcatccgattaccccactgctgtacacatcatgacctgggataaacccctgggccgttggcctaagaaatatgttgcttttcatcctgtataactgcaactttgtgccggctcatattatgttctagagagctcatgtacaccatggtgaccatagttaataatactggattgtatgcttaaaatttgctaagagagtagatcttaagtcatcaccaaaaaaagtaactgtaagatgatggagatgtgttaattagcttgacggtggtaatcacaatatatgtgtatatcaaaacatcacattatacaccttaaatgtatataatatttgtcaattatacctcagtaaagccaaaaagaaataaatatattgcttttcaccctctggcaagtggagggccctgaacagagccctctaaagaacagcttgtcctaatcatcacgtacctgatgaacctctgtttcccctgcagccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatgtaagcatcttagcctctcagggacgtggagaactgacctgtccttgttgtcgtcaccaagggcagctccacagcatgtatattaagaacacgggttctaggatctctggatcccaatcccactgcttagctgtgcacccttgaacacaacttaccctctctgaggctctttcctcatttgcaacttggagataataatagttcttgataagtttatcttgagcattttatttagatactgcatgtaaagcagttagcactgagttttggtttatggtaagcaccctgtaaatgttaggatattaacagcaacccataaaaacagcgaacttatccgttctctggtagaaaacattccatgttgacatttgggcatgtggccaggagccaatttctaccgtacactgggtttagttgccattgaccatttcctggaaatacttctgcgtgctcctgatgggagcctctatttggctgtctctaagccaaaaccatttgtgaccatcaactctgtactttagttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccagtaagtgtgaggtccgctgcaagggtgagcatgggcagcagctctgcccagctggttgcctggcatctgcagcctgcagttcagtgggtcatagagcaggaaggttacctactagagaaacaaacagaagcaaagtcctgcaggctcagcaacttcttttaatgaaaaacaaactcaccctcttccccagcattctttccatgtgtcagagaagcagaggtttcttgaacgggcttaggagagtctatgacaagggagggatttgaaagttgatcttaattgttgcctgtggttcatcttcttacagtcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattggtgagccacgcagcccaggatgaaacgacccaggagctttgctcttttactgaatgctgcagtcagcattcgaggagattccagcttggttagtcctgagcgatttgattgctctaagatgcaggtggacaacataatcccaacaagttatcggttccctataccctataatatcttacactgtgttaagtgcccagcatggcagtatggcagcttagaccaaccatttactgtgactgtctctctctccttgtctcagatggatgcctgtccaatccctgctttgccggcgtgaagtgtactagctaccctgatggcagctggaaatgtggtgcttgtccccctggttacagtggaaatggcatccagtgcacagatgttgatgaggtgaggaactgatggggctccgagtttctggatctaggaaagcagctaacctgtgcagtcgcttccttatggcagtgacttctaaacatgatgcacgctcttatttcctccatagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtacagtcaactagacgagtaaaccagaggacaggagagctgtccttgaccaaaataactgggagcgggaggaatgtaatttcatacccttcaccaaaaaaaaaagggcgaggagatgaatgtacggtctagttttagaaacgtgattagaaaatccatggtaaatcctgcaggggaaaaacagtcttccatatttaaaaatgctgctctggaataagttgtgagcagatggacttgtaaacgcctaggtgctgagcaaattcaagaaaaataaacataaagcaaagtttgcttatagcctcagggagaatggggagggacagaggtaacccacactcttccaaatggagcctctgtctactcagagatgacagggatctggattcttgtttccatgatatctgaggattctcaaaagctctgtgtaacagcagcatggtgtaccctcaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggtagagccaggtcctttgtgtgcctccagaaagagggcccatcaccttatcaaaacacaggctaaggaattttaaatacttaaagtttggacatgaaactgaaagattaaatctcccaaagggagaggggcactaattcctattaaaattaaatcacttggtgccaaaaacatgaacagggctagctctttttgggcaactgtgttccatagtcaactcttctgtccaaaaaaaagttcagtactctatccaaataccagcaaagcatgacttgtgttcacaacatctataaagcctagagcatttacacaaattctgtatgtatatcccccaagaaaagttttattttcttgtcttcccataaaaaaaatatgctgttactgagccaaattctgttctttttctatttggaattccaggtacactccctcgtgcatgagctcagcatgaggaaggcaactgaagcagtgatatatatcttctctttcctaggataattgccccaaccttcccaactcagggcaggaagactatgacaaggatggaattggtgatgcctgtgatgatgacgatgacaatgataaaattccagatgacagggtaaaaacagttttctatccctttttcatcttttcagttcagcaacagcctgaaacactttgggattcaaggaaattacatggctatagcaaaaaatataccaaatcaatacacaggataattagaaattattcattgtgttccagtagtttaaggatgtagatgttgccaagagaatttttaaatgagggttttgtttttcatcagaactgtttttctctgtacttgagaaattataatgcataaacaaatgccactttgttccctagattcatttcaaatgtcacatcgaaattacagtaaaattgactttgggcacactatgaactgagatgatgggattatattctacatctcactaacttctaacccacagggatccatttttttaactatgtccttttaacttttgtagtgatcgttttacactgagtgatcaattagcctatccactaggtagaaagtattgctgattttcacagttttagacatattatgcacatggtttgaggcttgagctgttttcaaggacaacattgttaagtgctccatttcttctctttgcaggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtaaggtgctgcctgatcagagggcccgcgggagacagggacatgcacagctttccaaacgtacttctgtctagtcctggctattagtatgcactttggtggaaacatccaaggctggagagcccagctcttatttgtcccttgttctcttcaggtatcctcaatgaacgggacaactgccagtacgtctacaatgtggaccagagagacactgatatggatggggttggagatcagtgtgacaattgccccttggaacacaatccggatcaggtaggtggatggactcctttcagagtctttcagtaaactgttggaatatccctttcatggcctttgaaaaatgagcttaacaaagttcaaacactttcattactgtggctccctggcttctcatagccaactggaatacgttcatgtaaataatttgcctgatgtgagctccttgcacagagtctcacagatctttagcatgaagatctggtaacttcagtcttaccttctgtaagaagggctactggacttgctcagcatcgtctaccattgttaagacaaattatacaaagcagacatctgtgactgacaactgtacaataatattaatcatatgtaatactaagagatggaaaaataaatgtgcataagcaaaataaccattttcatggctaatgatgtaatctagctataagaaaatcatcaaactaaggatacatccaaaaccaaagtaatttatccctgtcataaacattacccagacagtatacaacacagggaccagtggtctgtttccatcttttcaggacttttcagagaagtcgacgtgtcaacagttttacaactgaaaccttctaaggaaattctctgttataaagtatccagaaaatttcatttaacattaagatgtaaacagttaacatttcatttcatctttagtaattaaatatcactctatttgaccttatttagaaagttttatatgtaaaacatttttaaattatcccttctcagatttttatacgaaaacaaagataaaggaaaaaaacttttaaaatgtaattgctactattgctatctttctgcaggagtgtgtaaatagacatgacaccccactggctgtatcaaacaatggagaattttccctgtggtgggggcataagttatctttaacatgaatggtttatactgcaatttaccctccatttacatctctctttcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggtgagtcatgggagccactttctaagacagggactgctggcacagctgtgtagattgaagaaatgaaaccaaggctcaaagcatttgacaggatgaagggaccaaatgccaacttagacaagatagtgacatttctgacaccagtaataataatagcactttagaattttgctgaactcttgcttttttgacctcaggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggtgagtagcgagttcttagatcctaagagactgatgcatacatggggaaaaacaaatataaaacctggcagttgtacctatccctgtgggtgctgaggatgtctaggaacatgatggagaaccttctgaaggctgcaggttttaacctggctctgggctcttcttccaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtaagaagcaaagccctggaacagagagagagcttatgggtgcctgactagcactggggatgctgtgctttgaccaagactctgaccagggagtcttagaaagttcccagcatcaccagctgcagcattgaactctgctttgtaaaaacataatagtgttgaaaagggagcttgaccaagaattgccctgcaaatcctaaggtgccttcagccttttcaaacaaaaaaacctccttccctcctctctgtctgctttatatgtgtgctcagtggcacacaacaaatatgagaggacttggaaaaattccccattgcagccctctaacttagatcagctcagtacctttcaagcattgtttctgatggaatgaaatagaaatctttacctgaaggagctgtgtttcaacctttccttttccttttccttcaggtgcgcaccctgtggcatgaccctcgtcacataggctggaaagatttcaccgcctacagatggcgtctcagccacaggccaaagacgggtttcattaggtacgatcatactgattcactttcacttacagtcacactgagggacaaaaagacaaaaagtattaaatagcattgtcactaaacaagattttttttccctgcagagtggtgatgtatgaagggaagaaaatcatggctgactcaggacccatctatgataaaacctatgctggtggtagactagggttgtttgtcttctctcaagaaatggtgttcttctctgacctgaaatacgaatgtagaggtaagagcaacatcaccatgaatgtacactggacatctctatttcagactaatatcaaggatgacggttatgggggagtccagtgtaaagactgttttggagacagggttatttctattttgcttttgaggacacaaggacaaaaatggaataatgcctaggcactgtggcttatgagttcctgagtccttagctataatattagtttgcttagcaatctctgtgctcctcatacacagtgcaaaggtaagctgcagaaaagctcctatataatttggacttcattaataatactgttctttacaattatatatttatatatcttactgcttattgttttatgcatgccatcacagcacgagttagcattcccaacttttccctgtatacaaagaagggagaggaatgttgctttcatattggcatgttaaattaatgttcactattaaacttagcattttttttcctcattcttttttacttagtcattacttaagctcgctgagtccaacactggctctaccacaaaataagtgcttaatatatatttactgggccaaggccagatacctaaaagatcaatgacagtagagaaagatggttgattttactcaaaaaaatctaaattattaatgtaatttttgagtccaaatttttaaaataagactccctaaactgttaacattgaaagcctttggaaagcataatatatgttctggaaggttcacgctgtgtcggtctcctagcatcaatgtcagctaataaaattaaatgctaatgtgcttgaacaaccttaaaattaggcttttgtcattagaaaagtagagctattcctatgtggttaacttattaactaagatgtctatgcttttatgaattagttttcatttgtatatttatttatatttgtttatttaacagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttatacttactgtatccatgcttatttgttctctactggctttatgtcatgaagtatatgcgtaaataccattcataaatcaatatagcatatacaaaaataaattacagtaagtcatagcaacattcacagtttgtatgtgattgagaaagactgagttgctcaggcctaggcttagaatttgctgcgtttgtggaataaaagaacaaaatgatacattagcctgccatatcaaaaacatataaaagagaaattatccctaagtcaagggcccccataagaataaaatttcttattaaggtcattagatgtcattgaatccttttcaaagtgcagtatgaaaacaaagggaaaaacactgaagcacacgcaactctcacagcgacattttctgacccacgaatgatgccttgggtgggcaacacgattgcatgttgtggagacacttcggaagtaaatgtggatgagggaggagctgtccttgcaatgttgagccaagcattacagatacctcctcttgaagaaggaataataagtttaatcaaaaaagaagactaaaaaatgtaaaatttggaaggaatccataaatgcgtgtgtgtctaaatacaaattatcatgtgaagaaaaggcccaagtgtaccaataagcagaccttgatttttggatgggctaattatgaatgtggaatactgaccagttaatttccagttttaatgaaaacagatcaaagaagaaattttatgagtaggttaaaggtctggctttgaggtctattaaacactagaaaggactggctgggtgagataaaatcttccttgttgattttcactctcattctataaatactcatctttctgagtagccatgatcacatacaaatgtaaattgccaaatcattttatagtaccaaggtgaagaagcaggaactagaaagtgttgataatagctgtggagttaggaaaactgatgtgaaggaaataattctttgaaatggcaaagaattaaataccatcattcattatcagaagagttcaacgtttgaagtgctgggagataattctaattcattcttggatagtgaagcaaaactgattgaaaataccaagataagacagaaaaagtgactggaaagaggagcttttcttccaggcatgttccagtttcaccctaagactgaccttcaaataatcaggttgtactgaaataaaggacttgttaaaaattaaaattatgtcatcgagatgatagcttttttcctcctccaacagtttattgtcatgtgttgtgggagagctcgagtgaagagcaataaactccaggtcttataagaatgtacatacaataaaggtggtgccagcagtttttttttttctaaagagtcacatgtagaaaagcctccagtattaagctcctgaattcattccttaaataaattggctctctctctcttctataatttctttttctttttatttttgagatgaagtcttgctctgtcgcccaggctggagtgcagtgacacaatctcggctcactgcaacctctgcctccccggttcaagcaattctccctcctgcctcagcctcccaagtagctgggactacaagcgcccgccaccaagcctggctaattctgtatttttagtaaagacggggtttcaccttgttccggacaaacactaagccctaaagggaaatccaaaataaaaacatctatttttaataacactttctatctaaatcagggtgactttttaaaaaaaatccggaagctttttgttgaattacgttacagacttagttaccagtccttgttagagttaccttcagttgacatgctgtgaatggtcccacctcttttatggcagaattcattacttaaaataactctattttcttcccccttacctaaataacagaaaggctcactatgtcccaaatatcattggcagaagcaaactataaagtcataagccctttgcagtgcaagtctagaaataatttt

SEQ ID NO: 8 - DNA, 5722 bp, Homo sapiens
ggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaaaagatttaaacaggatggtggctggagccactggtccccgtggtcatcttgttctgtgacatgtggtgatggtgtgatcacaaggatccggctctgcaactctcccagcccccagatgaatgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccgcaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcatcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcaccttgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttccgtaattactaggtagttttctaattctctcttttggaagtatgatttttttaaagtctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattccttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagaatattcagattgtgtagatatgctatttaaataatttatcaggaaatactgcctgtagagttagtatttctatttttatataatgtttgcacactgaattgaagaattgttggttttttcttttttttgttttttttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatattt

SEQ ID NO: 9 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 10 - DNA, 92422 bp, Homo sapiens
aatttgggggctcttgagagatgagagaaacaacgactggagggattaagaagaactcttaggagcagcttggcctaaagggtaagagttgtgtgccttaagtcagacccaccagtaattcttcaaatattaatatttattagatccctagagtgttccaaaccctttgaacattaaatctgtgtgatgcctaaattaacctgtttgtaaaacagggatgacccagtatttacaagattgtggtatttacaatgtcggagagaaagaacttggcacacaggaggcactcagtaattgttcaaccccttctcccatgtatgtggcctctgcctgcctttccagcctcaacctgttctcaccctcatgttcactggcacaaccctgcttaaaagttacagtatttctgaacgtcaggtaaattgtgcccaccttcagtgctggtccttcaatctggaatatctttcttccctatgcagctgctccctcaaaaatgagcttaactcaccttccctgtgatgtttttacaaagttaggagctctgggttcttgtagccccttgtgcatatttgaattgtagcttctctccctagctacagaccttttgcctttcacagaagagggctagcttcataggtttgggacctgtgcagccacacaaggctctgtgcttaaaaaggctcctcatttggctgatgctcttccgtcactgtcttgaaattcttaagttttgaacaaggggctctgcatttttattttggactgggcccctcaaatcatgtagctcgtcatgtctgtgggcctaatgagcacagactaaatgtttcttgacgttaaacctgacagtttctcagaaaaactattaaagaggaaattttggtcttggaaatgctatgatccaaacatatgactgggtctcagagttagaacttgggtgctgggtcttttttctagaaggcacagtagtctttctgggacaccctcgcagtcatgcctctgagttctttcctctattccactgagatggtcggatcttcttaactaaaactcaaaagaaacctctgcaacttttcgtaaaattactattcaacaaatatttgctaggcactcccttactgtttacataatcattcaaattacagtgccagcctgctaaagtcttttttacatgtttcaaaaccaagagttctaagaaatgaaaaagagatatgtcagaaatgagaattcattaataataactgtataattggaaaatgcataacatatgtaccattaaaactgtcgaaacatttattgagcagctcctatgtcttagacatcataaggttctggaacaatacagaaatatataaaacatgaattctgtcctcgggagtttacagtgcacaaaggagatatacacacaccaaggtgtaacaagtcagataatcacgagtagggttagaaatataagcaaaatactacagaaacacaaaggagggacagtgcatcctctgtgtgatcagtggagactatctgttgagtcttcagtataaagaaagtaaagtaattcaactttcagattgtaatggaacctccctgttttgagggttagaaatgtcaaaccatagactgacaaaggcaaggcagggggaaagaaaatcctaagccataaagctgagggtccatatactacattcaggcatttaaaacaatcatacagaaagaatgtggatttcctgagccttgtttgtagcaggactgagaggagtgaatgaacattcttagtggacacataacacagttactcagtttggccttcttccgtactgctgctactacatacagtgatttcatttgcattaaagagaaattaaatctcttcccttagtgttgaattgagacagaatagcatgtcattgcatctgatgaaagaatccttacacacaggtttattagtgcatagataatagcaatcttttattgctgcagttcctcttgagagtctgtatctgttagattgaatttaaaaaactctaacatagctgattgcacaaacaggctgaatactgttccagagaaccacacagaaaattgaagccttcagaaattatgcctggtatggttagttggctcctgtgccttttcctggctgttttagagtattgttcagagttcatctaaatcataacttcagattcctaccgggaagaatactgtctgcaaaacatccatgtttgtgtgggatggcctgaatcacagctttataatgagtatcccagacagtgttttccccaaaaaagaaatcttatttttcaaaagatgatcattaagaatggtttcatctggtgatagtcatctgaactcaggcaacctgaccttggcatccatacccatccttcatgtgtttcttaacttgggagccacagtgcaaggtttgttaggctctttttgtctgggtgattaagaatctgggaaatggtgctgttgggaaagatgaaaggtttgtgacaaatgatggtacagtatatggtcccttatgggaaaattaaattgtaaagtcttaaatatccctttgaacttgaagattctgtaatttgtgttcagatataaaatccttaaggaggggaaagaacaaacactgaatagaaaaaaggagagctgagttcttgacttggcctctgattagccatatgcgcttgggccaatcattttacattgatacctctgctcattcatttataaagcaaaagttcttttatctctgagatgctatcattttatgtgtaatgaacatttaataatttctgacatttatggagcaatttctacatgccagaagttgtttcaagtatctaacataacttgctttatctccataatgatttcatgacacaggtaccttcctaaggttaactaacatctttgtgtccctccttgaaaatagaagaaaatatcagtacccatatagccagtaggtggtagaaccaagatttgaacccaggcagttttggcttcagagtttatactcttaaccactgtgtcatactgcctcatgtagcctcctgtgtagtccattaaaaagcccagtttcacccaaagtggagatatcaatgccatgatattattgttattatttatgtaaataatatttgtttagtattacaccattaaacgaattcattaagcagcttctgtatgcctagcacactgcaagcctgtcagggaataaaaatttatgttcgcctgtaatcctagcactttgggagggaggatcacttgaacccaggagttcaagacaagcttggacaacatgatgaaaccttgtctcaacagaaacacaaaaattatagctgggcatgatggtgtgtgcctatagtctcagctacttgggaggctgaggcgggaggatggcttgagcccaggaggagaagattgcagtgagccgtgattgtgcaaaaacttatgttcaagcttttagaggcacagttgatgaaacaaggcccacatatttgaactaactagagactattaggaagtaccattttgtcaagaagacaagtctagtatggttttagacaaaggcttgaatgtactgcaatgcttggcaaaggcctaatgaaagaagatgatagctgatctcgaagggtgggtacgttgcatttagattggcacagagtaaggcattgcatatgggctgtgctatataagtgaaggcatagaaacaggactgactatggcaaggaatctgaagatgagtaaacgaatgctgggtttggttataagctggagactgctcagggagagtattggttgactaacttgagaaccagaattggaaggcatgggaatttatagttttgagaaagtaaagacaagtaaaagtccaaatacaatattagtggatttacatagtcaaggatgggaggtagacagctattgttacatggacattataagaagaagcattttataaacccataaactatattaaataaagtaaaagtgtggtggaaaatgctgctgaatattattggaggaaggtcacttggcacccttccgctgagcatttttgatagaggaggttgttagaaatccagtggcaagggattactggagaaaagggtataaacgataaggacaggtctaagagcatgaggaatatgactgataaaggaagaagagaaatgggacaggactgaaagactcaaagtttttgaaagagacgacatggtgaatctacacacatatgaggacaaaattggctattatgagtaaaagtctgaagatgctggagagaagaaggatgggatcctagaaattgccctaagagttgaggctgagatccggagcttgagcagcggggctggcaggcattgaaaatgagagacatgctctctttctgacactggaggaagtttcaaggggaactatgggatgctgagtagataaatcagttaccttcaaatttccctattacattagaaaaaaaataaaagtgcctgagagaaagagattggggctttaaaaagacagctgggcacagtggctcacacctgtaatcccagaattttgggaggccaaggcaggcagaccacttgaggatcacttgaggtcaggagttccagaccagcctggccaacatggtaaaacacaatctctactaaaaaaaaaaaaaaattagctggatgtggtggcgcacacctgtaatcccagctactcaggaggctgaggcaagacagtcgcttgaacccaggtggcagaggttgcagtgagctaagatcgtaccactgcactccagcctgggcaacagagtgactctgtcacacatacacacaaaaaaaaaggcggaataatttagagactatatcaggggttaataaaaagtaaatgggagcattgctgacagtcgtaaaggtcaaatgtaaattagtattaaggcgataaatcaagggagagccatgggcaggctaatgattaggtgcctctttaaacagatattctttaaatattcattcagtttttttaaatggaaataaagttggccccagaatgtaacaagatcaatttatattttaccaattattttaaaagttttatttggaaatccggattttagtaccacttaaaagttggtcctgaggatgatagaagagggatggtaccctaggagttgccccaggaattgaaagaagatgaactctgtagcttcagcatgatgggggctcagccatcctgacgtctagctaaatttgtacagttgtgtggcttcctctaaagatactttgttacccagaagcataaaaagccaagttcatgtatttggtagctatgtgaaggtgatcaggcagtaatatatactgatagggattcattttgctcctccagcatagtaaacctgctgtcttttgtatgaaaaagaattaaatgaaaccaaaatgagaatattataatctgacattgtgctgaatttatgttgaggaaataggacaattatacttaaaaaatgaggtttgtaaagggtcctaggtggaaataaggaccttataatggaagtaaataatggaaataacggaagccttatttctatacttcactcaaagtggtaaaacctaatataccaatagactgtgatgttaaatatgtatattataatactgagagtagccactaagagaactatacaagtgatatactcaaaaatactaaataaatcaaaatagtttttttttttttttttaagtccaagtaacccacaagaaagcaaggggccagcaggggagtgataggttggaagtggggaggtagtcagagagatgagaagttgagaataaacagaaaacaggtaacaaaatggcaggcttaaatcctaaaataccaagacttattctaaatgtaaagttgatctcaatacaccaattaaaagacagaaattggcagagtggataaaaaacctaagctaaatgtatgctgtccctcagaaacttccttcaaacatgacattggtagatggaacctaaaaggatggaaaagttataccatgcaaacattgatcaggaaaaaaaaagtagtatgcgtgtatgtgtgtgtgtgtgtgtatatatgaaggacattacataatgataaatggatcagtccacaagacaacatattaatcccaaatgtgtgcaccaaacaacagtgcttcaaaatacatgaagcaaaaactgatagaggtgaaagaagaaatagacaaatccataattttaactataacataagctatagttatacccgacactcagcaattgatagaactactggacaaaaaataagcaaagatatagatgaattaaacaacaggatctaatagacatttatggaatgccccacccagtgacagcagaacatacattatttttaagagcctatataacattcaccaaagtaggctattatgagtcataaagcaaacctcaacaaaagtttttaatgaaataatgcagaatatattacttgactgtgctggaattaaactagaagtcaataacagaaagataacagtaaaatctccaaaaacttggaaattaacatacttctacgtaatcagtgggtcagagaggaagtgtcaacagaaaagaaatatatacaattgataataaaaatacaacatttcctattaagagaatgacaagaagccacagactgggagaaaaaaatttgcaaaacatatatctgttaaaggacttgggtccaaaacatacaaataactcttaaaacttacaacaagaaaacaacccaatcatactggattaggatagactgcaaatccagtgattggtaactgatatggtttggatttgtgtccctgcccaaatcacatgtcgaattgtaatccccactgttggaggaggggcctaatgggaggtgattggatcaaggagggattccacccttactgttctcataataatgagttctcatgagtcctggttgtttgaacatgtgtcgcatctcccctttctctctcttcctcctgctccagctgtgtgggacgtgcctgctttgccttcgccttccaccatgatcgtaagttttctgagaccccctcagccatgtttcctgtagagcctgtggagttgtgagtcaattaaacctcttttcttcataaattacccagtttcaggtattcctttgtagcaatgtgataacagcctaataaaggtgaccttataaggaaaaagagacatagcaacacagacgtagacccagacccaaaattggtgaaatctgaatgaactctgtggtttgtaccaatgtcattttcatgattttaatatcgtacaatagttacagtagatgttacaattgggggaaacatccctgtacatttattttacaacttcctgtgagtctatacttaattgaaattagatgtttttaaaaagaattaaaggtaatgtaatggcatgtgatgcaaaaaatgggcacatgaaatatccagagcattcaaccagatggcagctaaaaatgtggaccatctgccttcatggaactttgtatttcggacatcttttcatcagcaagaccatgccatttaaatgttttcccagaagtgatgctgagctatgttcagtggccacatggctctctagaatcagtaacaatgaataaggatggacagccgggagtaaagatgctctccaggaaatcatcttaggactgggtaaccccactacacccctgggattcagtcaaccttggaaatcactggaatccttaatataaactacaaacaatccagcattctctgtttggctaaagatacatttcaaagttttctttaagaaaaaaaagcaatatattaaaacgctttgtttgaatatatgtatatttgctggatcaatagttcttacagccatattactaaaattaagggctatggcataataagaattactataattctaaacccattaacagtttatagcttatccatttcttatactatccctgtgaggaataattatatcaagctttatttgtttacaccaaacaggaaaaaatagatgtatttattcatatttcctccattggattagccaagtctactagggagcaaaaatagtaaattcacatgcctcctttttttatggtagtaattgaaaaagttatggatatgatcccattttctgaaatgtttaaaaatgagtgtataaaatgatcttattttcagaacttaagtcattatgtgtattgtttatagggagttatattacttttccagattaataggctgccatcgataatgaagaacaaataaatcctgccttgtgaaatgctgccattttaaaatttgcctgaggaaagattcattattggaggattctttgctttatacataaatcgctttaaacagcaaagtctcctagcatttaaaaatagtccatatataaaaattctctcaatccacaatcatgtcagttgtgtaaaatgagatatatctggtctgatacctcagtttatggaaattcctcttgaaatgtcagtcattactcacatcagtgctttaaatgccatcttagcaaactaaaagaaattagcgtacattttgagtaatggaagtgagttaggaagaggaaagaacagggtccaatgagtttgaaatggtgtttacagttgggtattgttcatagcttttggaactgtcttgcttcccctattcatcaaggtgctgaggcaacaacattaaaatcaacccaagaagtatactgagtaggtatatgggagaatggcatattaacttcttgataaaatgtctcagtatgtaaatacttatgaattattattaatttatattactgtacttacaccatttgattacacctgcataaagctgcatactcatttttacttcaaatgcaggaactaactcttatatcccccacagcatttagttggcacataatagatgcttatagttgatggacacaaaacatattcaactattacaagaacagaacttttattgcttcttcagtttcaaatgtctcaaagttaccctttgcttttgaccacaaagtctacccctccttatggtagtttcctcaggtgtctgtgtcctctctttccattgcattttcttctcccacatctacccagtaactggagtcttcctccttttgctgccccttgactacaaggcatttccatcttttggcttcctaatatgtcatgccttctgttattgccaccacagccttccatttcccagctctcatttccatcctagggatgggacagtaaggctgtcatgaataagcataatttatatgcctttttctccatttgttggtatggataattaagtgaccttcaactttcattcaaggttaatcatagcgctgaaaaccaaaaggataagatgagagtgagcaaatcaagtccagagaggtgtatttccagtgtggaacagtaactcagtgtaaatatccaggagagaggcttccaagagagcgtatgtgtgtgcactggtgaggggaggaagggttacgggcgggaattgtggtagaagcttcctgaggccaaagaaaacattcccagagggtctctattttaaagaaaagattttctttctctgattttttttatatgtagatgtcttcattatatatttggacctgtttttaaaacagcttagttttagatttattcttcgagataacttgtacctatggacatcaccaacagcttacaaaaattagaccagttgactctcaataagacttctcacctgatccatgagcatcatctctttatttggatttcataagccagatgatatcttaaaattggcaaatataattacaactccgttgaacatatttttctccagaaaatgttttgagtacaaaaacgacatctagaggtgactgtcaatatatcttttctttgcacgtactacatatttgttgccgtgttttagaaatattttagaagtttgggtaacagaaccggcagtggcatttattgaattcttcctcacaataactgaagggatagtattatactcgttttgcagatataaccctttcaatccaactaatatttattaagtttactaaatgctggcaccagtgataccaagatcaatcagatgtagctcttgttactgaatggcgtacatggaggagataaacacagtaacgtccactcttgtgtggtgagtgctgttggagattaattaagtgttatgaggttatagaagaggaattattcacccatctgcagagggcttggggatgaaacagtgtcagtgaagcactcctggaggaggtgtaccaggggaggtgttccagacagaaaacagctgaagtaaaaacatggaatagagcaatagtatattgtggtctgagaactacgtataattcaacatttcaggagggtaaagtgagcggtagataatgatgagaaataagactgcaggcacagtcagaggcctatttacccacacaaaggaatttggagttggaggcaccaagggtaaatatcttgtttcaaaaagtgattacatgaggatttttgcccagttctgtttgtctccaaagccatgatttttacattaaaccatatttacttcatagacattcttcaaattaaccatttaccaaagatgtaaataagttataagaagaaaaacaatactaatttggaagataataaaagcattatttggaaaaagccagtgtagtatctctattttcaatttgaactttgagtccaatgattagacattttctagtcccatattttataccataccatgcagtctgcttttgttaactatcattaatagtgaaatgacaaactgaccagcatctaatactggggttacactggtgggtaccctacaacaggcgttttgttctttggtatctaaccactaccttcagtagctgcctgacaaagatgtgaatctgaggccacactacctcaagggcacctctaccaagacacatctgctgggcctaatgaaaacagaggtttctcctagccctagcattggagactgacataccctcaatggatgtcaggggagacccagtcactaaggatgactctcctgcccctaacccattagcatgtcccaaaaagaaacaggagaccaaagacctttgccttgtataacagtcatgctgcctcggaaaagctgggaagtcccctcccagtgacccccatgctctatcctaatgtgaacgatgttcatgctttttcatcaaatcttttaaatagaaagggtgcagagaaagtcaactgtaatcacttggtgcagagtgcagggagagagagcatttcatattcaagaagaaattgtccaagcagtaaatggcattgtggtgagtcctgtaattggagatagtagagaaacactggacagctgaggaaactccagaagggagaaaaaaaaaaaaaaaaaggaaaacctaagaacaaaataaaagagtctaaaatttacacagaatcagaaaaagacttctgaacaaaatagcattgatggtgctgggaaacttaattagtgcataccccttacagcaggaggtgggtagaggaactgttatgttaacgaaagatcatttgaaaaaaaaaaaaacaaaaacatgttgtctttctctttaaaattttaactaaattccctgattcttaacagaacagggaatatacatttttcactaaggaacaagtgtgttttcttaaaattccatattatgatctgttacaggtgagtgggtggcaagtagaatattgtggttaactcctgggatggagaaagggtaggggaatcgatattattaagagagggatataggagaggcttcagtaatttgcagtttgaatactgcaaacttaagtagtaggaactaatgtttctggagttattccttatacctttataaatgcctaaatactacaaacatttcataaacttttaaaagtgaattggagattcgggcaatatttagggaaatgagagggcgcaagaaagagatcagaggggcagaggatgagaagaagaggagaagtaagtagggagagagagaaaaagggaaggaatggttttgaacctagtgagcttttggaagggttaggttagaggtcacatcatgctgttttccttatcaattcttaccctgattgagtagctctctacctgtttctcaccaaatggttcaagcagctgccatggtattctcatctctacttcccagccacaaaacagctccagccaacataaagcagttcttccagtagaaaaaagaaaaaaagagcacctgcaattccctcaagaagaaaagaacaaaaacaatgagtatatatgtatgaatgaacctaaagcagctctaaactactcccctttggggacagaagcagatgccttcgccaaattcaaaatgaaatcccatatctctgaagacccccttcttatgttactgtataatttacatgcaagttgtctgcatagttaatcgtcctctatagcatctggtatttatcatcaaaagccgcttgttgagaggttctggagaagctcttcatgtatcccgggttttaaacatttttctacaagtctgtttctttaaaattaaaagtgtagctgagcacagcgaggatctggtagtaagtagtagggagggtcattcatacttgggatgttgctaatgtgacctgcatgattcagggccagagaggtgaatgaagataaagggaactaagcgttacagccaaaagggttctgaaaatctcctagtcagattcctcagtttccacatttgacagtagctttcgcagggaaccccatccaccatgactgagctgaaactagaagccaggtttttcaggtgtcattcagggttgatttagggttttggttttctcctgcaaaatggagaaaatgggaaaattttagattatccatgttagaaggatgatgaatgtgtgtaaatgatattgttcagattctgtgagtgggaccagcaccacattggactggcctagggaagtcaggattctgccttctaggtaggtgctcccatctgttggtgacctggaacatctctctgtgttcattctggagatgcagatctgggtcccgcagactcagacctttgcaggacactagggtggccccttgttgcttctcatctgctatagtctcctgccactgtggcctacgggtttgatttagaaattcaggaaagactagaagcaagcaaacagataaaaggttagtgaagtgtttatccactagagcaaagccacaacttttcgtattatttagctcttattattctaaattttaaattgttttttattgtttcacttaccatgtgtaactttcacttttcaattacacatggcaagtgaaagaataaaaaacaatttatttattttttatttttattttattttttgagacagagtcacgctctgtcacccagggtggagtgcagtggcgtgatcttggctcactgcaacctccgcctcccgggttcaagtgattctcctgcctcagcctcctgaatagctgggattacaggcacccaccaccacacccggctaatttttgtatttttagtagagagggggtttcgccatgttacccaggctggtctcaaacttttgacttcaggtgatccaccctccttggcctcccaaagtcttgggattataggcatgagccaccacgcccagccaaaaaaaacaaaacaaaacaaaaaactaaaatttagaataataataagagctcagaaaatgacagtaattaaacaaaggtcacacggaaaagttgcccttgatatatcttgggacagggtgggttttccttattgttcttagctccttttgttaatgtttattttagtgccttaatgctactaaacagaaaattcagaaaacattgtagatgtcttccttttacgctaatattctccagcttttcatacatttgcaaaaagaaatagattatggatttctcaaggtttagccgcttgttatttccgtgtgtaatacaaataagcatagttaaaggttaatttaattagcaaatttgcagttatctgaattaagtgtactccaaatttagtttacaagacctgacaagtttgtgaataagttaattcacttctaatgtattttaatatacccaaaccacaaagcatcttaaactttgtaaaaagagtgtgcacggaggaatgagcactagatgtgaattctagagaactggggtccaggctctgctgtgtattggcctgagcagttcacttaacttccctgagtcttggtgtcctgttttgtataaaggggaacctgatccctcttctgcctggctggcagagcactgtgagactctggagaaatgacatatgttagagatgtttataacctggaacacggtacacggtatgttttgtttgtggcattttttttttttttttttttttgagacggagtctcactctgtcgcccaggctggagtgcagtggcgcgatctcggctcactgcaacctctgcctctcaggttcaagcaattctcctgcctcagcctcccaagcagctgggactacaggtgtgcgccatcatgcccagctaatttttgtatctttagtagacatggggtttcaccatgttggccaggatggtctcgatctcttgacctcgtgatccactttcctcagcctcccaaagtactgggattacaaacgtgaaccaccatgcccggcctgtggcattgtttttaaactaaagcatgtattatagttttcctgaaaagtaaagatttccagaaaaggaattgggaaactcttttaccctcatagttataaaatatggaggtttccttcctccaggttggtcagcttccctcttgatatggtttggctttgtgtccccacccaaatctcatttgaattgtactcccataattcccacatgttgtgggagggacctggtaggagaatcatgggggtgctttcccctgtactgttctcatggtagtgaataagtttcacaagatctgatagttttatcagggatttccgcttttgtgtcgtcattctctctttgcctgctgccatccatgtaagaggagacttgctcctttttgccttctgccatgattgtgaggcttccccagccatgtggaactgtaagtccaattaaacctctttgttttgtaaattgcccatcagcagcgtgaaaatggactaatacacctctgctctagttctttttggaactaggcctcttccctttggttgtcttgccactcactgtcagattggacacttctccacactggacctcaacatcaccctgctggcagtcccacaggagttaaaatgtctgaagagggaatggcccaaaaaggagtctcactgccattgcaagagctggtttagaataaagggggcagggtggatgatgaaggattacttaatgggtacaatgtatgttatttgagtgatggatactctaaaagccctgacttccccaatatgcaatctatgcatgtaacaaaattacacttataccccatatatttatacaaataaaaaggaattttaaaaaagaataaaggggtatgctgttcattcatttaataaacatgtattgagcaccaactatgtgccaggcactcttctagatgcctgaggtgcataaatgaacaaagtggacaaaaattccttcccttgtggagcttacactcaagaatatgagaccaaggagatacagtctcagaaggagcagagacaaggaagagtcaatgatgcccagtgaacaatgggataaagcttaggagtgttcatcctcattcataattccatagcccataaacgtgcagacccagacttgaccataatgtgactagatactaacagggagtaattaagtttatttttttcatatgaatttcacagacatatacaatagcatggttaaaagaactaaattaattttatggtagtaattttttccaactctgatattttgtgtcagctcctacttgtcccagccatgcctatctgtggtctgcgggccttcaaagggcagcctttgtgttgcccaaagccagcacagtggtgctttgtgcaggggcatccctgtgcctcctggaattcgtctgccccatgaagctaggtggctggtgttcgtggccagtcctctaagaagcaggtgtccccactaccgtccaccatgtgtgtcctgtctgcagaacgtggctactactcagatcctgtttctcactcaagttctacctcaaggctcgcactctgtcaagtgatgtacttcttggggtccatctgcctcgggggtggaaattgtgaatagaactgtagcccagagaatcattgagagactaggtataagccagtggcagaacctcaaccaaaatgccgggcttgtgacccctagccctgggctcgtgcacataacacacagtgctctgaaatatcattgtttggggcattattgtcacttttcttctactgatgtggatggcatgttggattttatgaatatcaattattcagattcttctcaaatagtaattagttggttttgagagtaattaaggcaatagatgctcctacaagaaaagagaatgaaagaccctgatttgtgtatttggggtaagatcttacaacaaactgggcccaggtctggaataaatgtggacaacttctagcacagcataaaagcattaaaaagtttattaacacatcatgtcggtgtcaatgaaaatcaaattggtataattttcaaagaagaaactaatgatactttaaggctgtgtttatagtaatactatctttctgaaagatactttgtttatgtactatattaacatagaaaaatagtaagcagtttccagctacaatcagtggtttaaattaagatgtaaatgtgggtctaccagtgagaaactaaggcaaatgtctccatctaagacagagttgccagataaaatataagatgatgcccacttaaatttgaatttcatatgaataacaggtaattttttcttataagtaaaaatttcaagcttaaccgggcttcccacattattgtttttttttaattttgttttgagatggagtctcactctgtcgcccaggctggagtgcagtgacacgatctcgactcactggcttcccacatttttatttgttaagtatggcaactataatctaaaggacaaaatttgcttccaaacactgcccctgaataagatccaaagtacatatgatagccaattaatttggaaaatacctcaatacaatttcaaatctcacagaggcagggtttctcaacattggcactattgattttggggcagttaattctttgttgtgaggggctctcgtacattgttcaatgcttaacagcacccctcgcctctaccaccagatgccagtagcataccctggttgtgacaaccaaaaatgtctagacattgtcagatgtctggtggggagggggtcacaaaatcatccctgggtgaggacccctgttctgagggaagaagcttatttataagtacagtatatgtattaatatcttagtagaaaactaacttgtcctgatcttgaagaaagaagaggaaagccatattttatgtagcctttattttaaaatatcatgggagaagcaccaatggaaatggctgaggaactagagagctggtagacgtgggattattttgcagtctgaggctctatgacagaagcttctgtttgctccagcagctttgagagaaaaaggcagtagaaagtactcatgacacggctcaggttcccaatatagacttcatccaaggctttgaaagaacagttagtacagagtctgctgatttctggaaaaggagtgttttatttggatagcaataactgaaattccaacctagaccacctagctgaataattatatgtgtgtgtccaaaaaacaaattgccaagagatgcattgagaatcagaataggaaaactgtttactgtttgatttttcagaaaataattcaacagggaattttgctaaagtgttgttccttctattttacccaagaaaacatatgaatttattttgctgtctaaaccaagaaactaatgtggttatacatattatggggaagtttttaaggaattaacgtttagagtctttgtggctctaattactgctcttcggttgtgtagtgagagtcactaagcattccttcctccaaggaatttccagcataatcctgttttccactcaaattctttgtgattgccagattggagtgaaaaatcaaatctacttccaatgaagaaaaactcaaaaaaaaaacagtgattcaaacaagtttccaaagagaatacatgaacttcttattctgcctttattaattgacatctacaattttgtatttttctcaggataatgcaaaaaaaattcttttgaatcattctcactaaatttaggggccaagtgtcctagctatattgttttttaaaaaaagttatgttaaagcagccagacttggtggctcaagcctgtaatcccagcactttgggaggaggaagcaagaggattgcttgaagccaggagttcaagaccagcccaggcaacatagtgagaccctgtttctacccacaccctaccccctaaaaaaaattagctggatgtgcctgtagtcccagctactcaagaggctgaagcaggaggattgcttgagcccagaagttgaaggctgcaatgaaccatgattggaccactgcactgacaaagcaactcagttctgatctacagttcattcttggataaacataatacacaacacctagaaatacagcaaaaacccattacccttaagacaaagtccagaatccttactgcagtctacaaggtcctaaattatctgattggagcagactacactttccccaactccaagttatatgctaagctgctctaagttagagatcaaaaaaatagagtggctcaaatgagatcaaagttgataattctctcacctaccagggtagaagtatgcagtggtcagtggttcagagtttgtggaaagtttgatccatgaagccatctaaataaggtactttgacccaagtactttctatcttgttgcttcccaccctttaagacattctcctcatccacatggtcaaagctcactcaccactaccctcactctgtcccaatctgtagaaaggtaggcagagaggccagcaaatagctttcttttaaatttgtgttgctgggtgggtcccccaggaaacagactctgagatggagattagtgtacaggaagttcactatggggtgctccatgaattatcacccactggaggaaaggaaggaagcagaactgggaagatggaaaagttaagctgcagtacagtcacaacaaggtctcatccaaattgatacggggcgttgaagctgagctggtccttcagagctgggatgagaccagatattggctgcaagccattccaggaaagggatgtgaccttgaatgagatgacttttcttagctgaggcaattctcaaagggagctgtcagccagtagccttgcagttggacagatggcagcacagcagagcatctgggacaggatgtgacctggaaatagcattgatatggtttggctgtgtccccaccccaatctcatcttgaattcccatgtgttgtgggagaaatccagtgggaggtaattgaatcatgggggaaggtctttcccatgctattcttgtgatagtgaataagtctcatgagacctgacagttttaaaagggggaatttccctgcacactttctctctttgcctgctgccatccatgtaagacgtgacttgcttctccttgccttctgccaagattgtgaggcttccccagccacgtggaactgcaagtccattaaacgtctttcttttgtaaattacccagtcccatgtatgtctttatcagcagcatgagaacagactaatacaagggtaaaccacttccattcacccctcattatctagaatgtctagaacagccaccatagttgccacggaggctgggaaatacagtctctagctaggaggccctatgcctagctagcactaaaaggaagaagggagaagggatactggggagcaatcacaagagggaaggaaaatcattctttgatttgttcagcttattgactgttgtagatcaaaaaccacttcattgaggaaagcaacccaccaaacacagaagcagaaagagagctaaaaatgggataagaacaactgagaaactgcttatccagtaatatattttaaatagtgaggactacaccaaacttaggtaggcttcagaatgagccagggacaactccccagaactctgcaggaaggggctcctgaggacacacgtccaacctgcacctgaaaccccaagtcacaaggacaactgatatttagatgcttttcttcctaagacctgctacatatcagaaaaagtcacttaaggtaaaagataaaatatgctgagaaacgattctgtttcttaaaaaaaaagaaaaaaaagaaagaaagaaacccaaacagtaattgtattgacccaagaaagcactattaaaatgtatcactttctgttagtgttgcttggattttcaacataataatgttctgtttaaaagaaggagaccaaagtatgcttattacctgaatgacagaatattctgtgcaccaaacccccatgacatgcaatttccctttatataacaaacctccatgtgtacccctgaacctaaaataaaaattaataaataaataaaatttaaaagtaaaaataaaaataaaggaaggagaccaagaaatacttaagggcccactccccttctttctgaacaaaatagccagaccagcctctgccactgattgaaccagggattggaaactaacataaagcagccaccacctctaggatgagcataagattatcagtagcctgtgaggaaacttggtaataaaactctgactggtagtgacactgtatgaggtagtgtgggcatctaaccaaccaatgtggtggtcctcttgtggacaggagagggtatgtggtgtgtacaacaggaaaaacacattaaaactctagaaacccaaacagagggagtaggaggggccataaaatgtctgacttctcaaatggctgatgagctcctagaggtgtccttggtctcttccccagaggcaaatttcaactagaggttgaagctaagaagcttatcaagactcagggcttctcacttttcccctcacaggcccactccaaggctctgatttcctgggctctctttgccttggcttcctggccatcagtgtccatcacttgagacaatctgagtgtccccacaccaaacacctgaaatacacctaataagaaagcctcatcccatctgcaaagtatgctggaactaagatccagattcacaactcaggaaatttcactctacttaagtagtggcattctatcttcagattaaagtgtgtttgaaaatctctacccctactgactggggctgtcacacgaggtcccacttggccccgtcttgggactctcctgcacagcacttgactctgctagacatcctctctcctctcccctgtgccctcatccccatcatctcccagaaactcagtgctggaacacatgagacttctcagctgcatatcggcaaggccaagggatcctcaaaacaaggcaagtttctgccctctcccgccatcaaaagtgtgtaatttctttgttttgttttttgttttttgttttgttttgttttgttttttgacacagagtctcgctctgtcacccaggctggagtgcaatggcgcaatcttggctcactgcaacctctgcctcctgttttcaagtgattctcctgcctcagcctcccaagtagctgggattacaggtgcccaccactacgcctgcctaatttttctatttttagtagaaacaggtttcaccatgttggtcaggctggtctcgaactcccgacctcaggtgatctgcctacctcagcctcccaaagtgctgggattacaggcatgagccaccatgcctggccaaatgtgtgtaatttcttaaacccaaaggggtctagccaggatcctgcattgtcatattttaagaataaagtgatatctccatgaagggccccactttgagtcctatttatgtttgcctctgggatttagctgtagctgggcatgtgtgatagtgggtaggtgacagcctgcccagggtatatttgcaccctggccatgtagcatggggttgagaaagcaggctccagtattccacaggtctgggtttaaaccccagctctaccatttattaggtttgtggccctggaagagtctgtatcccctctaagacttcatctcctcgtttgtaaaatagagatataataatactttcacaaggctattgtgagaattaaatgatgtataattcgttcttggcatagggcctaaaacgtagtaagcactgataaatgacagttctaatagtagataccccataactaagcgtctttgaaggaagtttaccataaggtaagaagaaagtcaacttgatccagtaacaaaaccccagaaggagagattccagacatagcttggagcccagctcttggctatgtctccttgcccagcttgcctgttattcatcactgagtcccatgtagaggtgggattgtaagtaaattgtgaaaagaatggtttcagtaaaaactccttaaggctttgggatacagcacagtgttgtcttgataccaccaccaccacttcctacaatacatatgtcccacctaaccaggaaagagataacgtctcaaactttgctttaaatgctgcccagctttggaatctcacagtgtccaaagcatcagaggatactcaatggatgtaacggtggcctccaacagatatgaccatggtcttcaggtgatgtgccaatggtatgtagtagacacagctctggttcccaacagaggtactgatgattccagcagtcatgacagtggtccctcgcagatgtggtgggatctctggaacaagcagaataatgttctgtggatacagcaggacccttggaagcatcaacattctcagactgattgagatgttagggagttcagatatctcttaggctcagcaaaagtggtactcgagcccaaggatgtggtgttagccagtagttgggggaagagagggagaaagtgtctgataccatggagtagctgcaggtatttatttatgaccatgtgtgggttatagcccaaggtacagacatttggggtatgaggaatgtgaagaagagactttgaattggacaagagtgaggaaaatagccaaggaactatggagtatacttctattacctggggtcaccataagcataaaactaagagcttcatttacagccctgtatttcaatgagaaagtggaaagagcaccggaccagcagtctggagaaccaggtgttaatccaagtctgtcacttaatttcttcaaaccttattgtgtatagaaaggaggaaaaagagtagcactcaataactgacgtcttttctttgataattttatttcatcactaccagattcagataaagccaaagtcttgctaacactccttgccagttgaaatgtggtgactctcaaaaacttggaaatgtcaccattctggtaagaaatcttttaatggaaactttacatcttctaaaataattaacattcagtactcaaccttagaacattttccaaatttagatgagaagagatgaagcaaggaaaaaaggtgtaaccctatcatacttttccttttggcgttgaaataaatatccagttaggggatggggaaagttttggtttctttagtactgagttggtggcagcaagagctaagtaggggctgagcatctcaggtatgggcttcccttagatgatccagctatcaggttctgccaaggtgagtatgcccttgtggggcttgcaaggcctagtcatggtgggagaccctctataaaggcctacaagtacttagggaggtaagaggacaccccctcaagggctgaagcctgtctcacctttgagggcagtgagatcaccagtatccctgtgagctgtataacaggaaggataagtccatgttctggtgccgatccctatgccttttggaacatttctccctctgataagaaacctccttataatactttgtttcactggaacaacatcatcctcatcattagtaaaatgattgctggttgtcaacattcacggagaaggtccctggggaaggtcagctgttgcagggaagactggcatgctggctcccaaggcaagtgttcctttccatgatgaaggcaccagggaccagggtcaggccttcagtctcagagatgcagaaactcttgtcagtgcctgaaagaagctgaagaataataagcagactgccaatgttgtcattaaaaggcaaggtatcttttacgaggtgagcattagcatcagcctggcaagtacattcttagacaaactgcacggaagccaatttaacagaaagttgcttggcaagcaacaagggtgatgtttaatggggtcagcctataattacatgatgcatccatttccaccatgtgtttctgtctgcctcttggaccagaagggctgtctttggggccaggaagacccttccctggacagtattcacgcatctactgccagactttgcctcactgactcttcctgtgtcacacattcccaaacctcaatgactcaccaattccctttctgattgacatggtcttgccttaaacgtgttgatgtctttaatcaagacagattaaagtgaagtttcttttttctggaagatgtgactgcatactgttttgaccgattatatgtgctcagctggcaaagggctatgtcccttcatagcctaattcttggggtcaccccattgttgccttcctcccatataacacctacactgtagtgttgaaatgtcatcatgttttattttatttcatttttattttctgtgttctttttctgtgttccactgatttttttgagtgggattggtctgcgaaatggtattatttttagcacaaaatgaatccagtgagacaaaatcttgtaacaaattcaactgtttcactgtatgaaagcagggcaggttctttactgatgacaatttctgtttgagaaaccaaactgagttcttcataaatgatgaacaaataatatacacatattattgtatttaggattatagtattgctagccctaaaaaatgatcaatgaagtattggcaatgtgaggtgataattttttgtttctccttttcataatagattttgcctgtcctgttagtgatttgcactgcccgcagctacccaaaacctcaagcatagacaaactgcttaaaattccaacgtttgatgaactgattcaaaacggatagattaacccataccacactccctgtgctgacactaatgaaaaaacaaaacagcaaaagagtttccacttcccagatgtcaatcttctatttgctttaatcattttatatttatttattgatccattcaattatgttggtttattcctacttaagttgttccaaagcatttaagacagatgataagaacacaagataaaaatcaggatcaagaaaaataaaaataaatgttagatgacaaagatgggaggaggaattagaattacaaagatgtgtagaatgctagttcctacataattattaaaatggaggccaatgttcaagctgggcttcctggtggcaaaagcatacatgaaattataataggtacaaaataataccgtaaagaaaacctccccaaataaagaaagctttttcttggcactcgaatctgaaagaaattttttatttagataagaaaatctcagtgatatggataacacaataccacaatacaatctaacaacatgcaacccaacaaaatgtgtgtttttttaaggaattttgttctttgctcaatttcttccatctactaatttttaaataatgcatcctctgtctattcatttgaggttaacccaaatattttaatatttaataaaatctaagtttaaatctttttacctgcttctgaaaaatacaaggggattataagactaatcaaattgcttcccatgccactttatttgttgttttagttgagtgtgttagttctattttgctttttaattctacaaattaaacactattattattgtcatcaccattgttttatatggccagtgttgaagtgtatttatttataaatgtctttgctcaccatttcttcttgaatttcagactttcaatttggattcattttctgtgagcctcaagtatatccttcgaaattttcttttgtgaggatctatactcagttttgtttgtcttaaatgtcttttattttactcttattcttaaacgatctttttactaatcgtgggatttgggattgatctttttttttctttgacacgttgaaaatgatattctggcttccattaatgctgttggaaacactgctgtacctctaatttttattcctttgtggtgatttgttatctttggctacttttaagatagatcctctctttgaccttgttgttctgaacttccactctaatgtgccaaggaattattttatttgtatttatctgcttgtgatttggcttatcatctttcagcaattctgaaaaactctcagccatttttccttggaatattgtctcttttccattctttctactgtgtttttccagagctccaattaggtgtatggactttttcaaaaactctttctttttctcttattatattttgaaatctcatttctctgtactatattttatatgatttctttagatttatcttatacttcactagttatctctttgtgtccagtctgctgtttaattcatacattgattttaatttaaattatatttccatttacagaagttccagttcaaatatacttttttttttttttttttttttgagacagagttttgctcttgtcacccaggctggagtgcaatggcatgatctcggctcactgcaacctctgcctcctgggttcaattgattctcctgcctcagcctccctagtagctgagattagaggcacctgccaccacacccggctaatttttgtacttttagtagagatggggtttcaccatgttggccaggctgttctcaaactcctgactttaggtgatctgcccacctcggccttccaaagtgctaggattacaggtgtgagccaccgtgcccagcccaaatatacttattttttaaacattttttatttcttgtcattattttcaaatgtctgttttatttcttaaagcatattaagtatatttattgtatagtttacttctgatatttctagtatctgcaagtctgtgaaagtgtgatgttgttgtctgctatttcctctggctcttacttggtgtgccttttgcctagtatactttgtctttgtgtgttgggaggggagttcatgttctttagaatgttatctgcaggattttttgagaactggttaaagttggtgtccttcagaaaggattttcttttacttcgattgcctgggaggactaccagttctctctgtgattgtttttttttttttttcttgtcatgctttccacttccactgcatttctcactggctcaactatagtagcatgaatttggaactacaaaaccacaagaagcaacatcaactatgaagtatcattgataaaaatatttaacttgaatctaatcatgagaaataattgaccaaatcaagactataggacagaatctaggacattctatagagcctggattcttcaaaaaagtcatacacacacacacatgtacgtacaccttaatttataagatagaaatcaaaagaaactacagagatgaacagcccaatgcaaggcatgcccttcattgatttctggatttttttaagtcataaaagatatttgggagtcacttaagaaaattagaatgcaaactgtatattaaatggcattatggaattatcattacttttcttaggtgagataaggatattgtaattatacaagagatgtccttcatctaggagatggctgctgaagtattgaagggtaaagtgtcctgtgcttataactggctttcaaacagacaaacaaaaaaagctggtccaaaatggcagtggcaatggatgtggataccccaagaagtaccaacagcagtgctttgaagtaaaaaagtgaaatgcagtagctagccctctgggcttgggatgttgtggttaataactgtgccatctgcaggaatcacagcatggatctgcattgaatgtcaagctaaccaagagtctgccacttcagaagtgtgtaccgttgcacggggagcctgtaaccgtgcttttcacttcactgtctctctcactggctcaaaacacaacagctgtgcctgttggacaacagacaataggaattcccaaagtatggacaccagaaaaagaattcttccatcaagcttaactgttttgtgattcatttaatgacttgcccttctgtcacttaattataaattagatagaactgtgtcttttctgctttgtctttgccatttgctgttctcgcagccacattgtattctgtgtcaaataaagtccagttggattctagagcgaatgctgtctcctgtgtatgtgaacaaaagtgaacgtaaatgaagggtcccctcttccaaggctgaaaagccaccttttaaaagtgaaatgtgccgggcgcagtggctcacgcctgtaatcccagcactttgggagccgagacaggtggatcacgaggtcaggagatcgagaccatcctggctaagacggtgaaaccccgtctctgctaaaaatacaaaaaaaattagccaggcgtggtggcaggagcctgtaatcccaactactcgggaggctgaggcaggagaatggcatgaacccgggaggcgaaggttgcagtgagccaagatcgcatcactgcactccagcctgggtgacagagcgagactccatctcaaaaaacaaaaaacaaacaaacaaacaaaaaaagaaagtgaaatgtgtgtccattagagccagaacaggattgtcctgtgagtgcatcgcttagtcagaggactccaactgtacaagggggaatgtggatcgctgaccctgaaaacagcagtcagccctgtcttcctctggtgtgatagcagcaggcagctgaatgaggaaagaacgtgggattcagagtcatcacacccagctctgagtcctggttgcataggctggctactgatggcaatctcgggcacgttgcctgtctaccctgagcttttctcctctataatgcaggcttctaaggactacatggcattacatggggcacagaaagaggctttataaaatgcaaaaccactatagaaatgtaaacattgcatgcatatgacatgttgctttctcttcttagcttagaaaaataaattttaaaaaattgtaatatgtaatcttggctggcaaatgtatgaactcaatttaaaaactgatgtcacctaaaattgaattaacacaagagttcaagtttcctttactaccactacctcatcacaacttgttaaaatgttttgggccacagaaggaattgccaattctagtactaatttggtagctatagagaatcttcaaccgagaaacagttttgttgcagtggttctcgaaatatggtctgaggattcctgggggtacctgagaccatttcagggggttcacgaagttctccctccaactgcaagtccatgtgaaactagtttttcttcatatactttaaccaaagcagtgcatcaaatgaagaagcagacttgagaatccaactgtcttccattgtcagacataaaagagatttgcaaaaaggtaaaacaatgctagtcttatcactaaattttttcattttgaaaaatagttgattttaataaaagtatgttaataagtgaaaaaacattatctatacacagacacatacacacatatgtataggcatatgagataaagtagtgactctaagagcactgttcatagtactttgcttcttagagacagggtctctgtcatccaggctggagtgtcgtggtacagtcgtagctcactatagcctcaaactcccagactcaagggatctttgcatctcagcctctacatgcgccactacatccagcttagatttttgtattttttgtagacacggggtctgtgttttgtatattttgtagagacggggtctgtgttgccctgactggtcttgaacgcctggcctcaaagaatactcttccttcggcctcctaaagtacaaggattacaggcaggagcagctgcacttggcctcattgtacttttcattcaacttttttgtaggctcaaaatttccacaaatttttaaattttgtgggaaagtgtaaaaacaaaccagaaaatcacatgaattcccctagaggtgttctcctctactccacagccaaaatcgggagaggcaaatttcttgttctctccttttgggaggagggtttatgtctaagctaccctacactgagggtgcgactcctgatgtctcagttttctatgaaaggtttcctactgggcttttcaccctaggatgtctttttcttaatctcttcaacccattatgctgatacaacagaagtttaaggtcccaggttttatcaggcactttcagggtaaaatctagctttggcactcgctttccctctcaggactcaggctttcattttatatttggtctctatacgtttcttgctttcttgccagctcaataattcatttaattttaaaatttctaccccaagtttttagttattttcactgggaggatcattcagggtatgtcatcacattacactactagaaacaatgcaacagcattttaaatggaagttaaattgcaatcacttttcttccaattcttgcagagattcaaacagtggttacagccacaagcaagagattcaaatagtggttacaaccacaagcaagttttgaaaaaaaaattctttaccataatactgtaaaaaagcttgtggccctaaggcataaatccagcatgttcacctttcaattacacagttgaaaatatagttgtctcaacacaaaaaggtgctctatcaaaccaatctttctccctaatttgtgcagctcatttattcaaattattgtgaattcagaattctacattctgtacaaaaaaaagtatgaagataggccagatcatgagaaaacaccttctcaaaagtcataaatacgttcataacaatgtggaaatagccctgttgacaagtgcagtacatctcttttttcatgctttttaatttgcaaggacatttttatataagaagcaggtttctaaagccaatgtcattcatggtttcctttttagtgtagtgggcttgggatcctcgggattagactactgcctgaggatggtccactcccctttactcaagcctccccttaagatttaggatagacctttcccctggataactgagtaggttatcatcaatttccttagttgcaagaacaccagcctcaattatttattaaagaaaaataaagaaaaaggtaaacagacatgattctgcaaaaaacaaatctcagaattataacatcacatgaaaacccagtccctgattatcatggaataattattcccgagtgtccattacaggttgtagatgctggatatttttatcctgagaccttgtttggcctgaaccagcccttgaactctatttgtctcatctctacattggacaatatacctagctgtacctgcatatacacatgtgagatatatcgtatctctatataaagtaacacacactccacctgagtttggagctcagtatttgctctgttacaaaagcagctgaaatcactcattctacttgtagtcagggcaattaaaatttactctaagtcctgctgtattttccttccatttacaagagggttaggtgccccttccctaacagagtatgctctacttgttcaaagattgtcaacaataacctacagtttccccgtttcctcttatttaagctttgggtgataatagattaacaaactcaattacaaatggaaacatctgaactccactgtcttaatttatttagtgttgctgtaacagaatatccaagactgggtagtttattttaaaagaaagagagagaggtctatttggcttatgattgtggtggctgaaaagttcaagtttgggcatccacacacatgtggtgatggcctgaagctgcttcaactcttggtggaaagcaaaaggggagctggtgtgtgcaaagagatcacatggtgggaggggaagcaagagagaaaccaaggagccaggctctttctcacaacccatcccattctaatccatcccacgagagtaagaactcgctcacctttgtggaagggcattaatctattcctgaggaatccatccccaggattcacacacctctcactagggcccacctccccacaccaccgcaaaggggatcaaatttcaacatgagtttgggtgggacaaaccatctccaaaccatagcacccacaaaaataaagataataataaggaaaccaccatgtgccaggcactgggttaagtgtttatattcatcctccaactaagaacagtactattggcttttccactcacctgaaaacaggtcccattagcatcttcatcttatgaatgaccactctaaggaacagaaccattaaatggcttcccaaggtcatatagtcatggagccagtattaaaggctgttgcactcctaaccaggacactaccccttcttgtttttaccggccactctcagaaatgtttcaagatcatgcatcctgggaatcagtttcaggtccctgaagtcagaagcccaatgctgttcattgagagccatgatatgttacgcaccaccctagagctggttcttaacccataaggtagctatcgttcccatttcccagtgaggaaagtaagacaccactcctttattcatccaagagcgtaaaggacttccccaaggccacacagctagtaagtggctgggccaggtttcaaacccaggtgtggctccaccgtgctgcctctccagatcatacatagcattatgattccttcacgggacagagctatatcatgatgtagcatgctggtgaccacaaccattttggcaatggctttctaaaaatacagagaaaaaaagcaaatactaaaaaactgagtttttggcttcacataatatgtgaagaaggaagtacgttaaattaattacaagaaatgcattctcatggcctcaattcacgtagtgtgccccagaagcaaagttttatgcattgattagacattcactctccctaaggcagtgcaggcaagtagaaggacactgtgtctagggtcagaagacctcagttcgagtccactttctggcatttccttgggaaggttgttcaatctccctgaccttcagttttctaatctgtgcagtggaaattataactatagtacctgctgaaaaaattggtgcgttataattataactatggcactcatctgtcccacctcattgttgtaaagattagatgagatcaaatataagacaaagctccctatctgaatgagatatttaaagttggttattattatgatgtgggtttctaggcagtaaaaacttaccatactcttccacagaaaaagaaaaagatgacgtgtggatatatgtttacttataataaacatgcatatatatgcatgcatgaatatgagtacaaggacatatgtattagaacattgtggtgtgcaaaagaagatgagaaaaactagtcataatctcgcagaagaagccctgataaagtatgctagggcaagtctgcaggcctctgattttagctgcttaactattggatacacagacagtgttgaaagattgtgttcaaggcactgcagccctcagagaactagccccaaagcccatgaccccacaagcaactttagggagctgctgtggatgaagtacctggccccactggaaaggcactagaaaatgagctcttactttgcagggagtggtcaggggaacatcaaggaaagaagaaggaatcatcaatggggcttggggctcactcaggaggagctcttcctgataggtgctttaaaggaatcagtgctcaaaacccctggatgtgatgagctcattcatccttgagggaccctttgtcgcctctcacctgtccttctgaggtcctccacgcgttccccttctctgttcttattccaaattcctctactacatttactcaccctggatttctgggtaaactttattagatttgttacatctattttcctttcccattagtcaatttttaaatgtggtagccaaaataatcaacatcactagtaattatacccatactcaaaaactcttctggtgaagaggcatggcagaactccacagggaaatcctattgtgaggattatataggtcggaaagtcatttttaactgacatgcatgataaagcacatgctacataccaatggaatttaacgttcaaatggaataataaactatgtttgcagtatacatttctagatccatacatgaaatcacatttccattaaacagttttcaagtcagtgaccaattccctcctacttctcctgaaatttatatgaacctggatcattttaataccactggaaagaaacactatacttagaaggagatgaacaacagatttaatattttttcgtttgcggtactaggaggcaagctggtaactctaatgccctgtgtgcctcgactcctagatctgagtagtcaataagaaagtagggaagggactaccagcaagaaaagaagcccactaatatttccttctccttccttctgtgttttcttctgcttaataattgagtctaatattaatggacaaggattgccagaaaaggcatgatctagaacaggggtcagtaaccttttctgtaaaggaccagagagtaaatatctgagcccttcgcaggccacatgggctctgttacaactactcaactgtgccattgtgccacaaaaatggacacagcattgctgtattccaataaaactttaattatgagccttgaaatttgaatttcacatgattttcacatcacaaatattaaattccaatcatttaaaactataaaaattattcttagctcactgatcctataaaaacaagcagtgggccagattcagcctataggctatagttgccagtccctggtctaaaacttttaatacattaagagtattcttctcattaaaaccactgaaattgaagtcattttctctctttctgcccatttacttttccctaagactttaatttttttccccacactctcattcccattctgatttaccaggacattacttctcctctctacctcacatatttgttttattgcactataaacctcactggagaataaaactatttgccaaagacccacctgtaaacacccagcactgcagcagccactaacaaagaggaagacacagaacctgtggccaatcatgtggacattggcaataatccattcccttctccttctttcttcctggtagagtccacctcttataatagtctgaagaaaacagattgttacttttccaccttcctggcaaacaaaggggtatgtctgtgaccaactcctggacagtaagacctgaagagaagtctgttggggtgcttctaggaaaggtttttctcactggaaaaaagaaagctacaagagcagaatccccaccctctccttgtttcctacttttagatgatatcatgtaaagagacagtgtttagagctgcagcagccattttgcaaccatgagggaaatccaggaattggtccacaggcataacatcattaattcaccaaagcacctccacagctctctccccctggacttcctgttgtaggcaataataaatccccaagatgggaattttgttgccatttattacaccgacagacttcattcttgcaaagcttacattgctagaggagtgatgtgacacataaaatgaaaaacccgttacaaaaaaacataaaatatcaggcaggacacagcggctcacacctgtaatcccagcagtttgggaggcggaggcaggtggactgctcgagcccaggagttcaagaccagcctaggcaatatggcaaaatcccatttcgacaaaaaaaaatacaaaaattggccaggcatgtagtggacacatgtaatcccagctacttgggaggctgagatggatcacttgaggccaggaggttgaggctgcagtgagccgaggtcgtgccactgcactccagcctgggtgacagagtgagaccctgtctccaaaaaaaaaaaaaaaaaaaaaaggaagaaaaaacctaaaataccataactcctccctccttggcagctctattcgacctaaatcaaaatacgttcatatctctcccacagagttactgctctatatatactttctgctttacagacaaacttcttaaaagttaatctgtccttagcagggcacagtagtgcacacctgcaatcccagctacacaggagactgaggcaggagtatcttttgaacccaggagtttcagaccagcctgggcaacacagcaagacatccccaccccaccccacctccgccactgtctctaaaaacccaccaacttatctacccttaccatctccacttcctattctgtcatcctcactcctcaaatagcaatactctgcccttctttcacccacattactccatcaaactgctttcaacagagtcaccagtgacttcccagttgataaacccaagcacagtttttagctcttatctggcctgacctctccacacaggtcaaaatagtcgatactatacactatacctcccccgcaccatactttttttttttggtttgtttgttttgagacagagtcttgatatgtcgcccaggatggaatgcagtggtgtgatctcggctcactgcaacctccacctcccgagttcaagcgattctcctgcctcagcctcccaagtagctgggattacagacgcccaccaccatgcccagctaatttttgtatttttaatagagatggggtttcaccatgttggccaggctggtctcaaactcctgacctcaagtgatccacccatctcggcctcccaaagtgctgggattacaggcgtgagctaccatgcccggcccacaaaaccatactttccagattttcttttgcatccttaagtatttatttctggcttctccttctactaggctcttaatactagtgttccccagtgttcctgtacccccctccctgttttttcttattttacccactctttttatatagtctcatctgtacctacagctttagtgaactaattagaagtaatttgtaattagcagcactgattttcctgttgaaagttaactactaaaatccctcgatgtgacagttcttttagttaggacaaacatgcccccaggaaatgaagtaatactattcatttgcttgcaaaccattttttccccatagatgaacttcagcccaaataaaatcaaactatccaaaattcccagctacttgggaggctgaggcaggatccaaattcatgtaattttcgtgtattttgcaagtttgttttatgccatattaattcattacactccacatcatattttcttagcaaatacatctagacacctggcactcagtaagggatattcctggcacgataatcattgttatcattagacattgcaggaaccaccatatggatggataaatgtgttgtttaatgaaggcaagcaattactttaagagatcctagttgttataaactctctctggacttcaagtttggtatatacataattattaaatgtatacataattattaattgtaacatgtcctcacaattgatttctggctatttttctaaggattttacactttatttagtaaaatatattaaagaggttaatgataatttttttatagagacccttagattgaaaatgactgatttttcacacctcttttcttgctttactaatcgtcagtgcctaattgctaaagcaaaatatcttctaaattctggggaggaaattctactttgggccaggcgcggtggctcacgcctgtaatcccagcactttgggaggccgaggcgggctgatcgtgaggtcaggagattgagaccatcctggctaacatggtgaaaccccgtctctactaaaaatacaaaaaaaaaaaaaaaaattagccgggcatggtggtgggcacctttagtcccagctactcgggaggctgagataggagaatggcgtgaacccgggaggcggaggttgcagtaagccgagatcgcgctgctgcactccagcctgggcgacagcgagactccatctcaaaaaaaaataataggaaattctactttgaattgcacactaatcaaaagactccagcttcatttacagagaccttatctgtgctaagtagtttttggaatagcttttttcctgtcacttaagcaccttcctttattattaatagtaaactaaataggagtccaaagtctgcctttcccagtgctagagtataaatgtcatcacacggtagaatgtaaacatcttttttttttattatttttatttttttggacagggtcttgctccgtggcccaggctggaatgcagtggcatgatcatggctttcactgcagtctcaacctcctgggcccaagcaattctgtcaccccagcctcccaagttcctggggccacaggtgggcaccaccatggctaactaattttttaatttttttttttgagacagagtctcacactgttgcctgggctggagtgcagtggtgcaatctcggctcactgcaacctccacctcccgggttcaagcgattctcctgcctcagcctcctgagtagctaggattacaggtgcccaccaccatgcctggctaattgtttgtatttttagtagagacggggtttcaccatgttggccaggctggtctcgaactcctgaccttgtgatccaccggcctcggcctcccaaagtgctgagattataggtgtgagctactgcgcctggcctgattttttaatttttatttttatagagatgaggtctcactatgttgcccaggttggtctccagctcctggctcaagtgattctcccaccttggcatcctaagtgctgagattataggcatgagcctccatgcctggcccctttatccatcttttaatgttaataattatatccatttctgtttccttcaaaaattcaagtggaaaacctgacataaataatggtaacttttttattatacctatatcaattaacacatatgtataaaacttctacttagttgtgccaaatatcagtgcacagtatgggtgatttcccccacttgcgaagaatggtgctatagtcaccgcacaggagcaggatagatccccactctcaaaattgggtttggatatcaaaatcaataatgccatcacacatgcagcaaaggggtttgaaaggtttattgctcacataatacgtttttttgggggagagtaagatgggcttccaagctggtctgaaaactagagagaacaagaaaggagaatggctttgggattcttattgtggttaggagtggggctgtggtgagggttcctgtacatgggctagggcttactgttggaactttgtgctagtgccaaggaaggagcacctaggcattcttatcagtttgctcagatgtggggcagagaggaagggaagtggtggggcttgagaactgtctgcagtcaaacatcaaaaattaagtcatactctgtattacaacaattagaaaagaaataaaatgtagtatatcagttctctattgctacaatattgttacataacaaacaacccccaaacctcaatggtatacaaaaataaatgtgtcctgctcacacatctggagtcaactaaacagctctgctgatcttgcctgaactcattcatctgtcagcttgatcttggctggctttgccaggggtgggctataccctgctctatgtctctcatcctccagtaggccagcacaggcatgctatcatggcaaaggcaaaggtgcaggagtaaaccagcccaataacacaagcactttttaagcctctgcttgtggcacttttgccaaaatcctgttggccaagcatgttacgtggccaagccatgagatgtgtacacagaaaaataaaagatgtgtacacagaagagcaaagaattggggccatttttgtaacctactgcacctattaatggattactaatatgtcacaactcttatcctagattacagtattaagagaaggaaagtcaattcaatgatatatttactactaaatgttaggtactgtacttggaattagagttacaaaactaaatgagataatcctaaggagctcacagttggatactggaaaaaaaatcaaacaaattaatacattattaaaataaatgaaataaaatccaatgtaagtacatgaatagatattttaataagaagtggcatggaaatgaaaataaactcttttgtctcctttatcatatatttcatagctaattaaattaattttattataatcaaatttactgtagacctgaggacataaagtttactgagactccctagcgaatggctgcataaaaatgcaagccctctaaaagcaaaaccagattgtaaattctgttaggctaacagaaagtcatttgattcttttgtttttccgttacacttagtagtcaaccaaaatttattgtgtgcctatatgtgtgtattagtcagagttctctagagggacagaactaacaggatagataggaaggggagtttattaaggagtattgacgcacgatcacaagatgaagtcccacaataggccgtctgcaagctgaggagcaaggaagccagactgagtcccaacacctcaagtagggaagctgacagtgcaacctttagtctgtggctgaaggcccaacagcccctggcaaatcactaatgtaggtccaagagtccaaaagctgaagaacttggagtctaatgtttgcgggcaggaagcatccagcacagaaaaagatcaaggccagaagacgcagcaagtctgctcttccgtcttctcctgcctgctttattctagccatgctggcagctgattagatggtgcctacccagattgagggtgggtccgcctttcccagtccactgactcaggtgttaatctcctttggcagcaccctcacagacacacacaggagccatactttacatccttcaatccagtcaagttgacactcaacattaaccatcatgatgtgcaagctattttgctgggaatttgctaggatcgaaaattcctagtttttttgttgtttgtttgttttggttttgaatctttaatgagaaaaaatactattgtgctcaagaacaccgtgcgtctggtgtgtcattgggtcaagggttggggacacacatggcagaattcagaaacagggtaacatcaacaaaaatacacacggtcataaaaagaaaaactggcacctggcatgatcaactctgtttcctcatcattcagcagcatgttggggatcctgcggctgatggggaacacacgtccagactctgggcactgcagggtgccctccaacacatctacctccagcagcacgtggtgcattgtcctcaccatggagattcctagcaaatactctaggtattttaaatagaagaaatttaatacagggaaatgattacatatatagtagaagaactgagatgtcaaacagaagagtgaggcaatccacattttagcaactgcagtaagtgacctccaatgtcatggctggagggtgcaaatggtgagaaggcagaagctagaatcatggtggaccccacctgcaagagtcacaaagaagatgcatccattcaggtcacagagaagtaaatactctagtgtctaccttctaccttcttattgctctctagtctcccattttatctagactgctgacaagcatgcagtggaaggtgagcaccagatctgagaggaaacaggcagtgtctggtggcacactacacaagaactctcacaggcatccactaggcttgcttggtgactcatttaaggagtaaccctattttatgtatcagagtagtctatggtgtgatatagcatattgctgcttttgatgtttggtttcatttgttccctgtaggcacctaatcactgttttttaaattccccaggagattctggtatacccaatcagctgagaaccactgaatagtggctttcaaaattttgagcatccaaatattgtcttcacagaaaatcttaatctctgaagtctactgtaaaaacatagaaaggcaaggctgttctgcttgaagcgtagcttgggagcctagattcctctcaaacccaggccactcctgggaacatggtctggagatttgaatgttcccaaatctagtttatctcattagtcgatatttatgcacatcctctgtgtctctcagtgttctctcatgtgtagacatatgccccattccaaagagtgactgtagtgtaatgtggcattaaaggaaccctggatagacctgtattggaccttacatattagagagctctgcagatagctcacaaatctccaagtcgctagctgtaaattgagtgataattggtgtcaggttattttagcacagtttggggataaaatggtgcattaatctcccagtttgtggcttattaaactttgaaatggaattatttgagacataaaagagatagcaatactttgctggatgttctccattagtcgagattgttacagaggagaaatttttgccactgtttatcaaatattttttcctctagttaatccatatcattccgggagctttgcccaggaaaaggacgaattacttgcgttcctccgatctctttcaagttctactcccgtacctttaaactgtcacacggaatctttggtgacagaggaatagaggtgggcaagcagggcgtttggaaaccatactgaaaacgaattatccataaagcacagattcaccagcctagaaatggctctgtctggatggattccttgagatgtgttttatctccattaaaaacacaacaactttcaaactttcactgtgtctcaaaatgatagagaaaaaaattcagcctttcaagtattcaagaaaagcttgaagaaataaaagaaactcgctgctctctgggccactttaggaggcagagcgttcctggagaacttcaggattcattcgcaagggaacccaaggtgggcattcaaagccgatccaaaggatattcttctttttagaaaacaaaaggagcgcaaggagtaggggactgaaggtgcccaaagctagtgggtcagtgtcactttcttcctccctttcgcccctttcctcccgctcctttctcccctgtggtctctgctcagcacccctttctctccagccccgactgcccatcttcgcccctaccctcgcgcgtcggggccgtgtctacacccaccccaggacctggtccgtccaggctccttcccatcctcacacccgcgcctttctccctgcggccccggctcgctgctccagctgcccagctcttcccaggacaggggatcccgccaagggggccgcccccgccccggccctcccccgcccggccgcaccataaagcgcccggccgctgccgcggagcccagcagccagctccccagcaccgcacggcggggacgcgagcgcgcccccgacggcagcccggacgccgagcacgggtcacctgcggcgccggcccgggcgccgaccgaggttcaacgcacggcccggggacccccaggcggggccaacgccgccgtcgcccccggcctcgcggggagcaggaagagccaacatgctggccccgcgcggagccgccgtcctcctgctgcacctggtcctgcagcggtggctagcggcaggcgcccaggccaccccccagggtaagtgggttcgggtcgggcctgggagcgccgggcaccgggtgccccatctgctgagtgagtggagggacttgctcggccctgtgctcctgtggccttgctcagcccctctctgtccctcccgggcccaatcaaagcatattgtgattggaggtagtgattagtctagaattttccgaaattcgattatccaatgatgagaacaattgcctaaatagttgggggatgcaaaattgtttcagactatgcaaagatgtggggtggggttgccttcaaaacttgctacacggtcctagacctcttaacatgttaggctctggtgtagggtgaattccgggtcctgcaccctgtgccggttccctccaggcagccttgctccctaaatccttcctcaggcggagcgatgtatgcccagagtctgctcttgacatccctgagaaagacgcagaattactctacttgccctgccacctgcattttctgctctgactttgcttgaacgacaaaaagaaagtttatttcaaggattggcttgatgtcttaatttctgaggtaaataggaagggctgagaaactggtctttttcaatgaaatcagttctttgcagcaagatggatgcagctggaggccattttcctaagtggattaaggcaggaacagaaaagcaaataccatgttctcacaagcgggagctaagcagtggatagatatggacataaagatggaaacagtagacactggggactccaaaagtaggggggaaggttgggaaaactaccaattgggtactacgttcactgggtgatgtgttcactagaagctcaaatcccaccttatgcaatgcacccatgtaacaaacttgcacatgtaccctctgaatctataatttaaaaagaaaaaagaaagaaattgatctttttctatgaaacggtaaaaattcaacaaataatgtgaaaagatacttggacaatcctcaaaccccttggtgaaatggggtgaaatagccattgggtagaaagcagttaaacagaattttttgtaaagaccaaaaggaagacatgatttaagcaaagaaagatcttagggctataactggccctaactggtcaggcgtgagcttcatctgtttgaacttggtgcctgggttgatttattactgactgtggaagtaaaactggaagagacgcacaacttgaagtaaaagttgtttttcctcttgtagccagagcaaagactgcctatgagggcgttctcaacttccctgcctctcgctgctggaatcacccatttaaactgcatttcgccaaataactcattttcattccagaccagacaaccagaactgtggtttcaggggttagagtactgctaacttttagtttgtgttgaagtatttgaaatgctgtagtccgtgccccatacttctaggaaggtaaaaaaggaagcttttaaacacttcaaaagtcttattgtgatgatatttggcaagttcaaatttataggtataatttttttgtgttatatacgttcttgacttttgaacatacagctttatggtgtgccttaggtatgtctcagacatacctgaccattttatggagatttgaactcggcctatacaacctaaattatctgggtaatttagttgctgtagatgatctgattaagttaagtttctgatagattcttttattgcattatctagtctgcaattatcaggcttaacaggataatttgattatctccttcttcccctgagcttcattgttggtcttaaattagtatatattcaatttacataccatggtagctgcgctcattggtagcagtaatttcacctctaacagtagagggttgtgctttgatcctggcacttaaacgtaataacattaatagaaaatggtagtgtctcatttccttttcaaaacactctctatgttcattactaactaatcccatctctgccgcttgagagagatgcaaatgaatcagacgaaccatagattgaaataccgcaagtttagcttgagttacatcatgaacctgatcatagtcaaaggaagaatcaagagtactaagaattttgttttgaatgtagttcccatactttaaatacatatctcttttctgaagatgttttgtcctgaatggaactttaaaacatattgccttatttccctttgccctcattagtggaaatgtaaagtttccaattctcttgaaaattaaaaaatccaatttcccttttgaaatgcagtaggcaacagtgtttatggtttagggatataggcactaagaggagcacagtaaatgttctgctttccaacacatctcatgttaatttcattattgtgtaaggacccctggtacatgtgaaaaatacagtatagatccagtttgaagacaaattgtcttttaaaaggggtaaatattcttgagtaactaggtttttgcctgtttgctttagaacttttattcattcttattacaaaagcaatacctgcccattgtagaaaagattaaagttagataagcaaaaaataaaataaaaagaaaagaggaaatttaaaacttatatttatgcaacccggggatagcttctattaacattaacatgttgttgtattgtctttcatttcaaatatttttctatgcaaaaatatatttcaaatggaattacgttatacatatagttttagaatccttttttcttaatgtatcatgaacatcttttcacatctgtaagtattcagctacattttaaaaataggtagtattctgctttttggaagaatcaaaatgtatttagccagggttgtaatatagaacccttaagttgttttcggttttgatattgtaaatattaatttattgaacaagtgcaggtgtcacaggatggtttgggatgagtttaaccatcccctcaatctcagccaagcccttctctttttttttgctaaggacagttaatgttttcatagcaaaatgctatgccaacagagctcaggtttacctctcttccaattagattttcacctaattctttcctaaatagaaattctagaattaccactagtgtctaatattcttgtacctaaatcatttaccatgttatccattatttccctaggataaattcccataagcagaattgctagatcaaggagcacatatgttttagggcctttgatactgttgtttaaactgtcttcaaaatgtttatactagtttaaatgttacacagtggtacagagagtgcccatttccctgaaccctcacaaacagttgagtactgttgtatttactaattttccaattcgatggtgaaaacaatacgttattgtttaagttagcaactcctgcatcattaggtaaatacatgaacatttatttatatacttaatggtcattgtaattctttttatgtaaattgccttttcatattccttgcccatttttctattaagatgcttatcattttattgatttgtaagaactctttatcaattgtcaatattacccatttaataaccagattattatatccaagagacagctgtaggtgctgaagtttctgcagcctatggaattgttcaaatgatatttgcgtctgctaatagatatactaaataacttccactgagtattacccaaatttggcctttgccttttaaatgactctgggcatactctctcatagctttcataaatattacaaagtttgtgagagaaagtactgcttgcttgatcaaaagcaacttatacaaatacaaatgcagattctgatttttgtagagcaattctatcagtcaagagacagaaaccacatcattttgttttgttttgtttccacattgggatttttttaaacagagaaatgttggagaattgtaaaggcacaaagggaacacttaggtctcacaaaggtggcaaatcactttctaccagcacatctaccaccatagccatgttttctgctgcatttcctgtcatttgtcactgaccaggaaaacacaattggctctggtcctggaggtgcctatgggactccgtgacagaaattcactgcttggggacatcagtaaagcaatggcttggatgctttcaccagtcccccttgtactttaagcctttagtgcttttcctggtgggcattttcagagctacttgttcagccaaacccaattatgtgtttgcaaattcagtcctctctcttgagggctacacccagtgatgaatattgtgctgtatttcttctggctcaaatgtccagccactataaaagtgacagggaagaactggtgagtttggcaatacggaaaagggattacaagattagtttacattactttgtagcttactgtcaaattgcaccccccccaaacaaagaaaccctgttttccccaaaattgaatctttcacttataccccttcttctcccttcttcccagtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaaggtaagcaaatttgctgaaattattttcttaaaaatctcctttttctattccaggattaggggtatactgtctttgagatggtttccttgtacaattatattctcactcactagtcatttcagtttttaatactgaattcttcttactgtacatttacctatattggcgggggggtggggggcggttcaggaagaaagtgacattgggagaaaaaaagaattgctgtgaccaagatgctttttgacattgtctcatcctgatgttattctgaatccagttctggaacagagaggacaagggacaattcagccattatcttaggcagctcaggctgctataacaaaataccatagactttgtggcttaaacaatagacatttacatctcacagttctagacagtggtaagtctaagatcatggtgccatctcagtccattttgtgttgctataaaagactaccggagactgggtaatttataaagaaaagagatttatttagctcacagtcctgcaggctggaagttcaagggcatggccctggcttctgacaaggactttcttgtgtcacaacgtggtggagaaggtcaaagagggagcagacacatgggaagaggggaaaacctgaggggcaccgtagctttataataacccactctctcaagaactaattcattcccttgagaactaatccattttcacaagagctgaaactcactgtctcgagaacagcaccaagccattcatgagagatccaatcccataattcatatgcctcccacaaggccctacctcccaacaccgccacattggggaccaaatttcagcacgagttttgacagggacagacataccatatccaaaccataccacccgcagatttggctccttgtaagggcccagttcctggattgtagatggccatctttttattgtgtcctcacatgatgaagacagacagcaagctctcttgtgtctcttcttataaagtcactattcccatcatgagggccacaccctcaggacctcatctaaacctaatcatctcccaaaagccccacctccaaataccatcacactggagattaggagttcagtatatgaatttgatggggcgggaggaaggggacagatacaaacattcagcccatgacagccaccccatactattcttatgttccctttgaagccagtgctcagtagacctagctcctaactccttaagcacctacctggccgtgatggaaagaatatacctcaaccagtagaacaggatgaatagggctcttttctccctgtgcgtgccaatttacacaggcctgtcactggtttgatccctctgctgtcatttgttttgttgccgtagtgagttactataagcactgagaagtgagtcccaaagaaaatggtcactatttttcattgccttctcctccactccttccccgggggacaacacctgttcctgtttattaaggaatgtgacagatgcgtgttcctgctgcttcatgcatttaataaaataggagggctggttcttctggctgggttttatgtttatttcagtttgctttacaaagtggacacttcctagtgcttcaaactctctcttagattatatttgtgattgtacacttaaatctgcaggcattaggaaatgaaagaagtttctaattcttaactttcataggactctaaagttaaaagtataaatgaatgggactttaaccccttcttttctgtcagattccaacatgcttcttgcctgcttccacgctactcccaagtctgctttctctgtatgtgcctcatagcctgtcaggggccttgaggaggtgtcagataataaagtgagtctgaggacatggccagcagatgggtcaaccgttcctgtccccctttccccatcttcagccctaatgaggggggattctgcccatgtctctgccttgaatcaaacagccccattttgcctagcatctgctgaaggaactgctcatcttctgacaacccagctactcagtttgtgatcgtaaaaatcaccgttcttcatctgtggatcagcagcttctcctaacctgtctatttcagaggcattgcaaagatttttctttcggttcatccagcttaatggccagaagttcctctgcagagtaaaaaactgctccaagcttttgccctcactgcctctcaccctctatttctaaattggccagtatgtgtgagcccattgtggaaagtctgttgctgggaaacacaaggaagcaacttcccctgaacggatcctttcagtttgtatagacaagagtaacactggatttaatgggcagtaggtagagggtaaggggatcgttagtatgtttcctctgaagaatcaccagtaatatgagtcagctgtaaataccagttgatcaaatattttaggggctgtaatcccagcactttgggaggctgaggcaggcagatcacctgaggtcaggagttcgagaacagcatggccaacatggagaaaccccatctctactaaaaatacaaaaattagccaggcatggcagtatgtgcctatagtcacagctactcgggaggctgaggcaggagaatcacttgaacccaggaggcagaggttgcagtgagctgagattgtgccgctgcactccacacttcagcctgggtgacagagcgagactctgtctcaaaagaaaaaaaaaattagaggaaaagatgaaagggttgagatgaagaactgaaagatgaacatctccctaccctccaacctattaggaaagataaattgttcttagaaactgtgggggcagaaggagtcctggaagaatctagaaatgagaattctcataaggcaaagagggatttatgctgttattagagcttgaagagttgaagaatgacacagagaagttgtttcactggagcagtcggaaaaaatacgaagatttgccagcactggtgaatataaaagtttcaggaaagagaagtccattctgactggaacaataaaaagaagtcactgggtccatcagatccaaggaaaagacagagcctgtggttgagactccccactgcagtgttcagtggtggccatgctgacttgacataattctccccacaaccctccacaaattatctacctggacatggtaggctccagacagcattcatctggccaaggttcagctgatctcctcttttgctgatttgcgtggaaaacggtgaaacagaagaaatccaagtggatctctactgacttaaaacacctgagtaggaaataaaacacacccttccctccattaaagtccaggactgggagatgaaggagggtgactacacagcatcaaagagcagaatttggtcatctgggcctcatatgacctcctgaaacaaattaaaatgtagctaatgaagactgcaaagaaagagtttgccactgagctctctgcttctactctcaccctctccaagtcagcctaccccaggccacctcattactgttcacaaacataatttggatcaagatacatctgacctctgagacctctagtggccccctttccaaattgaataatgtacccatgtctcagcaaggcattcggggcttccactctgcatggccccaagcagcctttccagtttatccctcattccagcctccatgcagcctgagcttcaccaagataggctattgccattccttggggccgcacatgctgcctctcctccatccttttgttcctgctgtctgctccctcgacacatatcctgcatcacctcttgacacccaaatcctacacattcaagacccatctccagctccttctgatattctttctaatatgactttgtacctcttaaggaagacattcatcttagctcaccttgtacttgaattacttctaggtcttctctccatttggttgggagctctcttgagacagggactatacagcccttatctttgcatctgctgcagcacctagcaaggtgccttgtacatagaagtagttcatggatagattgaaaagaataaatggaggctgagaagagtagacttgtttgtgaaaaatgacatacaccattgtgaaattctgggattccacaatatctgacacatagtaggtaataaaaaaaaaattatttatttatttatgagacagagtcttactctgtcacccaggctggagtgcagtcgctagatatcggctcactgcagcctctgcctcctaggttcgactgactctcctgcctcagcctcccgagtagctggaactacatgtgcgcaccactacgcccagctaatttttctatttttttagtagagatggggttttgccatattggccaggctggtctcgaactcctggcctcaagtgatcctcctgtctcggcctcctaaattgctgggcttacaggtctgagccaccacactcagccaaaaaaagatttttttttttaatgaataaatattgaagggcttttggaataggatgtaagaggatacaggtagtatttctggtagaagtgatctgccaggttatgagtaatacagcctttaaatcttgatagaaataagcatcctgcagaaaccacctggctggatagaagatatggctgatgctcttctagtatctggcgtaaagacaactttccagtttgtatcctgagtttgagtcacaaactgcatcaccatgaatacatctctgaatctctcttagcccttgttgattgcctataaaatggggataataatagctacctcacaggctggttgcaagaactgaattatataaggtataataactttcttcagactatgaggcttaataaaattttcatactcttaaacagtattagagacagatttccactatttaggcaactgttttctaatgatgatggtttggtgaactcttggcttgccttgatgacaatttcacctttcagaaaaagaaacacaagaactgcttctttagtcttagtatgattgtctcagaacaacttggtgctgtgaagtgaaggaaacccaaagtgtttgctgtaatgaccagagagtgtcagtgaaactccttagcctaaggataggacatgtcaaatagttcagagtagtggttctgtagcttgcaagctaaagagaaatatagattaagagagatggtagagcttaaaaaataaaattatagacacaataataagtggaaaaaatgaggggcctcaaacattaataataatatttatggagtcttttttttttttttttgagacggagttttgctcttgttgcccaggttggagtgcaatggcgcgatcttggctcactgcaacctccgcctcccgggttcaagtgattctcctgcctcagcctcccaattagctgggattataggcatgcgccaccagacccagctaatttttgtatttttagtagagacagggtttctccatgttggtcaggctgatctcaaactcgcgacctcagatgatctgcctgcttcggcctcccaaagtgctgggattacagccgtgagcaaccatgcccggccctatggagtctttttaatgtaccaggactgtgttaaaagctcaaagcctatttccccttttaactttcacaatattcctatgacatgcccttgaattagcttaattttaaagtggtcatgtgcagaagatggaaagaattttaaaatttgacacaaaactataataattaggacaaaaaagctaaaacctaattgagtcaaagttggtgattcacacataagataataaaatggatgtttttagccatgttcagaataagatatggaagatatggaagaaataagcctacctcctggaataggtaatgggagattaacaggtgatagaggtaagtcatagatggctcttttgtttctgtcttacccatcaaggggagtgttcttcaaattgtcaagaacagttaaaagaaaacaaaaatccaagctagatgaggagatagcagaaaaacgcccactctctttagtgagattaactattcaggagcagatgaattacacattgggatatttagtgaagaaagtcatagtggagccactgttagttcatttagaaatcatggagaacagaaaggtcctaaaagacaggacaggcaaatgtttccatttttataaagaagggaagggtggattctgaaaaccacaggctactgaaatttaggacagagctccagagggttatatgattattcatgtattttgggattgcccagaaaagaaatctgggattttttctaagacaaagtggatttactaagaataaatcatgccaaacttttttttttcagtaaagtttcaaagctgatagattaaaaaaacatgctgtgagcataatgtatctgacttcagaaaagaattggatgaagtcgcttattatacctttctaaataatataaaaaagaacgagctaggtaatagtaccgctaggggttgctaagtcactgtatagcaaattatctagtggtttttgacagggctctgtcttgggtattgaactgtccaacatgtcagtgacttgagcaaagacagcattttatcgtgtttgcaattcaaagctagaaaacagttgaacatgttggatactatcattcatatttacaaagtcctaccagataatacaatgggccaaaatcaatcagatgaaatttaacaagaataaatgtaaagctatcaactaggagtgaaaagcaaattgcagaagttcaggatgtgagacatctgtcctaagagttctctaatcaacaacaaatcctaatcagtgttgaggatagtgcctgacacataatggatactcaaaaatatttgccgaatgaatgaatgaacagatactgtgatgcaactgtttataaaaagctccttacattttaggtttcattagtgcatacatatggccaggttaaagaagctaagagtcccactgtactcttaattgtccaggccacatctaatactgtgtctagtcctggacacatttcacaaggacactgtcaagtcaaagattaccttgaaaagagtcaccaaaagagcacctatctggaccccgtttttttgttttgtttggttttgttttttgttttttgttttctgttttttgtttttaccacatttaagtagaaaagttaaaggaactagagaaatgaagaagccagacagaagacatgaaaaatatgaaaattgccttacaatatttgaaaaaagggctaggtgccgtggctcatgcctgtaatcccagcactttgggagtctgaggcaggagtatcacttgagcccacacattcaagaccagtctgggtaaaaaattaagaattttttttttttttttttgagacagagtctcaccctgttgcccaggctggagtgcaatggcgcgtcctcagctcactgcaacttccacctcccaggttcaagcgattctcctgcctcagcctcctgagtagctggggttacaggcaggcgccaccacacccgcctaattttttttgttgttgtcgttatccttagtagagatggggtttcaccatgttggccaggctggtctcaaactcctgaccacgtgatccacccacctcggcctcccaaagtgctgggattacaggcatgagccaccgcacccagccaaaaatacattttttaaaaaattagcctggctcgctggtacatgcctgtagttccagctactcaggaggctgtggcaggaggatcacttgagcccaggagtctgaggcagcagtgagccgtgatcatgccactgcactccagcctgggcaacagggcaagctcttgtttcaaaaaaataaaaataattaaaaataatttttaaataatatttgaaaaatgtcatttggaatgaagatcagactaaatctctgtctagctctgaagagtaaaattaggatccattggtagaagacagaggaaacagatattggctctgtattaggaaaatcttggagagtgatccaaaagtgaaatggacttctctgcaaagtaatgagttccttgttactggaaatgtgctggtagaggctgcacttttattgaaggaatttgtgaagtgagatattaagctggatgacttataatgctaatattttgtcattgtaccaaggctagagaagtttcataggcacttaagaaattgctttgcacctctttcctgtaacatgaccttgggcttctgtgcacactcactgctttgttgtctactagatagttgcatactatgatgaatctcatactttttcaaaacacaaagtcctctgcagatgtttagcataattgctgtgtttcctttcactagatagatgtgtgtgtgtgtgtgtgtgtgtgtgtgtgtgttactctgtatactttacatttattcaaattgccataagaaaactttgaaacagactgcatttatcacagctaggagtcacccagagtactttcagaaaaagcaatttgctgatttaccttttgtatgtatttccaaagcactaaagttacaaagacctagtgtatagggcatgaagatgaataagcaaaggcaaacaagaatatccatcacaagagacattaatgtcaaggaggcaatttttaaatgtacaattatttaaaaagaagaggaaagattgacaatgatggcttccatcatatgtttaaaagtgccaaagagcaaagccatttgccaaacaaagcaaagagctccagactacctttgtgagtttgctgcctgcaggccaggccactctggactcctttgcccaagcagacaaaatattatcatttccagttctttaaagctggcaccctgcttcataactttgacattcaacatatcataatatttttgactaccaggtccacttttcaaaaagcatcttttgaaaattctttctgaagatagcaatatttcaaatcttcattgcctgggcttgctttttcatctcctgccagtcactgaaatcgtatttaatcagactgggctatgtggttcctgtgtccggctggtcccaagcatggaaatgtagctcttggacagtgccataaaatcaggcaatcctgagtagctggggttacaggcaggcgccaccacgcccacctaattttttttgttattgttgttatccttagtagagatggggtttcaccatgttggccaggctggtctcaaactcctgaccaaattgtcaattacacaaagatgccaagtttagagggaaaggatttatataaccaacaaagtgttccacattttaaaaaagacataattgtgagtaataaaagtcagagtggacacactctattttatctattttttttcacttcccataagtagttgtagtggtgccctcagatactatgggatgtgggaaaatcagtaagatatagacttggcagcatgaagggacttaaggtggtgggaagattagggccagtcctgggataaggagttgaaacattatttagaatccactagggagttattgaaggatttcatttgcttgcttgctttgaggaggcaataacgatcagaacagtcctataagaagttcaatctctcagtccaaaaagtccgtgcagaaagagacctaaagtaggaaggggagtcaggaatctaacagagcagcccagaggtggagcagagtcagagacaccgtttgagatccgagcacccactgactgacagccttcagttccaatccaatctgcactgcattctgggtactaaccctgcacaataaccagctgcaattgccaagaaaacctgtgcagctggtggtgagtgaaaaccaaggatgtgaactcaatgtgctgcagctgaagaaacagagttgtacctggaaccgcccagtgggttcaccttgcccactgcctacacagaacccatttatcaagacaggggaattgcaatggagaaagggtaattcacgtagagctggctgtgtgggagaccagagttttattattactcaaatcagtctccccaagcattcagggatcagagtttttaaagataattttgcaggtaggggcttgggaagtggggagtgctgattggtcaggctggaaatgaaatcatagtgggtcaaagttaggttttcttaacgtcttctgttcctgggtgtgatggcagaaatgattgggccacattactggtctgggtggtgtcagctgatacatccagtgcagggtctgcaaaatatctcaagcactgatgttaggctttacaatagtgatgttcccccaatttggaggtatttggggagcaatttggggattgctcagactcttggagccagaggctgcatgacctctaaactgtaatttctaatcctgtagctaatttattagtcctgcaaaggcagactggtccccaggcaagaagagggtcttcgggaaagggctgttatcaattttgcttcagagtcaaagcatctactgaattccttcccaaagttagttcggcctgtgcccgggaatgaacaaggaaagcttaagtgttagaagccagatagagtcggttaggtctgatttctctcactgtcataatttcctgagttataatttggcaaagacagtttcatacccagtgactgttttcctgagtgctttccagggtggtggccaggcagaggtgtggtcgagtcgggaggtgggatggttccagcctcaggggcctggcttcttcttccttcctggagggacaaaatggtcatcttcctcctgctgtagtgttttcccctgcgttttgttccttctgctatgtgtggatgtgttgggataagatagggcaagatgagcaccgggaaagggggctggcaagaggaagggaaccctgtctgttctcttccccttcccagtgatgctgcctctggagaaggtgaggaaaactcgcacccaacaggctgcaggctcctggggccagagctttctgctttttccagactccatatgactgatgtgaaaacgttgagtttcctgagcctgcttgccctctgggtagtctaactgctctgataataaaataagtgtagaggcactgaagtaaagaatttgcaataaaaaagaaaatgaaaaatggaagccataagagaaatgagtattggatcaagtgtgaagaatgggaaatgtcaaggcacttgataatttttctgctaacaaacagacattgtgaaggtggaagtttccatggttttccttccctgcgttttcttttgctattgtcatgagatctaggggcatgagggatttgtcatttgagtgttacattgcaacaaaaagcaaggcagaagtacagaatccacttaaatgagagtatgaacacaataggaggttttgctacctggaattagatgttgaattgctgctctctaaactttattttctatataaaaagctttaaatacaatcatttaattattcatttaaagtttatattaacaacatttagtgtgcacctgctatatgtaaagtatcccactaggtactattcatcaaattaaagccagctatttagttatgaaaataaaccatggcccacataccctgtgaagccctgttaaaagcaggcaacatgcccaagaaaatatgtttcctttgaatttcacagaaatcttagtttgaggaagaaaaatcaagagtttttgttataagccactatgtgtatgctctgaaacagttaatgtagagaacagaggtagtggccataactgaaataaggagatagtgagtgagtgtgtgtgtgacctaattcaagtttggtggtccaagtggtatttttcttggcctgtaaaaactacaggtatttgcaaaactgttctaaatagtaatgtaatagaagttttttaaaaattgagttagtgcatatgtttggactcaatttctccctcttttggttactatatatggttctaatttccagctgtgcagttactattgtaaattctcaacattaaaaaaattttcagatacaaattatttttattgatacatactttttacatatttaaggggtacatgtgaattctcaacaattaatattaaataaaaggagaagaattctgttctatcccagattagaaataccttagaatttctatttgtgtctatttccctccattccattattttcttcctcttcataccacatttatatacgtcttaccatttatttatatttatttaatactttacttcaggaaacttagtattgttctggaatcagaaagcccttctagctagtctgtcttgccaggtctttccgattcttctccctcaagaagcaagtggtggtgaccacgtcgacataagagatttcccgtctgactcctcccaccatctgtgtaggtcacagaacctcacgccaactggctctcttcagactgtggtcaactggaaaatcgagtcagatttcagaagggtgggaaaatgaacctagaaagtctaatgtggagaaagccttaaaatatatccttcatgttccacaatagtgattattatctttggcgatactgcaacataaataggtattcttggctagttcatctctggcggcaccatcttggcatttacactctacagaaaaggtacctgaggagaaagatggtccaagttacttgccacagatcacacagattaacagcagcacagtctgggtgtatatgtgtaatatatatctttttattatttttatatacaagtaatggttgtttcttgtagaaaagttaaaacaatgaagatgagcaggaagaaaaaaataccatccaaacacccagcaaataaccatactaatgtagacatattttcttctaatcttctaggtatgtatgtatacatatatgtacatatatatacacatgcacacttttttctaaacccatattgacctataacctccttttttttttacttaacaaaatactgggaatgtctttttgtgtttttaatattcagctattccatagtattccattttataattatactgtaatttaagtagccagccctctccttttggacatatatgttgtgccaagtgttgattacaataagtatttctgaaatgaacattctcacccatacatcttggtgctgcttttttttttttttctataacccaggacagtgctgaagtgcttcttttctttaatttcctcagttttagttcctagaagtggcaatgctgggctgttgggtttacacatgtttaaggttctgatgcatgtgactaaattaccctccagaaaagttctgctttaagttctacttaacgctttcccaccctgggacagctgaagactggccagagctttgcagctgcaatgatgtgtttaccacctcccttatccagcaacatttgcaattagcctccgaccttgttcctgggattgtttccggtgaaaattagccatgcctggctaatttttgtatttttagtagagacagggtttcaccttattggtcaggctggtcttgaactcctgacctcgtgatctgcccacctcggcctcccaaagtgctgggtttgggaatggtacctgtggatgcacctagaggtgtgtttagacactagctgaaatcactttggtctttctagcaaaccttagccaatgggctccactcacaaagggtcagcttccaagagcagattttagctcagggcctcttatgttcaggacaataatggagtaaaaaaacaaggctgaaaaccagaagatatgtgggcttaagcagatatgtgaatagctacccacataggaaaaacaaaacaaaacaaaacaaaaacaaaaacagaaaataaggtgggagtccttcttttacaaccatggataccttttcttttcttttctttttttttttttttgagacaggatcgctcttttgcccaggctggagtgcagtggttcgatcatggctcactgcagcctcaatcccccaggctcaagcaatcctccacctcagtgtcccaagtagctgggactacaagtatgtgccaccacacctggccttttttttttttttttttttgaaatggagtcttgctctgtcacccaggctggagtgcagtggtgtgatcttggctcactgcaacctccacctcccaggttcaagcaattctcctgcctcagcctcctgagtagctgggactatgggcgaacaccaccatgcctggctaatttttgtatttttactagagacggggtttcaccatattggtcaggctggtcttgaactcctgacctcatgatctgcccacctcagcctcccaaagtgctgggattacaggcgtgagccactgcgcccagcctggccaatttttttaaatttactttttgtagagatgaggtctcactatgttgcctaggttgatttcaaactcctgaactcaagcgattctcccacctcggcctcccaaagtgctcggattacaggcataagcctctgtgcccagccaatacctcttattatctaactgagcattgttaaattcagggatgggttgctgagggacgatatgacagattacactcgatttgatgtgtgctgactaaggcctgtgcatgcctagcattgctttagtcatggaatgaatcacactcaggttcagattctagtcctgccacttcctatgtccttgagcaagtgccttcatttatctgagactcactttccttgtccatagttattccaattgcatttggttgcagtgagtagaaattaagtatttaggctgggcacagtggctcacacctgtaatcccagcactttgggaggccgaggcaggtggatcgcttgagctcgggatttcgagaccagcctgggcaacatggcaaaaccctgtctctacaaaaaatatataaattagctgggtgtggtggctcacacctgtggtcccaactacttaggaggctaagacagaaggatgacttgagcctaggaggcagaggttgcagtgagctgagatcgtcccactgcactgcagcctgggggacagagtgagatcctgtctttaaaaaaaaaaaaaagggaagaaatcaagtatttaaagcacctaaggacagcccccagccccatggtagtgctcagcaagtgtttggtgaatgaatggattcatgctctacgcttatatagagtttactttgctttcagattgggttctgcttgctcattgtacttatcactatatgagaattgtattgtttattttcaaatttgccacatattgtctgtttctccatcaacacaaaacattctcaggagaagagttggttcccagtgcctgaaaacagtgcctagcacacagaagatggtgaatcaatatttgtggttatttctcactcacattccgtccagcaacccagtcttgttaccaggaggctgttcagcacaggacacttatcacaccatttgttgaccctccacttccccctctgccacttatcacaccattggttgacctgtgcttgcttccagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggtaggaacccacaaaccattctctgaagtggaaaaatgagctgccagtgcctagggagtgcagaggagcgtgctgagaaattcctgctgcaggtcagtgggtctaatgccgcttgcacatcagaatcacctgcggagcttgaaaagcaagtgatagggggctgcccctaggacaaactgaatccgaatctgtcgggtggggcctagcatatttttatgctaggggcatcagcatatttttaaaataccaattctaatgtgatgccgaaattgagagcttgtgctttcaatgataagtagagactctggatacaattgtaacgttggaaaggaagggcaaaagtaaaaataactacctcagggtgatgacattatctattttcaattttctataatgaaaataaaaaagaatgattagaaactgccaagctttcataacagcattttcacaggtcttgtcagaacatccactaacaaaagggagcttagcttagctgcatttggtcttaagataccatcacctctgtcttctgttttactccaggttgatggtatttttattattctgtcattgcacctgctgtagaaattgccacatacatttcttttagaattttgtgctaggcagtgctttgttttctataaagaactagtattctttgtcctttccaataatgtataactggttaaagttcctgaatttcatccgctatataagccaatgtcaaatcagtaactatgttcttctgtaatggtccatattatttttgtctgctacctctcaatcctcactttccatttgttaattcattatattaatatatgctttagttacaagctactctgagccctttgtacagagaagcagggtgtaagcaataatttccattgcaagtaggaatgctgtgcagttttagatagtaatataaaaactatgaatctgatcctagaagttttgcccttgaacaacaggaagcatttttaatcagaaattccaagaaagctaaaatccgaggtttttggggatttttgtttttcagtcatatatagagagatttttaatgtagatttacatatggaaaaaaattaatagaaagatatgctaaactctctttctgcatccccatagtagcgcatgtaatcagaatgacagtcagaaattgtgtcactgcttatttcagaaattagtgttcaccaccaaataagtcaatttctaaaagtcaggactcacccctgacaaagtcatcagaaactgtacttactggaattttctcagaagctctacctccttagaccatggtagtgatcacagtggctcagtctcataatcacctactcactcattcagtcagtcaacaaatgggcacattctaggcattagtgctaccatggtgggcaaagccagtcattttcctgactctgcggagattatagtctgatgtccaatacaatatgtgttaaatagcacatgttgcctaaatcttagctaaatcaaaaagcagaattcctctgaaaatccttagctgtgctcatttctaaaataaggatgatgacttcactttggcagttaacataaatgttttgttgtatatagatataataaaggtgattgcaagcagcattcacaagataaaagggcaaggacattgactttttgactaacaatgaataatatcaacccgtaatataaaaatatgatgccaactacctaatgatcatagaagatatcatttattcattctcatatcccaaagaagtcagaggctttgctagacccttgagccaagacaaccaaaaaataatactgtttaaaattggggttaacaccttatttattattatctaaattccataaacaaattacagacaattcttaagaggctcctgccacaaaataactcctttaccatgtaggaaaagagcctttcgttttctcagctgtcttggtgacaatgccagcattttcctaggctactggctacttacatattcactttagtcaaaaagatagggctggcaggctttaggaaaaaatgtgatcctacaaaattatgtaagatgtcagagggtccaagtatacaggactacttgattatgaaaattgcgtgagtaggcaagcacattggcttttgaacagtgtcagcaatctgtggtatgaatgtgatttcttccaggacttcttggaagagctgaagctggtggtgagaggctcactgttccaggtggccagcctgcaagactgcttcctgcagcagagtgagccactggctgccacaggcacaggtgtgggctcttgggcagtttgcatgccttcatcaacacaaactccaaagaccctgaataggctgggtcccatcaccgaaaagtgggactgtcaacagagcagcccaacaaaacgtatctatgaaaatcagtctcttgaacaaaatccagggcacagaatattttcattatgcttttatttttatgattcacatggaatttagacttagtcagtggcatcattatcattattttttttagctgaattaaagaaattaccccaaattaaattcaaagattctgggtttttgaatcatcttgtcaggatgatttcctggggaataatttggttttgagtcacttagaaatgaccatcactggctgaaaactctttgccttttgctgttcctacaggggactttaaccggcagttcttgggtcaaatgacacaattaaaccaactcctgggagaggtgaaggaccttctgagacagcaggtaacaagcgggacatatcatcagaaaagccctcgtcttcttagagcagctccccacaagctagtggagctgcaggcctgcccaggcacgggggcagcctctgagccagatctccgggggcccacgcagccccgcacgccagggctctgctggaagttctctggaaatggggtccaagctcgtgatgaacacaccatggctctaagcagagcgggagccatcaagggctgctaacgctactgtaagaagtgttacagagagccaggtgcggtgcctcatgactgtaatcccagcactttgggaggctgaggtgggcagatcacgaggtgaggagctcgagaccagcctgaacaacatggtgaaaccccgtctctactaaaaatacaaaaattagccaggcatggtggtgcatgcctataatcccagctactcaggaggctgaggcaggagaattgctggaacccaggaggcggaggttgtagtgagctgagatcgtgccactgcactccagcctgggcgacagactgagactgtctcaaaaaaaaaaaaaaaaaaagtgttacatagatagctccacatggattctttcattcctggatgatatcaggcattccttttcaatccttttttccccttctcatttttaaaggttaaggaaacatcatttttgcgaaacaccatagctgaatgccaggcttgcggtaagtgcttttctagtaatgggctcgcctgagttggatgatgcaggctgatgaagggcttgcggtgaggctgtgttgaccgcagtttctgaaggtctaccacatagttggaggggagggaggaaaagttcaataggaaaccaaattttgttttgccttctgtatatcttcctggcggtgaatacgcctgtggatgattgtttttctctaggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgaggtaaaagttcacttagactgggaggggatccctaagtatcctcctcaccctgcagcttttggaaaaagaagccaagctgatgattaagggctgacttgggctgtcctccaggctccattgaaaccacttgcttggagaccctgagacaagggatggcggaaggagacatgacccaaggttatggctgcgtttgtcatttttcctctgtgtttccaactcttctactggtccattggctacagtgctgaggtttctgctttacacgctgacatttgcattggttatttctcttaaatagtctgtgctacaattttgtttttcccccacttttgcaaatgtggaaggggaggatttttctaagggtatatttcaggagtcaccgtggatttcttcaggcttcctaaaataaaggagcagaggggaatctcacaccaacaagggggaggccctgtaacctatgagtaactaaacgtgcccagggagggccccagaggaggcacatccagcaaataccagctatttgaccggctggcagagccccagaagagactgtttcaagaccctggttaggcaggccagcctagtggctgaagcctgcaatctcaacactttgggaggctgaggcaggaggatcacatgagcccaggagtttgagaccagcccgggcaacaagacaaaaccctatctctacaaaaaatatgataattactggggcatggtggcatgtgcctgtggtcccagctactctggaagatgaagcaggaggattgcctgaggcaggaggttgaggctgcagtgagccatgatggcaccactgcactctagcctgggcacaaccctgtctcaaaaaaacaaacaaaaaaaaacatggttaggcaatcaccagtccagttgcaaagcaatgttcatagcaggtagcaaggctgccaccaaaatgtgccttcaatgtgcctttctcctcccacccagcccaaggaaggctgaaaggagaagcgcaggggagagatgcaggtacataacttaggcacactgactccctggggctcctgcaggcccatggtaatgttagtaacactcagctgattaccttcgctagtagtaagcaattaagacggtctctagtaatccctcaattggagaaacaactgcatacatttcactgaactgcatctggtttttatctgtgaggtagaattttcctctttctgaaagggcaggaagtggtcatgttttttacattagttgaagtggtttaaaaaaaacaaaaaacaatctccaagattcctagagccttggaatagcatggacttgacaggacatggaggcaggcacaggccctgagggattacatggtaatgaagaggcaccggccctggctttgccttctcgaggtctttctgattgagtagttggtttgctgatgcttttggaggcatctaacagccagctaagtggtccagggacatagtcctttcacagagatttgcaaattccatgtcagaatatcagggagcttattacattgagaggtaggctggttaatatcaaggaccctttcaacatgcatgtgccattattctacagcacagcaaagcctactaaaaagcaacctcagattgtgtggtcagggattcagtgggtaactggagttggaagcctttatttccttttattattttttccaccaaaagatagttaaatgactaaacaagtatgtgcaaatagatgaagaaaagtttgagagtgttttctcggtgtggctaaagactttgaacttttttgtctccagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtaggctgaagtcatggtttctattcatttctcatcttaacaaaacaactgtggttggttcccacctctttggtataaacctttacctccatgagcagtcaaggccatgtggaatgtcatggtgcaaaatgagcaaattaggaagtttttcaaccatttctgagtatttacatttataaaatctagaagggaggaagcttttaagaggggataatagagtatcactagaaaaacagtaaatggggtaatatggctattggcataatttcagtcttattttgttgatagctgttgagcattttgtcctgcagaataagttgagaaccacggagacaaaagctttgaaaaattaggtcacttttgtagagtcattgttgccaatcgaagctgtttcagagatgaggggattttaagagtagaaataatcccccaaggcaacctcccctttttcttatgtaagctataaattgccataaagcacttgagtacttcttgacagtctccatttatttcccaatgctctactatattctggtcattatttaactgactgactgtgaaattctttttgatgtttttagcccttaaaaatggttgttctataaatatattttactaccttagggaaattattagctttgaagaagggcaggggtttattatttcctagctgcctatcaaagggttctagagggcatgatttttttaagttattgttatactttaagttctagggtacatatgcacaacgtgcaggtttgttacatatgtatacatgtgccatactggtgtgctgcacctgttaacttgtcatttacattaggtatatctcctaatgctatccttcccccctccccctaccccatgacaggccctggtgtgtgacattccccaccctgtgtccaagtgttctcattgttcaattcccaactaagagtgagaacatgcggtgtttggttttctatccttgcgatagtttgctcagaatgatggtttctagcttcatccatgtccctacaaaggacatgaactcatccttttttatggctgcataatattccatggtgtatatgtgccacattttcttaatccagtctatcattgatggacatttgggttggttccaagtctttgctattgtgaatagtgccacaataaatatacgtgtccatgtgtctttatagcagcatgatttataagcctttgggtatatgcccagtaatgggatggctgggtcaaatcgtatttctagttctagatccttgaggaatcgccacactgtcttccacaatggctgaactagtttacagtcccaccaacagtgtaaaagtgttcctatttctccacattctcttcagcacctattgtttcctgactttttaatgatcgccattctaactggtgtgagaaggcatctcattgtggttttgatttgcatttctctgatggccagtaatggtgagcattttttcatgtgtctgttggctgcataactgtcttgagaagtgtctgttcatatcctttgcccactttttgatggggttgtttgatttttttcctataaatttgtttaagttaagttctttgtagattctggatattagccctttgtcagatggatagattgtaaaaatgttctcccattctgtaggttgcctgttcactctgatggtagtttcttttgctgtgcagaagctcttgagtttaattagatcccatttgtcaattttggcttttgttgccattgctttaggtgttttaatcatgaagtccttggagggcatgattttttagactagaaaagggaggccagctagacatataggaatgataagaagctgttaagagtcctctaaagacaaaatctctaagaacaaaccaaggaaacataaggtacttggtgtaaggtagtaaggagcacttctcaatagatttccagtaacaaggcagacagaactgaagtggaagggagccaccttgctaaatttaacagcaaatgttactgcattccaaagtcaaagaaaataaaaggaaatggcgcagtgtccagaaacaaagagaaaactcaaagccagaactgtgactagagctgatgccacagcagatcctgggaggtgggagttgggtccaggtcttagtaacagggacctggcttttaccacctgtgtggagtggatgcagcaatgtagtctgggaactttggaaaggcctaacacccctgcataagtccaggaccctcagagaaccaccttatcaattacaaggagacaggaaacctttagtagccctaagaaacaacaaagaagctctttagctttaatacatggttgacaaaaatgaaaattgcctacagcaaatcaaagcaccacacttgtaccacatacagttatgagttccaaatctgttatacatataatgcagaaacacaagccaaaaaattaaaataataaccagccatgaagaagtgaaatccccagggctctgactcaagtcaaaggcacaccactctgtaaagacatgccaaaattcagggccaatggaatgcccacaggcaaaatttgggctactgaagatatgcttataaacaaaaatggcagtttatataaagaaactcccatgaggaaacatgataaatgcaaagtaaatataaaaaggtaaagaaataaataccatacttaaagaatagggcaatgccccaactgggcaacatggcaaaatcccatccctacaaaaaatacacacaaaaaaattagccgggtgtgatggtgcatgcctgtggtcccagcaaccttgggaggttgaggtgggaggatcacctgagtctgggaggtcgaggctgccgtgagctgtgattgtgccactgcactccagcctgagcgacagagcaagaccctgtctcaacaacaacaacaaaaaaatatagggcaatgtgagaaaaacagataagtttgtaaaacagacaaataaaacttttatcaatggaaaatcgttgaacttaatagatgggcaaaacagattagacacagcttagcagggagttcatgggctaggaggtgaacctaaagaaatcatctagaatgcagcacagagaaataacgacatggagcatataaacaagaggttaggagtcaaaagaagatattaacaagaaagttcgatattcatccaattgtagttccaaagaaagactagaaagcagtcgggggaagctgagaatgttccagaattgaaaaaagctaagaatcctcatttaggagaactgcattgattcccaagcaagattaaaaaaaaaaataaaactttcctaacctaaaaaggtgatggaacagatgaattatacaagttgtcttttctgaataactttcaataccatgaaagcaattctcatttgcaattagattaagtacaaactggtacaaatgcaagggagatggcacaaataaccctcagattaccattctacccgaaattccgcatcttcacttcacacaaagatagcaaaacaaaggagatttatttgcatttctatttaagcagcatgtctcgctaaactttctttgaacttttctgcactaggtctgcactgacattgatgagtgtcgaaatggagcgtgcgttcccaactcgatctgcgttaatactttggtaagtatttctcacagctgttgttatcaaagcagaaccggttattaaaacaagtgtatgtttatagatcatagcttccaaacacacctagaacatgtgggcataatatatttgattggcagaataaaggcaaaagtttgtgctgaaaaagtgtttaatatgagtaattttaaaagtaatttttatgtaagaataattttataagtgccaagttctttattttctatcaaagttgcaatattaggctattagataactatttattgagtaaataattattactcctgtattttaaaattgtgaagagcttggtagtttatattaatagttcagtattttactggtgactacacaatccccaaatggcattatttattttcataaaactttcaatcatggggtcaggagatcgagaccatcctggctaacatggttaaaccccgtctctactaaaaatagaaaacaaaattagccgggcctggtggcgggtgcctgtagtcccagctactctggaggctgaagcaggagaatgacatgaacccggcaggcggagcttgcagtgagccgagatcgcaccactgcactccagcctgggcgacagagcaagactccctctcaaaaaaaaaaaaaaaaaaaaacttttcaaaacttttcattgtattaaattaaagtgcttagcacttagggatgcctataacttaagtattcagatatgggttttatttctgaaagttcctgtcaaactgtgtccaagatgagagatgtaccagagttgactatcaagaaaagaactactgattacgtgtatgactatttgaaagttatccagcctctaggttccttgtttccttgtaagtaaaaacactttacatttatcaagtcacttattttaatcctcacaacaacctcataaacagggattattattatccccattttacaattgaagaaactgagggacagaaatattacatcattaagtccaggcatggtggagcatgcctatagtcccagctcctcaggaggctgaggccgaaggatcatttgggtacaagagtagtttgaggctacagtgagctgtgatcgccactgcactctaacctgggtgacagagggagaccctgtctccaaaaagaaaaaaaagaaatgttacgtatttagactaaggtcacacatcttccaggttggctacagagcctgagttcttctccgcaatgctatattgcttaggaatagatggggatttattgcttctcagcttttaccaggacaaagtagtctgcaaatagatttgcgagatcttggtgtaaaggtaataactgagccacctagagagaaggaaagtagacagtaaatagaactccaggaaaccccaaaaacgaaggactaggccaaggaaaaagagcagaagccagaaaaaggagcgaactgatagcacagccagggaggctgaaagatcgagacaagtcattggaaagcatgcattggatttggcaactaagaggttgtgagtgacttaaatcattaaacaatatatatacttcaaatggatgtcttttacgacctgtaaatgttatgccaataaaactgcataagatgaaaaagatctaatgtacagcatggtgactgtagttaataatactatattatatatatgaaatctgctaagagagcagatcttaagtgttctcactgaaaaagaaaaaaaggaaggtaactatgtcagttgatggatgtgttacttaactgactgtggtaatcatttcacaatgtatacgtatatcacatcatcacactgcacactttaaatatattcagttttgtcaattatacctcaataaagatgaaaattttaattttcattttttaaacatattgtataatatttattacatatatagtgtgcagtgtgataatatatattatatatataaaagctaaaaaaacctggcccctgaagtgtcattgcagcggagctgacagctcatttgtcagtttctcagcctacctcatcagaaattctcaatgcaggccaccagcatttctctgtgcacccattcctttgtcattccattgtaaaagagccaaaggaacagcaataagaaaaattaacttgctttttaagcagatcatctccctgtccccagagtaaacttctcacaccacctcccaggacttcactggaacttttgacctgtcccctatgccctccacggtggcgcgggcaactcagtgtcattggttcacagtccagccttcttccttcacttctattccttctttgtctgagtggccggacgcagcctccagtctgatggtgttttcatcaacattcacatgatcttacatgtaagcaaagaagaaggcatggatctgatgcctgattcctgccagcaatgagtacctgtgaaaatatacacaataactgtacctttgctcaaactgtacctttgcccacagctttcagctcatcacctgatctttgttccttgcagggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtggtaagttgttttttgacttcctctatcatttttcctcttccatttttaacaccacctctctccagtttctatgtgcttcagggtaaaagtactccaaaatgaaagcattgcaattaaatgagcatggagtgtaataggaacagggtccaccgagtttgtggggagcagcctgacatccatctaaccatcagctgccttttctctccctcttggctggggggcaaccctgtccacttgggtacctggatgccagagcctccggtgaagccccagctcccaagcaccttctatggaaaggcccagaagccagcagcagggaggtgaaggaacattcttctcccctttccttcttctgccccatctacctgcctgtataatgacaacatacctgctgtcatctctaactgtcctcatccttacaacttccagagtctgtctgccttgacgctcctgttgggggtgggccatcgtgtagaggtggggactgtcatagtccctccgggagaaaccttccgtctttggcaggtgactggcagggccagacggcgccatgctgtatgtctacctccactccaatcagggacagctccatgaatggtcacgcttcctgtgcattctccagccacggagctggtctggggaggggaggctgggaagagagtagagcagtcacagcagggcctgacatgggtcccatgctgagcttgtagtgggccctatgtgaaggagagggcatgctcctgccatccattcccaaatccaaggatccagagtcctcagcctgccatccattcccaaatccaaggatccagagtcctcagcctgccatccattcccaagtccaaggatccagagtcctcagcctgccatccattcccaaatccaaggatccaaagtcctggggatcctaggcactgaaaaatcagcagacttcagctgcccccctcctctgcacttcctgggatgccaggccctcggccggtggcccgtcagactctggggtcactggttcacagtgatcacccctgagtagcaagatggattctcggagcccttttccatctttatctcaaagttatgcttctttatctttatgcatacacaattttccacaatgagcctaccacttttcaaataggaaaaataagagtcaaaaatgagttaataaataaatataatgaagtcagccctccttggcaatacagatattctgtaacaaggtcagatggaagaatgtggtgtatcagagggggaataaccccagcaatttcctgaattcctcatggttgggcttgtttattaccaaaaagcagtcattgagaaacaagtgtatatctctgttgaaatccccattttctgtgaacaaattgacaactcccctgggatgatgggtgatggggctcttaatatgtttttgaatttgtaataaggcacaacgacagatggtaaaggattaataaacttggcgtgactactaaaataattgaatctgccagaaaatagctcattaaaaatgtatttgttcctagaggacattttttcctcattaaagtgagtcaaggatcagcctcgtggatcagccttccaggccttccaaaggccctgctggggcaagtgagcaggaaccaggagcccgggagggtctctctggagttctgcctcctaaatcctggttgcagagggagagcacagatgattgagaataagcaggctgtggctgaggaagtcactggtctgaaagcttttgggtggtgaggccaggaaatttaacctgtgctcacataagtctgatttcccttggcccagactctgctcctcagactctctccctcttccttcctttacctcccgtcccaggtcagctttctctccctctgcgtttccagttccctgtgaccttctgggtcccctcctcccatctcccttctgctgctctgcctgttcctggaagtgggagtgggagggagagttttctctctgagtctccctttatatctttgaatagtgaaccattacactattcaagtgcaattaaatagtttctaagccccacccaacaaggctgagtgactctgggccctccccctgggattgagcaaaggagcagagaggcccttgtcagccaccagcttcctgccaggctcagcttcccaggcctctgatgggctttccctttacagtgtggagtcggttgggctggagatggctatatctgtggaaaggatgtggacatcgacagttaccccgacgaagaactgccatgctctgccaggaactgtaaaaaggtaaggggtgtggggtggcagtaggcacacatgcactgtggctttccaaagtcacatcttctatggggagaggttctgtggatctaatttaaaattgtacttgtaaagtagctgcatctcagaggaagtgaaacagccaccaacaataaagccacagtgtcttgaggtcagaaacagttactggcttaacacaaatgtaggatgtcactcggaactgcattttccccctaaggacaactgcaaatatgtgccaaattctggccaagaagatgcagacagagatggcattggcgacgcttgtgacgaggatgctgacggagatgggatcctgaatgagcaggtacctgcttcgctgggagggcctgtgaattgccacgtaccagggatgatgggagaaagcctgtgatgtcaactatgtatatgaatcatcccaaatgttagtttccatgcctgcattccacagcactgcagctttttaccctataagattcacagatgtttgaaatataaaactgacaaacttgggccaccagagtgctgatgcttcacaacaatggaggaagtggatttagtaaaagtctgagtgatgttctgtcctttcatcttttaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggtagatttatcttgcttttgtcttttatttggaattcagttgaccttgttccattgttctctgagagagtaggaatagaatgactgattcggagccttccagctgtgggtggctcattgtggcctgcatctggaagacccaaggtggacttgtgtctatggaaggagaccccacagcccactccactacctaatggctctggatgtttccaggactctgtatcattgccagaaactttgtatgaccccttcaagcagtgtcttgtcagagtgtgtctttatggggtatctcccttcccatttctacctgtcctcactctccactatcctttctacatgtgagagacccagaagtggagaactgaatgagaaggcattgagcagggtcacagactatgcatcacaggctacacatcccattgtgggcccttgaaggaaaaacctttttgaaataccacctgccccaacacacacatacacacacacacacacacacacacacacacatacaccactccagaacatgcacacccccctaaaacatgacctcaccttctgcaagcctaactgatgacccatttccagtggtatatgctggtaagcaaatcacagaaagccctagttggtagcattttccagtttccatgatgtaactccgcattgtcctcattgctgatttcaagctaccaatgtgacatcatcaaacacaaaactgaaaagagagggacacaatggacccagctccagcacaacactgcctagagccctagggtctcagtcatagctcagatacccacgagatcctccctctccagggtcagccatacagccatcatgatcccctcctcgtggttgctatcatagcccttgacatcttggacaaattagaattcagtcgataaatacctctgagagctgttcatttttaagtctcccagaatccgtcctttgaagctacctctggataactgcagaaaagggtagtctctgtgtgacactggcccaggcagaagtgactcaccatgcgcctgatctctccagcacctagaagaagtcagtgacattcccctgactcaaggtagcatttctttctcacaggaataaaaaacattctggacaactgcccaaaatttcccaatcgtgaccaacgggacaaggatggtgatggtgtgggggatgcctgtgacagttgtcctgatgtcagcaaccctaaccaggttagtaggatactggggaattcaaactccaggctgaattcctctatgcaaagactcatttaagacgggtgtctagaaaaacagcagagcctcttagctgttgccacttacctaacaaagcagaggtggaaaaatgacacactgtgggcctgaagatggtgggatgaacaccacacaagatctgaagccctagggactatctcccaggctgtcgattgcctgtgaggttagtaacacgaacttcatgatgctcaatacaagtatttttcctgcagaaaggaaaacggagcatggaaattaaccattgggagccttgtgtccctttagtccctaaacccctacacagcctatgtatggacatttcagattaagttcagccagggatgtgttgcatctcccttctcactcctcctccctgtctggataattgggtatgatgtgtcatttatttttaaagccctgagagaggggagtcatggttgccctctgtgactcactcatgattttggccaaattgcaaattgcagaagcaaagcaaaatcaacctgccttctccccatgcctatggctggctccctgtgacccgtgccagagttccatgcctccacctgccaccatgctaccccagcgcccctttccaatcagctcatctggtgtgtgggcagcccatccatgtagcttgatattttagaaggcttttttgaaaattcagcattgtgcctacaccaatatggtgttgtccttttgcagttttgcaccaccccggttccagagaaataattccccaaatccaatgatgatgggtgaggtctgccttctctggaacattcggtacatgcaggcccaggaataaataacatgtgctgttctctttgcagtctgatgtggataatgatctggttggggactcctgtgacaccaatcaggacaggtatggcatgtctcccaactgcagagagacagatgcaaacatccggagagggtttttggtttgtttttttttttgaggcggagtctcactctatcacccaggctggagtacagtggtgcaatctcggctcactgcaagctccgattcccaggtttatgccattcttctgcctcagcctcccaagtagctgggactacaggcgcccgctaccatgcctggctaatttttttgtattttcagtagagacggggtttcaccatgttagccaggatggtctcgatctcctgaccttgtgatctgcccgcctcggcctcccaaagtgctgggattacaggcgtgagccactgtgcccagcccggagagggttttaaatttcagaacttcctgctgctccattccttgggatctttctgggtggactgagctgctggtgggataatagccctgcctctggtcagaattaccatgaaaattctccctgaaagttctgtatagggtataacggtgtcagaataggttactaggcactccagcaagtgagatctatttcaggagaggtaatatagcatcatgcttcacagatgggctctggaataagacagctagatataaatttcagctccttctcttacatgccagctaaccttaggcaaattacctaatacttttgcctcaattttctcatctgtaaaatgaggatgacaatagatctagctcataggaccatcgtgaagattgagttgatccatggaaagtgctcattatggtttctgttatatacatagcactcaatagacattagctcttttatttttatattctttcagctcaggaaaacttcaccatcatcccaaaatagaaggctgtcagtttgctgagggattgctattatttcctggtccttgaaatttttttaattcagttttccttatttaagtggaattgttctgtggaagtgagttttgtgaaccctcctagctcaacacgatggagtcccacattaagactcaaggctggatcaactgatcagattctggaagaacatcaccaaaaacagacgtgtgtcactcagccctagcggcacctgcaacccaaggttctaaacccttttgtctccatagggaattcttacaaaggcagccaggacaaggatgagagggaaaggattctaaaagagaaagcaaaaaaagatgcaatttctctcctgatacctggatcagaggttgccctgcagaaaaccagttcctgacccaaaccttactcaaggacagctcctatgaagccacagctctctctctctttttttttttttttgagatggagtcttgctctgtcacccaggctggcgtgatctcggctcactgcaacctccacctcctgggttcaagcaattctcatgcctcagactcccacgtacctgggattacaggtgtgtgccaccatgctggctaatttttgtatttttagtagagacagagtttcaccatgttggccaggctggtctcgaactcctggcctcaagtgatccacctgcctcatcctcccaaagtgtgcatctctttcattatgctctgtgtccttgctgcaaagtacatatagtcatccctcagtgtcccagggtattggtcccaggatacccctctgcccccaggataccaaaatcctcaaatactcaagtcccttatataaaatgatgtaatatctgcatataacctatacctgtcctcctatatgctttaaaaatcctctctagattacttaatacctaatacaatgtaaatgctaggtaaatagttattatactgcattggtttcttatttgtatttttttggtgttattttttattgttctttttcaaatcttttcaatcctcagttggttgaatctgcggatacggaaccgcggatacagaggcctgactgtactagctaagtgaacttaaacatatttcccagtctcttcacatctcaacttcttcatctgcaaagaggagataaataaatgcatcgcacaggttatggtgaaaaatcaagataatgtaggtaaagctcccggcacagtgctggcccaagggtgacatgcagaaaacaggggccgtgttattattggtatcacagtacaaggaaaggcacatcagcaacagagcctgacatcagatgtccctctcctcttccgagaggaaggtgggactgtatcctgacaactgccctaaatgatgtgcgtcaagaatgttccctggtgcaggtcatcttgcactctgactactatctacattaatttctaaaatatcctgtcccaccagcctctagatatttgcccaaaacaaatgactcattctgctatgttttaattcccacaaagttgaaccttggagaagaggataggaactgcttgtttttgcataaaaatgttcagggctacatagaacggctgctaatggaaatagctcagtcttgtgttttataaagcagacctgctgcatgcacagggattctgctgtttgagccacatcattttctgcttgtcaaggctgacttcactttcactggaccctttctgtccccctgtaccatgtggtaggtgtggccctagagcatggtctggtccctggcttccctctccaaaagggtgcaggggcagctcctggcccaggacaggccgcaggtggaccaggtctaaaggagtgctcattcttttccagagtaaccatgagaggtctcagactccaggacacctcttggaggcagggcagactcatcttcaggtggcccctcagcccagaaagtcaggtggacagagaaggagagttctgagaagtcctttagaccagcactgagcccggcctacacactctcctcgagaaggcgtggccctgttcactttgcccttctccccatggcgttcaggtctgaatcctcattggatccactagccagggattgtgaagagtaatataacggaaaacactgtagggcttcctaagagtcagcactttttcatatattgaaatttaatatgcaacattctgattttcatcatttaatctccataacaactctatgaggtaggtactattctcattttacagatgaggacactgagccatagagaggtcaggtgtcatgcctgaggtcacacgctgggcacagtggctgagccaggatttacactgaggctgtctggttccactgcctgtcattgttcccctcacacatcctctgagttgagtatcatctagcagttcccagggactgactgataggagcatctggtcataaatcatagaaatagacccttatcttctttagtaggaaatgactcacttgttttaagaggttggggatgggaaaattgtctctgaggcccatagggcagccttatctaagggaatgaccccagtgggtttgattttaagagccaaccctggtttaaaaaaaacctcaagcacagactctccttcctaaaaatggatccttcctgttccctgctgaactgtgagccacatcacctgtcctttctcctgcagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagtaagcaggctcagcccagagctgcacagcacccctgggctcccttcagccaggcacatgggggcacgcctctctccaggcaccaactcagaatatccccagctcccatcagctgcttctctacacccagcttctctgcatagctgcagcttgtgtggacagcagatatctggtagacaccagtctccaactcagtcaggactgagctgctttgcatcttcctgaacaaccctaggatgccatcacacaggtcagggaagcagccacacactgtggctcagtccccaaagggcttggagagctgctccaccatcttacctgtcacctgccaccttccactgggagtcccagaaaaggagcaggatagagagaatggcacagtagaagtggtatatgctgggttcacaaacacagatgcctgcaggtgccaggcagataagaaaatgtctgcaccaagccagacatgagacagcagggagtgttggggactgaagtgacctggagagggcatgcccatccacagcagtgcacatgcaaaaatgccagcacgttagctgaacaacacacacgtgggggccaccagaagttagtgttagagatattctggtaacaataatttccctgttttctacacattacagtccagaaagtgctgtagacatttgcttatgtgaggtcaagcttaagatccaccagttggtgaactcaactgtaggtaataagaacttcttgaggacagagtggagggcaactgctcaaattgatctttcaaaaaactctgggaggactggcctggtcacccagtacaaggactccagaatggggcctggcaccagctcagacatgtccccagggctcctctcccaacactgatgggaaatcatggccctggttgtgggagcttgagcccttctgttcctcatgcagcccccttccctgccaaggagtggcggtgggtgtgagcgctattgagctcctgtcctttctccaccccactcaggcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccaggtgagtgtcacatgggcggcaatggctcagccttgcctctcaacagaggcccttctgatagtggtaggtcatgtttagagggtgcagacaataagccaggcattcttcacattcgctcttattcctcaacagccctatgacagactacttcaattatgcccattttacagaggatgaaattgtctgctacagctttcatgattgtagcagctttaatgatggtagcactgtggtaagtagctgagatgactggaattgggacccaggcagtctgaccctagaacccacacttctgatctctgcagcagactgccttctgcatatcctcctcattgtgtaactaagaagtgcaggcacagagaggcttagtagattttccagagtcacagagcaagtcacattgattagaagtatatgttgtatgctccccagttaagggcctgtccaatcagcttaagtgacagtctcagcaaacttatttctattcagctttgagcttttttttaacctttacaagcacatgctttctttgagcacataacacaatagagcaactatttcagataatgaaatctttatatggaggtcagacatcactggccactcactcatatggtgcctgaggttttgagcttccttaaggttacttggccccttcaagactgttctgaactccctcaactctctctgcagggcatggagattgtacagaccatgaacagtgatcctggcctggcagtgggtatgtccagggcctcagttgccactcacatagaattcttccagctactagtgtggtttgtctattgttctaatcttatctggtccctacagggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggtattggtggtttgaagtcattcatctccctttcttctggacctctcatcttttttcagatattgtgttttcccatgtggtacttagttaatctaaaaaaaattgatgcatttatgctaacgttagagtcagggaagactacagttgacccttgccttcattcctgtgtgccagctacattctaacctctcatggtgggaatcccagcctacatgaggtcagagagagggaaggtttagagtgcatcttcaggcacactgtgttggaaactggctaataaatttcatcgataggtccaactttgtacaggagggctgatgggctacttctgtcactcctgctaggacacaaggctgacttaagtagccagacgtttgataggagccctcattgggcaaatgtagggagcaatggatgtgcagcaggaacaaacatggaatgtgtaatggaatactgggcaaggcttagtagtaaaaggttggggtcacctccatagaaatgacatttaagtgtttatgctttgttctgaatccaaggcagccggattataaccatgactataacctggatcatcacttcagatccaggaagcaggaagggtggtgcctgtgtcctgtcctaaaaccttgcttgtgtcatcatgcaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcaggtaggtagaggccccatctccttaccttgctctagaagcaaagcattctccgttctgcatcccagagccttcatttccctgagcaattctgacctaggatagtccccaaggacatgccaggacttttgtgaccgcaggatggggaacctagaaaagaggtgggagagaagatattcagggtcagaccacccggacaggacagtaggaccaagatggggatagaaggagcagagctgaggggacgacccagcgttgtggggttggtgcatttggatggctgtgcccttctgccatgctctgcagagaagcctgagggtggacatgccaatcggtatgaggtaagaggtgtggcagggtctcttctccacaccctcccctgtggcacctctgggccctgtgtaatcacttactagtgcacatttcaccctgactgcagcactatgtcccttacagtctgtcttccctgctggatcacaaactgcatgagagcagcgcttgtatctttttttttttttttttttttttttggagacggaatcccactctgttgccaggcaggagtactgtggcgtgatctcagctcactgcaacctccgccttcctggttcaagtgattcttctgcctcagcctcccgagtagctgggattacaggcacgcgccatcacgccaggctaatttttgtatttttagtagagatggggtttcaccatgctggccaggctggtcttgatctcctgacctcgtgatccacccgcctcggcctcccaaagtgctgggattacaggcatgggccactgtgcctagccatagcttattattttatcttgatctccccagtgcctagcacaaagcctgacatctactacacgtggcatgaactgatcatcatgtaggccaaagtagtctgacttctttctttataggacttttttgttgttgttttttaagtattctcgggagttaggaggcttagggctgtgtctttgaaatgtagatagtgttcttcttcatttgaacttgcagatcaacacccatcctagcctatgtaggatatttaggaaggagtactgagtgcattctcctcatcagggcaggaaagcagccttgtggaaaaaagaaaatcatgagggcaaattcatgttttgtttctctttgtgacattatgtcaaatgtactctttttgtctcacagtgagatgatcatgttctcagccctattaagcccaatgccccttttttattataaatattttgaaatgcccccttttccgtcccaaaatggaatcatagaaattacaacctacctacacgtataatctagaaaatcaatgtgagccctaacaatatcatagagaaataagaataatttataacaaaataatttaaatatttgagcacagccacaccagaagacaaaatgaaacagacacttgcacccatgcctagaatcactacgaaggtgacattctaacagactgttgtgtcagtgacttaaatgtttgttacactggttgatgacagcaatgtaaatttctcaaatggtacacattttagaatgttaattttcaaacaaattttctcaatttaccctggaaaatatagtttatattaaaatttgtgcaaaatatacttgctgtttacaagtgaaagtgagtcaggttctaggccctgatagttgctctttacctaccctggtgtctgaggaacactccagaagtgtatggaatgcaggacaggttttgattgttctcgccagggcattgctcgccatccagcatccctggctgccatcactcagtgccactgtgacaacccaaagtgtccccacagattcctccaactctacttagggagtgctgtctcctgtggagaatcattgctctgagattccaccaaaagatataaatcagaccaaaggaaaagccaaagtttttaagcatggtatctagacaaggcaactcacatgacaataagacacaaaactttctcactcaagatagcaggtttggtttggtttgatttttaactcaagtttgatagtgtgaccaacatcatgttaggccttgtgagagacagaagttgggaagaaacaccccccacccccctccacacacacacggacacacacggtccctgccctcaaggacttccagtctagtaagtaagtggcaacagctacagtacaatccaggtcagcgaaaaatggagcctaaaacagagcaaagaaagacgcaggtacgtataagaagtggggcccctgggcctcacagtgggcactttaccccgggttggatttcatggaggcccctccggccgtgttgtccgcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatggtaatgtgcattctcgttactgttcaacattgttactagaattagtcaaacactgccaccttatttttataccgcacttcccccccaaaagcccaacgctcataccacatagtcattaaaacctgatagtccctgtgtaccagaagtaaaaagcagatcacatttatcctattttatccatgtggaaattaagacccatagaatttaactgcatggtttagggtttggggttcagtgagaaatccagtcagatttaatttacaggaagcctttattttcatgtggttaaaacataatcacagatgcaaaatccattctgcctgctgattttctgcacggccgggaaggagcactcctgggctttaagccaacggtttgcaaagcagcctgcaggagaaaatggcggcgagggcctgcggggcgtcctgggcgggctaacagcgccccctacaggacgcctgagccgcgggcgggggtccggggtccggggtggaaggagcctcgctaacctccctgtgcccattcctattgcagacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaccgtgttgctcagacattgctctcacgtggctcaggctttgaggtgcccgttctcggagggatccctgttaacttgcagggtggtagtttcttctcagacccggtggccgataagatgagcctatgcgtgaatcacaaggcccaggccagaggtctcagctggggtccccccagtttgctccatacaaaacgcctaaccgttccatgctcaccttggcagcctccttatttcgggtaaggggtctcagaaacacgtgtttggggtggcaccttccctagggaggcgtttttctgacctagtcctgggacagatttgcccaattctttctgatgctgtcatcccatcccacaccttccacagacttctattctggccccttcaacgtaattcttaagcttatttatatatttggctccccctagtggactgtaagcatattgacaataaaggatcatgtcattacaa

SEQ ID NO: 11 - DNA, 3365 bp, Homo sapiens
ctggtccgtccaggctccttcccatcctcacacccgcgcctttctccctgcggccccggctcgctgctccagctgcccagctcttcccccgcccggccgcaccataaagcgcccggccgctgccgcggagcccagcagccagctccccagcaccgcgcggcggggacgcgagcgcgcccccgacggcagcccggacgccgagcacgggtcacctgcggcgccggcccgggcgccgaccgaggttcaacgcacggcccggggacccccaggcggggccaacgccgccgtcgcccccggcctcgcggggagcaggaagagccaacatgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaaaccaaggaagcaatctgtaactgcttttcggaacactaaaaccatatatattttaacttcaattttctttagcttttaccaacccaaatatatcaaaacgttttatgtgaatgtggcaataaaggagaagagatcatttttaaaaaaaaaaaaaaa

SEQ ID NO: 12 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 13 - DNA, 4167 bp, Homo sapiens
atctctctcgggtggagtcttctgacagctggtgcgcctgcccgggaacatcctcctggactcaatcatggcttgtgtgagtgtggggacccccccccaaggtccaggggatagggcaggaactgatggccagaggagagctgggcagatcgggagcagattctagccccagctgtgtggcctggaaccagtgccttctcttttctggacctcagtggccacatctgtaaaatgggggtgggcgccatggtccctcaaggccttctctgcattgataattgtctggattcctccagggtctgaaagcacagttatttctgcccagggttgacattctgcagctctctgagaagtgagcgtgggaagggtgtggccaactgggggacacccaggccactatccctttccccctcctccaccccaaagagcctcctgtcccctcccccctgcagctgtcccggtcaccaggccagggcagagttaccctctgctccagagaacgctgaaaagttgccaggacccgagacaagctgcccaggatgggcccttctaggtcgggggtggagggtggttgtgtccaggctggtggcggggggggcgggggaaattcccttccaccacccccaagctgggaggttggggtggcagggaggtgagaatcttcctcgggccccagggaaagggttcaagtttctggcagaagaaaccactcaaaccagttaactcttgcccacccactcggggtgccagggaacagcagtgcccagcagtttgctcaatcttgttaaccctgagccagcccagccacagcccgactgcagggctattctccctaatgcagagaggccgtgtttttcagggtctcctctctagcccctgggcctctttgcagagaggggcttgaaggagaatagtggggtcgcccgcccctagccacttcctctccagcagaggggccggcccctccattcagtgtgacggtgggccaagtgtcggcccctccccagcctgatcctctccatctgcgatgggacagagcaccccatctcccaagtcactcttgagtccaaaattcccaagccaatctgcaaaatcttctagagcctgtcttctagaaccttcacgttacagactgaagccaaccccggtgggaatagggactttcccaggaccacatagacaatcggaggctggaaattacagctcaatcctttccccaggcttccccttggcttggtcagaggatgccgggcgggaacaaccccactcccacccccagccacccccggacacttcgagcagtggaggccttgtcctctaacccggctgggccggggcttgtctgtgcagggtctggtcgccagcaacctgaatctcaaacctggagagtgccttcgagtgcgaggcgaggtggctcctgacgctaagaggtgagaagtgaagtcggggtggtgggcggcagggacgggcttggtccagcagggagggcgtggccggccaagcccacatctcctccctggccgggagcgggttaacggccagccgccgatgctgcgttttctgggtgactcacttcccccgcagggtctgggcgcccccaccgttgccgccccctcccccgctccctccctgctgtagcctcttaaactaaaccagctgcagcctcgtcatctgtaataccttgactcccccgcccccacccctttccggtctgggcgggacctgtcgctggggagggcggggagaggagtggggcgggcgtcaccgccgccttccccctgagtccctccttcctgcgtctggtcattcattcattcactggctcagtccggtccttcttccttcacttctcattcactcagacggctgccttattttctcggcagttaggtgaccttggaccagtcaaccaacctcacctagcctcagtttgttagaaaaacaaggggggaggtggttgtgtggaggaagtgagatgcggctggcgcagtgacagtgaggaggatgaggatcagctgatattgttaagagccgcacacttctcatgcactaatttcatttcaaccaaaccctcagaggcaggtattgctattatcaccactcaacaaatgtatttattattttatttatttatttattattttattattttatttatttattttttgagacacagtctcaccctgtcgcccaggctggagtgcaatggcgtgatctcggctcaccacaacctccgcctcccgggttcaaacgattctccggcttcagcctcctgagtagccgggattacaggggcccaccacaacgcccagctaatttatgtacttttagtagagacggggtttcaccatgttggccaggctggtctcgaactcctgacctcatgatctgcccgctcgcctcggcctcccaaagtgctggggttacaggcatgagccaccgtgcccgggagtattattattattatttgttaattcgccccataattactaagcatctttttctggtgtgccccacttgtgctgggcaccgggaatacagagatgtacaagacaggacgggaggtcaccatctggagggagtcactgaccttgaccaaacgggctaggatgctaatgacttcaagaatcaagcgagccctcctgtgcagcccccattgtacagatgagcaaacaggggaagaggggcaggagcaggtggcatggccagagctagaatccaggtttcttgtctctgttagtgagttcttccagcaaggtgcgttcatgggatactgagtgacagattagtcggtcagtggggctggagctggccgaggtggcctcatgcccacccgttaccccccagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgggctgcagaccggaaccggggaccagggacaggggctgggtgggctggggcggggctgggttagtgactagagaccttggccctgcctgctctttcccctccccttccctcccttcctgtgtgatggccagtgtctgcccctctttgaacctcagtggttgattacaataaaacgaaggggaaaaaaaaaggctgggcttggtggctcatgcctgtaatcccagcactttgggaggccgaggcgggcggatcacctgaggtcgggagttcgagaccagcctgaccaacatggagaaaccctgtctcgactaaaaatacaaaaaattagctaggcgtgatggcgcatgcctgtaatcccagctactcaggaggctgaggcaggagaattggttgaacccgggaggcggaggttgcagtgagccgagattgcaccactgcactccagcctggccaacaagagcaaaactctgtctcaaaaaaaaaaaaaaaaattagccagcgtgctggctcatgcctgtaatcctggcactttgggagaccaaagtgggtggatcacctgaggtcaggagttcgagaccagcctgaccaacatggtgaaaccccgtctctatataaatacaaaaattagctgggcgtggtggcacacaactgtagtcctagctactcaggaggctgagacaggagaatcacttgaacctgggaggcggaggttgcagtgagccgagattatgccactgtactccagcctgggcgacagaggagactccatctcaaaaaaaaaaaaaaatctatcataggattagagtaaaaagaaagaaaaaatattataaatgtacctccgcaggctcagccacaaactggggctgtgtcagggccacatgaggaacgggttctggaagggcccatggcatgtgggcccggctcactgctctcctctacccccaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaa

SEQ ID NO: 14 - DNA, 556 bp, Homo sapiens
atctctctcgggtggagtcttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctccctctgaaaaaaaaaaaaaaaaaaaaaaaaaaaaaa

SEQ ID NO: 15 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 16 - DNA, 527 bp, Homo sapiens
acggctgcccaacccggtcccagccatgtccaacgtcccccacaagtcctcactgcccgagggcatccgccctggcacggtgctgagaattcgcggcttggttcctcccaatgccagcaggttccatgtaaacctgctgtgcggggaggagcagggctccgatgccgcgctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctgagcagaagcccaggcgggcccggggccttggctggcaaataaagcgttagcccgcagcgaaaaaaaaaaaaaaaaaaaaaggccacatgtgc

SEQ ID NO: 17 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 18 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 19 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 20 - protein, 68 aa, Homo sapiens
SPYSSDTTPCCFAYIARPLPRAHIKEYFYTSGKCSNPAVVFVTRKNRQVCANPEKKWVREYINSLEMS

SEQ ID NO: 21 - protein, 93 aa, Homo sapiens
VGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 22 - protein, 36 aa, Homo sapiens
MCMPCFTTDHQMARKCDDCCGGKGRGKCYGPQCLCR

SEQ ID NO: 23 - protein, 40 aa, Homo sapiens
MCMPCFTTDHQMARKCDDCCGGKGRGKCYGPQCLCRGGGS

SEQ ID NO: 24 - protein, 40 aa, Homo sapiens
GGGSMCMPCFTTDHQMARKCDDCCGGKGRGKCYGPQCLCR

SEQ ID NO: 25 - protein, 1413 aa, Artificial Sequence
  fusion protein
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDPGGGGSVSKGEELFTGVVPILVELDGDVNGHKFSVSGEGEGDATYGKLTLKFICTTGKLPVPWPTLVTTLTYGVQCFSRYPDHMKKHDFFKSAMPEGYVQERTIFFKDDGNYKTRAEVKFEGDTLVNRIELKGIDFKEDGNILGHKLEYNYNSHNVYIMADKQKNGIKANFKVRHNIEDGSVQLADHYQQNTPIGDGPVLLPDNHYLSTQSALSKDPNEKRDHMVLLEFVTAAGITLGMDELYK

SEQ ID NO: 26 - protein, 729 aa, Artificial Sequence
  fusion protein
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRYGGGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNDHQVYIMADKQKNGIKVNFKIRHNIEDGSVQLADHYQQNTPIGDGPVLLPDNHYLFTTSTLSKDPNEKRDHMVLLEFVTAAGITHGMDELYK

SEQ ID NO: 27 - protein, 1432 aa, Artificial Sequence
  fusion protein
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDPGGGGSMAEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAKTTPKLEEGEFSEARVQSELTQPRSVSGSPGQSVTISCTGTERDVGGYNYVSWYQQHPGKAPKLIIHDVIERSSGVPDRFSGSKSGNTASLTISGLQAEDEADYYCWSFAGGYYVFGTGTDVTVLGQPKANPTVD

SEQ ID NO: 28 - protein, 1435 aa, Artificial Sequence
  fusion protein
MDFQVQIFSFLLISASVIMSRMAEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAKTTPKLEEGEFSEARVQSELTQPRSVSGSPGQSVTISCTGTERDVGGYNYVSWYQQHPGKAPKLIIHDVIERSSGVPDRFSGSKSGNTASLTISGLQAEDEADYYCWSFAGGYYVFGTGTDVTVLGQPKANPTVDGGGGSNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 29 - protein, 1211 aa, Artificial Sequence
  fusion protein
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDPGGGGSMCMPCFTTDHQMARKCDDCCGGKGRGKCYGPQCLCR

SEQ ID NO: 30 - protein, 1211 aa, Artificial Sequence
  fusion protein
MGLAWGLGVLFLMHVCGTMCMPCFTTDHQMARKCDDCCGGKGRGKCYGPQCLCRGGGGSNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 31 - DNA, 51110 bp, Homo sapiens
ctcattctatttattcttttaacaacgatttgttgagtacatactatgtgtcaggcaaaggggctagcagtaaagaagactcatatggtccctcccctcattgagcttacagtataggaaacagttattactgaacacgaagacagctatataaacataatttcaataaaagatggtaactgatatgcaccaaagtaagactaaaacacagagaagtgcacttaacttatactggcattatcatgggccttgatttactgtgggatcctttgctgttatgttcccttacgttgaaagatgcatctggccctacacaaagcacagactatgagtaggtaggcacatacagttctaagccaaccccttggggcaccaggagacagcactgtctcatgtcgttgcatgacacaccaccatggggcaaaacatttgctgaatgaagttacaataaggatacaattgtatctacataaacatttaaagatactatcatatcaatacctactatgtggcaggatcttgatgttgtgtggggaggaagataaaagtagatgtatcgttgagacagaaatgagggcaagggaaattttttatgcattgagagaggaagtttgtgaattttttcgctggccatggagtcagggagacttcagtccaaccaccgtccctccctgagcttgtgaccaagcctcactttccttatctataaaggtgacaagagtgatttttttaagtttatttatctgtaaaggggaaactgtagcaactttgctattatagggtaaaaagaaaaaaggatgaaaaccagaacaagggagagagacaagagagagagcagtgtcacttataactgtgggcaaagtgctcaccattcctctttccctttgtttcctcatctcaaaaagggagatcataacatttatcctttctgaggttgttatgaagattaacttaggtgatatagctaatgtgtacttcctgtgttcaaaacattctaatatgtattatcccctccctctctacccaagaaactccttacatacagataagatgagctcaacatccaaattaaaatgctttgaaaagttaaaaggcaaacctttaaaatgattaaatactgaaaagagcttaaggttatctcagagagaggataatcaaacctaggcaggctgctttggactgtctctcctgggatatgcctgcttttgccccaccaccaaaacataccccagctagattcccaagagcagcagtggtcacccaggtggcctctcattccattttctgcacaaaaagacgttaagcatatagctcagtagctcccagtgtaaaacagaacatcacccgatctccacctctgaaggttggggacagtcctttctagtcactcttcagctttaggggggttggttggtatcagggacagctgacagccttatttagccaaccagctgctctgtgatacacaatgtccctaatgctcaaagtctggtggatgattcttttgtatccacataagcagcagttggaggagaatcgatgagtccctttggttgcctcggggtgtcaaggctgccattcaaatcattaccaccataattattaaccattgttttagtgagatgtctgccaggcaattgtttttatttttcaatcattaaaaaagcaatcaaatttcactagagcaatgcctgcctcacgccgcatcaacacatttattaaaccccttctgtttgccgagctcaatggaaagtcttggaggagggaatacttaaaatgctctcggatttaaaagaattgataatgctgtggggaagaggttcacacaaaaaagcaattacaggaagaagctcgatagtatacaatacactggtacagagtgtcatagacagtgccactttcatacgctggattttatttctgtggcaatgggatgcttgggaggagccgcactgtgtagaggatttggagaagtggggtattgtggtgggaaattgctttctttcccaggaggtaggaggaaaacaatcaaggaggtggacaggatgtgcactccattagagcagccaccagagcctgactttttgataagagagtacatcagttaggataacggttaaaagtatctttaaaagacttttgcttcaggatgaatgatgtggcctgtgtgattcagcgataaattcaaaagccttgtccctattgtggcttgcggccacatttcgaacccatttttcaagcatgttaaacccaagcgcagcgcagagggctgcacatggggcagtcacaaaccaagctcaataaccttgctggtggggatgtgttggatacgctgctaatgcctgtttgcgacaatgctcgctagtcccggtggtggcggtgttcacaggtaacaatgtttaccaccgtgaatggaacttgtttgattaaccctgatcagaggatgaaaacactaaagaaccaagtgagaaagagggaagagaaccgcatagggaagagcagagcgagtagacgagccgaacgcagagcccgcgaggggcgagtggaagctccctgcgggcaggtacccgaccaccgccctgccctgggcgtggcggcctcgggctcagggaccgcttcggcgctagacggccgcgtccggaggaaacgggcgctggtgaaagcctaggtgtcctggtccacgcgcgcagccggacgtcgggtccagggagagacgcgggctggggcgggacgggacccggcccctgaagcgcgagggtgggagtgaaagcaaagaggagaaaagtagagagagaggacagtggggcccagcgccgcgcgaaaggcaggaaccgacccgcggacaggaacgggtgcaatgaggtccccggcgagcgtgggaacacagggttcagcctcctgcggcgggcgagcacgcggaccccaggggcgggcgggtgcgacaggacgtgacctggaagcgcggcgcgtgccaccgccctggagcaggcatccggcctccgtggagcgggcaggcgggccccgggtctggagcctgcgactggggagggcgccgcgtcaacagcagcggttgcggggcggggccgagtgcgcgtgcgcggctctcgcgggcggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggtgagtggctcctcgctcccagccctgcggctgctgtcgcttcgcccccgcgggcgtgtgggctgcgccccagccagcccggcggcgccctgaagagggtggccggggcgcagaacactcgggccctgagcgcccgaagtgcagacgtgggagggccccacggggaatcgggcgccccccttcttcctcccttcctttccctggtcgtcttcttccccctgggtgagagcggggctcatctcctccacccggttctccatctccaaatgcacacacacaggaagactccaaaccgcgcacctcgcgcaaaaattaactagcaagaaagggcgcgatcagaggcaaggggctgcagctgctagggatccccttctttcgcacccctctcccttctcagtgcttagacgcatttggggttgagggagggagagtgggagggcccgggcctcttgcgatggaagtgcgcattttggcgaagtcggtgagaaggggtttctgccgtttgcctcccacatgaacataggagcgaagaggacgtaaaggacacaattaagtttcattttcaactcagcattcaatcagaagaatcttccggccgtgtaatttttgctgtcgtttttaatcctaaaaataagcttgctggaaactcttcctttctcgtgaccctcacgccccatcagccacttgcatacattctaaattgtacgttgaagtttttcccactttatttgggggaaccgttttaaaagtaatctggttttcgcctgaaataggaagacagtaacctccagtcaaaacatgtgcagcagaatgggatttggtgtttttcgacgccaaagaacctccacccccccacccccaccccgagcttttggaatccatttcgcttttgtaaaacgtgtgcttcgtctgtaaaaactgagcaaggaatagaaacattcgtagctctacagtgagtgcctgtcacacttcacatccatagagccttttgtgaacttttataagattcagaattcagcttgagcaccagtgacagcaattgttacatttattctagaatgctaaattaattcgattttaaactgattattagcctcggtgtgccgttcctaaaggcttgtgactttaggtatttccaagatgcccttaagtctctgcttaccactttcctccctccccgagcatcctggatgttgggactgtgaatccaggtctccgttatctaaatggttgatgttagtgtttcctgtcatcacgtttagtatgcttgttgcctttactatcattatagctaaaataatactgctttcagagatgtgttgtataatcgcataataatttcagaacgccttcatattgaaccagatatgaagtgatacagtatcatttattcaaactgcctaaaaagtaaaaagtataaaccatatactatttttaaaacaggtaggttagatttaaatcacgtagtttagaactgttggaatggtactttgagtgataggatttatgttaggccttctttagtaatagttaatatcatgtaattagcactcgtttacacacaattttaatgtgttcgaatccctagagtcatcgaattcagaatttatagtattttattttacttagttaatattaacctaaaaaaaaagcaaaatacaggcattatgcagttgagttgtgttaagtgtggactgtaaaacaggatagtatttgttataaaatatgtttttgtatgtgtttaatatatagcttcaaaaggacatgtatgaagaaagatgtctcagcacataggtagttataaaccaagggtttgagagactgactttagaatctagaatgagtaagaaagtgatggatctgtacattcatttgtattgaagtttgattttgctttcagtttcgttaattaagccccgggaatcagggacctttcctggcaccctctacagtgttagtggcctttgtggtaaaagaagttatctcagatactcatttcatgcaattacaggcaaactggaaggcaccttaatggtatgcaagatgtgaaatgaattactatgttgcattccactctgcccccactcatgtacaattatactttgtttaaaagtgcatagtttcagtggtatttattagctagcaaataaaactttaaataaataatgatggtgttgtgaactgtcttttcagtggttgaatgattcctctttgttcaaaatgagttgtttttttttttaaatcagggtacatatgattaaataaaatttttttccatcgtctaagtctagtagtaatttggcttgtttaaataaaaatgttttctcttaaagaatatattatttttaattacaagttgccattttaaggaagtaaatgcctatttaaaagtacgtatttatggggccgccccagcagtctagaggcagtgttttttaaagcattacactaaatgcctaccattaggacacttgctcatgctgtcatctgtgttttggcagatgttttatctttgtcactgagttgttcatggagatttgaaaggccaagttctaaagatgagagatagatgactttccccccaaattgatacatattctaaatccaaaataatcacgcatattcgcaaaactagatttgcatttcatagtatgaattcagctatagctagtatataggcattgcttttaatatagtaatgttctttttgtggcataattttcaatcactttccctgtcttaggtttctgcctacctttttatacaagataaacatttctattttcatatcttatctctcagtctgattttaataatatcgtcttgggttatcataaagttactttcactttgtatactagtgtagatatttcttctgtgataaaaagaacaagaataatagtgtaggatcagttttgttaaactcattgtagtactaaagtagaaaatacagagcacccaggaaatttgattctgatatacctagactaagacaatagaatctgggtttttcctctttacgattatacagtgttgaaacagaaatcaagctacacccacctctgaatatttcattccaaatccatggagcagttgcataagactcagaatctggtcaatctcatggtttgagattcagattaggagaattaatgtgcatatttgaatatatatcacacttgtaggttaaaaggtaaagcaaggggtttggctagttcaaggcctgtcaaaaccacttttttccatttcaagcaagtatgagcctatactacacaccaagcagtttgcaaagccctctttggcatatagacaaatttcagacagtatgtcagtatgtctcttaatgacttaagatgtagtatatagacagtagggatatcttgtttttattccctcatggtacttaggtgcttgttaaggccataattctggatgttattgactttaaactgccctgccctttttaattctaagttggcctcttccattcttataaggccacttttaaattcattagggggattgcgttgggtcagaactaagtacaacttgtggacaccccactctaccccagacctttattttagtgtccattgaggagagatctgacttcatcctttttgtacgtaacccagtgtagctagagactccagttcttcaagccagacactagttctaaggggcaaaagtaggtaggattgctttctggcatgtgtctttactgatgatctttgcttctttacctaagaggctgctgagacttcttactccattcctgaacatccattcccagcagataattaacttcctactctactgcaaaaaatagtggtgtgtggtttggactccctctgcttccctcccaccattactaaactgatctataacatcttagataaccttcgtctcattctcttccgtcatccagcttcagaagaaaacctactcacttttccaacgaaaggacaacctgtctaccaccttttcacttctaccatcttggagacctgctcagtcagcataattcttcagtcttgcatcttagatctttccctctccattgactccttccccgtagcttgcacacatgatcaaattttaccccatattaaaaaataaaacaaaaaacctcaagaaccaaaatgtccttgcccttgttcctgcctgcacattgtcagttgttgccttctctgctttcttacactgccaaatttattgtaaaagaatcactgccacttaatatattttaagtgcttgataaagccatcttgtttctctctcatttctcaaacatagtgtgcatcagatctcactagtccattgacagatggaaagatattctccccaaatatttaagcctcttttctctataattagcctaccatctaactagctacacatactattttggttcacaattttaattttcccctctttatctcccctgcttcccacaactaacttctgttgattactgtaagttttaggtcttctctccagttgttctttacctggactattgcagtagtctgttaacggatctccctttctcttttcccttgctgccctcagtccttcctttacactgctgtcaatgttttttttttaatgaaattaaatctgagtggtttacagttgtttatgttatcaagtctaaactcagcatgacattcaaggaagccctttagaatttttcttattcatcttttccaacctcatttcctagagctcacctaaataattctctgctttcagttcttcactttctttatctggctgtacacgttactctgcctagaaagccccttctttcccataacattcttactcatcctttaagacccagtttaaacatcaccctctaggaagatcattcatagacacattcttgtgatttttaaggaacttttctatccaaccatacaagtgacttgagattttccatgaaaatatgcagcttcatgatttcataatcaagtctatacaagtgagaagcagtggcaggttctcttgaaatacagaagaaaaatctatcttctcccacatgatttttagatttttcttctatggaatttatactttaaactttttacattcacaaggaaggtattgaatcctactttctggaccctgtgttagattcttaggataccaaatgaagacagtgtcttactttctgagcctggagagatcagttaagtaaaatagtaattacacagtagtaggtacagtgacaacatttgaacacaaggcaatgggagcaataaggaggggaagaatacaattgagtaatggtagggaaggggaacaagattgaacaactcaactgtgctttagtaggaagagcaaaagttagcaaactgtagctgccatttattgagtacttgctgtatatgtgaaaggtgatacaaaaacatgttatcttattaaatcttaacaatatctctatgaggtatataccatcactatgcacattttatagatgaggaaactgaggagcagaggtaagtaacttgcctaaggttatacagctaagaagtacatgaggtgaatcttaaactagggtcgaccccttaatgtgtgcacttaactattatacaccctatgtactcagtggggtaatagtgtataacagttaagaggctatttaggtttgaggaacagtatgtgctaaggcatggggatgagagtgcatttgcatagtcatggaattgcaataacgtcattatgattggagcttagtgtggaaatgagggagtacaggaggtggggctggaaaggatcttgaaggacctcgtttgtattccatgctaatgtacttagaatttagcctgaaaagggttgagggtgttgttaaagtatcttaggcaaaggagtgcacatttgcatttagaacagttattttgggaactgtagaaaatacatcagggccaggatggagaaaccttgaggatggaggcagggagctggttagaagggtatggcagtagttcgaggggggaggatgagtgtctaattcattcattcaacagacattggatttaggaactattaattaggttgaacctactaagaattctccatggctaaggagagggagtaatctaatgtgattcttaggtccaggtctggttcactggtagatgatagaaaatactttagcaaagtgatttaggctgagggtaagaagtgggggtacgtaagtttttggacatactgactttgaagtaactgtattacatccaagtggaagtgcacagcagacagttggctgtccaacatctgcagtttagaagaaaggcagtgcatgattcagttttttatttaaaagtttgcaggtgaagaaaaccatgggagtcagggaaaatactagggggcagagggtactgaatctgaagcccttagggacactaacatttagtgtgagtcaaggaatagatttcaacaaagaaagcaaactaggatggggtggacaaagcaggaggaaattcgtaatcaggagataccatggaggccaagagagaagagcaagaaagagactgcccactggctcaagaactgtggaaaagtcaagacagaaagtgaaccatgcatatttgatttttcacaagggattggtggtgactttggcaagagctgtagtaatgaagaagtaggggtgaatgggtggaggccacgtaacagatttaaggagtgaattggaagtgaggaaatgcttatagattgttaatttgggatgctagggagagagaaagtatgagggattttttttaagttggcaaatattcaagcaatttttaaagacacacacaaaaacaacaccttgtaaatggaatgggggatgtgggaggctgataatcaactaaggaaagtcgttgaggagactgatgggtatggaggcagaaatggaaactagctttcaggtgatgggattcatttcagaaaaggaagcaaaaaaaaaaaaaaggtgtggatagttggggttacaggtaggtttatagggaaggagtattggaagcttcaggattctcctctgaaggtttcagtttgtattaagaaatagggagagaggactgttggagagtatgggataaagggcagaagggagtaatttaagagttgttaaaaagaagcgaacatttacatgaaatatgtaaaatggtaaagattgaaggcccggaggagcagggactatgacattcttctgtttttgcctattagcaacatgaattttcttagaaatctgtaagaccaattactcttctgcccatccataaggacgttgtccatgcagaaaagataacacttgtaaccttgtattatatacttatcatcgcccatttgagaattgtaagctcctaaaagataataactatattttttcatcactatatccccacatcctatcacaatatttcatcacaggtaattcttgacaaatgttgattgcatttttaaaatttctaacctgaacttgtgtgctgtgaccaccatggattgagtcttctctgccactacaaagctcttttctagactatgatatgagatggtttgggctgatagtctatattcaccaatacttgtacagttccaatgaaggtttcaagtctaatacttttggcatttgatataaaatcattttcccattttatttgctaatttatctataactctggcattactcttggttacatttgtctgctgctatctgatttagactgctataagcatatcttgtttattcagagttttcttattcatgttaattatctgtgtttctttatttgctctctacattttaaatagtttcttcactttgctgttttatgagaagggagtaggcaaaaggaaaaaaccccaaatcagacagttttactacttaatagttttttaatgcatctttatagagattgaagttgtagttaacagctagagtgatatttttggcctgcccttatttattaattactagtgcaaaggttattcaaattgtggtttatccaggtcaattttactgttatttttactaatagcatttattctacttaaattgcttcagctataaaatgtttttattgtaacaaataatgcagtataattattgctttctgtattcctttgaaaattcattctctaaacatatgttatgaaatggtggattgttcaccaactacttcttacttacttaattaaagcctttgcaaaaagtttcataggatgactgagttcttcattcgtacctcttttttttaagaataatctcttgaaagtcaaaccatgattcatacaaatataaaatgaacatgtgtcaaagattttatttcactaattaattaacaagcaaactagcaagaaggcaaaacccttttaaaagaaaattggaaaaacagacacatttataggcaatcaagaatgcctaaatgaatttgctaataaatgcaaaattggtcaatatcctcagggcagtaaaatgtagtcttttggaatcctctcttccaccaggtggaaatcaattgcagaacaagatttatttttatctgtatggacagcaatccatttgaattactctcagttttctacaacttatgaaactgtaaaacagcccagtcaaagtcagtgaaaggcacaggcttctatagagtcagataattccaaagcgtctgttagacattgcccagcacttgactgaaaggatacccagtagtctttgtatccctttcaaaatcttcaccatattttctgatactttccttcctttataatttgaacatcttcacccctttattcttctctggtatactatgtcctctctccctcagtgtttctctctcaggggaaataattcacaattccaaagttttaaagaatgcaacggaagtccaggttttgccttgggcttcctagtatttgggtggctagaaatgtagaaaactgggaagaggtgagctgtggatgcccacaatataggttcagactgcaatttcccagaaatataacaatttggactagtcaaagagggcccatatcattacattaaaatgccagattatctagtttttatagtatcaccctacatttttacagcatagtattgtttagagagcacttgccgctatgttttccatgttaatgctcaacacagttctgtgaagtagcccggtgtttgtttttgatcctcctacttaagagatcctcctattttagagagtaaaccaaggcatagaaaagtgagttgcttaaggcagcatattaaaaaggggcagaaatagtatttttactcaggtgttttgaacattgtccagtacctatattccagtatgcatccttgcattcaattcatggggtatttattgagtaaacatagtgttcttacaatagaaacattctatgcctctgatttttattccagtttatgtagaagtaaatcttaagtgtgaactattaacaaagttgatattttatttatatttgttagtaatttgtgttttgtttttgtttatgttttgaggggaaggccaagtagctacttaggtaaaagagttgctgagtggctgaagaatatggaagacaactacaattcctacacattcttgtacattttagttgaacaatgagtgattacatttatttacccagtgcctcttctataaggcaacaactggtagtttatcctattgagaagcgtagataggatgcttattagcagtaactgctcttggtttcaacttgcatcttacttagctttttcaccgttttgtggtttcttggaggaagaatacccataatatacatttggagactgttgttctgtagtgtcaatgaaatgtgggggtgggaaaatgtcattcaagactcccatacaaaatgtctattgctgcctatattttgctatgggaaagtagccacagataatgttttttttttcctcattagtattttaagattttccatcctagtggaaagatatgatttgattcatcctatttactttgtatattaaagtacagtagaacctgccactttttttggaaatgcagcataaggataaagataaatttcatatcagttcagcaagttctatttagcagtgtgttgaagttgagactgaataaaatatttggtttggttttctgttcaaaattttaccttgataaggacaatatttttctacatatatcagtaggcagtaatgattacttcaaagcttccaaagccagatactacacctgcatgttccaacatagttgctgaatttattcccaagatgcatgtaatgtatactttgtattattgagaatgaataaagaaaagtcataatgatgccttccagctgtgcaagttaatattaaaatataatttgtttgcatatttcacctaataggtcttcttcattgctatactgtttacttaagtgaacaatggaaatgttgctgtttatcttaaggatttgtaacatgcctaagatcttacagtacagacttctataattaatgaaacatttttctttttcctttccaggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttggtaagacttctatgaaagcttctttttttatttgtcctggtgcaacctgatcctctttcgagaggaggccaaatggggataggtactcctttgaatcaaaaagcaggctgttattaataaatgttgatgtaatgtttagcaagtttaagattgtgatttctatcctatttgttaatctactcttcatggtaaaacacatttactatttaatttgtattacttgttaatttgtatattgtgtggttctcaaacttttggtctaaggaccattttatttggctgattggtcctggggtaatgtactcttcatttctgtttgctgccatccaagtgctttctttcctgtgactaattttcaagtctctttccaattattcagccttctgttttttgtgttttttttttttttttttttttttttttaaccttacactttgaccaagggatgacaagtcccagccatgctccctctggagagaatagaggagagtcaggagattaaatcacgttatcatggatgatttcttcagattttctgtgcttagccttactttggtcttctttttgttggccaaaatatgagagaactagagattcaaattcatttattcaaattcagacttgatgatagcaccatgattctgccctttttatcacaaacttgggttgctgaggtgtagaaggtagtaaaattagctgaagtaaatagtctcctctccttgctgtttcctgctgtgctagctgagttcttcaaccccatatgagtatgttctttctgttttgaaatctctatttagaattacctgtcatattaccgatcatggcttaaccacattttgagaaccttaactctagataggttcagttctgttcagttggatcaactatttgcctagcttttataaaataatctactaccagattaggaatggggtgtttggtttgatgaaatgtttgcacattacttaggattgagaatcaggaggtctgagttacagcccctgttcagcaacttactattcacctgtgtgatcttgagaaggtgcttaaccgagtccaagttacctcatgtccgaaatggagatgacaatagatacttgcttttatgtattctacagtctaatgagcataaaccaaagtgtgttgtaaatgttagttgttttcctgggatgtttattttatgaagtaaggtatcaagttgttgaagttagcctagagcattgtgagagggatttgtgtgcagtgaggaatgggcaagggatggctccgagtaggcaaggagaatttgaaaggtctttctgctcagaaatctctcttacattgtagcttttactattgatattaatttgggcaaaggagctttttttgttgttgttaagtatacctttaatgaagggtgtaatcagtcagtcatatccactgcaactaactggtattagatcatctaatacctagttgaaatttttctgaggtaatgtacaattttgtggaaatgaaagaataagatatgggggaacagtaacttttggggggtaatgcaggtcactgagaggtaaactggaaaactcaaaatatgagatttgaaaggccagaagatgaaatgaagatggaagagaaaggaatgccaggatctgagataaaaacagtttattattttgcgtgtgattgaaagactgttttatctttgtttgcaaaataccctataaaaataaaaactagacttattttgggagccattaaaaggagaaagtattgttttattatagagaatgtagaaaattctaccagaggtttgagacatttggctttgcatccattttaattttttgaaataattattgatttacaggacgattcaaagatagagagcttccatgtgcccttcacccagtccccccaatggtttcctcttatgtgacacaatatgaaaatcaggaagttcacaatggtgcagtgtgtgttaatggttcgctctcattttatcacgtgtttatttgcgtaactacctacacaatcaggacatagacctatccatcataacaaacatctccctcatgcttattcttactagtcataccgccacctcccactgtccctaacctggcaagcaccaatttgtcctccatatctataattttgtcatttcaacaatgctatataaatggaatgatacaagatgtgaccttttgagaagggctttttcactaagcacaatgcccttgagatctatccaagtttttgcatctatcaatagttcttttttccttacttttttttttgctgagtagtagtccatagtatgaatatatcacagtttgtttaaccacttacctatagtaggacattttggttgtttccaagtttgggctgttacaaatgaaactgctctgaacaattgtgtacaggttcttgtgtagacacagttttcatttctctgggataaatgctcagaagtatggttactgggctgtatggtaagtatatgcttagtttttaaagaaactgccagacttttccagagtggctatttcattttacatttccatcaacaatgtaagagtgatctggtttctccacatcctcaccagtatctggtgccactatgttgtaaccatttttaactgaaaaaaacaacaaacaaacaaaaaccagttaaaaagttatctgcacaaaaaggttaaatggggcaggactcttactatggagtataagttcttttctaataagaatacttacttgtcacggactttgaggatttaagcattagaaaccatttttactatgtcgtgatttttgcaaatacaggcataaatgagaaacagaatttgctcaatagagaagtgaattctacttaatagaatgcagataatagtgaccattctttgtgcctttttaaattttttggaggactaggactgaatacatggaaacactataagatatagttttatatacctctgtgcctatgtatgatatagtccattagaaggagcactggacttgaaattagaaagctgttttctagacttcactgtttaatagcttaagaaaagttggataaaaaactcaacctatctgtccctcaaattcttcttagtacagtaaagatggcatcaccatcatggaaatgtttgagatataaataatacctctcttacttaacaaaattttactaagtgcctaccatgtgccagtcaccgtactaggcatcacagattctgtgatgaataagacattattgctgatttcaaggaagtggttgcagtacaaatatgaagagtgaagtgacctaacatttatttgcacatgtgccacatactttatgtaagtcgtgtcctgttatcctctcaagaactctaggagatagatggagttcttaccatttaacaaatagggaagcaaaagagattcaaggtattgttcaaggtcagagtaatagccaagaattaaacctagatctctttggcatcaaaacccagtatttttaccaccgtaatatgtggttcatgtgaaaacaccttgtaaataactaaatactgaatgcaaatactagtaatcataaaatttatcattaatatttctgttctattagcaatacaaaaacttgaaaacttaaagtttatttttctctaagctattagagttttgtttagaaaggtcagttcaagttccgcagtgttgccaattccattattatcaacaggacatatggtctgtttataatgaagacatgaaggcattgcaagatctttgtattagttttctagtgctgcataacaactacaaatatagcagcttaaaacactacccattttttagctcaaagttttgtaggtggcaagcctggtcatggcatgactgggttctctgatcaaagtcttaaaagactaaaatcaaggtgttgcccagggcatactttatttggagtttggggtcttgttctaggctcacataattgtgacagaattcaattccttgtgattgtaagactggggtccctgtttctttgctaagtatctgccaaggaattgtagcagctcctagagattgccctcatttcttgccccatggccccttgcatatttaaagccagcaaaggagaatcttcctcttgttgaatccctctcacatattgagtttattttgccaggaagaacccagacccttttaagagaccacttggttaggtcagctctctctccctcaaaaataatctctctttcttaaagtcagctgatttgggatcttaattacatcagcaaactcccttttgctgggtgatataatcatgaaaatgaaatccaatacattcacagtcctagtctcccacacacaaggggagggaattataaaagatgcatggctagggcaggggttttgggaccaccttagaattctgggtttgattaaataaataatgggactgtagcctagcaagtcgacacatcagaaaagccatcacaatcctgaaacatcaagtaaaaatttggtttgcattttaggattgtaaatgttactgtggtgtatgcgtgtgtgtgtgtgtgtaacctggtgttccctatgtttgtgggagtttgaaggagacactttgttgataggaatggtgtcttcacttttttaggttgtctcatttttgtacagtgataagacaaatgaggtcctgggttttaataacacttcagcttgaaagcaaaaattaaacacttattcattgactacacccttgtaatatcacttctttgcctttccactagcaagaagttcattttcgtggaagccattctgcctgtccaggaattggaggagagtgatagacacagttgtcagccatagcttgggtagaataaggatgtgaatgtccttggcttatctttattaatcttgtgatggaaaaatatctgacattgttcttagtccattttaagcttaatttatgttcttagtggcatagaaattcagagctgaaagaagtatcatgtctcactctccccttgaggaacaagagttaacgtcatctgacagtactaccataacaaatcaatagcttaataggcatataagtggctttatataaaatgttggtttttttcccccagcatctcagttggttcttaaatatctaattccatgatcctcaaacttttcccactgtaacaaattagagagaggaggaacatgttcatcagtgactgtagttcaaatacagcagaaatgtgcagcagtgatttcaaactgagagaatcctggatgcccttctccattgtgtgccccccccccccacccgccccacatatcagggaacaatttaaaatcctggcacaataatgagaagggagagtgacaaactgataagttccagttaagaatcacttagacaggccaggtgtggcagcccacgcctgtaataccagcactttgggaggctgaggcaggtggatcacctgaggtcaggaatttgagaccagcctggccaacatgataaaaccctgtctctaccaaaattacaaaatttagctgggcatggtggcgcctgcctataatcccagctacttgggaggctgagacaggagaattgcttgaacccgggaggtggaggttgcagtgagctgagaccacaccactgcactccagcctgggcaacagagtgagactccatctcaaaaataaaaaaaaaagaatcacttagacagtattttttgttctgtaatcttcctctctgtcattgaaatgtctcatatttcttgtttcacatagaatctgggagtcatcaagtagtcatctagtctatctcatcatctaatttacgaattatttctatcacaccccaagtagctggcgattcagattttttactgaaagcttgacatgatgagacattattacttcccagaggatcttggtctgttatcaggcaatttcagttcttccttaaatcaagctaaaacttgctactattctcactcactgggagggctgtagtatttctctcaagtttatacctctctgtgatgaaaatctctgttcttttagccattgctcatggtttcgggatccccattctagtcatcttcctttggagcaattccctgttagatcttcaaatgtgatgagtaaaactgagctcaggactgcgtttgttggtcacacccttctcccttccaaatgactgctgctgtaactttctgacaggtcctgcttccctgtaatccattctccatgtggctgccaaagaagatttgaaataagtcaggttatttttcttccctatttaaagccttccagccgcttgtcattgctcttagaataaaatccaaactctcccaacaagcttttaagactctacatggtcttgccccagcaaactcttcccatttgatctcatcactgaattccagtcacaatggccatcctttggtttctcaaagttgccgttcctttggcatctcttcctccctgtcttcacaatgtaggctccttcccatcctaggcttagtttcgttcctctccctagaggcctttccttactgcctcagcttctcccactctgcacactcagggtcctctgcattgttcactgctggccttcaagagcctagaggagttcctccccatggtgggctttcaataagtgtttgtggaataagtgaaaaatgagtggtcacaccaggatcgaatgcacctcttacttttgttaatatattcaaattaatattaatatattaatattctattaatatatcctaaaattttgttgtgtataggggcagggaccaatgaaagaacatttcattgtcagctcatcttgactatgagatcagatattggctatattttgaaggtagagtcagcaggatttgctgtttaactagagtgggatgtgggagaaatcagggagtcaggtataactccaaggttttcagcctcaagcaactgaagaaatggggtcaccacgactgaaatgaggaaaatgagagggggagtttagtgggggatggggtgatgaaaatatcaggattcattgtggaacatgaacagttaagattaactgaagagcctgaattcactgttcctcagtttctacaactataaggaggggttaataatttctcacatcataattgtgaggatttgaggagttgaggtacacaattaaaaacaaaagcacaggggaagtaaggaaaaatacatcaattaagcatggctattagaccatacagccttaccaaatccattggggattagatacaaaaatccaagcatgatctccaaaattttttcctggtagggttttaggctatactgaagtgacttttctttggcataagaagatattcagttatacagttggaaataaaggtattgatttggagtatccaaaaacatctctcagtagagatccacaaccaaagaagcataaaaaaaagtcttccattcactcgcaaaactgtcttatgaccattgcaaccctcaacagcaaacatatggagtctctactacatatagaggaaaatgctagattctgaaaagctaatatctataaaacagagtttatgatgttgttatattctggggattgatgtaagcattttaccaggacttattatagcattataagcttaacacaagaaatatgtggctatcattatggagtgaaaggctggaaaattcctcacctgtgacctgagagatagtgcgtggtagaatttgaaagaagtgttgatttcagaggaatgctagtgcttgcttagggcagtattcagaagaactcttccaatcacacagccccttgtacaggcaaactccggaatactttcagtacatttacttggctttattgttagagcagaaacttcacagctaaaattatggtcttaggggtttataagatgcaattaaacttaatttttaaaagttctcacaaatcttatttgaagtcttaatatcttaatttcctttatataggatatgtggatatatttttatgataaaagtaacatgtgaccaacttaataacagcttcgttaatatcctaaagccactcatcttactttacatataaacagtccttctgaagcctaattatgcatggataattgcagagtgagtttgggaaaagaccaaccgggcacatctctctccattttaaccacaatgagaagagagtacctagtggtgcatcttcctcgcttaggttccctgagtctgtctttacaggaagacttcattgttacttgaaggtacattcttggaactttacacacccagccactcaacacatgaatacatacttattttagttaactgagtacttagtgagtgctaagctttgttttaagccctttgcatgtagtttattaactttgttaatagttacaacagtccttcaagatacatgtactgtgttatactagtgtaacagttgaggacaccaaagaacaaagaggtgttgacacgtggtcacggttccatagagtgttaagttagagttgggttcaaaccctggcagtgtggccacagagccttgttctcagccactgcactgcactacctcctcccgtgaaacataagaaaaatgtgagaaatgcttaagtaagtgtagtttttattcataaataaaatttacataagtacattatgtgtaatttgttttatgtatatgtgtgtatgtataaataaatacatgtaaaaataaggccacagttttaatttttttccatctctataataaagcatgtattatagaccattagcagaatttaaagtgttatagtaaatattaattgtgacttttgttttcttcttccccagtttcatggttttctccaaatgaaaatattcttattgttattttcccaatttttgctatactcctgttctggggacagtttggtattaaaagtaagtattatttctacttttcatttatgtttcagtgatgatatagttatttctacgagacattgtcagcgaaatatttaaagttgtactaggaaaagtgctattatgataaatatgagtatgtaatttgaatactactagtctccttgaagtatatgttgtcgcccacattttgctgcagttcacttttaattcctaagaaggttgttttcacttggtgtttttttaatctcttaagaatgaatagtaggaatattagtaccaacaccttaaactcatgtcacattttaatattcacagaacatctacacacacattatgttattaggtaaacaggtggtgacagcctgcattagttttaaggtaggacgttatactttggagcatttagattcccctctttttattttcccagtttgattttctctgtgtacacgtgttcacccttggaaaagtccagtcggaactatgttttgtcatcctctgcgtgcagttctgcagcctctaaagaagcagccaccagagagttaggttctttgatcttgctttcctataatagtaacgtaaccagacttctgaaggcagatcttgatgctgcattagatttagcttcaacaacacagaattgtcattactaggcaaataggtaatatgcattacggttaatgtttaatcaaccatattttcatattttggtaaagaaaatttacaaaattaatgaagttctgaggtgacagtctaaacttttaagctttttaaatacaagatttattccttctttccttgtagcatcctgaagaccagtaaacatttatataagcaaggaataaatactgcttatttaatttattctgcaacctttaaacacacaaaagctagtaaactattgcagtggattgccctgttgtatattttatgaattttacttttactcagcagtttaagctgtctatatctatggtggtgtataaacatggaagggagatgactgattgattatatgttttaagcgcttttctcagtgtatggcattctggaaatgcttagtgatttcagaaatgttctcaacttttgtctgaaaggaaaaaagggggaagaaaggggtggcagtggcaactgtcaagacattttataacttttactttcaagatagtgtctagacttcttttggaaattttcttataatctcttagttttttatgtcaagaaaaggactggtgtagcattaagacctattctggcatcaatgatattagggaaagcttttaaccatattggcagagccagactttaagggctaagtcatatgacttgggcagggaacagcctttttcaaagatcatgaaattatttcacatagtgcgattttttgagtttggcttgatggatgtttgctgatccagacgttatcattggtgacattatttttagtgaaatgaaccaggagtgagtttgttcactgttggctgtatttttacaaaatgagctttacaatattttttctgacttaaaaaagtcatacatatcactttagaacacctcgtacaaagtagggagttatttaaaaaaaaaaaaaaaaaactcatctgtagtccccaaacctagaaataaactattggtatattctggtgaatttcctgtttccctttctgtctttttctattcatttttgttatcttttttttaaaaaaaggtaaccataaatggactcgtacagcttatatgcttgtacgttctgatgttcccccatgtcttcagaaatcttgttatagctgcctgtgttctacttttgtggatgcactataatttacttaactctcatcttgatggattttaaggatgtttccaatttttttgatactatgaaaataactctgctaaagatatgaagcctggaattggcagagatattttaagactctagatacatactggcaaaatgttttccagaaacgtttgtatcaactactattgcagatgagaatacctgtctttcacctcagttataattctgatattataacgctaaaatctctgcaaatttgatgggtgaaaaagcatcttattgtaattgtaatttttgtgttagtaaggtggaatgttcttttttatattttttgtactggtcatattttaaggaacgtgcttataaacctaaagaaatattttggtgggaattttttgttttggctcatcttgaaacaggtagatgtgtgtgtatgtgcatggaagaggtatgtctatacatgattcacccagcctgcgctcacatttaaaggtgttgatgataatagtagctaaccatttgtggagctcttgctctgcttgacaggttctgtgcaaagtactctatatctgaaatgacatttatttctcccagaaactctatgggcatagacactgttgttattcccgttttgtagataagaaacaggcacagatagattaggcaatttgcccgcaatcacccagccgtttcctgatagtactgggatttgaactagtactgtttaccactgcactgtactgcctccccgttttgcatttattttgaggattttatttccatgaagggtgaacctatatctaagcacataatactgagtagctaaaacttattaggagagcagaatgttgacctgatttgtttacttattctgcaaatacctagtgacagggtgcctactgattgctggacaccaagctacatgcctgaaatgtgggtgtgatgtgcatattgccctggttttgtagcacccacatttaagccagggagacacgaatgtatgtgcatcccatgccttgtcacatctttgagatagtcatggatccaaatctaacttttctgttagtgcctctgttgatggcctgagcattccaccaaatagaaatagaaagcacctctattctccacctgcttagatgtatattttttagaatccagtatggtaaatcttctaaagcattcataataactcagacccatgacttttattttatagatttctagttctgcttaacttttcctgattaccatttatggtctgtacttggctacaggggatttctctcagtggtttttccagctctgcatgtgagtctttgtggtccaagacaaacctggacctttaccaggctaaactttcagtaaagacagcaggttatgccctcatttgctcactctgaggagaaagagtttctttataccagagctgtatcttgaaagatgtctcaggatgccattggtcctactgaggaagaagcctgagaagactcttaaactcccagagcccagccagcacttggtgagccctggaccacgtttcaaagataaaggcctcttacagggaaatgttcctaaataccttcacctttagcttagctttaacttaggaacttttaagcagaatctctatgcttagcaaacagctcagagattgctagaatcaaacaccaaggcttaactgatagtattgaatttcagacgctttgtttgtgttctgaaaactacaccaactcacagtttgccactctactgacaattaagttcctggctgattttcaggattcttctttctcactctgatatcattttaagtgctgtccacctagtcctccagttcctgcaggattaaggtccaactgtatgtaaagaactggctaacattttgaaattctttgagataggcctatctgttctttctttgcctttgtaactttgttttatacaggcaatactggcttcgcacaaaaccgcaagaccataaacatgaccatgtaagctgaaattctgcaaaacaatcttcatgatgaatgggaaaaactattattatattgttccatgacctttaaaattttttttgttgttaaaaccttaaaaactctgttattatcaataacaacggcattgggaaatgaaaaatagtaaagctagtatttagtatgtggtaaattaaatcattagaaacatcgagaatgaaagtgtgttatcaagagtagtttgaacaacacttgtgtgttcttcttgcataactttggatacagagcaagcatcttctctatggcttggtgaattgtcatacccctttctaagtttggatcggtttctaccgttttatcctttgcactctcaatgtcatgaaagaattcctttaatgtttttttgaatgtttaaagtttattttattgccagtcacttcctctgggacatctttattctttttgttacatcctcctcactttcctcatttatatcagtaagtccaccttcgctgagttcttctggcttcatctctagagtttcttgcattgcagtaatgtcgacattcgcacaatcagctatttcttctcttactccatttatggtctattcaaatttcactcctagcatttgtcagtttttatttctttactctttcatctttcttgcctagtttcctcttttgatttatccattataaaatgtcacatgggtttatcactgggaaacaaggaggtaacaaaactccatactttgctgtctgtgcatgacctaaatatcagatgtacagtgactagtcaccaacagtcttcaaaagaagtgacatggttgatcactgatcatgatgggacatctgctatttacatagttatttttgaactgaagaaatagcagtgaagttgtactttatgcagttactcagttaatatattgtggtaattgaaatttggactgtttatgagggatttatttgattaaaccatggtaactggaattcatatcagaatagtgcaaagtgaggactgctgtacttgattccaaatttaaaactgtattctaggcatctttaatttttttttttcaactttcccctccctgctcaaaattactccacagagattttggttacggctcagatgtctttgagtagatgttcctgtagaaatcattgttagaaaattcaaggaaaaaagttctcattacactaccttgagatcctaccaagtccttgagttttgacttgaggacagcttaataatgaaagtaattcagccatgaaagcatatttaaaataagtaatggaacaggaaatttcttcatagattagaaaaattattctgaaaaagtgaagacatagcctatcttggactaggaaatttccatccaagaaagtaaaatatacaataaatattatcacaaagaatcattctgtgacctggttgatggcccctggtaataagactttgttattaagtttactgtgaattctttttttttttttttttttttttttttttttttgagacggagtctcgctctgttgcccaggctggagtgcagtggcgtgatctcggctcactgcaacctccgcctcccaggttcaagcgattcttctgtctcagcctcccgagtagctacaggctactggcgtgcaccaccatgcctgggtaatttttgtatttttagtagtgatggggtttcaccatattggccaggctggtctcgaactcctgacctcgtgatccgtctgtctcggcctcccaaagtgctgagattacaggcattagccaccatgcatggccccttacagtgaattctgttcaataatcttgaactcccagtgctttcccttggtcctgtccatataatgatcacattctgttattaaataatgtgcttatgtgctgattttattgtaggaggaattgaacaaatctatagctcagcattttactcttttgtagaaatcccttgaccctttccaaatccagacatcacaaccttctccatatactcaaaaattgtgaaatccacaaggcagggaaagttgataatgaaaaaaaggaagtttggtctgttggacatcctcttctttcatcttttatgttcaaagtacttgatgcacaaagcctggactcttttatctctgtcctatagactgatgtctagtcagtattcattgtgacaaaattgtttcattagagcaagttggatagtgtatggagattattataaaatgatttaacttgtcttctgtcaagtaattttataattggtattaatgttcatttttttccagtaccagtttcagaagctttcatttgtgtatgcatgtgtgtgttaaataagtgtattagatacttgaaaaatagtaatttaaatttaaacaatttaaaaaatgaaattgtaattttatgtgtaaaggtgtctgatgtgctttattctgcactgaaaacaatattcatttacagctcaacagacaccaaatatattctaaaattactttcctagagttatcagaaaacagcactgtaataatgaaatcaaacccatctttctttatgatttattcttagtctgatacgcatcagcctggtaagccttcctgtctctctgcttacttaccaatcactccaaatgtcatgtctttgggcaggcattaaattcttgggttttgggttttgttggatggactgcagtctgtgtgagcctatatgggtgtgtcaaatccagtctttggggtgtcatggaaacttagcatgatagacttgattttatccccaagttgacttggtaatttcattagatttcatcagtcacaacctgcattttatcttgtatgtgctgtctattggtcacaaaatcagaaaaccttcttgtccattcataacattagctgttttttcaggtggctagagggacatgtcattgcttcatctgcatgaatttgaaagattaaatgcataaaggaattttcttagagtagagtaggccttcaccatctctttaactgggaaagaagttttgggagtaacatactcatcactcacacccccctcccctcaaacacacacactcacccattagaatgtaagggccttgaggcaggccttgtgtttgcttttcgtgttcactgtcagcatttagaatagtgatagtcatatagatggatagtgctcaatcaattattgtcaaataaagtaatctacttgttccttgatttagactagcaaaaggggctggtacattgtaggtacacagtaaatatttgtagaacagatgaatgaaccaacccaacagatttcttagaagtagcctgcttttggttactaattattttatagaacatataaaagaaaattttagaatacctaatttgttcacaaaaatgttatattgtctcccctgatacttggtattcctgtgtcaggctgagcactaaggttatacattttttgttaagcatgtaattactgttatttctcatgtactttattttgtgtattgatttcagtagttgtagctgagggtaatccttaagagcatgcagattttaaaaattacatagattgccttgccaagccgtctgaaagtcatactagaaaatttgataagagcctgctgttaacaacacattgagttatttttatcttaatgctaagtggagaattacttgaatttattttttttgctgcatttaatgttttgatacattcaataaataaggataaatccacttactgaaggacaagaaaagatcaataagagatccttgaggttattgggttctatggcatctattatatcaaattccttgccctttcactatactgagaaatttgacgactgggctagcaacaagatacctaccccacattagctgtggtacctgctgaaatgtgaccatgtgagcaaagaaaatgagccaaaagaaactctttctgaagattcactgaagaaaacctggagatactctttcattagtagaccccacagagacaggtggagtgtccgttatccagaatgcctgggaccagaagtgttttggatttcagattttttcacacttgggatcctcaacatgtgtatttgttggaaatggctacttaatttaaggaaaagtttaaggtggcctggaaaataagtagtagttgaactaatcaacaggaaccaaaactactttcaatatatagtgtacttatactcaaagagagacggaccattttatggtggaaccctgtctatggtagtattttgtgatgttttatttttgttgctattgtttgcactgttttctcctataacagctcttctaagccttaagaggataaattttatatgagattcaaactcttatttttgttaagaaggatgtaagtcaccaggcatggtggctcacacctgtaatcccagcactttgggaggccgtggcaggtggatcatgaggtaaggagctcgagatcagcctggccaacaatgtgaaaccccgtctctactaaaaaaaaaaaaaaaagaattcaaaaattagccaggcctggtggcgcatgcctgtagtcccagctactcgggaggctgaggcaggagaatcattgaacctgggaggcagagtttgcagtgagccgagattgtgccactgcactccagcctgggtaacggagtgagagactctgtctccaaaaaaaaaaaaaaagaaaagaaaaagaaaaatatagaagaagaacataagtcaacctttttccatgaaattatactgtactctaaggcaaattcttgttgcttgatcttagtagcttttttatgcattgacttaaacctggaagagtttcttatgaatgtttgattgactgtgaggtgcatttgaaacagcttctactttataaccctttgcagaactttcaagctctatttagataacaatatatatgggattaaaatggaaaatgtgacatatgtctagagaaagttcctttttctgtgtatgtgccttttgtgacttatacactcagtttcattctagccagcatttgagcctagaaaatagggaagttatgaaaactctgtgccttataagaaggcaaggctatgactgagtattagacacataagtccagggctgggcggaagtaatgaatcaaataaaaacttggggaacttgccataggatgttttctgattaatatgggtttctcttctctgattaatgtggatttcagttactcaactgtgtaactgaaatccacattaataaattggttatttattgcttaggacttatctccccataaagagactaaaattgaggttataaattatgggtagatagaaattccagaaatatttaggtacctgtcatataccttaaacatagatttttatcacaatcatttaggggcttatttattgcttttactttatttaatgtttgtcactgtagaagaaaaaaaactaaatgctaaatataacatttaaaatattttccccttcatgacagccaccaattgattactgtcatggagactatctctatgtggatgacacacagcagcatttcaatcacacgctgtttctttccgctactcagtttgcttttaggttgccttaaacaacttccttggtgaaaaaatcaacttcaatattaaccaaaatttaaaagattcatatatagtaaaagaactaatattcgcagattgacccattccattctttcagagaaggtatgagatacttgacagtggagctagcagcagaaacaataacatgtatgatgaatcccattgagtctttgcagtttatttttattaaaatattttaattgaacaatttaagctttttttcttcatcaggatttcacaagggtgtaatctgcccagttttattctgtcatttttttcaaaacaaaataactagatttccagattgtttactaattttttataaggtaggacaaaattctctttttctcaatattgttaattaacccattatttcccattagtattatgtacatcagtgttgtgttgtcacttggagtggttttgctcatcgggggcattgacaatgtctgaaaacagtttgattaacataactggcatctagttactagcatctagtatgtagaggccagggatgctcctcaacattctatattatgcagagcagtgtcccccctccccccaaaaacttatctagctcaatttattagtaatacatcaaccgagaaagactgatgtatagaacctcatttgttagtgtaggaaaataaggtgtcacctataaattcaccatccatatttatactgccgtgacgttatccatttgcttatgaaagagatgtgaggtgacttgatgatattaaggagttgttcctcataagttattacattatagtacttctgtcagtttgtctctgtaccttacaagttattaaaatggcttcacttgtgattgagttcatataattctttgtttttcttttttaagcacttaaatatagatccggtggtatggatgagaaaacaattgctttacttgttgctggactagtgatcactgtcattgtcattgttggagccattcttttcgtcccaggtaagatgtgcagttcctaggcaggaacgcaggaggtagatgagtgcattccaaggtgaggagggctgcattagtttcctagggctgttggaacagatgaccacaaactgggtggcttaaacaatagcaattccctttccatgctggaggccataagtctgaaatcaggacgtcatcagggccacactccctctcaaggctgtatggaagaatccatttcttggctcttctagcttctggaatttgctgaagaatgacagtgtttatcattccttagtttgcggcagcataactccaccctctgcctctctggtcacattgcctcctccttcctctatgtctttgcctctgtgtttttgttaaatctccctcagcctctctcttatgagcacatttgtcattggaattagagcccactcatataatccagggtaagctcctcctttcagatccttaacttcatcatatcttttgccatataatgcagtattcactcttttgctgtattaggtaatattcacaggttttagggattaggaggtagaaataattttagggccatcattcaactcactacagaggcaaacttcattggccaagacatgaaagtagaatgaatgtgggaaccaagtctggaaattggcagttgcatttggaggatgggaatgtgagtgggaaatgaggatatgtagcacatgtaaagaaaagcaaaggaaggctgggactcaacctaataactatagcacaccatcgtttgtggagaagaatgcagtgcaggtgtaatatttaggaacatgggcttggcagctctattggtatctgaagttagtgaagacagcaggaagaaggaggtgaattcagaagacaacttgaaggaacaattgatttgactttggggcatactgaatacagcagatgaagggaacagtaaaagatgaatacacagtttctcactggaattctgtaaactagttaagaaggaggcagagccaattcaaggtttcagcatgttgaaacttaaatgttgagatttagatgtcttaagtttggtcttaatcaaaaaatagtcactaattttgtgtgaggttttcagggaaacgtatatttttaaaattttcacagttgtcaagcacagaatttagattagtacatttaaaaagtatgtaggaaaatattttaaatgtttttatttattgacaggtgaatattcattaaagaatgctactggccttggtttaattgtgacttctacagggatattaatattacttcactactatgtgtttagtacaggtgagttttcattgttacagtatgattttgtctacctttttcatttacaaaacagcagttttggtgaaaatgctcatataaattttttacaactcaataagagtaggtttattaaaagatttttcatgctattcttagtgacattttcccattcatattaattttaaggttattctaggttagctgttttgtaaaaagtgactttcatatgtatttgatgccaatcagcataattttaaattatgccatataacttcttaatgattattttcatattctaatttcagttttttgaagttataagtggatgttaaacgcagtctttctcttcttttgctaagcttctgcttctgcattaagaagactggttctataaaatggaattatgaatcacaaatagtagaaacatatttgttttaattattagcaaaccttaatactgtagttttaagagatggtatggaaatccaaactataatcagtatcattttcactgcattttgaaagtagatcactaccatatttagttattactattaaagagtcacatttaacacttggtcttagagcctatctctggctcgttgtacattggaaaggtattagtttgttataaaatgacatctagagatagagagcatgatgtttacagtcaggtatttgcataatggttccctcagcaagagcaattccatgtgcatgtggagaagacattcataatagccattcatattgtaaaatgcacaagtgtggtaaaagcagcattgttctaagatttagggtaaaaacttccagtccagcttagctgactgtgaaaattaaaggctgccgaatgtgtgaatttggagctgactacttgtgtggaagggttagatcactgagttaacatctaccgtcaaacaatgaactccagagtcagtctttggtcttaggagatccctgattataccaaatgcaagtggaagttattgctttttaaatactcttgacatgcttctgttacatccttttccttcctccagcgattggattaacctccttcgtcattgccatattggttattcaggtgatagcctatatcctcgctgtggttggactgagtctctgtattgcgggtaagagtcatctttctgtagacctaatttgggttacttttggacagagctcttcttcctttttctttttctttctctctttttaaaaatatatagacttgatttttttttttagtgcagtttttggttcatggcaaaattgagtggaaagtacattcacatggatatttttatgtggacataagttttcaattccatttgggcaaataccaaggagtgcaattacgggactgtaaagagtatgttgaaataaactgccaaactctctgtatcaaaatagctgtaccattttgtattatcaccaccaataaataagagtttttgttcattcatatacttgacagcatttagcattctgagcttttctttttaaacttccaatttaccccagtggtaatagtgctttcctcctcgctacaaagatattagctgtatatatggcttggtggctgattgttctagcacccaaactgatatgcctgtatttgtggaagagcttttaaaataactgggctcaaattggttggagccttagacttgaaacaccagttcccatttcttgatatgataaggtatgtgttatgcaaaggagggctttgttcttctaataattttgagtcattttactggttaagttaataaacatatatggataatttttgttttttgatcgttagaataactctcttaaaacttgggattattactgttttttagtaagttatttcatatgcttttctaatacagaattttatttgtttttacagcgtgtataccaatgcatggccctcttctgatttcaggtttgagtatcttagctctagcacaattacttggactagtttatatgaaatttgtgggtaagtcaatcttattttcattaacctatgccaataatttcagatatatcacttagaaaatgctttttagtttgttcttccagtttaggaccaaaaatgagaaaatacaattggagtgattcgaggataattaaagagggtagaagacatataggatttttagttggttcttccagtttaagaccagaagtcagaaaatacaattggaatgatttgagggtaattaaagagggtagaagacatataggattaatgaaaatttggtttccaaagtagtttaaaggaaaatggctttatctattagaatgtgtacctttttatactaagtaaaaggggagagatctttgaggatccattttaagtaatagaataggatttttaattgttccagtgtttctgtgatagagctgtcctgcacagacctgtttgtttgtcacttgctctttttcttgcagacatagacaccccagacaggaattaaaattcacaatctatcaattttgttcatttaaagagcagtgacctctaatgcatgactttaaaacagttctagttaaaaccaatataatgaaaacattgagtttcaaaatttaggcttttactccttttaaaatcaattattagtaagtatggaatttacttcattgtttctaacttgtatatttaatctgccaattttcaagtaacatttctgcataaattcttattttttattgagatatatgtacacagagagatattttcaattgtgcctgaaactaatgttatcttacctaagctcaagatgttcccaataatgtaatttatattagtttccgttttttaaaaaaattatatttttatgaaataaaacatactcttaaccacctatcaaaataatcaaaagttataaattaatggagtaaaaaaatagtgtttctgctttgctttaggtaaactttgctgtatgtgtttctaaaacttaatacgaaacttgaattgttatagtcaaataatttctcatatgactcacataatagtttcaaaaaacttttacctttatttctgaactttggttcttgatgattgttaattgaattcaattctgtcatatattctgtgtctttctttaattaatgcttattagataaataattaaaatacttaactaaaatctgcgtatccttagcatatgagttcataagtcttagttgttgctcaatgaaattttctaattttataccacataatgccataaaatacaatggagacatctaaagcagaatggaattcatgtggtagctacagtgaacatcttgaatgttggtgcatattctatttttgttacatcttccaatcaccatgtgtctggttctggaagatgacactcctggttttgttgctccccacaaatgcctgagaatagtgtgtgatttgcagtatccatacaactctggtgaagtagtatgagatacctttggctgacgggcagcacgctcttattttttcctcactatctgggttgtcctcccttttactcccatgccaccccatgccttccatatctagcatagaatgatcttcagtacagttgccagcaggtctggtgacaatgtctcaagtggaactaagcattgtctatctgccacctccttaacttcactctcctgccttctccatcacttacgttcctccaagcctgtgaaaccactgtactgtacctgtcactgttctgacattaaaattaaaatgacttaaatcttgacaagtaccccaaattattttttctttgtcataggttagcatataagtatactatatgctaaaatttatgctatatgtttaaaatttagtgcaattttattgatagtgtcctaattttattgatagtatcctaaattaactttttaaatcaacttgtctgatgccagggttcagagggacacctacagtcagttgaaaggcaagaagagacaaggtacaggaaagttgctctttagataacatggtagactaggaggaacattaatatggtttgcttatataatctgactgtgtaaatctgaatctatgtaacattaaggttgcaaatttgagcgtttatattaggaagttaaaattttaagtgtctccaaaataatttttactcattgcacgtgttctgttttagaaaagcctaatgattgtgttttgatttaaatgcaataaaatcctcaaatagttaaaaatccaagctttctcttcaagaagaagttaatgtcgctatgagatttttaacttttataatttttattatttccaactttaaatttgtagccttaatttgctattttaaagagtaggcctttcactttctacaactttctgtgaaagtgactttcactttctattttttaactttttaaactgtgttgtattttttttcttttattggaagcattttaattttataagatgagaaaaaggactgggcacaataacttaatgtgaaagcatagaaaagattacaagaacctaaccaaactcactaaagttgggcttgttgtttgtagagaacgtttatataattataaggatcaatactttctcatttttaaagccattaccagttagttcaatataagggcatatagtgttttgatacaaatcaatctggtagcagtaagtaccatatttaccacaacatcccagatattttagaatgatgcagatgcagaatatatacgtagaatttatatctatgtatagatacaaattcagatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctagggtaagttaaagtttattaaatgaattgtgaatgatcatttgagggattagactgaggaacttggtaattgagatattttgctatctgttttgtctcacgtcaaattaagagaatgttgaagtcattgcatgacctttgcatgaatgggtccagttctattttaaaacctgtgtttggtcattttagtgtcaatgggatggaataaatgatttcttaagattgtactgacttctcacacccaaaactggaaagtaggaataatggctatattatctctgcaatcagaaggaagctgattccaatatatcacctcacctgttggattcattggatgtgcatacacagaatgacaatttcaggcttaaaaatgaggagaaatctatactaagttgacatcactgataattataatctataaaataaatgtaaatattgctgaaaacatctgttcggagttatgattcgatcctctcccatacaaatgttttataaacattttttcccttaaaactgtgcttaaggtttcattgtaccttagataccttattaagccatctgaggaaattgcaagaaaggagtaattttaggagggcataaatgaagagaaaagcatatttataaacatagacttatcacagtgacaggcccaagaggtatgttgtggacataagctctggaaaggattatattgtacttagcttcctataacggagtgatgatagatgtatctggaatgccaaagagagtcttctgtctgtggccggaggtagaggtcacatatgcttctaagtctgacaagcctcattgtgcctaaagggcaggtggggcgggtatgtgtgtctaccacaggggtatattctagaaagattggtgccatagctatgttggtcacaagaggccagcaacttaactggacctgtgaatcctaacacattttctttcccagttactgagttcaatttgcgatacttaaagatgattctgttttgcttccacctcttcactgtgttatttattctgttgttgctatttatgcttgcactttcatatttttagaagttagaatttcttgagctgagagtggtgaagtgggaaattctgtttagaaaaataatattaagagaataatacagttattattaaactattaacccgacttggcaagctttgctttaacatttacaggcttatttcgttgttttgttttttcttttttttttgactgtaggatttactgcttactacgtaatttaaatattagcatatataagttttactataaaatgacatgacataaattatatttttatgtaaatatattttaaatatttttcagaaagctgtagaggaaccccttaatggtatgtggttatttcactcttaatcctttaccagatcataatttgatctggcccgcaaaacagttagaatgccctgtctatgccttaggaagaatctaggtttttttcctctttttttctttccttggcatctctactcttgattattcatcaagaattatgggctgggtgcggtggctcacgcttgcgatcccggcaccttgggaggccaaggcgggcagatcacgaggtcaagagattgagaccatcctggccaacatgttgaaaccctgtctctactgaaaatacaaaaattacctgggcatggtggtgtgtatctgcagtcccagctactcgggaggctgaggcaggagaattgcttgaacccggaaggcagatgttgcagtgagttgagatcatgccaccgcactccagcctggtgacagagtgagactctgtctcaaaaaaaataaaaaagaattatgaatattacttttataatattctcaccactgggaaaaatgcactattctgtgtctaagtagctgcttaccttaaccaagtgatatttgggcaaggggatcgttgccttttgctactggttgagacgaagcatggcaccccctagtagagaaggatcccaattacttccaatttgtgatgtacacattttagaaagatacaggctattgccacagagatagaccaaaacatctcattttctttctttgtaaacctagatctgatttcccaactaagtctgtttcctttgtgaatgctgtgggtatgatccacagaaaggctacataatgaaatgatagctttacaattaatttggctgtagagttgtagactagttagcatatcattgcatatttgtttatttagaaatgatttccaattgtggaacctcactaactgcctgcttggcttgttactaatcctagcattcaaagaatcaaaaggaatgatgaatgatggtaagtataaaatgcacttaataattatagatcagttaaaacatggacattggaaaacaaaaaagcttcttgaaaatgtggctcttttttagtaaaagggacactgtcagatgataaaggttcacatttcttgatgtatacaacttaaatctactttgctaaaaattgcaaaactactactgtaaaaactgtagggtgtcacgaatcagactccagtcatatggctcccagcaaagagaaattaccactttttgtaaaatgtttttcagattccgtgtctggtgactgtaactttaagatgccttttataaggcacataaataatctggcacaaatctttatcattttgacagagtttcttttatgcttgtgttggtgattttgttgcatttaacccatggggacttaacatctctgcttttccaatcaggagcttggttctaaccttttgggagatgattaaagagagaggttgagagagagagagagagagagagagagagagagagagagagagagagagatgtttcgtcctcagcttttgcttccattttttttttaagaactctgggctaataacttctaatctttatagaatatttcaaagaaatatatttgttcttaaagatacataggtttgagatattgagtgctacaagcatttattttggttttaccttaacatattatgattcctcagttttgttggcatttagtaattatgtttatgtttttatcttatcaaaaaatgtcttcttacctttgatatttataatcactcctcggtcatgtaaatagtttgctttatattttactgttttaaagtctgtgaccttacctgccctcttctgtagcaaagtgcagcatttaactcaggaagctatattccccccaagtgtcattaatatttgcataagattaaaaacactccagtcggccgggcgcggtggctcacgcctgtaatcccagcactttgggaggccgaggcgggcggatcacaaggtcaggagatcgagaccatcctggctaacacggtgaaaccccgtctctactaaaactacaaaaaattagccgggcatggtggcgggcacctgtggtcccagctacttgggaggctgaggcaggagaatggcgtgaacccgggaggcagagcttgcagtgagccaagatcgtgccactacactccagcctgggtgacagagcaagactccgtctcaagaaaaaaaaaaaaaaaaaaaaaaaaaaaacactccagtcatgcattggtgaacaaagtttaaaacaacgtgtattcagcatggagtcacagaatgatcctacttttgtatgtttgtgtcacagcctttaaaagcatgtcttgttatataagccattaccctcctaaaaaagactatagttcacaggaataagttaaaagacataacaaaacataaaatgactagtaccaggaattgtgaccatggtttgttctggtaactgtggcatggcatggtttgttccaggaattgtggcatggtttgttcatgtttacattctgatgtcctattttttttttttaatttctatgtcctttccttttccttggtggtgtcattgttctgtagctgtatgaagaaactaaacttttctccattttcaggaaagcaatctaagaatcttgagtgcctcttcctttgttaatttctcttaagatgtgacttttttaaactactgcatcaggaaatattgtaaaacagttttgccttgaatatttgtgatgaaatctacgatgatcttcaagattctcttaattttgctaatattcagctgatcagaatttgtttttaaaatgtctggctggtgggtacttcccactgacaactgcttattgcttacagtatgtctgccttgtcaatgaatgaggttcagggtgcttcctagggatcagagtcagtaccatttttctctttcatctacagctgatcagatgtttattttacttacattaaatgaatgatggagatccaaagtgaatattatagaatattattctaggatcaacatcttttgctttgaaaaatcaacatctcttggcttttcctcagccaacccagcaaacagagattatcagactctgttgattttttactttcatttggcattggcctttttcttactgaaattaaaaaggctaatgatttgcctggtttctgtctctgacctttgcaggtctattttcttaatttttagatactatatatctgaaactttttttaatgtgtcaactttttaatggatagaaaatagacacgaatagtgattatgtgttcatttttcaattttccagaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 32 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 33 - DNA, 15496 bp, Homo sapiens
gagctcctctgctactcagagttgcaacctcagcctcgctatggctcccagcagcccccggcccgcgctgcccgcactcctggtcctgctcggggctctgttcccaggtgagtcggggtggggattgccgtcgggccagttctccgaagccccgggaggaccggctcccgggtcaggtcatgcatgcttaggtagctgtttatgggaaggaggggctagagacagcgattgaaaggcaacagccagtaggttcgaatccagaccctgcatacctccacgtgtggccttgggctatagattgcagctttaaaaaagggtagggggttggagatggaggggaggggcgggcctcgttttgttgcccaggccggtcttgaactccgggggtctagccttacctcctgcctcagcctcccgagtagctgggatgagaggtgtgaaccaccgccttgcttggctagattgcgtctcttacagtttctcagctgtaaaacgggaaacgttatagcggccacctggcagggtatcttggcccagcgcagcacctggccccaggactcgatcatgatggtttgggaacttggctctgtgccaacccaacaaggcttaagggacccccacccccctcaagatgtatattctgttcctcatcctctctgcccctggggaagtccagggctgcttctacttgggggaattccagagctgacttatccgtggcccaaagctgagaagtgggacgccccagcacaccctcccccagctccagcccagctagggaagagggaaggggtcagagggtctttcatggtggtgtaagtttggggaaccaggagggtgggagattgacagcttggttaacagctcaacaaagcctgagatccaggccagcacggtagttcatgccagtaatcccaacactttaggagccccaggcgggcgaatcacttaaggtcaggagtttgagaccagcctggccaacatggcaacatcccgtctctactaaaaatacaaaaattagctggcatggtggtgggcgcctgtgatcccagctgctcgggaggctgagggaggaaaatcccttaagcccacgaggctgaggttgcagtgaaccaagattgtgccactgcactccagcctgggagacatagcgagattctgtctcaaaaaacaaagcgttctgatccggactcagacccagatcgcactgctttctagctgagtaaccatttctctctatgaaatgggaatggtcccagaatctcccttggagaatgtatggagccagtgtcctcacacccccatccaagatagaacaaatctgagacaggaatctttgagtgaggcagtgctgggctcagacattttttcccaccttcggaggcagcagaatctgagggacctgatccaaataagccccttctttctttcttttcttttcttttttttttttttttttttttgagacggagtctcactctgtcgcccaggctggagtgcagtggcgtgatctcggctcactgcaacctctgcctcccaggttcaaacgattctcctgcctcagcctccctgagtagctgggactacaggcatgtgccatcacacccggctaatcactgtgttagccaggatggtctcgatttcctgacctcatgatctgcccaccctgcctcccaaagtgctgggattacatgcgtgagccacagtgcccaccccgtaagccccttctttcttacctgcaaggtagccagttgctacccatcctgtgctgagttacttgtattagcaagggatggggtggctatactcacccaccttacagatggggaaattgaggcccaaagagggggaaactacgtgtctcagggagtgaggagccagtctgattcctggagggctgactgtctccacctgacttcttaggagggaggagggcaccaacttcacattaaaatctggttggacacagtggctcacacctgtaatcctggcattttgggaggcttaggcgggaggatcacttgaggccaggagtttgagaccagccttagcaacatagtgagaccccatctctacaaaaatgtttttcagggccaggcgcggtggctcacacctataatcccagcactttgggaggctgaggcgggcggattacctgaggtcaggagtttgagaccagcctgaccaacatggagaaaccccgtctctactaaaagtacaaaattacccgggcgtggtggcgcatgcctgtaatcccagctactcgggaggctgaggcaggagaatcgcttgaacctgggaggcggaggttgcggtgaactgagatcgtgccattgcactccagcctgggcaacaagagctaaactccgtctcaaaaaaaaaaaaatgtttttcaaatattagccgggtatggtggtgtcctgtagtcccagctacttgggaggctgagatgggaggatcacttgagcccaggagttcaaggttacagtgagctatgattgtgccactgtattccagcctgggtaacagagggagacccgtttaaaaaaaaaaaagtgatggctaaagtccttccatggctccctattgccctcagtataaagaacacatgtggctgggcgtggtggttcacgcctgtaatcccagcactttgggaggctgaggcgggcggatcacttgaggccaggagtttgagagcaggctggccgacgtggcgaaaccccgtctctattaaaaatacaaaaattagctgggcgtggtggtgcttgcctgtaatcccagatactctggaggctgaggcaggagaatcacttgaacccgggaggcaaaggttgcagtgagctgagattgcgccactgcactccagtctgagtgacaaagcgagactccatctcaaaaaaaaaaaaaataaaagaacacatctttagcatggccttcagtgctcacgggatcttcctgaattaatctccccctcttcatccttgctcactcagctccagccaccctgccccgggacatctgtacttgcctggaacttatttcccttttctccggacagccagccctttctcgtcatttagatctctgctgaaacattaccctgtcaccaaagcactgtctattctatcaccctgttttgtttttgtcaaagctcatattaacatcagttattaattatcttgtttgctcataatttttttttttttttttttggagacagagtctcgttctgttgctcaggctggagtgcagtggcacaatcttggctcactgtaacctccacctcccaggttcaagtgattcttgtgcctcagcctcccaaatagctaggactacaggcacgtcccaccatgcccagctaatttttgtatttttagtggagacggggctttgtcatgttggccaggctgatctcaaattcctgacctcaagtgatctgcccgccttggcctcccaaagtgctgggattacaggcgtgagccaccacacccggcctgctcatgaattttctctttaacttccacatcgaaggcaaagtattgtcttgttaaggctgtgcctccagcacccagcacaggctgggcgcacattcccttgatgaacctgatttgtaatgcctgtcgcctcttccctcgtttcttctaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactgtgagtaactgagcccggagggctggactaggcagacccggtgggagagacgtgcaggggcacctgcagaggcctgggggaatctttgccacttgctcgtagggtcaaggaggggctccttgcagggcaggtggggacatccttggaaagtccctttgtgaatttctttgggtacaattaaagtatttacaggctgggtgcggtggctcatgcctgtaatcccagcactttgagaggctgaggctggcggatcacctgagatcaggagtttaagtttcgccaacatggcgaaaccctgtctctgctgaaaatacaaaaatcagccgggcatggtgtcaagcgcctgtaatcccagctacttggaaggctgaggcaggagaacgcttgaacctgagaggcagagattgcagtgagccgcgatcgtgccagtgcactccagtctggataacagagcaagattccatctcaagaaaaaaaaaatgccatctctctatgcctcactctttgaacatatgacacggtcctgcttcagacactttaataaaagatgcaaattaagccaagtgtggtggcttgtacctataatcccaactactccagaggctgaggcagaaggatggtttgagcccaggagtttgagaccagcctgggcaacagagtgagaccctgtttctttcttttttttttttttttttttgagacggagtctcactctgtcgcccaggctggagtgcagtggtgtgatctcggctcactgcaagctccgcctcccgggttcacgccattctcctgcctcagcctcccgagtagctgggactacaggcgcctgccaccatgcccggctcatttttttgtatttttagtagagacggggtttcactgtgttagccaggatggtctcaatctcctgacctagtggtccgcccgcctcggcctcccaaagtgctgggattacaggtgtaagccactgtgcccatccaagaccctgtttctaccggaaaaaaaaagtaaataatttagctgggcatcgtggtgtgcacctgtaatcccagctgctcctgaggctgtgatgggaggattgctttaacccaggggttcgaatcctaggagttcgaatccatcctaggcaacatagcaaaaccccatttttatttaaaaaaaaaaaaaaagatatgagttaaaactagccctgggatggcatttttcacatattggtaacaaacaaaagaattgatggccgggcgcagtggctcacgcctgtaatcccagcactttgggaggccgaggcgggcggatcacaaggtcaggagatcgagaccatcctggctaacatggtgaaaccccgtctctactaaaactacaaaaaattagccgggcatggtggcaggcgcctgtggtcccagctactcaggaggctgaggcaggagaatggcatgaacccgggaggcagagcttgcagtgagccaagatcgtgccactgcactccagcctgggcgacagagcaagactccatctcaaaaaaaaaaaaaaaaaaaagaattgataacagctgtgctgccaaggctattggaacgtaggaggtcctaggacagtgctgttgggagcataaataagcccaaccctgtggcgggaaattgggcatcagttctcaaaatgtcatgggctgggcacggtggctcacgcctgtaatcccagcactttgggaggctgagggaggcggatcacttgaggtcaggagttcgagaccagcctgaccaccatggagaaaccccgtctctattaaaaatacaaaaaaaactagccaggcatagtggcacatacctgtaatcccagctactcgggaggctgaggcaggagaatctcttgaaactgggaggcagaggttgcggtgagctgagattgcgccactgcactccagcttgggcaacaagagcaaaactccatctcaaaaaaaaaaaaaaaagaaataaaagaaggtatgttgaatatgagtggtatgccaccctcacattagggaagggcagtttcggggaggctgtatttatgtataaaatagccctaaaaggaagtgggagaaatgacaatattagctggctatgagaagagaggctgggaggctgtgggagagggcttgggtgtggagaattctttttgttttttcctttttttgagacagagtttcactcttgttgcccaggctggagtgcaatggcacgatctcagctcaccgcaaccttcacctcctgagtttaagtgattctccggcctcagcctcccgactagctgggattacaggcatgggccactacgcctggcaaattttgtatttttagtagagacagggtttctccatgttggtcaggctggtcttgaactctgacctcaggtgatccgcccgcctcggcctcccaaagtgctgggattacaacgtgagccactttgcctggctgagaattctttttttgttgttgtctttttgagatggagttttgctgtgtccccagcctggagtgcaatggtgtaatctcagctcactgcaacctctccctcccgggttcaagcaattctcctgcctcagcctcccaagtagctggaattacaggcgcccagcaccacgcccggctaatttttgtatttttagtagagacgggatttcatcatgttggccaggctggtcttgatctcctgaccttgtgatctgcccgcctcggcctcccaaagtgctgggattacaggcgtgagccactgcccccagccgagaatttctctttgcgtccttcctactttggggacttcgaatggtgggaaagagttatcaaggccaaaataaggaattcaaatgaaaacaaaacaaaatcaaagaagaaaaaacagaagagcactgggcaggctaggcacgtggctcatgcctataatcccagtgatttagaaggccgaagtagaaggatcgcttggaggccaggagttggacaccagcctgggcaacatagcaagaccccatatctacaaaaaataaaaaacctaaccaagcgtgctggcatactagtagtcccagctactcaggaggctgaggtgggaggatcacctgagcctgggaggtccaggctgtagtgagccgtgatgacaccactgcactccagcctgggtgacagagaaagaccctgtctctaaaaaataaaaactggccaagtagctttgggattagccttgggttccagtcccagcaaggcctttaatagcttgggacatgacttctgcatttactttgcaatcaggtgagacctcctctgatggggaaaatgacacggtgagtgacaaaggatgttctcctatcattgtgtcagggcaaggaagcctctgggtaaatgatcaaatgatcagctttgcttctgatttggagggtgggtgagcagatgctgaccttcccaggtgagggaagtccccgaacattcccagcagcttctggaaaccccagggaaacctctttgaaggtcttttctgcatctctgcctgataggtctttttttttttttttttttttttcttttttttgacacacagtcttgctctgtcgcccaggctggatcactgcaacctccatctcccgggttcaagcagttctcctgcctcatcctctccagtagctgggattacaggcacctggcaccacgcctggctaatttttgtatttttagtagagacagggtttcgccaagttggccaggctggtctcgaactctagacctctggtgatccacccgcctcagcctcccgaagtgctgggattacaggcttgagccaccacgcgcggctctgcctgatagctgagagcatagaactccaggtttgagacctggctctgccacatttctccctctatgactgtgggtgccccactttgccttagtttttacctctgtgaaatggagcagatggctggcacaggtagcaaaggagtaaaagttatgtgggagggtggtacctgagagagactctagcttggtcttgccccacccctggtgtaaacataaagaagcctccctggatggctcaatcttctccaaaaaggttagaggtgtaattcctagaggaggcgaccactagctgggctttgaaggatgtgtaggagttcataaggacaggcattctgggcaggaggaacagcctgggcaaaagttgggagcagggagaaatcttgatggaggcaggaggaggaggaggtaggttggtgtaggccaggtgcagtggctcacacctgtaatcccagtgctttgggaggccaaagcaagaggattgtttgagcccaggagttcgagaccagcctgggcaacatagcgagaccctgtctctaagaaaaaataaaaaaattagggtacagtggcatatgcttgtattctcaactactctggaggctcaagtgggaggatcacttgaacccaggaatttttttgtttttgtttttgtttttttgagatggagtctcactctgtggcccaggctggagtgcagtggtgccatcttggctcactgcagcctcctccacctcctgggttcaaccgattctgctgcctgagcctcccgagttgctgggattaaggtgcccaccatcatgcccagctaatttttttgtatttttagtagagatagggtttaccatgttagccaggctggtcttgaacgcctgaccgcaagagatcctcctgcctcagtttcccaaagtgctgggattataggtgtgagccactgagcctggtcaagcccaggaatttgaggttacagtcagctatgattgcaccactgcattccagcccaggtgacagagagagacactgcccctaaaaaaaaaaaaaaaattgattgatgggaggaagggtgaggttggcagagccttgaatgccaggtggaggagctgggactttccttcttggggtgatagggagtcatggagggtttctgagcaggccagggattagatagctgaaggctggatttactggaagccaatgagcagttggctatggtccttgtccacgcggcccatgttgtgggcagtgaccgtattcaagaagggaaggacagacaagtatttgaatacttcagtgaccaggatttggtaaaggactgcaggtcagggtcaagaagaggtgagagcaggacagacttcctccccgctgcaccaggcagctgagctgggtttcctctaggggctgaggtttgagggtacctcaagttctgcaagagtctataggaggtggtaagagagaagagctggaggtcagagttttcttgactatatatatatatatttttttgtttttgtttttaacagcttaacagctttctgttttatttttagagacagggtctcagggtctcactttgtcacccaggctggagtgcagtggtacaatcgtagctgactgcagcctcaaactcccaggctcaagaaatcctcctcccacccttagcctcctgagtagcagggactacaggtgtgagccagcaggaagcccagctggttttttttttttctttggtgttttttgtttgtttgtttgagaccggagtttcgctcttattgcccaggctgaagtgcaatggcaggatcttagctcaccacaacctccgacccccaggatcaagctattctcttgcctcagccacctgagtagctgggattacaggcatgcgacaccacacaaggctaattttgtatttttagtaaagacagggtttctccatgttggtcaggctggtctcgaactcccaacctcagatgatccacctgcctcggcctcccaaagtgctgagattacaggcatgagccaccgtgcccggcctttttttttttttttttttttttgagacagagtctcactctgtcgcccaggcaggagtgcagtggttcgatctgggctcactgcaagctccgcctcccgggttcactccatcctcctgccttagcctcctgagtagctgggactacaggcgcccaccaccacgcctggctaattttttgtatttttagtagagacggggtttcaccgtgttagccaggatggtcttgatctcctgacctcgtgatccgcccgccttggcctcccaaagtgctgggattacaggcgtgagccaccatgtctggcctggccaggctggtcttgaactcctgacttccggtgatccatctgttctggcctcccaaagtgctgggattacaggcataagccaccacgccatgccgaagcccagcttgtttttaatttttttttttttttttggagaaatgaggtcttgcaatgttgcccaagctagccttgaactcctggcctcaaatgaccccgccttggcatcccaaagtactgggattacagatgtgagccaccatgccccagccttgctttcttgagatacgatttagaataccataagattcatcccttttaagcacataattcaatgacttctgtacaaacaaccatgactacaatctaattttaaaatatttcaatcactctaaaaaagaaacctcctgcttatgtacagcgactctgtctacctcttaagtgaattctcctacctttaatagccctattttacagttcaggaaactgaggttcagagagacaaagtcacttacccacagcaaagaagcaaggctgggtatcaaatgcaggacccccccggtcctgatgctttttttttttttttttttttcctctgagagagactctcactctgtcactcagtctagagtacagtggcgcgatctcagttcactgcaatctctgcctcctgggctgaagtaatcctttcctcacaagtaaacctcagcctctcaagtagctgggactacaggcacacaacaccacgcctggctaatttttgtatttttaggtagagacggggtttcactatattggccaggctggtcttgaactcctgacctcaggtgatccgcctgcctcggccccccaaagtgttgggattacaggcgtgagccaccacacgcagcctttttgttattagactctgtcattactgacttttttttttttttaatagaaacagggtctttctttcccaggctaaagtacagtggcatgatcacagttcactatagccttaaactcctgggctcaagtgatcctcctgcctcagcctcccaagtagcagggactacaggtgtgcaccaccacacccagttaaccattcattcattcattcattcatttattttgagatggagtctcgctctgtcacctaggctggagtgcagtggcacgatctcagctcactgcaacctccacctcccaggttcaagagattctcctgcctcagcctcccgagtagctgagactacaggcgtgcaccaccatgccagactaatttttgtatttttaatagagacggggtttcactctgttggtcaggcttatctcgaactcctgacttcgtggatccaccctccttggcctcccaaagtgctgggattaaaggcgtgagccaccgcgccctgccaacctttttttaatttttcttagagatgggggtctccctatgttgcccaggcttgtcttgaactcctggcctcaagtgaccctcttgccttggcctcacaaagtgctaggattacagcctgagccatcacacctggccaacaggtttttttttttgttttgttttgttttttaaagaatgtctaggccaggctcatttactttcacctgtaatcccagcactttgggaggccggggtgggcagatcacttgaggtcaggaattcgagaccagcctgggcaacatgctgaaaccccgtttctactaaaaatacaaaaattagctgggtgtggtgacacgtgcctgtaatcccagctactcaggaggttgaggcaggagaattgcttgaacccaggaggcagaggttgcagtgagccaagatcatgccatcaccctccagcctgggcgacagaaggagactcagtctaaaaacttaattaattaattaattaaaaataaaaatacaaaaattaacctggtgtggtggtgtgtgcctgtaatctaagctactcaggaggctgaggcaggagaatcccctgaatcccagaggcagaggttgcagtgagccaagatcgagccactgtttgcccagtctagtgcactgggctgctgaatttatttgaccagacacctagcaatagactttgaagttcttttccacttttcactctaagatgctgctgtcatgaataaggaatattttgatccccttcacaaacactcggggccctcttaccagttttcactgaagatcttgacattcctatctgcttaggtgtctgggcgtgtttgggggagatactgaagaggtagggctcccaggcaggtgcagttcgtctgttaggcaggcagcaaggtccacttcaccagacacccccacctctgttttcctgcagggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttggtgaggattgaagaagccagcagggagaaggtgggggtggggtatcctgcaatgcggtgcctgtggccacaggatcttttgagatgggtgtggccccggctaaggggtgcatgtgttctaggcgtatgtgacctaggctgctgagtggccctggaagaggatctcgcaggagggggaatgaaatgccccagagaagggcttcgggacgtccatccctgtctgctcacacctttcttctctccctagtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagtaagaaggggcaggggcggagtggggcttcttgggggtgtgacctgaacccggggcggggctcactgtgtgcctattccaggctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtgtgagtggggctgctggtcaatggcccctatcccccaaggcccaatctccctgaaggtcccataaggtcttgcctccaagtcctgcccccacccacctccatgtcatctcatcgtgtttttccagatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctgtgagtgagccggcgggcagagctgggtgggggcaggggccatggacctaatgcaatcctcaccgcctgttgtatcctccccacagccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 34 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 35 - protein, 8 aa, Homo sapiens
RFYVVMWK

SEQ ID NO: 36 - protein, 4 aa, Artificial Sequence
  linker sequence
GGGS

SEQ ID NO: 37 - protein, 5 aa, Artificial Sequence
  linker sequence
GGGGS

SEQ ID NO: 38 - protein, 18 aa, Artificial Sequence
  signal peptide
MGLAWGLGVLFLMHVCGT

SEQ ID NO: 39 - protein, 247 aa, Homo sapiens; identical to SEQ ID NO: 2

SEQ ID NO: 40 - protein, 135 aa, Homo sapiens; identical to SEQ ID NO: 15

SEQ ID NO: 41 - protein, 555 aa, Homo sapiens; identical to SEQ ID NO: 6

SEQ ID NO: 42 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 43 - protein, 1170 aa, Homo sapiens; identical to SEQ ID NO: 9

SEQ ID NO: 44 - protein, 9 aa, Homo sapiens
NLVPMVATV

SEQ ID NO: 45 - DNA, 20 bp, Homo sapiens
gtcttcagcgtggtgtccaa

SEQ ID NO: 46 - DNA, 20 bp, Homo sapiens
cgcactcgaaggcactctcc